SEQUENCE LISTING (189 sequences)

SEQ ID NO: 1 - protein, 12 aa, synthetic construct
  REGION 1..12: note = IL-23R inhibitor
  BINDING 2..11: note = 1,3-dithio-propan-2-one bridge
  BINDING 4..7: note = 1,3-dithio-propan-2-one bridge
  MOD_RES 10: note = 2-methyl-L-leucine
DCSCWQCWWXCR

SEQ ID NO: 2 - protein, 12 aa, synthetic construct
  REGION 1..12: note = IL-23R inhibitor
  BINDING 2..11: note = 1,3-dithio-propan-2-one bridge
  BINDING 4..7: note = 1,3-dithio-propan-2-one bridge
LCSCWQCWWLCR

SEQ ID NO: 3 - protein, 12 aa, synthetic construct
  REGION 1..12: note = IL-23R inhibitor
  BINDING 2..11: note = 1,3-dithio-propan-2-one bridge
  BINDING 4..7: note = 1,3-dithio-propan-2-one bridge
DCSCWECWWLCR

SEQ ID NO: 4 - protein, 12 aa, synthetic construct
  REGION 1..12: note = IL-23R inhibitor
  BINDING 2..11: note = lactam bridge
  BINDING 4..7: note = 1,3-dithio-propan-2-one bridge
DESCWQCWWLKR

SEQ ID NO: 5 - protein, 12 aa, synthetic construct
  REGION 1..12: note = IL-23R inhibitor
  BINDING 2..11: note = 1,3-dithio-propan-2-one bridge
  BINDING 4..7: note = lactam bridge
DCSEWQKWWLCR

SEQ ID NO: 6 - protein, 10 aa, synthetic construct
  REGION 1..10: note = IL-23R inhibitor
  BINDING 1..10: note = lactam bridge; the lactam bridge is on the terminal -NH2 of the Asp and the terminal -COOH of the Arg
  BINDING 3..6: note = 1,3-dithio-propan-2-one bridge
DSCWQCWWLR

SEQ ID NO: 7 - protein, 12 aa, synthetic construct
  REGION 1..12: note = IL-23R inhibitor
  BINDING 2..11: note = lactam bridge
  BINDING 4..7: note = 1,3-dithio-propan-2-one bridge
DKSCWQCWWLER

SEQ ID NO: 8 - protein, 12 aa, synthetic construct
  REGION 1..12: note = IL-23R inhibitor
  BINDING 2..11: note = lactam bridge
  BINDING 4..7: note = 1,3-dithio-propan-2-one bridge
  MOD_RES 2: note = L-ornithine, also known as 2,5-diaminopentanoic acid
DXSCWQCWWLER

SEQ ID NO: 9 - protein, 12 aa, synthetic construct
  REGION 1..12: note = IL-23R inhibitor
  BINDING 2..11: note = lactam bridge
  BINDING 4..7: note = 1,3-dithio-propan-2-one bridge
  MOD_RES 11: note = (2S)-2,4-diaminobutanoic acid
DESCWQCWWLXR

SEQ ID NO: 10 - protein, 11 aa, synthetic construct
  REGION 1..11: note = IL-23R inhibitor
  BINDING 1..10: note = lactam bridge
  BINDING 3..6: note = 1,3-dithio-propan-2-one bridge
ESCWQCWWLKR

SEQ ID NO: 11 - protein, 11 aa, synthetic construct
  REGION 1..11: note = IL-23R inhibitor
  BINDING 2..11: note = lactam bridge
  BINDING 4..7: note = 1,3-dithio-propan-2-one bridge
DESCWQCWWLK

SEQ ID NO: 12 - protein, 10 aa, synthetic construct
  REGION 1..10: note = IL-23R inhibitor
  BINDING 1..10: note = lactam bridge
  BINDING 3..6: note = 1,3-dithio-propan-2-one bridge
ESCWQCWWLK

SEQ ID NO: 13 - protein, 12 aa, synthetic construct
  REGION 1..12: note = IL-23R inhibitor
  BINDING 2..11: note = lactam bridge
  BINDING 4..7: note = 1,3-dithio-propan-2-one bridge
DESCAQCWWLKR

SEQ ID NO: 14 - protein, 12 aa, synthetic construct
  REGION 1..12: note = IL-23R inhibitor
  BINDING 2..11: note = lactam bridge
  BINDING 4..7: note = 1,3-dithio-propan-2-one bridge
DESCWQCAWLKR

SEQ ID NO: 15 - protein, 12 aa, synthetic construct
  REGION 1..12: note = IL-23R inhibitor
  BINDING 2..11: note = lactam bridge
  BINDING 4..7: note = 1,3-dithio-propan-2-one bridge
DESCWQCWALKR

SEQ ID NO: 16 - protein, 12 aa, synthetic construct
  REGION 1..12: note = IL-23R inhibitor
  BINDING 2..11: note = lactam bridge
  BINDING 4..7: note = 1,3-dithio-propan-2-one bridge
  MOD_RES 5: note = 7-methyl-L-tryptophan
DESCXQCWWLKR

SEQ ID NO: 17 - protein, 12 aa, synthetic construct
  REGION 1..12: note = IL-23R inhibitor
  BINDING 2..11: note = lactam bridge
  BINDING 4..7: note = 1,3-dithio-propan-2-one bridge
  MOD_RES 9: note = 3-(2-naphthyl)-L-alanine
DESCWQCWXLKR

SEQ ID NO: 18 - protein, 11 aa, synthetic construct
  REGION 1..11: note = IL-23R inhibitor
  BINDING 1..10: note = lactam bridge
  BINDING 3..6: note = 1,3-dithio-propan-2-one bridge
  MOD_RES 8: note = 3-(2-naphthyl)-L-alanine
KSCWQCWXLER

SEQ ID NO: 19 - protein, 12 aa, synthetic construct
  REGION 1..12: note = IL-23R inhibitor
  BINDING 2..11: note = lactam bridge
  BINDING 4..7: note = 1,3-dithio-propan-2-one bridge
  MOD_RES 9: note = 3-(2-naphthyl)-L-alanine
DKSCWQCWXLER

SEQ ID NO: 20 - protein, 12 aa, synthetic construct
  REGION 1..12: note = IL-23R inhibitor
  BINDING 2..11: note = lactam bridge
  BINDING 4..7: note = 1,2-phenylenedimethanethiol bridge
  MOD_RES 9: note = 3-(2-naphthyl)-L-alanine
DKSCWQCWXLER

SEQ ID NO: 21 - protein, 12 aa, synthetic construct
  REGION 1..12: note = IL-23R inhibitor
  BINDING 2..11: note = lactam bridge
  BINDING 4..7: note = 1,3-phenylenedimethanethiol bridge
  MOD_RES 9: note = 3-(2-naphthyl)-L-alanine
DKSCWQCWXLER

SEQ ID NO: 22 - protein, 12 aa, synthetic construct
  REGION 1..12: note = IL-23R inhibitor
  BINDING 2..11: note = lactam bridge
  BINDING 4..7: note = 1,4-phenylenedimethanethiol bridge
  MOD_RES 9: note = 3-(2-naphthyl)-L-alanine
DKSCWQCWXLER

SEQ ID NO: 23 - protein, 12 aa, synthetic construct
  REGION 1..12: note = IL-23R inhibitor
  BINDING 2..11: note = lactam bridge
  BINDING 4..7: note = 1,4-disubstituted 1,2,3-triazole bridge
  MOD_RES 4: note = Azido-L-lysine
  MOD_RES 7: note = L-propargylglycine
  MOD_RES 9: note = 3-(2-naphthyl)-L-alanine
DKSXWQXWXLER

SEQ ID NO: 24 - protein, 12 aa, synthetic construct
  REGION 1..12: note = IL-23R inhibitor
  BINDING 2..11: note = lactam bridge
  MOD_RES 4: note = Azidohomo-L-alanine
  BINDING 4..7: note = 1,4-disubstituted 1,2,3-triazole bridge
  MOD_RES 7: note = L-propargylglycine
  MOD_RES 9: note = 3-(2-naphthyl)-L-alanine
DKSXWQXWXLER

SEQ ID NO: 25 - protein, 11 aa, synthetic construct
  REGION 1..11: note = IL-23R inhibitor
  BINDING 1..10: note = lactam bridge
  BINDING 3..6: note = 1,3-dithio-propan-2-one bridge
  MOD_RES 4: note = 3-(2-naphthyl)-L-alanine
  MOD_RES 8: note = 3-(2-naphthyl)-L-alanine
KSCXQCWXLER

SEQ ID NO: 26 - protein, 11 aa, synthetic construct
  REGION 1..11: note = IL-23R inhibitor
  BINDING 1..10: note = lactam bridge
  BINDING 3..6: note = 1,3-dithio-propan-2-one bridge
  MOD_RES 4: note = biphenyl-L-alanine
  MOD_RES 8: note = 3-(2-naphthyl)-L-alanine
KSCXQCWXLER

SEQ ID NO: 27 - protein, 11 aa, synthetic construct
  REGION 1..11: note = IL-23R inhibitor
  BINDING 1..10: note = lactam bridge
  BINDING 3..6: note = 1,3-dithio-propan-2-one bridge
  MOD_RES 4: note = 1-methyl-L-tryptophan
  MOD_RES 8: note = 3-(2-naphthyl)-L-alanine
KSCXQCWXLER

SEQ ID NO: 28 - protein, 12 aa, synthetic construct
  REGION 1..12: note = IL-23R inhibitor
  BINDING 2..11: note = lactam bridge
  BINDING 4..7: note = 1,3-dithio-propan-2-one bridge
  MOD_RES 8: note = 3-(2-naphthyl)-L-alanine
  MOD_RES 9: note = 3-(2-naphthyl)-L-alanine
DKSCWQCXXLER

SEQ ID NO: 29 - protein, 11 aa, synthetic construct
  REGION 1..11: note = IL-23R inhibitor
  BINDING 1..10: note = lactam bridge
  BINDING 3..6: note = 1,3-dithio-propan-2-one bridge
  MOD_RES 7: note = (2S)-2-amino-3-[4-(2-aminoethoxy)phenyl]propanoic acid
  MOD_RES 8: note = 3-(2-naphthyl)-L-alanine
KSCWQCXXLER

SEQ ID NO: 30 - protein, 11 aa, synthetic construct
  REGION 1..11: note = IL-23R inhibitor
  BINDING 1..10: note = lactam bridge
  BINDING 3..6: note = 1,3-dithio-propan-2-one bridge
  MOD_RES 8: note = 3-(2-naphthyl)-L-alanine
KSCWQCHXLER

SEQ ID NO: 31 - protein, 11 aa, synthetic construct
  REGION 1..11: note = IL-23R inhibitor
  BINDING 1..10: note = lactam bridge
  BINDING 3..6: note = 1,3-dithio-propan-2-one bridge
  MOD_RES 8: note = 4-fluoro-L-phenylalanine
KSCWQCWXLER

SEQ ID NO: 32 - protein, 11 aa, synthetic construct
  REGION 1..11: note = IL-23R inhibitor
  BINDING 1..10: note = lactam bridge
  BINDING 3..6: note = 1,3-dithio-propan-2-one bridge
  MOD_RES 8: note = 1-methyl-L-tryptophan
KSCWQCWXLER

SEQ ID NO: 33 - protein, 11 aa, synthetic construct
  REGION 1..11: note = IL-23R inhibitor
  BINDING 1..10: note = 1,3-dithio-propan-2-one bridge
  BINDING 3..6: note = 1,3-dithio-propan-2-one bridge
  MOD_RES 9: note = 2-methyl-L-leucine
CTCWECWWXCS

SEQ ID NO: 34 - protein, 12 aa, synthetic construct
  REGION 1..12: note = IL-23R inhibitor
  BINDING 2..11: note = lactam bridge
  BINDING 4..7: note = 1,3-dithio-propan-2-one bridge
  MOD_RES 10: note = 2-methyl-L-leucine
GKTCWECWWXES

SEQ ID NO: 35 - protein, 12 aa, synthetic construct
  REGION 1..12: note = IL-23R inhibitor
  BINDING 2..11: note = 1,3-dithio-propan-2-one bridge
  BINDING 4..7: note = 1,3-dithio-propan-2-one bridge
  MOD_RES 9: note = 3-(2-naphthyl)-L-alanine
  MOD_RES 10: note = 2-methyl-L-leucine
GCTCWECWXXCR

SEQ ID NO: 36 - protein, 11 aa, synthetic construct
  REGION 1..11: note = IL-23R inhibitor
  BINDING 1..10: note = lactam bridge; the lactam bridge is on the terminal -NH2 of the beta-alanine
  BINDING 3..6: note = 1,3-dithio-propan-2-one bridge
  MOD_RES 1: note = 3-aminopropionic acid, also known as beta-alanine
  MOD_RES 8: note = 3-(2-naphthyl)-L-alanine
XSCWQCWXLER

SEQ ID NO: 37 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 8: note = 3-(2-naphthyl)-L-alanine
XSCWQCWXLER

SEQ ID NO: 38 - protein, 11 aa, synthetic construct
  REGION 1..11: note = IL-23R inhibitor
  MOD_RES 1: note = (3-Aminomethyl)benzoyl
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
XSCWQCXXXER

SEQ ID NO: 39 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
XSCWQCXXLER

SEQ ID NO: 40 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 8: note = 3-(2-naphthyl)-L-alanine
  MOD_RES 9: note = 2-methyl-L-leucine
XSCWQCWXXER

SEQ ID NO: 41 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-arginine
XSCWQCXXXEX

SEQ ID NO: 42 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = 2-methyl-L-arginine
XSCWQCXXXEX

SEQ ID NO: 43 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = N2-methyl-L-arginine
XSCWQCXXXEX

SEQ ID NO: 44 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
XSCWQCXXXES

SEQ ID NO: 45 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = 4-amino-L-phenylalanine
XSCWQCXXXEX

SEQ ID NO: 46 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
  SITE 9: note = D-leucine
XSCWQCXXXEY

SEQ ID NO: 47 - protein, 11 aa, synthetic construct
  REGION 1..11: note = IL-23R inhibitor
  BINDING 1..10: note = lactam bridge
  BINDING 3..6: note = 1,3-dithio-propan-2-one bridge
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
KSCWQCXXXER

SEQ ID NO: 48 - protein, 11 aa, synthetic construct
  REGION 1..11: note = IL-23R inhibitor
  BINDING 1..10: note = lactam bridge
  BINDING 3..6: note = 1,3-dithio-propan-2-one bridge
  MOD_RES 7: note = (2S)-2-amino-3-[4-(2-aminoethoxy)phenyl]propanoic acid
  MOD_RES 8: note = 3-(2-naphthyl)-L-alanine
  SITE 9: note = D-leucine
KSCWQCXXXER

SEQ ID NO: 49 - protein, 11 aa, synthetic construct
  REGION 1..11: note = IL-23R inhibitor
  BINDING 1..10: note = lactam bridge; the lactam bridge is on the terminal -NH2 of the (4-Aminomethyl)benzoyl
  BINDING 3..6: note = 1,3-dithio-propan-2-one bridge
  MOD_RES 1: note = (4-Aminomethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
XSCWQCXXXER

SEQ ID NO: 50 - protein, 11 aa, synthetic construct
  REGION 1..11: note = IL-23R inhibitor
  BINDING 1..10: note = lactam bridge; the lactam bridge is on the terminal -NH2 of the 4-(2-Aminoethyl)benzoyl
  BINDING 3..6: note = 1,3-dithio-propan-2-one bridge
  MOD_RES 1: note = 4-(2-Aminoethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
XSCWQCXXXER

SEQ ID NO: 51 - protein, 11 aa, synthetic construct
  REGION 1..11: note = IL-23R inhibitor
  BINDING 1..10: note = lactam bridge; the lactam bridge is on the terminal -NH2 of the 3-(4-Aminophenyl)propanoyl
  BINDING 3..6: note = 1,3-dithio-propan-2-one bridge
  MOD_RES 1: note = 3-(4-Aminophenyl)propanoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
XSCWQCXXXER

SEQ ID NO: 52 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
XSCWQCXXXER

SEQ ID NO: 53 - protein, 12 aa, synthetic construct
  REGION 1..12: note = IL-23R inhibitor
  BINDING 2..11: note = 1,3-dithio-propan-2-one bridge
  BINDING 4..7: note = disulfide bridge
DCSCWQCWWLCR

SEQ ID NO: 54 - protein, 11 aa, synthetic construct
  REGION 1..11: note = IL-23R inhibitor
  BINDING 1..10: note = lactam bridge
  BINDING 3..6: note = disulfide bridge
  MOD_RES 7: note = (2S)-2-amino-3-[4-(2-aminoethoxy)phenyl]propanoic acid
  MOD_RES 8: note = 3-(2-naphthyl)-L-alanine
KSCWQCXXLER

SEQ ID NO: 55 - protein, 12 aa, synthetic construct
  REGION 1..12: note = IL-23R inhibitor
  BINDING 2..11: note = lactam bridge
  BINDING 4..7: note = 1,3-dithio-propan-2-one bridge
  MOD_RES 10: note = 2-methyl-L-leucine
DEQCWQCYWXKR

SEQ ID NO: 56 - protein, 12 aa, synthetic construct
  REGION 1..12: note = IL-23R inhibitor
  BINDING 2..11: note = lactam bridge
  BINDING 4..7: note = 1,3-dithio-propan-2-one bridge
  MOD_RES 8: note = 3-(2-naphthyl)-L-alanine
  MOD_RES 10: note = 2-methyl-L-leucine
DEQCWQCXWXKR

SEQ ID NO: 57 - protein, 11 aa, synthetic construct
  REGION 1..11: note = IL-23R inhibitor
  BINDING 2..11: note = lactam bridge
  BINDING 4..7: note = 1,3-dithio-propan-2-one bridge
  MOD_RES 9: note = 3-(2-naphthyl)-L-alanine
  MOD_RES 10: note = 2-methyl-L-leucine
DETCWQCWXXK

SEQ ID NO: 58 - protein, 14 aa, synthetic construct
  REGION 1..14: note = IL-23R inhibitor
  BINDING 2..11: note = lactam bridge
  BINDING 4..7: note = 1,3-dithio-propan-2-one bridge
  MOD_RES 9: note = 3-(2-naphthyl)-L-alanine
  MOD_RES 10: note = 2-methyl-L-leucine
DESCWQCWXXKENG

SEQ ID NO: 59 - protein, 13 aa, synthetic construct
  REGION 1..13: note = IL-23R inhibitor
  BINDING 2..11: note = lactam bridge
  BINDING 4..7: note = 1,3-dithio-propan-2-one bridge
  MOD_RES 9: note = 3-(2-naphthyl)-L-alanine
  MOD_RES 10: note = 2-methyl-L-leucine
DESCWQCWXXKNG

SEQ ID NO: 60 - protein, 12 aa, synthetic construct
  REGION 1..12: note = IL-23R inhibitor
  BINDING 2..11: note = lactam bridge
  BINDING 4..7: note = 1,3-dithio-propan-2-one bridge
  MOD_RES 9: note = 3-(2-naphthyl)-L-alanine
  MOD_RES 10: note = 2-methyl-L-leucine
DESCWQCWXXKG

SEQ ID NO: 61 - protein, 14 aa, synthetic construct
  REGION 1..14: note = IL-23R inhibitor
  BINDING 2..11: note = lactam bridge
  BINDING 4..7: note = 1,3-dithio-propan-2-one bridge
  MOD_RES 9: note = 3-(2-naphthyl)-L-alanine
  MOD_RES 10: note = 2-methyl-L-lysine
DESCWQCWXXKENG

SEQ ID NO: 62 - protein, 12 aa, synthetic construct
  REGION 1..12: note = IL-23R inhibitor
  BINDING 2..11: note = 1,3-dithio-propan-2-one bridge
  BINDING 4..7: note = 1,3-dithio-propan-2-one bridge
  MOD_RES 8: note = 3-(2-naphthyl)-L-alanine
  MOD_RES 10: note = 2-methyl-L-leucine
DCQCWQCXWXCR

SEQ ID NO: 63 - protein, 12 aa, synthetic construct
  REGION 1..12: note = IL-23R inhibitor
  BINDING 2..11: note = 1,3-dithio-propan-2-one bridge
  BINDING 4..7: note = 1,3-dithio-propan-2-one bridge
  MOD_RES 10: note = 2-methyl-L-leucine
DCQCWQCYWXCR

SEQ ID NO: 64 - protein, 14 aa, synthetic construct
  REGION 1..14: note = IL-23R inhibitor
  BINDING 2..11: note = 1,3-dithio-propan-2-one bridge
  BINDING 4..7: note = 1,3-dithio-propan-2-one bridge
  MOD_RES 10: note = 2-methyl-L-leucine
DCQCWQCWWXCENG

SEQ ID NO: 65 - protein, 11 aa, synthetic construct
  REGION 1..11: note = IL-23R inhibitor
  MOD_RES 1: note = (3-Aminomethyl)benzoyl
  BINDING 1..10: note = lactam bridge; the lactam bridge is on the terminal -NH2 of the (3-Aminomethyl)benzoyl
  BINDING 3..6: note = lactam bridge
  MOD_RES 3: note = (2S)-2,3-diaminopropanoic acid, also known as 3-amino-L-alanine
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
XSXWQEXXXER

SEQ ID NO: 66 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = lactam bridge
  MOD_RES 1: note = (3-Aminomethyl)benzoyl
  MOD_RES 6: note = (2S)-2,3-diaminopropanoic acid, also known as 3-amino-L-alanine
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
XSEWQXXXXER

SEQ ID NO: 67 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = lactam bridge
  MOD_RES 1: note = (3-Aminomethyl)benzoyl
  MOD_RES 6: note = (2S)-2,4-diaminobutanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
XSEWQXXXXER

SEQ ID NO: 68 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = lactam bridge
  MOD_RES 1: note = (3-Aminomethyl)benzoyl
  MOD_RES 3: note = L-ornithine, also known as 2,5-diaminopentanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
XSXWQEXXXER

SEQ ID NO: 69 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = lactam bridge
  MOD_RES 1: note = (3-Aminomethyl)benzoyl
  MOD_RES 6: note = L-ornithine, also known as 2,5-diaminopentanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
XSEWQXXXXER

SEQ ID NO: 70 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 4: note = 7-methyl-L-tryptophan
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
XSCXQCXXXER

SEQ ID NO: 71 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 4: note = 7-phenyl-L-tryptophan
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
XSCXQCXXXER

SEQ ID NO: 72 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 4: note = 7-(naphth-2-yl)-L-tryptophan
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
XSCXQCXXXER

SEQ ID NO: 73 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 3-(3-quinolinyl)-L-alanine; also known as (2S)-2-amino-3-(3-quinolyl)propanoic acid
  MOD_RES 9: note = 2-methyl-L-leucine
XSCWQCXXXER

SEQ ID NO: 74 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 6-chloro-L-tryptophan
  MOD_RES 9: note = 2-methyl-L-leucine
XSCWQCXXXER

SEQ ID NO: 75 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 4-aminotetrahydro-2H-pyran-4-acetyl
XSCWQCXXXER

SEQ ID NO: 76 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = 3-(3-pyridyl)-L-alanine
XSCWQCXXXEX

SEQ ID NO: 77 - protein, 11 aa, synthetic construct
  REGION 1..11: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = 3-(4-pyridyl)-L-alanine
XSCWQCXXXEX

SEQ ID NO: 78 - protein, 12 aa, synthetic construct
  REGION 1..12: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 12: note = 3-(3-pyridyl)-L-alanine
XSCWQCXXXERX

SEQ ID NO: 79 - protein, 12 aa, synthetic construct
  REGION 1..12: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 12: note = 3-(4-pyridyl)-L-alanine
XSCWQCXXXERX

SEQ ID NO: 80 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-arginine
XSCWQCXXXEX

SEQ ID NO: 81 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
XSCWQCXXXES

SEQ ID NO: 82 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
XSCWQCXXXEX

SEQ ID NO: 83 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-arginine
XTCWQCXXXEX

SEQ ID NO: 84 - protein, 11 aa, synthetic construct
  REGION 1..11: note = IL-23R inhibitor
  BINDING 1..10: note = lactam bridge
  BINDING 3..6: note = 1,3-dithio-propan-2-one bridge
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-arginine
KSCWQCXXXEX

SEQ ID NO: 85 - protein, 11 aa, synthetic construct
  REGION 1..11: note = IL-23R inhibitor
  BINDING 1..10: note = lactam bridge
  BINDING 3..6: note = 1,3-dithio-propan-2-one bridge
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-arginine
KTCWQCXXXEX

SEQ ID NO: 86 - protein, 11 aa, synthetic construct
  REGION 1..11: note = IL-23R inhibitor
  BINDING 1..10: note = lactam bridge
  BINDING 3..6: note = 1,3-dithio-propan-2-one bridge
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
KSCWQCXXXES

SEQ ID NO: 87 - protein, 11 aa, synthetic construct
  REGION 1..11: note = IL-23R inhibitor
  BINDING 1..10: note = lactam bridge; the lactam bridge is on the terminal -NH2 of the 6-Aminohexanoyl
  BINDING 3..6: note = 1,3-dithio-propan-2-one bridge
  MOD_RES 1: note = 6-Aminohexanoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-arginine
XSCWQCXXXEX

SEQ ID NO: 88 - protein, 11 aa, synthetic construct
  REGION 1..11: note = IL-23R inhibitor
  BINDING 1..10: note = lactam bridge; the lactam bridge is on the terminal -NH2 of the 6-Amino-4-oxahexanoyl
  BINDING 3..6: note = 1,3-dithio-propan-2-one bridge
  MOD_RES 1: note = 6-Amino-4-oxahexanoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-arginine
XSCWQCXXXEX

SEQ ID NO: 89 - protein, 11 aa, synthetic construct
  REGION 1..11: note = IL-23R inhibitor
  MOD_RES 1: note = trans-4-Aminomethyl-cyclohexyl-1-carbonyl
  BINDING 1..10: note = lactam bridge; the lactam bridge is on the terminal -NH2 of the trans-4-Aminomethyl-cyclohexyl-1-carbonyl
  BINDING 3..6: note = 1,3-dithio-propan-2-one bridge
  MOD_RES 2: note = L-beta-homoserine
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-arginine
XXCWQCXXXEX

SEQ ID NO: 90 - protein, 11 aa, synthetic construct
  REGION 1..11: note = IL-23R inhibitor
  BINDING 1..10: note = lactam bridge; the lactam bridge is on the terminal -NH2 of the (4-(2-Aminoethyl)-piperazine-1-yl)-acetyl
  BINDING 3..6: note = 1,3-dithio-propan-2-one bridge
  MOD_RES 1: note = (4-(2-Aminoethyl)-piperazine-1-yl)-acetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-arginine
XSCWQCXXXEX

SEQ ID NO: 91 - protein, 11 aa, synthetic construct
  REGION 1..11: note = Sequence 91
  BINDING 1..10: note = lactam bridge; the lactam bridge is on the terminal -NH2 of the (4-(2-Aminoethyl)-piperazine-1-yl)-acetyl
  BINDING 3..6: note = 1,3-dithio-propan-2-one bridge
  MOD_RES 1: note = (4-(2-Aminoethyl)-piperazine-1-yl)-acetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-arginine
XSCWQCXXXDX

SEQ ID NO: 92 - protein, 11 aa, synthetic construct
  REGION 1..11: note = IL-23R inhibitor
  BINDING 1..10: note = lactam bridge; the lactam bridge is on the terminal -NH2 of the 3-Aminomethyl-phenylacetyl
  BINDING 3..6: note = 1,3-dithio-propan-2-one bridge
  MOD_RES 1: note = 3-Aminomethyl-phenylacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-arginine
XSCWQCXXXEX

SEQ ID NO: 93 - protein, 11 aa, synthetic construct
  REGION 1..11: note = IL-23R inhibitor
  BINDING 1..10: note = lactam bridge; the lactam bridge is on the terminal -NH2 of the 2-Aminomethyl-phenylacetyl
  BINDING 3..6: note = 1,3-dithio-propan-2-one bridge
  MOD_RES 1: note = 2-Aminomethyl-phenylacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-arginine
XSCWQCXXXEX

SEQ ID NO: 94 - protein, 11 aa, synthetic construct
  REGION 1..11: note = IL-23R inhibitor
  BINDING 1..10: note = 1,4-disubstituted 1,2,3-triazole bridge
  BINDING 3..6: note = 1,3-dithio-propan-2-one bridge
  MOD_RES 1: note = azido-L-lysine
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 10: note = L-propargylglycine
  SITE 11: note = D-arginine
XSCWQCXXXXX

SEQ ID NO: 95 - protein, 11 aa, synthetic construct
  REGION 1..11: note = IL-23R inhibitor
  BINDING 1..10: note = 1,4-disubstituted 1,2,3-triazole bridge; the 1,4-disubstituted 1,2,3-triazole bridge is on the terminal -NH2 converted to the -N3 of the Azidoacetic acid
  BINDING 3..6: note = 1,3-dithio-propan-2-one bridge
  MOD_RES 1: note = Azidoacetic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 10: note = (2S)-2-amino-5-oxo-5-(prop-2-ynylamino)pentanoic acid
  SITE 11: note = D-arginine
XSCWQCXXXXX

SEQ ID NO: 96 - protein, 11 aa, synthetic construct
  REGION 1..11: note = IL-23R inhibitor
  BINDING 1..10: note = 1,4-disubstituted 1,2,3-triazole bridge; the 1,4-disubstituted 1,2,3-triazole bridge is on the terminal -NH2 converted to the -N3 of the Azidoacetic acid
  BINDING 3..6: note = 1,3-dithio-propan-2-one bridge
  MOD_RES 1: note = Azidoacetic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 10: note = (2S)-2-amino-4-(but-3-ynoylamino)butanoic acid
  SITE 11: note = D-arginine
XSCWQCXXXXX

SEQ ID NO: 97 - protein, 11 aa, synthetic construct
  REGION 1..11: note = IL-23R inhibitor
  BINDING 1..10: note = 1,4-disubstituted 1,2,3-triazole bridge; the 1,4-disubstituted 1,2,3-triazole bridge is on the terminal -NH2 converted to the CCH of the but-3-ynoic acid
  BINDING 3..6: note = 1,3-dithio-propan-2-one bridge
  MOD_RES 1: note = but-3-ynoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 10: note = (2S)-2-amino-4-[(2-azidoacetyl)amino]butanoic acid
  SITE 11: note = D-arginine
XSCWQCXXXXX

SEQ ID NO: 98 - protein, 11 aa, synthetic construct
  REGION 1..11: note = IL-23R inhibitor
  BINDING 1..10: note = 1,4-disubstituted 1,2,3-triazole bridge; the 1,4-disubstituted 1,2,3-triazole bridge is on the terminal -NH2 converted to the -N3 of the (2S)-2-azidopropanoic acid
  BINDING 3..6: note = 1,3-dithio-propan-2-one bridge
  MOD_RES 1: note = (2S)-2-azidopropanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 10: note = (2S)-2-amino-5-oxo-5-(prop-2-ynylamino)pentanoic acid
  SITE 11: note = D-arginine
XSCWQCXXXXX

SEQ ID NO: 99 - protein, 11 aa, synthetic construct
  REGION 1..11: note = IL-23R inhibitor
  BINDING 1..10: note = 1,4-disubstituted 1,2,3-triazole bridge; the 1,4-disubstituted 1,2,3-triazole bridge is on the terminal -NH2 converted to the -N3 of the (2S)-2-azidopropanoic acid
  BINDING 3..6: note = 1,3-dithio-propan-2-one bridge
  MOD_RES 1: note = (2S)-2-azidopropanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 10: note = (2S)-2-amino-4-(but-3-ynoylamino)butanoic acid
  SITE 11: note = D-arginine
XSCWQCXXXXX

SEQ ID NO: 100 - protein, 11 aa, synthetic construct
  REGION 1..11: note = IL-23R inhibitor
  BINDING 1..10: note = 1,4-disubstituted 1,2,3-triazole bridge; the 1,4-disubstituted 1,2,3-triazole bridge is on the terminal -NH2 converted to the CCH of the but-3-ynoic acid
  BINDING 3..6: note = 1,3-dithio-propan-2-one bridge
  MOD_RES 1: note = but-3-ynoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 10: note = (2S)-2-amino-4-[[(2S)-2-azidopropanoyl]amino]butano ic acid
  SITE 11: note = D-arginine
XSCWQCXXXXX

SEQ ID NO: 101 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-2-pyridineacetyl
  BINDING 3..6: note = 1,3-dithio-propan-2-one bridge
  MOD_RES 1: note = 4-Aminomethyl-2-pyridineacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-arginine
XSCWQCXXXEX

SEQ ID NO: 102 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-3-pyridineacetyl
  BINDING 3..6: note = 1,3-dithio-propan-2-one bridge
  MOD_RES 1: note = 4-Aminomethyl-3-pyridineacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-arginine
XSCWQCXXXEX

SEQ ID NO: 103 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-2-fluoro-phenylacetyl
  BINDING 3..6: note = 1,3-dithio-propan-2-one bridge
  MOD_RES 1: note = 4-Aminomethyl-2-fluoro-phenylacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-arginine
XSCWQCXXXEX

SEQ ID NO: 104 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-3-methyl-phenylacetyl
  BINDING 3..6: note = 1,3-dithio-propan-2-one bridge
  MOD_RES 1: note = 4-Aminomethyl-3-methyl-phenylacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-arginine
XSCWQCXXXEX

SEQ ID NO: 105 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-3-methoxy-phenylacetyl
  BINDING 3..6: note = 1,3-dithio-propan-2-one bridge
  MOD_RES 1: note = 4-Aminomethyl-3-methoxy-phenylacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-arginine
XSCWQCXXXEX

SEQ ID NO: 106 - protein, 11 aa, synthetic construct
  REGION 1..11: note = IL-23R inhibitor
  BINDING 1..10: note = 1,5-disubstituted 1,2,3-triazole bridge; the 1,5-disubstituted 1,2,3-triazole bridge is on the terminal -NH2 converted to the CCH of the but-3-ynoic acid
  BINDING 3..6: note = 1,3-dithio-propan-2-one bridge
  MOD_RES 1: note = but-3-ynoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 10: note = (2S)-2-amino-4-[(2-azidoacetyl)amino]butanoic acid
  SITE 11: note = D-arginine
XSCWQCXXXXX

SEQ ID NO: 107 - protein, 11 aa, synthetic construct
  REGION 1..11: note = IL-23R inhibitor
  BINDING 1..10: note = 1,5-disubstituted 1,2,3-triazole bridge; the 1,5-disubstituted 1,2,3-triazole bridge is on the terminal -NH2 converted to the CCH of the but-3-ynoic acid
  BINDING 3..6: note = 1,3-dithio-propan-2-one bridge
  MOD_RES 1: note = but-3-ynoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 10: note = (2S)-2-amino-4-[[(2S)-2-azidopropanoyl]amino]butano ic acid
  SITE 11: note = D-arginine
XSCWQCXXXXX

SEQ ID NO: 108 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-arginine
XSCWQCXXXEX

SEQ ID NO: 109 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = D-2,4-Diaminobutanoyl([2-(trimethyl-2-aminoethoxy)e thoxy]propyl)
XSCWQCXXXEX

SEQ ID NO: 110 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 7: note = (2S)-2,4-diaminobutanoic acid
  MOD_RES 8: note = 3-(2-naphthyl)-L-alanine
  MOD_RES 9: note = 2-methyl-L-leucine
  SITE 11: note = D-arginine
XSCWQCXXXEX

SEQ ID NO: 111 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 7: note = 2,4-diaminobutanoyl([2-(trimethyl-2-aminoethoxy)eth oxy]propyl)
  MOD_RES 8: note = 3-(2-naphthyl)-L-alanine
  MOD_RES 9: note = 2-methyl-L-leucine
  SITE 11: note = D-arginine
XSCWQCXXXEX

SEQ ID NO: 112 - protein, 11 aa, synthetic construct
  REGION 1..11: note = IL-23R inhibitor
  BINDING 1..10: note = lactam bridge; the lactam bridge is on the terminal -NH2 of the (2S)-2,4-diaminobutanoic acid
  BINDING 3..6: note = 1,3-dithio-propan-2-one bridge
  MOD_RES 1: note = (2S)-2,4-diaminobutanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-arginine
XSCWQCXXXEX

SEQ ID NO: 113 - protein, 11 aa, synthetic construct
  REGION 1..11: note = IL-23R inhibitor
  BINDING 1..10: note = lactam bridge; the lactam bridge is on the terminal -NH2 of the 2,4-Diaminobutanoyl([2-(trimethyl-2-aminoethox y)ethoxy]propyl
  BINDING 3..6: note = 1,3-dithio-propan-2-one bridge
  MOD_RES 1: note = 2,4-diaminobutanoyl([2-(trimethyl-2-aminoethoxy)eth oxy]propyl)
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-arginine
XSCWQCXXXEX

SEQ ID NO: 114 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 7: note = 2-[2-[3-[4-[(2S)-2-amino-2-carboxy-ethyl]anilino]-3 -oxo-propoxy]ethoxy]ethyl-trimethyl-ammonium
  MOD_RES 8: note = 3-(2-naphthyl)-L-alanine
  MOD_RES 9: note = 2-methyl-L-leucine
  SITE 11: note = D-arginine
XSCWQCXXXEX

SEQ ID NO: 115 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-glutamine
XSCWQCXXXEX

SEQ ID NO: 116 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-glutamic acid
XSCWQCXXXEX

SEQ ID NO: 117 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  SITE 11: note = D-histidine
XSCWQCXXXEX

SEQ ID NO: 118 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = 3-Aminopropanoyl
XSCWQCXXXEX

SEQ ID NO: 119 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = 3-Aminopropanoyl
XSCWQCXXXEX

SEQ ID NO: 120 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
  MOD_RES 12: note = 3-(3-pyridyl)-L-alanine
XSCWQCXXXEXX

SEQ ID NO: 121 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 7: note = (2S)-2-amino-3-[4-(2-aminoethoxy)phenyl]propanoic acid
  MOD_RES 8: note = 6-fluoro-L-tryptophan
  MOD_RES 9: note = 2-methyl-L-leucine
  MOD_RES 11: note = (2S)-2,4-diaminobutanoic acid
  MOD_RES 12: note = 3-(3-pyridyl)-L-alanine
XSCWQCXXXEXX

SEQ ID NO: 122 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
  MOD_RES 12: note = 3-(3-pyridyl)-L-alanine
XSEWQXXXXEXX

SEQ ID NO: 123 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 3: note = (2S)-2,4-diaminobutanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
  MOD_RES 12: note = 3-(3-pyridyl)-L-alanine
XSXWQEXXXEXX

SEQ ID NO: 124 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = L-ornithine, also known as 2,5-diaminopentanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
  MOD_RES 12: note = 3-(3-pyridyl)-L-alanine
XSEWQXXXXEXX

SEQ ID NO: 125 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
XSEWQXXXXEX

SEQ ID NO: 126 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
XVEWQXXXXEX

SEQ ID NO: 127 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
XIEWQXXXXEX

SEQ ID NO: 128 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
XSEWVXXXXEX

SEQ ID NO: 129 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
XSEWHXXXXEX

SEQ ID NO: 130 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
XSEWQXFXXEX

SEQ ID NO: 131 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
XSEWQXVXXEX

SEQ ID NO: 132 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = 4-methyl-L-phenylalanine
  MOD_RES 8: note = 3-(2-naphthyl)-L-alanine
  MOD_RES 9: note = 2-methyl-L-leucine
  MOD_RES 11: note = (2S)-2,4-diaminobutanoic acid
XSEWQXXXXEX

SEQ ID NO: 133 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = (2S)-2-amino-3-[4-(2-aminoethoxy)phenyl]propanoic acid
  MOD_RES 9: note = 2-methyl-L-leucine
  MOD_RES 11: note = (2S)-2,4-diaminobutanoic acid
XSEWQXXHXEX

SEQ ID NO: 134 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = (2S)-2-amino-3-[4-(2-aminoethoxy)phenyl]propanoic acid
  MOD_RES 8: note = 3-fluoro-L-phenylalanine
  MOD_RES 9: note = 2-methyl-L-leucine
  MOD_RES 11: note = (2S)-2,4-diaminobutanoic acid
XSEWQXXXXEX

SEQ ID NO: 135 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = (2S)-2-amino-3-[4-(2-aminoethoxy)phenyl]propanoic acid
  MOD_RES 8: note = 3,4-dimethyl-L-phenylalanine
  MOD_RES 9: note = 2-methyl-L-leucine
  MOD_RES 11: note = (2S)-2,4-diaminobutanoic acid
XSEWQXXXXEX

SEQ ID NO: 136 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-valine
  MOD_RES 11: note = (2S)-2,4-diaminobutanoic acid
XSEWQXXXXEX

SEQ ID NO: 137 - protein, 10 aa, synthetic construct
  REGION 1..10: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
XSEWQXXXXE

SEQ ID NO: 138 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 4: note = 4-fluoro-L-tryptophan
  MOD_RES 6: note = (2S)-2,4-diaminobutanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
XSEXQXXXXEX

SEQ ID NO: 139 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 4: note = 7-fluoro-L-tryptophan
  MOD_RES 6: note = (2S)-2,4-diaminobutanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
XSEXQXXXXEX

SEQ ID NO: 140 - protein, 11 aa, synthetic construct
  REGION 1..11: note = IL-23R inhibitor
  BINDING 1..10: note = lactam bridge; the lactam bridge is on the terminal -NH2 of the 4-Methylaminomethyl-phenylacetyl
  BINDING 3..6: note = 1,3-dithio-propan-2-one bridge
  MOD_RES 1: note = 4-Methylaminomethyl-phenylacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
XSCWQCXXXEX

SEQ ID NO: 141 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 2: note = N-methyl-L-serine
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
XXCWQCXXXEX

SEQ ID NO: 142 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 3: note = N-methyl-L-cysteine
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
XSXWQCXXXEX

SEQ ID NO: 143 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 4: note = N-alpha-methyl-L-tryptophan; also known as L-abrine
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
XSCXQCXXXEX

SEQ ID NO: 144 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 5: note = N2-methyl-L-glutamine
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
XSCWXCXXXEX

SEQ ID NO: 145 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 2: note = (2S)-2-amino-5-oxo-5-pyrrolidin-1-yl-pentanoic acid
  MOD_RES 6: note = (2S)-2,4-diaminobutanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
XXEWQXXXXEX

SEQ ID NO: 146 - protein, 11 aa, synthetic construct
  REGION 1..11: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 5: note = (2S)-2-amino-5-oxo-5-pyrrolidin-1-yl-pentanoic acid
  MOD_RES 6: note = (2S)-2,4-diaminobutanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
XSEWXXXXXEX

SEQ ID NO: 147 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = 1,3-dithio-propan-2-one bridge
  MOD_RES 1: note = 4-Aminomethyl-phenylacetyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 12: note = 3-(3-pyridyl)-L-alanine
XSCWQCXXXERX

SEQ ID NO: 148 - protein, 12 aa, synthetic construct
  REGION 1..12: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
  MOD_RES 12: note = 3-(3-pyridyl)-L-alanine
XSCWQCXXXEXX

SEQ ID NO: 149 - protein, 12 aa, synthetic construct
  REGION 1..12: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 12: note = 3-(3-quinolinyl)-L-alanine; also known as (2S)-2-amino-3-(3-quinolyl)propanoic acid
XSCWQCXXXERX

SEQ ID NO: 150 - protein, 12 aa, synthetic construct
  REGION 1..12: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 12: note = 3-(3-pyridyl)-D-alanine
XSCWQCXXXERX

SEQ ID NO: 151 - protein, 12 aa, synthetic construct
  REGION 1..12: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 12: note = 3-Amino-3-(3'-pyridyl)propionyl
XSCWQCXXXERX

SEQ ID NO: 152 - protein, 12 aa, synthetic construct
  REGION 1..12: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 12: note = 3-fluoro-L-phenylalanine
XSCWQCXXXERX

SEQ ID NO: 153 - protein, 12 aa, synthetic construct
  REGION 1..12: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 12: note = 3,5-difluoro-L-phenylalanine
XSCWQCXXXERX

SEQ ID NO: 154 - protein, 12 aa, synthetic construct
  REGION 1..12: note = IL-23R inhibitor
  BINDING 1..10: note = lactam bridge; the lactam bridge is on the terminal -NH2 of the (3-Aminomethyl)benzoyl
  BINDING 3..6: note = 1,3-dithio-propan-2-one bridge
  MOD_RES 1: note = (3-Aminomethyl)benzoyl
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 12: note = 4-Aminomethyl-2-pyridineacetyl
XSCWQCXXXERX

SEQ ID NO: 155 - protein, 12 aa, synthetic construct
  REGION 1..12: note = IL-23R inhibitor
  BINDING 1..10: note = lactam bridge
  BINDING 3..6: note = 1,3-dithio-propan-2-one bridge
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-leucine
  MOD_RES 11: note = (2S)-2,4-diaminobutanoic acid
  MOD_RES 12: note = 3-(3-pyridyl)-L-alanine
KTCWQCXXXEXX

SEQ ID NO: 156 - protein, 12 aa, synthetic construct
  REGION 1..12: note = IL-23R inhibitor
  BINDING 1..10: note = lactam bridge; the lactam bridge is on the terminal -NH2 of the (3-Aminomethyl)benzoyl
  BINDING 3..6: note = lactam bridge
  MOD_RES 1: note = (3-Aminomethyl)benzoyl
  MOD_RES 6: note = (2S)-2,4-diaminobutanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-valine
  MOD_RES 11: note = (2S)-2,4-diaminobutanoic acid
  MOD_RES 12: note = 3-(3-pyridyl)-L-alanine
XIEWQXXXXEXX

SEQ ID NO: 157 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = (2S)-2-amino-3-[4-(2-aminoethoxy)phenyl]propanoic acid
  MOD_RES 8: note = 3-(2-naphthyl)-L-alanine
  MOD_RES 9: note = 2-methyl-L-valine
  MOD_RES 11: note = (2S)-2,4-diaminobutanoic acid
  MOD_RES 12: note = 3-(3-pyridyl)-L-alanine
XIEWQXXXXEXX

SEQ ID NO: 158 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = (2S)-2-amino-3-[4-(2-aminoethoxy)phenyl]propanoic acid
  MOD_RES 8: note = 3,4-dimethyl-L-phenylalanine
  MOD_RES 9: note = 2-methyl-L-valine
  MOD_RES 12: note = 3-(3-pyridyl)-L-alanine
XIEWQXXXXEGX

SEQ ID NO: 159 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = 4-methyl-L-phenylalanine
  MOD_RES 8: note = 3,4-dimethyl-L-phenylalanine
  MOD_RES 9: note = 2-methyl-L-valine
  MOD_RES 12: note = 3-(3-pyridyl)-L-alanine
XIEWQXXXXEGX

SEQ ID NO: 160 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = 4-methyl-L-phenylalanine
  MOD_RES 8: note = 3,4-dimethyl-L-phenylalanine
  MOD_RES 9: note = 2-methyl-L-valine
  MOD_RES 11: note = gamma aminobutyric acid; also known as 4-aminobutanoic acid
  MOD_RES 12: note = 3-(3-pyridyl)-L-alanine
XIEWQXXXXEXX

SEQ ID NO: 161 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = 4-methyl-L-phenylalanine
  MOD_RES 8: note = 3,4-dimethyl-L-phenylalanine
  MOD_RES 9: note = 2-methyl-L-valine
  MOD_RES 12: note = 2,3-Diaminopropanoyl(3-pyridylacetyl)
XIEWQXXXXEGX

SEQ ID NO: 162 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = 4-methyl-L-phenylalanine
  MOD_RES 8: note = 3,4-dimethyl-L-phenylalanine
  MOD_RES 9: note = 2-methyl-L-valine
  MOD_RES 12: note = 2,3-Diaminopropanoyl(3-pyridylpropionyl)
XIEWQXXXXEGX

SEQ ID NO: 163 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = 4-methyl-L-phenylalanine
  MOD_RES 8: note = 3,4-dimethyl-L-phenylalanine
  MOD_RES 9: note = 2-methyl-L-valine
  MOD_RES 12: note = 2,3-Diaminopropanoyl(3-fluorobenzoyl)
XIEWQXXXXEGX

SEQ ID NO: 164 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = 4-methyl-L-phenylalanine
  MOD_RES 8: note = 3,4-dimethyl-L-phenylalanine
  MOD_RES 9: note = 2-methyl-L-valine
  MOD_RES 12: note = 2,3-Diaminopropanoyl(3-fluorophenylacetyl)
XIEWQXXXXEGX

SEQ ID NO: 165 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = (2S)-2-amino-3-[4-(2-aminoethoxy)phenyl]propanoic acid
  MOD_RES 8: note = 3,4-dimethyl-L-phenylalanine
  MOD_RES 9: note = 2-methyl-L-valine
  MOD_RES 11: note = (2S)-2,4-diaminobutanoic acid
  MOD_RES 12: note = (2S)-2-amino-2-methyl-3-(3-pyridyl)propanoic acid
XIEWQXXXXEXX

SEQ ID NO: 166 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = (2S)-2-amino-3-(4-methoxyphenyl)propanoic acid
  MOD_RES 8: note = 3,4-dimethyl-L-phenylalanine
  MOD_RES 9: note = 2-methyl-L-valine
  MOD_RES 11: note = (2S)-2,4-diaminobutanoic acid
  MOD_RES 12: note = (2S)-2-amino-2-methyl-3-(3-pyridyl)propanoic acid
XIEWQXXXXEXX

SEQ ID NO: 167 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = alpha-methyl-L-phenylalanine; also known as (2S)-2-amino-2-methyl-3-phenyl-propanoic acid
  MOD_RES 8: note = 3,4-dimethyl-L-phenylalanine
  MOD_RES 9: note = 2-methyl-L-valine
  MOD_RES 11: note = (2S)-2,4-diaminobutanoic acid
  MOD_RES 12: note = (2S)-2-amino-2-methyl-3-(3-pyridyl)propanoic acid
XIEWQXXXXEXX

SEQ ID NO: 168 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = biphenyl-L-alanine
  MOD_RES 8: note = 3,4-dimethyl-L-phenylalanine
  MOD_RES 9: note = 2-methyl-L-valine
  MOD_RES 11: note = (2S)-2,4-diaminobutanoic acid
  MOD_RES 12: note = (2S)-2-amino-2-methyl-3-(3-pyridyl)propanoic acid
XIEWQXXXXEXX

SEQ ID NO: 169 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = (2S)-2-amino-3-(4-fluorophenyl)-2-methyl-propanoic acid
  MOD_RES 8: note = 3,4-dimethyl-L-phenylalanine
  MOD_RES 9: note = 2-methyl-L-valine
  MOD_RES 11: note = (2S)-2,4-diaminobutanoic acid
  MOD_RES 12: note = (2S)-2-amino-2-methyl-3-(3-pyridyl)propanoic acid
XIEWQXXXXEXX

SEQ ID NO: 170 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = 4-fluoro-D-phenylalanine
  MOD_RES 8: note = 3,4-dimethyl-L-phenylalanine
  MOD_RES 9: note = 2-methyl-L-valine
  MOD_RES 11: note = (2S)-2,4-diaminobutanoic acid
  MOD_RES 12: note = (2S)-2-amino-2-methyl-3-(3-pyridyl)propanoic acid
XIEWQXXXXEXX

SEQ ID NO: 171 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = 4-chloro-L-phenylalanine
  MOD_RES 8: note = 3,4-dimethyl-L-phenylalanine
  MOD_RES 9: note = 2-methyl-L-valine
  MOD_RES 11: note = (2S)-2,4-diaminobutanoic acid
  MOD_RES 12: note = (2S)-2-amino-2-methyl-3-(3-pyridyl)propanoic acid
XIEWQXXXXEXX

SEQ ID NO: 172 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = Alpha-methyl-L-tryptophan
  MOD_RES 8: note = 3,4-dimethyl-L-phenylalanine
  MOD_RES 9: note = 2-methyl-L-valine
  MOD_RES 11: note = (2S)-2,4-diaminobutanoic acid
  MOD_RES 12: note = (2S)-2-amino-2-methyl-3-(3-pyridyl)propanoic acid
XIEWQXXXXEXX

SEQ ID NO: 173 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = 3,3-diphenyl-L-alanine; also known as beta-phenyl-L-phenylalanine, or (S)-2-amino-3,3-diphenylpropionic acid
  MOD_RES 8: note = 3,4-dimethyl-L-phenylalanine
  MOD_RES 9: note = 2-methyl-L-valine
  MOD_RES 11: note = (2S)-2,4-diaminobutanoic acid
  MOD_RES 12: note = (2S)-2-amino-2-methyl-3-(3-pyridyl)propanoic acid
XIEWQXXXXEXX

SEQ ID NO: 174 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = L-2-phenylglycine
  MOD_RES 8: note = 3,4-dimethyl-L-phenylalanine
  MOD_RES 9: note = 2-methyl-L-valine
  MOD_RES 11: note = (2S)-2,4-diaminobutanoic acid
  MOD_RES 12: note = (2S)-2-amino-2-methyl-3-(3-pyridyl)propanoic acid
XIEWQXXXXEXX

SEQ ID NO: 175 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = 4-methyl-L-phenylalanine
  MOD_RES 8: note = 1-methyl-L-tryptophan
  MOD_RES 9: note = 2-methyl-L-valine
  MOD_RES 11: note = (2S)-2,4-diaminobutanoic acid
  MOD_RES 12: note = (2S)-2-amino-2-methyl-3-(3-pyridyl)propanoic acid
XIEWQXXXXEXX

SEQ ID NO: 176 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = 4-methyl-L-phenylalanine
  MOD_RES 8: note = biphenyl-L-alanine
  MOD_RES 9: note = 2-methyl-L-valine
  MOD_RES 11: note = (2S)-2,4-diaminobutanoic acid
  MOD_RES 12: note = (2S)-2-amino-2-methyl-3-(3-pyridyl)propanoic acid
XIEWQXXXXEXX

SEQ ID NO: 177 - protein, 12 aa, synthetic construct
  REGION 1..12: note = IL-23R inhibitor
  BINDING 1..10: note = lactam bridge; the lactam bridge is on the terminal -NH2 of the 4-Aminomethyl-phenylacetyl
  BINDING 3..6: note = lactam bridge
  MOD_RES 1: note = 4-Aminomethyl-phenylacetyl
  MOD_RES 6: note = (2S)-2,4-diaminobutanoic acid
  MOD_RES 7: note = 4-methyl-L-phenylalanine
  MOD_RES 8: note = 6-fluoro-D-tryptophan
  MOD_RES 9: note = 2-methyl-L-valine
  MOD_RES 11: note = (2S)-2,4-diaminobutanoic acid
  MOD_RES 12: note = (2S)-2-amino-2-methyl-3-(3-pyridyl)propanoic acid
XIEWQXXXXEXX

SEQ ID NO: 178 - protein, 12 aa, synthetic construct
  REGION 1..12: note = IL-23R inhibitor
  BINDING 2..11: note = 1,3-dithio-propan-2-one bridge
  BINDING 4..7: note = 1,3-dithio-propan-2-one bridge
GCTCWECWWLCS

SEQ ID NO: 179 - protein, 12 aa, synthetic construct
  REGION 1..12: note = IL-23R inhibitor
  BINDING 2..11: note = 1,3-dithio-propan-2-one bridge
  BINDING 4..7: note = 1,3-dithio-propan-2-one bridge
DCSCWQCWWLCR

SEQ ID NO: 180 - protein, 13 aa, synthetic construct
  REGION 1..13: note = IL-23R inhibitor
  BINDING 2..12: note = 1,3-dithio-propan-2-one bridge
  BINDING 5..10: note = 1,3-dithio-propan-2-one bridge
ECWLCYYRNCNCM

SEQ ID NO: 181 - protein, 12 aa, synthetic construct
  REGION 1..12: note = IL-23R inhibitor
  BINDING 2..11: note = 1,3-dithio-propan-2-one bridge
  BINDING 4..7: note = 1,3-dithio-propan-2-one bridge
  MOD_RES 10: note = 2-methyl-L-leucine
GCTCWECWWXCS

SEQ ID NO: 182 - protein, 12 aa, synthetic construct
  REGION 1..12: note = IL-23R inhibitor
  BINDING 1..6: note = thioether bridge
  MOD_RES 1: note = (2S)-2-aminobutyric acid
  MOD_RES 7: note = (2S)-2-amino-3-[4-(2-aminoethoxy)phenyl]propanoic acid
  MOD_RES 9: note = 2-methyl-L-lysine
XQTWQCXWXENG

SEQ ID NO: 183 - protein, 12 aa, synthetic construct
  REGION 1..12: note = IL-23R inhibitor
  BINDING 2..11: note = disulfide bridge
  BINDING 4..7: note = disulfide bridge
DCSCWQCWWLCR

SEQ ID NO: 184 - protein, 12 aa, synthetic construct
  REGION 1..12: note = IL-23R inhibitor
  BINDING 2..11: note = lactam bridge
DESSWQSWWLKR

SEQ ID NO: 185 - protein, 12 aa, synthetic construct
  REGION 1..12: note = IL-23R inhibitor
  BINDING 2..11: note = lactam bridge
DESTWQYWWLKR

SEQ ID NO: 186 - protein, 12 aa, synthetic construct
  REGION 1..12: note = IL-23R inhibitor
  BINDING 2..11: note = lactam bridge
DESMWQMWWLKR

SEQ ID NO: 187 - protein, 11 aa, synthetic construct
  REGION 1..11: note = IL-23R inhibitor
  BINDING 2..10: note = lactam bridge
  BINDING 3..6: note = 1,3-dithio-propan-2-one bridge
DECWQCWWLKR

SEQ ID NO: 188 - protein, 11 aa, synthetic construct
  REGION 1..11: note = IL-23R inhibitor
  BINDING 2..10: note = lactam bridge
  BINDING 4..6: note = 1,3-dithio-propan-2-one bridge
DESCWCWWLKR

SEQ ID NO: 189 - protein, 11 aa, synthetic construct
  REGION 1..11: note = IL-23R inhibitor
  BINDING 2..10: note = lactam bridge
  BINDING 4..7: note = 1,3-dithio-propan-2-one bridge
DESCWQCWWKR

(19) United States
(12) Patent Application Publication
(10) Pub. No.: US 2025/0154198 A1
(43) Pub. Date: May 15, 2025

(54) PEPTIDE INHIBITORS OF INTERLEUKIN-23 RECEPTOR

(71) Applicant: Zealand Pharma A/S, Søborg (DK)

(72) Inventors: Henrik Fischer MUNCH, Søborg (DK); Niels Bjerre HOLM, Søborg (DK); Rasmus LETH, Søborg (DK); Bjarne Due LARSEN, Roskilde (DK); Jesper Mosolff MATHIESEN, Farum (DK); Octav CALDARARU, Søborg (DK)

(21) Appl. No.: 18/715,410

(22) PCT Filed: Dec. 1, 2022
(86) PCT No.: PCT/EP2022/084077

§ 371 Date: May 31, 2024

(30) Foreign Application Priority Data
Dec. 1, 2021 (EP) 21211815.2

(51) Int. Cl. C07K 7/08; A61K 38/00; A61P 1/04; C07K 7/06; C07K 7/56

(52) U.S. Cl. CPC C07K 7/08; A61P 1/04; C07K 7/06; C07K 7/56; A61K 38/00

(57) ABSTRACT

The present invention relates to compounds that are peptide inhibitors of interleukin-23 receptor (IL-23R) and to their use in the treatment or prevention of a variety of diseases, conditions or disorders, including inflammatory diseases, such as inflammatory bowel disease, such as Crohn's disease, psoriasis, and ulcerative colitis. The compounds have good physical and chemical stability in the gastrointestinal tract.

### FIELD OF THE INVENTION

The present invention relates to peptide inhibitors of interleukin-23 receptor (IL-23R), and to their medical use in the treatment and/or prevention of a variety of diseases, conditions or disorders, including inflammatory bowel disease, such as Crohn's disease, psoriasis, and ulcerative colitis, and other conditions and disorders described herein.

### BACKGROUND OF THE INVENTION

Interleukin-23 (IL-23) is a heterodimeric cytokine composed of a unique p19 subunit and the p40 subunit of interleukin-12 (IL-12). IL-12 is a cytokine involved in the development of interferon-gamma (IFN-γ)-producing T helper 1 (Th1) cells. Although both IL-23 and IL-12 contain the p40 subunit, they have different phenotypic properties. Animals deficient in IL-12 are susceptible to inflammatory autoimmune diseases, whereas IL-23 deficient animals are resistant. This is thought to be due to a reduced number of CD4⁺ T cells producing interleukin-6 (IL-6), interleukin-17 (IL-17), and tumor necrosis factor (TNF) in the central nervous system (CNS) of IL-23-deficient animals. Furthermore, in contrast to IL-12 which acts mainly on naive CD4⁺ T cells, IL-23 preferentially acts on memory CD4⁺ T cells.

The receptor that binds IL-23 is the interleukin-23 receptor (IL-23R). IL-23R is a heterodimeric receptor composed of IL-12Rβ1 and IL-23R subunits. Binding of IL-23 to IL-23R activates the JAK-STAT signalling pathway: activating the Janus kinase (JAK) molecules JAK2 and tyrosine kinase 2 (TYK2), as well as the signal transducer and activator of transcription proteins (STATs) STAT1, STAT3, STAT4, and STAT5. STAT4 activation is substantially weaker and different DNA-binding STAT complexes form in response to IL-23 as compared with IL-12. IL-23R associates constitutively with JAK2 and in a ligand-dependent manner with STAT3.

IL-23R is expressed on various adaptive and innate immune cells, including: T-helper 17 (Th17) cells, gamma-delta (γδ) T cells, natural killer (NK) cells, dendritic cells, macrophages, and innate lymphoid cells. These cells are abundantly found in the intestine. In particular, the gene expression and protein levels of IL-23R at the intestine mucosal surface are found to be elevated in inflammatory bowel disease (IBD) patients. It is thought that IL-23 mediates this effect by promoting the development of a pathogenic CD4⁺ T cell population that produces IL-6, IL-17, and TNF.

IL-23 production is enriched in the intestine, where it is believed to play a key role in regulating the balance between tolerance and immunity through both T-cell-dependent and independent pathways of intestinal inflammation through effects on Th1 and Th17-associated cytokines. IL-23 is also thought to restrain regulatory T-cell responses in the gut, favoring inflammation. Furthermore, IL-23R polymorphisms have been associated with susceptibility to inflammatory bowel diseases (IBDs), further establishing the critical role of the IL-23 pathway in intestinal homeostasis.

Therefore, IL-23 is thought to play a crucial role in the pathogenesis of autoimmune inflammation and related diseases and disorders, such as multiple sclerosis, asthma, rheumatoid arthritis, psoriasis, and inflammatory bowel diseases (IBDs), e.g., ulcerative colitis and Crohn's disease. Studies in acute and chronic mouse models of IBD have revealed a primary role of IL-23R and downstream effector cytokines in disease pathogenesis.

For example, psoriasis is a chronic skin disease affecting about 2%-3% of the general population. It is known to be mediated by the body's T cell inflammatory response mechanisms. IL-23 is among one of several interleukins implicated as a key player in psoriasis pathogenesis. It is thought IL-23 maintains chronic autoimmune inflammation via the induction of IL-17, activation of macrophages, and regulation of T memory cells. Additionally, expression of IL-23 and IL-23R has been shown to be increased in tissues of patients with psoriasis, and antibodies that neutralize IL-23 showed IL-23-dependent inhibition of psoriasis development in animal models of psoriasis.

Studies have been conducted to explore the potential therapeutic use of moieties capable of inhibition of the IL-23 pathway in treating IL-23-related diseases and disorders. Several antibodies that bind to IL-23 or IL-23R have been identified, including ustekinumab, a humanized antibody that binds IL-23, which has been approved for the treatment of psoriasis. More recently, polypeptide inhibitors that bind to IL-23R and inhibit the binding of IL-23 to IL-23R have been identified (see, e.g., US Patent Application Publication No. US 2013/0029907). Additionally, several clinical trials with ustekinumab and briakinumab (which target the common p40 subunit) and tildrakizumab, guselkumab, MEDI2070, and BI-655066 (which target the unique p19 subunit of IL-23) in the treatment of Crohn's Disease or psoriasis highlight the potential of IL-23 signalling blockade in treatment of human inflammatory diseases.

Protagonist Therapeutics, Inc. currently have a peptide PTG-200 in Phase II clinical trials for Crohn's disease. Protagonist also have two second generation peptides: PN-235 which has completed Phase I clinical trials and is expected to begin Phase II clinical trials for psoriasis; and PN-232 which is in Phase I clinical trials. Protagonist have filed several patent applications in the area of IL-23R inhibitors: WO 2016/011208, WO 2017/011820, WO 2018/022937, WO 2018/136646, WO 2020/014646, WO 2021/007433, WO 2021/146441, and WO 2021/146458. Protagonist also disclose another peptide, Compound C, as an IL-23R inhibitor in WO 2016/011208, WO 2017/011820, and Sayago et al., 2018.

WO 2016/011208 discloses oral peptide inhibitors of IL-23R and their use to treat inflammatory bowel diseases. The peptides are up to 20 amino acid residues long, and can optionally contain a single chemical bridge between amino acid residues 4 (X4) and 9 (X9). The chemical bridge may be a disulfide bond, a thioether bond, a lactam bond, a triazole ring, a selenoether bond, a diselenide bond, or an olefin bond. In particular, the chemical bridge is a disulfide or thioether bond.

WO 2017/011820 discloses peptide inhibitors of IL-23R nd related compositions and methods of using the peptide inhibitors to treat or prevent a variety of diseases and disorders, including inflammatory bowel diseases. The peptides are up to 20 amino acid residues long, and contain a single chemical bridge between amino acid residues 4 (X4) and 9 (X9). The chemical bridge may be a disulfide bond, a thioether bond, a lactam bond, a triazole ring, a selenoether bond, a diselenide bond, or an olefin bond. In particular, the chemical bridge is a disulfide or thioether bond.

WO 2018/022937 discloses peptide inhibitors of IL-23R and their use to treat inflammatory diseases, such as inflammatory bowel disease, Crohn's disease and psoriasis. The peptides disclosed are up to 39 amino acid residues long, and can optionally have a single disulfide or thioether bond between amino acid residues 5 (X5) and 34 (X34).

WO 2018/136646 discloses peptide inhibitors of IL-23R and their use to treat inflammatory diseases including inflammatory bowel disease. The peptides disclosed are up to 24 amino acid residues long, and can optionally have a cross-link between amino acid residues 5 (X4) and 10 (X9). The single cross-link between amino acids residues 5 and 10 may be a intramolecular disulfide or thioether bond.

WO 2020/014646 discloses peptide inhibitors of IL-23R and their use to treat or prevent a variety of diseases and disorders including inflammatory bowel diseases. The peptide sequences are 5 to 12 amino acid residues long, and can optionally have an intramolecular bond between amino acid residues 1 (X4) and 6 (X9). The intramolecular bond can be a disulfide bond, thioether bond, a lactam bond, a triazole ring, a selenoether bond, a diselenide bond or a olefin bond. In particular, the examples shown have a disulfide or thioether bond.

WO 2021/007433 discloses peptide inhibitors of IL-23R and their use to treat or prevent a variety of diseases and disorders including inflammatory bowel diseases. The peptide sequences are 5 to 8 amino acid residues long, and can optionally contain a bond between amino acid residues 1 (X4) and 6 (X9). The bond can be a disulfide or thioether bond.

WO 2021/146441 discloses peptide inhibitors of IL-23R and their use to treat or prevent a variety of diseases and disorders including inflammatory bowel diseases. The peptides are up to 14 amino acid residues long, which can optionally have a disulfide or thioether bond between amino acid residues 2 (X4) and 7 (X9).

WO 2021/146458 discloses peptide inhibitors of IL-23R and their use to treat or prevent a variety of diseases and disorders including inflammatory bowel disease, Crohn's disease, ulcerative colitis and psoriasis. The peptides are up to 14 amino acid residues long, which can optionally have a bond between amino acid residues 2 (X4) and 7 (X9). The bond may be a disulfide or thioether bond.

Notably, only single bridging moieties are present in the peptides disclosed by the above Protagonist patent applications. None disclose the use of two bridging moieties to stabilise the peptide inhibitor of IL-23R.

Heinis et al., 2020 discloses the development of proteolytically resistant therapeutic peptides for oral administration. The authors generated peptides as inhibitors of coagulation Factor XIa and other peptides as gastrointestinal-protease resistant peptide antagonists of IL-23R. The peptides generated as antagonists of IL-23R comprised of two dithioether bridges (specifically 1,3-dithio-propan-2-one bridges) between two pairs of cysteine residues in the peptide chain. The authors identified peptide 15 as the most promising candidate for further development as an oral treatment of inflammatory disorders such as Crohn's disease on the basis of IL-23R inhibition.

However, challenges remain with respect to identifying stable and selective agents that preferentially target the IL-23 pathway in the intestine, which can be used for the treatment of intestinal inflammation, such as intestinal bowel diseases, including Crohn's disease, ulcerative colitis and related disorders. In particular, the inventors have identified the gastrointestinal stability and IL-23R potency of the peptides disclosed in Heinis et al., 2020 could still be further improved. In particular, the most-promising candidate, peptide 15, exhibited a lower stability under a simulated intestinal fluid (SIF) assay and a lower potency for IL-23R in comparison with Protagonist's Compound C (Sayago et al., 2018).

Therefore, there remains a need for new therapeutics targeting the IL-23 pathway, which may be used to treat and prevent IL-23-associated diseases, including those associated with autoimmune inflammation in the intestinal tract. Furthermore, compounds and methods for specific targeting of IL-23R from the luminal side of the gut may provide therapeutic benefit to IBD patients suffering from local inflammation of the intestinal tissue.

The present invention addresses these needs by providing novel peptide inhibitors that bind IL-23R to inhibit IL-23 mediated signalling. The novel peptide inhibitors are also suitable for oral administration due to their stability in the gastrointestinal tract.

### SUMMARY OF THE INVENTION

The present invention relates to compounds which are peptide inhibitors of interleukin-23 receptor (IL-23R).

In a first aspect, the invention provides a compound having the formula:

R¹—Z—R²
wherein
R¹ is H, C₁₋₄ acyl, benzoyl, C₁₋₄ alkyl, or is absent;
R² is NHR³, OH, or is absent, wherein R³ is hydrogen or C₁₋₃ alkyl optionally substituted with NH₂; and
Z is an amino acid sequence of formula I:

X1-X2-X3-X4-X5-X6-X7-X8-X9-X10-X11-X12-X13-X14 (1)
wherein
X1 is absent or is selected from the group consisting of Asp, Gly, Leu, Glu, Ser, Cys, and Lys;
X3 is selected from the group consisting of Ser, beta-homo-Ser, Thr, Leu, Cys, Gln, Val, Ile, N-Me-Ser, and Q(pyrrolidin);
X5 is selected from the group consisting of Trp, Tyr, Ala, 1-Me-Trp, 7-Me-Trp, 7-Ph-Trp, 7-(Naphth-2-yl)-Trp, 2-Nal, Bip, 4-F-Trp, 7-F-Trp, and N-Me-Trp;
X6 is selected from the group consisting of Gln, Glu, Tyr, Cys, Val, His, N-Me-Gln, and Q(pyrrolidin);
X8 is selected from the group consisting of Trp, Tyr, Asn, Ala, His, 2-Nal, Dab, 2,4-diaminobutanoyl([2-(trimethyl-2-aminoethoxy)ethoxy]propyl), F(4-NH₂-(2-(trimethyl-2-aminoethoxy)ethoxy)propyl), Phe, Val, 4-Me-Phe, 2-Me-Phe, Bip, 2-Me-F(4-F), {d}F(4-F), 4-Cl-Phe, alpha-Me-Trp, 3,3-Diphenyl-Ala, and Phg, wherein the hydroxyl group of Tyr is optionally substituted with C₁₋₃ alkyl optionally substituted with NH₂;
X9 is selected from the group consisting of 2-Nal, Trp, 1-Me-Trp, 6-Cl-Trp, 3-(3-Quinolinyl)-Ala, Phe, 4-F-Phe, Glu, Cys, Ala, 6-F-Trp, His, 3-F-Phe, 3,4-Me-Phe, Bip, and {d}6-F-Trp;
X10 is selected from the group consisting of Leu, D-Leu, 2-Me-Leu, 2-Me-Lys, Trp, Asn, Cys, 4-aminotetrahydro-2H-pyran-4-acetyl, and 2-Me-Val;
X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, 3-(4-Pyridyl)-Ala, {d}2,4-diaminobutanoyl([2-(trimethyl-2-aminoethoxy)ethoxy]propyl), D-Gln, D-Glu, D-His, 3-aminopropanoyl, and GABA, or is absent;
X13 is absent or is selected from the group consisting of Asn, Gly, 3-(3-Pyridyl)-Ala, 3-(4-Pyridyl)-Ala, 3-(3-Quinolinyl)-Ala, {d}[3-(3-Pyridyl)-Ala], 3-amino-3-(3′-pyridyl)propionyl, 3-F-Phe, 3,5-F-Phe, 4-aminomethyl-2-pyridineacetyl, 2,3-diaminopropanoyl(3-pyridylacetyl), 2,3-diaminopropanoyl(3-pyridylpropionyl), 2,3-diaminopropanoyl(3-fluorobenzoyl), 2,3-diaminopropanoyl(3-fluorophenylacetyl), and 2-Me-3-(3-Pyridyl)-Ala;
X14 is absent or is Gly;
X2 and X11 are amino acid residues who together form a lactam bridge, a dithioether bridge, or a bridge containing a triazole ring; and
X4 and X7 are amino acid residues who together form a lactam bridge, a dithioether bridge, or a bridge containing a triazole ring;
or a pharmaceutically acceptable salt or solvate thereof.

In some embodiments, the invention provides a compound having the formula:

R¹—Z—R²
wherein
R¹ is H, C₁₋₄ acyl, benzoyl, C₁₋₄ alkyl, or is absent;
R² is NHR³, OH, or is absent, wherein R³ is hydrogen or C₁₋₃ alkyl; and
Z is an amino acid sequence of formula Ia:

X1-X2-X3-X4-X5-X6-X7-X8-X9-X10-X11-X12-X13-X14 (Ia)
wherein
X1 is absent or is selected from the group consisting of Asp, Glu, Ser, Cys, and Lys;
X3 is selected from the group consisting of Ser, beta-homo-Ser, Thr, Leu, Cys, and Gln;
X5 is selected from the group consisting of Trp, Tyr, Ala, 1-Me-Trp, 7-Me-Trp, 7-Ph-Trp, 7-(Naphth-2-yl)-Trp, 2-Nal, and Bip;
X6 is selected from the group consisting of Gln, Glu, Tyr and Cys;
X8 is selected from the group consisting of Trp, Tyr, Asn, Ala, His, and 2-Nal, wherein the hydroxyl group of Tyr is optionally substituted with C₁₋₃ alkyl optionally substituted with NH₂;
X9 is selected from the group consisting of 2-Nal, Trp, 1-Me-Trp, 6-Cl-Trp, 3-(3-Quinolinyl)-Ala, Phe, 4-F-Phe, Glu, Cys, and Ala;
X10 is selected from the group consisting of Leu, D-Leu, 2-Me-Leu, 2-Me-Lys, Trp, Asn, Cys, and 4-aminotetrahydro-2H-pyran-4-acetyl;
X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent;
X13 is absent or is selected from the group consisting of Asn, Gly, 3-(3-Pyridyl)-Ala, and 3-(4-Pyridyl)-Ala;
X14 is absent or is Gly;
X2 and X11 are amino acid residues who together form a lactam bridge, a dithioether bridge, or a bridge containing a triazole ring; and
X4 and X7 are amino acid residues who together form a lactam bridge, a dithioether bridge, or a bridge containing a triazole ring;
or a pharmaceutically acceptable salt or solvate thereof.

In some embodiments, the compound is not:

I3(isomer 3)H-DC(1a)SC(2a)WQC(2a)WWLC(1a)R-[NH₂];
wherein (1a) is a [2,11]1,3-dithio-propan-2-one bridge and (2a) is a [4,7]1,3-dithio-propan-2-one bridge.

In some embodiments, the compound is not:

I3(isomer 3)H-DC(1a)SC(2a)WQC(2a)WWLC(1a)R-OH;
wherein (1a) is a [2,11]1,3-dithio-propan-2-one bridge and (2a) is a [4,7]1,3-dithio-propan-2-one bridge.

In some embodiments, Z is not:

I3(isomer 3)DC(1a)SC(2a)WQC(2a)WWLC(1a)R;
wherein (1a) is a [2,11]1,3-dithio-propan-2-one bridge and (2a) is a [4,7]1,3-dithio-propan-2-one bridge.

In some embodiments, the bridge in X2 and X11 and the bridge in X4 and X7 are not both 1,3-dithio-propan-2-one bridges.

In some embodiments, the bridge in X2 and X11 and the bridge in X4 and X7 are not both dithioether bridges.

In some embodiments, X1 is absent or Asp.

In some embodiments, X1 is absent.

In some embodiments, X3 is Ser or lie.

In some embodiments, X3 is Ser.

In some embodiments, X5 is Trp or 7-Me-Trp.

In some embodiments, X5 is Trp.

In some embodiments, X6 is Gln or Glu.

In some embodiments, X6 is Gln.

In some embodiments, X8 is Trp, Tyr, or 4-Me-Phe; wherein the hydroxyl group of Tyr is optionally substituted with —CH₂CH₂NH₂.

In some embodiments, X8 is Tyr wherein the hydroxyl group of Tyr is optionally substituted with —CH₂CH₂NH₂.

In some embodiments, X9 is 2-Nal, Trp, or 3,4-Me-Phe.

In some embodiments, X9 is 2-Nal or Trp.

In some embodiments, X9 is 2-Nal.

In some embodiments, X10 is Leu, 2-Me-Leu, or 2-Me-Val.

In some embodiments, X10 is Leu or 2-Me-Leu.

In some embodiments, X10 is Leu.

In some embodiments, X12 is Arg, D-Arg, and Dab.

In some embodiments, X12 is Arg.

In some embodiments, X13 is absent, 3-(3-Pyridyl)-Ala, or 2-Me-3-(3-Pyridyl)-Ala.

In some embodiments, X13 is absent.

In some embodiments, X14 is absent.

In some embodiments, R¹ is H, C₁₋₂ acyl, or is absent.

In some embodiments, R¹ is absent.

In some embodiments, R¹ is —C(═O)CH₃.

In some embodiments, R² is NH₂.

In some embodiments, the length of the bridge between X2 and X11 and/or X4 and X7 is 5 to 10 atoms long.

In some embodiments, X1 is absent, R¹ is absent, and X2 and X11 are amino acid residues that together form a lactam bridge or a bridge containing a triazole ring via the N-terminus of X2.

In some embodiments, X1 and X12 to X14 are absent, R¹ and R² are absent, and X2 and X11 are amino acid residues that together form a head-to-tail cyclised lactam bridge via the N-terminus of X2 and the C-terminus of X11.

In some embodiments, the dithioether bridge between X2 and X11 and/or X4 and X7 is of the formula —S—L—Y—L—S—, wherein:
each S is a sulfur atom and is part of the amino acid residue at X2 and X11 and/or X4 and X7;
each L is independently C₁₋₄ alkylene; and
Y is either absent, C(═O), or arylene.

In some embodiments, each L is independently C₁₋₂ alkylene.

In some embodiments, each L is methylene.

In some embodiments, Y is C(═O).

In some embodiments, Y is arylene selected from phenylene.

In some embodiments, Y is phenylene selected from 1,2-phenylene, 1,3-phenylene, and 1,4-phenylene.

In some embodiments, Y is 1,2-phenylene.

In some embodiments, the bridge containing a triazole ring between X2 and X11 and/or X4 and X7 contains a 1,2,3-triazole ring.

In some embodiments, the bridge containing a triazole ring between X2 and X11 and/or X4 and X7 is attached to positions 1 and 4 of the triazole ring.

In some embodiments, the bridge containing a triazole ring between X2 and X11 and/or X4 and X7 is attached to positions 1 and 5 of the triazole ring.

In some embodiments, X2 and X11 are amino acid residues who together form a lactam bridge.

In some embodiments, the location of the amide bond in the lactam bridge is closer to X11 than X2.

In some embodiments, one of the residues at position X2 and X11 is selected from Lys, Arg, Orn, bAla, 3-(4-aminophenyl)propanoyl, (3-aminomethyl)benzoyl, (4-aminomethyl)benzoyl, 4-(2-aminoethyl)benzoyl, 2-aminomethyl-phenylacetyl, 3-aminomethyl-phenylacetyl, 4-aminomethyl-phenylacetyl, 4-aminomethyl-2-pyridineacetyl, 4-aminomethyl-3-pyridineacetyl, 4-aminomethyl-2-fluoro-phenylacetyl, 4-aminomethyl-3-fluoro-phenylacetyl, 4-aminomethyl-2-methyl-phenylacetyl, 4-aminomethyl-3-methyl-phenylacetyl, 4-aminomethyl-2-methoxy-phenylacetyl, 4-aminomethyl-3-methoxy-phenylacetyl, Dab, 6-aminohexanoyl, 6-amino-4-oxahexanoyl, trans-4-aminomethyl-cyclohexyl-1-carbonyl, (4-(2-aminoethyl)-piperazine-1-yl)-acetyl, 2,4-diaminobutanoyl([2-(trimethyl-2-aminoethoxy)ethoxy]propyl), and 4-methylaminomethyl-phenylacetyl, and the other is selected from Glu and Asp.

In some embodiments, X2 is selected from Lys, Orn, bAla, 3-(4-aminophenyl)propanoyl, (3-aminomethyl)benzoyl, (4-aminomethyl)benzoyl, 4-(2-aminoethyl)benzoyl, 2-aminomethyl-phenylacetyl, 3-aminomethyl-phenylacetyl, 4-aminomethyl-phenylacetyl, 4-aminomethyl-2-pyridineacetyl, 4-aminomethyl-3-pyridineacetyl, 4-aminomethyl-2-fluoro-phenylacetyl, 4-aminomethyl-3-fluoro-phenylacetyl, 4-aminomethyl-2-methyl-phenylacetyl, 4-aminomethyl-3-methyl-phenylacetyl, 4-aminomethyl-2-methoxy-phenylacetyl, 4-aminomethyl-3-methoxy-phenylacetyl, 6-aminohexanoyl, 6-amino-4-oxahexanoyl, trans-4-aminomethyl-cyclohexyl-1-carbonyl, (4-(2-aminoethyl)-piperazine-1-yl)-acetyl, 2,4-diaminobutanoyl([2-(trimethyl-2-aminoethoxy)ethoxy]propyl), and 4-methylaminomethyl-phenylacetyl, and X11 is Glu and Asp.

In some embodiments, X2 is Lys and X11 is Glu; X2 is Orn and X11 is Glu; X2 is bAla and X11 is Glu; X2 is 3-(4-aminophenyl)propanoyl and X11 is Glu; X2 is (3-aminomethyl)benzoyl and X11 is Glu; X2 is (4-aminomethyl)benzoyl and X11 is Glu; X2 is 4-(2-aminoethyl)benzoyl and X11 is Glu; X2 is 2-aminomethyl-phenylacetyl and X11 is Glu; X2 is 3-aminomethyl-phenylacetyl and X11 is Glu; X2 is 4-aminomethyl-phenylacetyl and X11 is Glu; X2 is 6-aminohexanoyl and X11 is Glu; X2 is 6-amino-4-oxahexanoyl and X11 is Glu; X2 is trans-4-aminomethyl-cyclohexyl-1-carbonyl and X11 is Glu; X2 is (4-(2-aminoethyl)-piperazine-1-yl)-acetyl and X11 is Glu; X2 is (4-(2-aminoethyl)-piperazine-1-yl)-acetyl and X11 is Asp; X2 is 4-aminomethyl-2-pyridineacetyl and X11 is Glu; X2 is 4-aminomethyl-3-pyridineacetyl and X11 is Glu; X2 is 4-aminomethyl-2-fluoro-phenylacetyl and X11 is Glu; X2 is 4-aminomethyl-3-fluoro-phenylacetyl and X11 is Glu; X2 is 4-aminomethyl-2-methyl-phenylacetyl and X11 is Glu; X2 is 4-aminomethyl-3-methyl-phenylacetyl and X11 is Glu; X2 is 4-aminomethyl-2-methoxy-phenylacetyl and X11 is Glu; X2 is 4-aminomethyl-3-methoxy-phenylacetyl and X11 is Glu; X2 is Dab and X11 is Glu; X2 is 2,4-diaminobutanoyl([2-(trimethyl-2-aminoethoxy)ethoxy]propyl) and X11 is Glu; or X2 is 2,4-diaminobutanoyl([2-(trimethyl-2-aminoethoxy)ethoxy]propyl) and X11 is Glu.

In some embodiments, X2 is Lys and X11 is Glu; X2 is (3-aminomethyl)benzoyl and X11 is Glu; or X2 is 4-aminomethyl-phenylacetyl and X11 is Glu.

In some embodiments, X2 is Lys and X11 is Glu.

In some embodiments, X2 is selected from Glu and Asp, and X11 is selected from Lys, Arg, and Dab.

In some embodiments, X2 is Glu and X11 is Lys; X2 is Glu and X11 is Dab; or X2 is Asp and X11 is Arg.

In some embodiments, X2 and X11 are amino acid residues who together form a dithioether bridge.

In some embodiments, X2 and X11 are each independently selected from Cys and N-Me-Cys.

In some embodiments, X2 is Cys and X11 is Cys.

In some embodiments, X2 and X11 are amino acid residues who together form a bridge containing a triazole ring.

In some embodiments, one of the residues at position X2 and X11 is selected from Lys(N₃), azidoacetic acid, (N₃)-Ala, Dab(azidoacetic acid), and the other is selected from Pra, Glu(propargylamine), Dab(3-butynoic acid), and but-3-ynoic acid.

In some embodiments, X2 is selected from Lys(N₃), azidoacetic acid, and (N₃)-Ala; and
X11 is selected from Pra, Glu(propargylamine), and Dab(3-butynoic acid).

In some embodiments, X2 is Lys(N₃) and X11 is Pra; X2 is azidoacetic acid and X11 is Glu(propargylamine); X2 is azidoacetic acid and X11 is Dab(3-butynoic acid); X2 is (N₃)-Ala and X11 is Glu(propargylamine); or X2 is (N₃)-Ala and X11 is Dab(3-butynoic acid).

In some embodiments, X2 is Lys(N₃) and X11 is Pra.

In some embodiments, X2 is selected from Pra, and but-3-ynoic acid; and X11 is selected from Dab(azidoacetic acid), and Dab((N₃)-Ala).

In some embodiments, X2 is Pra and X11 is Dab(azidoacetic acid); X2 is Pra and X11 is Dab((N₃)-Ala); X2 is but-3-ynoic acid and X11 is Dab(azidoacetic acid); or X2 is but-3-ynoic acid and X11 is Dab((N₃)-Ala). In some embodiments, X2 is Pra and X11 is Dab(azidoacetic acid); or X2 is Pra and X11 is Dab((N₃)-Ala). In some embodiments, X2 is but-3-ynoic acid and X11 is Dab(azidoacetic acid); or X2 is but-3-ynoic acid and X11 is Dab((N₃)-Ala).

In some embodiments, X4 and X7 are amino acid residues who together form a dithioether bridge.

In some embodiments, X4 and X7 are each independently selected from Cys and N-Me-Cys.

In some embodiments, X4 is Cys and X7 is Cys; or X4 is N-Me-Cys and X7 is Cys.

In some embodiments, X4 is Cys and X7 is Cys.

In some embodiments, X4 and X7 are amino acid residues who together form a lactam bridge.

In some embodiments, one of the residues at position X4 and X7 is Lys, Dpr, Dab, or Orn, and the other is Glu.

In some embodiments, X4 is selected from Lys, Dpr, Dab, and Orn, and X7 is Glu.

In some embodiments, X4 is Dpr and X7 is Glu; X4 is Dab and X7 is Glu; or X4 is Orn and X7 is Glu.

In some embodiments, X4 is Glu, and X7 is selected from Lys, Dpr, Dab, and Orn.

In some embodiments, X4 is Glu and X7 is Lys; X4 is Glu and X7 is Dpr; X4 is Glu and X7 is Orn; or X4 is Glu and X7 is Dab.

In some embodiments, X4 and X7 are amino acid residues who together form a bridge containing a triazole ring.

In some embodiments, one of the residues at position X4 and X7 is selected from Lys(N₃) and Aha, and the other is Pra.

In some embodiments, X4 is Lys(N₃) and X7 is Pra; or X4 is Aha and X7 is Pra.

In some embodiments, X8 is Trp; X10 is D-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.

In some embodiments, X8 is Tyr wherein the hydroxyl group of Tyr is optionally substituted with C₁₋₃ alkyl optionally substituted with NH₂; X10 is D-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.

In some embodiments, X8 is Asn; X10 is D-Leu; and X12 is selected from the group consisting of Arg, D-Arg, 2-Me-Arg, N-Me-Arg, Ser, Phe, 4-NH₂Phe, Tyr, Thr, Met, Gly, Glu, Asn, Dab, 3-(3-Pyridyl)-Ala, and 3-(4-Pyridyl)-Ala, or is absent.

In some embodiments, X8 is Ala; X10 is D-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.

In some embodiments, X8 is His; X10 is D-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.

In some embodiments, X8 is 2-Nal; X10 is D-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.

In some embodiments, X8 is Tyr wherein the hydroxyl group of Tyr is optionally substituted with —CH₂CH₂NH₂; X10 is 2-Me-Leu; and X12 is Arg or is absent.

In some embodiments, X8 is Trp; X10 is 2-Me-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.

In some embodiments, X8 is Tyr wherein the hydroxyl group of Tyr is optionally substituted with C₁₋₃ alkyl optionally substituted with NH₂; X10 is 2-Me-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.

In some embodiments, X8 is Asn; X10 is 2-Me-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.

In some embodiments, X8 is Ala; X10 is 2-Me-Leu; and X12 is selected from the group consisting of Arg, D-Arg, 2-Me-Arg, N-Me-Arg, Ser, Phe, 4-NH₂₋Phe, Tyr, Thr, Met, Gly, Glu, Asn, Dab, 3-(3-Pyridyl)-Ala, and 3-(4-Pyridyl)-Ala, or is absent.

In some embodiments, X8 is His; X10 is 2-Me-Leu; and X12 is selected from the group consisting of Arg, D-Arg, 2-Me-Arg, N-Me-Arg, Ser, Phe, 4-NH₂Phe, Tyr, Thr, Met, Gly, Glu, Asn, Dab, 3-(3-Pyridyl)-Ala, and 3-(4-Pyridyl)-Ala, or is absent.

In some embodiments, XC is 2-Nal; X10 is 2-Me-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.

In some embodiments, X8 is Tyr wherein the hydroxyl group of Tyr is optionally substituted with —CH₂CH₂NH₂; X10 is D-Leu; and X12 is Arg or is absent.

In some embodiments, X8 is Tyr wherein the hydroxyl group of Tyr is optionally substituted with —CH₂CH₂NH₂; X10 is 2-Me-Leu; and X12 is Arg or is absent.

In some embodiments, Z is an amino acid sequence selected from the group consisting of:

| | |
|---|---|
| SEQ ID NO: 1 | DC(1a)SC(2a)WQC(2a)WW[2-Me-Leu]C(1a)R |
| SEQ ID NO: 2 | LC(1a)SC(2a)WQC(2a)WWLC(1a)R |
| SEQ ID NO: 3 | DC(1a)SC(2a)WEC(2a)WWLC(1a)R |
| SEQ ID NO: 4 | DE(1c)SC(2a)WQC(2a)WWLK(1c)R |
| SEQ ID NO: 5 | DC(1a)SE(2c)WQK(2c)WWLC(1a)R |
| SEQ ID NO: 6 | D(1c)*SC(2a)WQC(2a)WWLR(1c)* |
| SEQ ID NO: 7 | DK(1c)SC(2a)WQC(2a)WWLE(1c)R |
| SEQ ID NO: 8 | D[Orn](1c)SC(2a)WQC(2a)WWLE(1c)R |
| SEQ ID NO: 9 | DE(1c)SC(2a)WQC(2a)WWL[Dab](1c)R |
| SEQ ID NO: 10 | E(1c)SC(2a)WQC(2a)WWLK(1c)R |
| SEQ ID NO: 11 | DE(1c)SC(2a)WQC(2a)WWLK(1c) |
| SEQ ID NO: 12 | E(1c)SC(2a)WQC(2a)WWLK(1c) |
| SEQ ID NO: 13 | DE(1c)SC(2a)AQC(2a)WWLK(1c)R |
| SEQ ID NO: 14 | DE(1c)SC(2a)WQC(2a)AWLK(1c)R |
| SEQ ID NO: 15 | DE(1c)SC(2a)WQC(2a)WALK(1c)R |
| SEQ ID NO: 16 | DE(1c)SC(2a)[7-Me-Trp]QC(2a)WWLK(1c)R |
| SEQ ID NO: 17 | DE(1c)SC(2a)WQC(2a)W[2-Nal]LK(1c)R |
| SEQ ID NO: 18 | K(1c)SC(2a)WQC(2a)W[2-Nal]LE(1c)R |
| SEQ ID NO: 19 | DK(1c)SC(2a)WQC(2a)W[2-Nal]LE(1c)R |
| SEQ ID NO: 20 | DK(1c)SC(2d)WQC(2d)W[2-Nal]LE(1c)R |
| SEQ ID NO: 21 | DK(1c)SC(2e)WQC(2e)W[2-Nal]LE(1c)R |
| SEQ ID NO: 22 | DK(1c)SC(2f)WQC(2f)W[2-Nal]LE(1c)R |
| SEQ ID NO: 23 | DK(1c)S[K(N₃)](2g)WQ[Pra](2g)W[2-Nal]LE(1c)R |
| SEQ ID NO: 24 | DK(1c)S[Aha](2g)WQ[Pra](2g)W[2-Nal]LE(1c)R |
| SEQ ID NO: 25 | K(1c)SC(2a)[2-Nal]QC(2a)W[2-Nal]LE(1c)R |
| SEQ ID NO: 26 | K(1c)SC(2a)[Bip]QC(2a)W[2-Nal]LE(1c)R |
| SEQ ID NO: 27 | K(1c)SC(2a)[1-Me-Trp]QC(2a)W[2-Nal]LE(1c)R |
| SEQ ID NO: 28 | DK(1c)SC(2a)WQC(2a)[2-Nal][2-Nal]LE(1c)R |
| SEQ ID NO: 29 | K(1c)SC(2a)WQC(2a)[Y(2-aminoethoxy)][2-Nal]LE(1c)R |
| SEQ ID NO: 30 | K(1c)SC(2a)WQC(2a)H[2-Nal]LE(1c)R |
| SEQ ID NO: 31 | K(1c)SC(2a)WQC(2a)W[F(4-F)]LE(1c)R |
| SEQ ID NO: 32 | K(1c)SC(2a)WQC(2a)W[1-Me-Trp]LE(1c)R |
| SEQ ID NO: 33 | C(1a)TC(2a)WEC(2a)WW[2-Me-Leu]C(1a)S |
| SEQ ID NO: 34 | GK(1c)TC(2a)WEC(2c)WW[2-Me-Leu]E(1c)S |
| SEQ ID NO: 35 | GC(1a)TC(2a)WEC(2a)W[2-Nal][2-Me-Leu]C(1a)R |
| SEQ ID NO: 36 | [bAla](1c)*SC(2a)WQC(2a)W[2-Nal]LE(1c)R |
| SEQ ID NO: 37 | [(3-Aminomethyl)benzoyl](1c)*SC(2a)WQC(2a)W[2-Nal]LE(1c)R |
| SEQ ID NO: 38 | [(3-Aminomethyl)benzoyl](1c)*SC(2a)WQC(2a)[Y(2-aminoethoxy)][2- |
| | Nal][2-Me-Leu]E(1c)R-[NH₂] |
| SEQ ID NO: 39 | [(3-Aminomethyl)benzoyl](1c)*SC(2a)WQC(2a)[Y(2-aminoethoxy)][2- |
| | Nal]LE(1c)R |
| SEQ ID NO: 40 | [(3-Aminomethyl)benzoyl](1c)*SC(2a)WQC(2a)W[2-Nal][2-Me-Leu]E(1c)R |
| SEQ ID NO: 41 | [(3-Aminomethyl)benzoyl](1c)*SC(2a)WQC(2a)[Y(2-aminoethoxy)][2- |
| | Nal][2-Me-Leu]E(1c)[{d}R] |
| SEQ ID NO: 42 | [(3-Aminomethyl)benzoyl](1c)*SC(2a)WQC(2a)[Y(2-aminoethoxy)][2- |
| | Nal][2-Me-Leu]E(1c)[2-Me-Arg] |
| SEQ ID NO: 43 | [(3-Aminomethyl)benzoyl](1c)*SC(2a)WQC(2a)[Y(2-aminoethoxy)][2- |
| | Nal][2-Me-Leu]E(1c)[N-Me-Arg] |
| SEQ ID NO: 44 | [(3-Aminomethyl)benzoyl](1c)*SC(2a)WQC(2a)[Y(2-aminoethoxy)][2- |
| | Nal][2-Me-Leu]E(1c)S |
| SEQ ID NO: 45 | [(3-Aminomethyl)benzoyl](1c)*SC(2a)WQC(2a)[Y(2-aminoethoxy)][2- |
| | Nal][2-Me-Leu]E(1c)[F(4-NH₂)] |
| SEQ ID NO: 46 | [(3-Aminomethyl)benzoyl](1c)*SC(2a)WQC(2a)[Y(2-aminoethoxy)][2- |
| | Nal][{d}L]E(1c)Y |
| SEQ ID NO: 47 | [Ac]-K(1c)SC(2a)WQC(2a)[Y(2-aminoethoxy)][2-Nal][2-Me-Leu]E(1c)R |
| SEQ ID NO: 48 | [Ac]-K(1c)SC(2a)WQC(2a)[Y(2-aminoethoxy)][2-Nal][{d}L]E(1c)R |
| SEQ ID NO: 49 | [(4-Aminomethyl)benzoyl](1c)*SC(2a)WQC(2a)[Y(2-aminoethoxy)][2- |
| | Nal][2-Me-Leu]E(1c)R |
| SEQ ID NO: 50 | [4-(2-Aminoethyl)benzoyl](1c)*SC(2a)WQC(2a)[Y(2-aminoethoxy)][2- |
| | Nal][2-Me-Leu]E(1c)R |
| SEQ ID NO: 51 | [3-(4-Aminophenyl)propanoyl](1c)*SC(2a)WQC(2a)[Y(2-aminoethoxy)][2- |
| | Nal][2-Me-Leu]E(1c)R |
| SEQ ID NO: 52 | [4-Aminomethyl-phenylacetyl](1c)*SC(2a)WQC(2a)[Y(2-aminoethoxy)][2- |
| | Nal][2-Me-Leu]E(1c)R |
| SEQ ID NO: 53 | DC(1a)SC(2b)WQC(2b)WWLC(1a)R |
| SEQ ID NO: 54 | K(1c)SC(2b)WQC(2b)[Y(2-aminoethoxy)][2-Nal]LE(1c)R |
| SEQ ID NO: 55 | DE(1c)QC(2a)WQC(2a)YW[2-Me-Leu]K(1c)R |
| SEQ ID NO: 56 | DE(1c)QC(2a)WQC(2a)[2-Nal]W[2-Me-Leu]K(1c)R |
| SEQ ID NO: 57 | DE(1c)TC(2a)WQC(2a)W[2-Nal][2-Me-Leu]K(1c) |
| SEQ ID NO: 58 | DE(1c)SC(2a)WQC(2a)W[2-Nal][2-Me-Leu]K(1c)ENG |
| SEQ ID NO: 59 | DE(1c)SC(2a)WQC(2a)W[2-Nal][2-Me-Leu]K(1c)NG |
| SEQ ID NO: 60 | DE(1c)SC(2a)WQC(2a)W[2-Nal][2-Me-Leu]K(1c)G |
| SEQ ID NO: 61 | DE(1c)SC(2a)WQC(2a)W[2-Nal][2-Me-Lys]K(1c)ENG |
| SEQ ID NO: 62 | DC(1a)QC(2a)WQC(2a)[2-Nal]W[2-Me-Leu]C(1a)R |
| SEQ ID NO: 63 | DC(1a)QC(2a)WQC(2a)YW[2-Me-Leu]C(1a)R |
| SEQ ID NO: 64 | DC(1a)QC(2a)WQC(2a)WW[2-Me-Leu]C(1a)ENG |
| SEQ ID NO: 65 | [(3-Aminomethyl)benzoyl](1c)*S[Dpr](2c)WQE(2c)[Y(2-aminoethoxy)][2- |
| | Nal][2-Me-Leu]E(1c)R |
| SEQ ID NO: 66 | [(3-Aminomethyl)benzoyl](1c)*SE(2c)WQ[Dpr](2c)[Y(2-aminoethoxy)][2- |
| | Nal][2-Me-Leu]E(1c)R |
| SEQ ID NO: 67 | [(3-Aminomethyl)benzoyl](1c)*SE(2c)WQ[Dab](2c)[Y(2-aminoethoxy)][2- |
| | Nal][2-Me-Leu]E(1c)R |
| SEQ ID NO: 68 | [(3-Aminomethyl)benzoyl](1c)*S[Orn](2c)WQE(2c)[Y(2-aminoethoxy)][2- |
| | Nal][2-Me-Leu]E(1c)R |
| SEQ ID NO: 69 | [(3-Aminomethyl)benzoyl](1c)*SE(2c)WQ[Orn](2c)[Y(2-aminoethoxy)][2- |
| | Nal][2-Me-Leu]E(1c)R |
| SEQ ID NO: 70 | [(3-Aminomethyl)benzoyl](1c)*SC(2a)[7-Me-Trp]QC(2a)[Y(2- |
| | aminoethoxy)][2-Nal][2-Me-Leu]E(1c)R |
| SEQ ID NO: 71 | [(3-Aminomethyl)benzoyl](1c)*SC(2a)[7-Ph-Trp]QC(2a)[Y(2- |
| | aminoethoxy)][2-Nal][2-Me-Leu]E(1c)R |
| SEQ ID NO: 72 | [(3-Aminomethyl)benzoyl](1c)*SC(2a)[7-(Naphth-2-yl)-Trp]QC(2a)[Y(2- |
| | aminoethoxy)][2-Nal][2-Me-Leu]E(1c)R |
| SEQ ID NO: 73 | [(3-Aminomethyl)benzoyl](1c)*SC(2a)WQC(2a)[Y(2-aminoethoxy)][3-(3- |
| | Quinolinyl)-Ala][2-Me-Leu]E(1c)R |
| SEQ ID NO: 74 | [(3-Aminomethyl)benzoyl](1c)*SC(2a)WQC(2a)[Y(2-aminoethoxy)][6-Cl- |
| | Trp][2-Me-Leu]E(1c)R |
| SEQ ID NO: 75 | [(3-Aminomethyl)benzoyl](1c)*SC(2a)WQC(2a)[Y(2-aminoethoxy)][2- |
| | Nal][4-aminotetrahydro-2H-pyran-4-acetyl]E(1c)R |
| SEQ ID NO: 76 | [(3-Aminomethyl)benzoyl](1c)*SC(2a)WQC(2a)[Y(2-aminoethoxy)][2- |
| | Nal][2-Me-Leu]E(1c)[3-(3-Pyridyl)-Ala] |
| SEQ ID NO: 77 | [(3-Aminomethyl)benzoyl](1c)*SC(2a)WQC(2a)[Y(2-aminoethoxy)][2- |
| | Nal][2-Me-Leu]E(1c)[3-(4-Pyridyl)-Ala] |
| SEQ ID NO: 78 | [(3-Aminomethyl)benzoyl](1c)*SC(2a)WQC(2a)[Y(2-aminoethoxy)][2- |
| | Nal][2-Me-Leu]E(1c)R[3-(3-Pyridyl)-Ala] |
| SEQ ID NO: 79 | [(3-Aminomethyl)benzoyl](1c)*SC(2a)WQC(2a)[Y(2-aminoethoxy)][2- |
| | Nal][2-Me-Leu]E(1c)R[3-(4-Pyridyl)-Ala] |
| SEQ ID NO: 80 | [4-Aminomethyl-phenylacetyl](1c)*SC(2a)WQC(2a)[Y(2-aminoethoxy)][2- |
| | Nal][2-Me-Leu]E(1c)[{d}R] |
| SEQ ID NO: 81 | [4-Aminomethyl-phenylacetyl](1c)*SC(2a)WQC(2a)[Y(2-aminoethoxy)][2- |
| | Nal][2-Me-Leu]E(1c)S |
| SEQ ID NO: 82 | [4-Aminomethyl-phenylacetyl](1c)*SC(2a)WQC(2a)[Y(2-aminoethoxy)][2- |
| | Nal][2-Me-Leu]E(1c)[Dab] |
| SEQ ID NO: 83 | [4-Aminomethyl-phenylacetyl](1c)*TC(2a)WQC(2a)[Y(2-aminoethoxy)][2- |
| | Nal][2-Me-Leu]E(1c)[{d}R] |
| SEQ ID NO: 84 | K(1c)SC(2a)WQC(2a)[Y(2-aminoethoxy)][2-Nal][2-Me-Leu]E(1c)[{d}R] |
| SEQ ID NO: 85 | K(1c)TC(2a)WQC(2a)[Y(2-aminoethoxy)][2-Nal][2-Me-Leu]E(1c)[{d}R] |
| SEQ ID NO: 86 | K(1c)SC(2a)WQC(2a)[Y(2-aminoethoxy)][2-Nal][2-Me-Leu]E(1c)S |
| SEQ ID NO: 87 | [6-Aminohexanoyl](1c)*SC(2a)WQC(2a)[Y(2-aminoethoxy)][2-Nal][2-Me- |
| | Leu]E(1c)[{d}R] |
| SEQ ID NO: 88 | [6-Amino-4-oxahexanoyl](1c)*SC(2a)WQC(2a)[Y(2-aminoethoxy)][2- |
| | Nal][2-Me-Leu]E(1c)[{d}R] |
| SEQ ID NO: 89 | [trans-4-Aminomethyl-cyclohexyl-1-carbonyl](1c)*[beta-homo- |
| | Ser]C(2a)WQC(2a)[Y(2-aminoethoxy)][2-Nal][2-Me-Leu]E(1c)[{d}R] |
| SEQ ID NO: 90 | [(4-(2-Aminoethyl)-piperazine-1-yl)-acetyl](1c)*SC(2a)WQC(2a)[Y(2- |
| | aminoethoxy)][2-Nal][2-Me-Leu]E(1c)[{d}R] |
| SEQ ID NO: 91 | [(4-(2-Aminoethyl)-piperazine-1-yl)-acetyl](1c)*SC(2a)WQC(2a)[Y(2- |
| | aminoethoxy)][2-Nal][2-Me-Leu]D(1c)[{d}R] |
| SEQ ID NO: 92 | [3-Aminomethyl-phenylacetyl](1c)*SC(2a)WQC(2a)[Y(2-aminoethoxy)][2- |
| | Nal][2-Me-Leu]E(1c)[{d}R] |
| SEQ ID NO: 93 | [2-Aminomethyl-phenylacetyl](1c)*SC(2a)WQC(2a)[Y(2-aminoethoxy)][2- |
| | Nal][2-Me-Leu]E(1c)[{d}R] |
| SEQ ID NO: 94 | [K(N₃)](1g)SC(2a)WQC(2a)[Y(2-aminoethoxy)][2-Nal][2-Me- |
| | Leu][Pra](1g)[{d}R] |
| SEQ ID NO: 95 | [Azidoacetic acid](1g)*SC(2a)WQC(2a)[Y(2-aminoethoxy)][2-Nal][2-Me- |
| | Leu][Glu(propargylamine)](1g)[{d}R] |
| SEQ ID NO: 96 | [Azidoacetic acid](1g)*SC(2a)WQC(2a)[Y(2-aminoethoxy)][2-Nal][2-Me- |
| | Leu][Dab(3-butynoic acid)](1g)[{d}R] |
| SEQ ID NO: 97 | [but-3-ynoic acid](1g)*SC(2a)WQC(2a)[Y(2-aminoethoxy)][2-Nal][2-Me- |
| | Leu][Dab(azidoacetic acid)](1g)[{d}R] |
| SEQ ID NO: 98 | [(N₃)-Ala](1g)*SC(2a)WQC(2a)[Y(2-aminoethoxy)][2-Nal][2-Me- |
| | Leu][Glu(propargylamine)](1g)[{d}R] |
| SEQ ID NO: 99 | [(N₃)-Ala](1g)*SC(2a)WQC(2a)[Y(2-aminoethoxy)][2-Nal][2-Me- |
| | Leu][Dab(3-butynoic acid)](1g)[{d}R] |
| SEQ ID NO: 100 | [but-3-ynoic acid](1g)*SC(2a)WQC(2a)[Y(2-aminoethoxy)][2-Nal][2-Me- |
| | Leu][Dab((N₃)-Ala))](1g)[{d}R] |
| SEQ ID NO: 101 | [4-Aminomethyl-2-pyridineacetyl](1c)*SC(2a)WQC(2a)[Y(2- |
| | aminoethoxy)][2-Nal][2-Me-Leu]E(1c)[{d}R] |
| SEQ ID NO: 102 | [4-Aminomethyl-3-pyridineacetyl](1c)*SC(2a)WQC(2a)[Y(2- |
| | aminoethoxy)][2-Nal][2-Me-Leu]E(1c)[{d}R] |
| SEQ ID NO: 103 | [4-Aminomethyl-2-fluoro-phenylacetyl](1c)*SC(2a)WQC(2a)[Y(2- |
| | aminoethoxy)][2-Nal][2-Me-Leu]E(1c)[{d}R] |
| SEQ ID NO: 104 | [4-Aminomethyl-3-methyl-phenylacetyl](1c)*SC(2a)WQC(2a)[Y(2- |
| | aminoethoxy)][2-Nal][2-Me-Leu]E(1c)[{d}R] |
| SEQ ID NO: 105 | [4-Aminomethyl-3-methoxy-phenylacetyl](1c)*SC(2a)WQC(2a)[Y(2- |
| | aminoethoxy)][2-Nal][2-Me-Leu]E(1c)[{d}R] |

or a pharmaceutically acceptable salt or solvate thereof;
wherein:
*= bridge uses the peptide backbone amine or carboxylic acid at the N- or C-terminus,
not the side chain amine or carboxylic acid
(1a) = [2,11] 1,3-dithio-propan-2-one bridge; (1c) = [2,11] lactam bridge; (1g) = [2,11] 1,4-disubstituted 1,2,3-triazole bridge; (2a) = [4,7] 1,3-dithio-propan-2-one bridge; (2c) = [4,7] lactam bridge; (2d) = [4,7] 1,2-phenylenedimethanethiol bridge; (2e) = [4,7] 1,3-phenylenedimethanethiol bridge; (2f) = [4,7] 1,4-phenylenedimethanethiol bridge; (2g) = [4,7] 1,4-disubstituted 1,2,3-triazole bridge.

In some embodiments, Z is an amino acid sequence selected from the group consisting of:

| | |
|---|---|
| SEQ ID NO: 106 | [but-3-ynoic acid](1h)*SC(2a)WQC(2a)[Y(2-aminoethoxy)][2-Nal][2-Me- |
| | Leu][Dab(azidoacetic acid)](1h)[{d}R] |
| SEQ ID NO: 107 | [but-3-ynoic acid](1h)*SC(2a)WQC(2a)[Y(2-aminoethoxy)][2-Nal][2-Me- |
| | Leu][Dab((N₃)-Ala)](1h)[{d}R] |
| SEQ ID NO: 108 | [4-Aminomethyl-phenylacetyl](1c)*SC(2a)WQC(2a)[Y(2-aminoethoxy)][2- |
| | Nal][2-Me-Leu]E(1c)[{d}R] |
| SEQ ID NO: 109 | [4-Aminomethyl-phenylacetyl](1c)*SC(2a)WQC(2a)[Y(2-aminoethoxy)][2- |
| | Nal][2-Me-Leu]E(1c)[{d}2,4-Diaminobutanoyl([2-(trimethyl-2- |
| | aminoethoxy)ethoxy]propyl)] |
| SEQ ID NO: 110 | [4-Aminomethyl-phenylacetyl](1c)*SC(2a)WQC(2a)[Dab][2-Nal][2-Me- |
| | Leu]E(1c)[{d}R] |
| SEQ ID NO: 111 | [4-Aminomethyl-phenylacetyl](1c)*SC(2a)WQC(2a)[2,4- |
| | Diaminobutanoyl([2-(trimethyl-2-aminoethoxy)ethoxy]propyl)][2-Nal][2-Me- |
| | Leu]E(1c)[{d}R] |
| SEQ ID NO: 112 | [Dab](1c)*SC(2a)WQC(2a)[Y(2-aminoethoxy)][2-Nal][2-Me- |
| | Leu]E(1c)[{d}R] |
| SEQ ID NO: 113 | [2,4-Diaminobutanoy\|([2-(trimethyl-2- |
| | aminoethoxy)ethoxy]propyl)](1c)*SC(2a)WQC(2a)[Y(2-aminoethoxy)][2- |
| | Nal][2-Me-Leu]E(1c)[{d}R] |
| SEQ ID NO: 114 | [4-Aminomethyl-phenylacetyl](1c)*SC(2a)WQC(2a)[F(4-NH₂-(2-(trimethyl- |
| | 2-aminoethoxy)ethoxy)propyl)][2-Nal][2-Me-Leu]E(1c)[{d}R] |
| SEQ ID NO: 115 | [4-Aminomethyl-phenylacetyl](1c)*SC(2a)WQC(2a)[Y(2-aminoethoxy)][2- |
| | Nal][2-Me-Leu]E(1c)[{d}Q] |
| SEQ ID NO: 116 | [4-Aminomethyl-phenylacetyl](1c)*SC(2a)WQC(2a)[Y(2-aminoethoxy)][2- |
| | Nal][2-Me-Leu]E(1c)[{d}E] |
| SEQ ID NO: 117 | [4-Aminomethyl-phenylacetyl](1c)*SC(2a)WQC(2a)[Y(2-aminoethoxy)][2- |
| | Nal][2-Me-Leu]E(1c)[{d}H] |
| SEQ ID NO: 118 | [4-Aminomethyl-phenylacetyl](1c)*SC(2a)WQC(2a)[Y(2-aminoethoxy)][2- |
| | Nal][2-Me-Leu]E(1c)[3-Aminopropanoyl] |
| SEQ ID NO: 119 | [4-Aminomethyl-phenylacetyl](1c)*SC(2a)WQC(2a)[Y(2-aminoethoxy)][2- |
| | Nal][2-Me-Leu]E(1c)[3-Aminopropanoyl] |
| SEQ ID NO: 120 | [4-Aminomethyl-phenylacetyl](1c)*SC(2a)WQC(2a)[Y(2-aminoethoxy)][2- |
| | Nal][2-Me-Leu]E(1c)[Dab][3-(3-Pyridyl)-Ala] |
| SEQ ID NO: 121 | [4-Aminomethyl-phenylacetyl](1c)*SC(2a)WQC(2a)[Y(2-aminoethoxy)][6- |
| | F-Trp][2-Me-Leu]E(1c)[Dab][3-(3-Pyridyl)-Ala] |
| SEQ ID NO: 122 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala] |
| SEQ ID NO: 123 | [4-Aminomethyl-phenylacetyl](1c)*S[Dab](2c)WQE(2c)[Y(2- |
| | aminoethoxy)][2-Nal][2-Me-Leu]E(1c)[Dab][3-(3-Pyridyl)-Ala] |
| SEQ ID NO: 124 | [4-Aminomethyl-phenylacetyl](1c)*SE(2c)WQ[Orn](2c)[Y(2- |
| | aminoethoxy)][2-Nal][2-Me-Leu]E(1c)[Dab][3-(3-Pyridyl)-Ala] |
| SEQ ID NO: 125 | [4-Aminomethyl-phenylacetyl](1c)*SE(2c)WQ[Dab](2c)[Y(2- |
| | aminoethoxy)][2-Nal][2-Me-Leu]E(1c)[Dab] |
| SEQ ID NO: 126 | [4-Aminomethyl-phenylacetyl](1c)*VE(2c)WQ[Dab](2c)[Y(2- |
| | aminoethoxy][2-Nal][2-Me-Leu]E(1c)[Dab] |
| SEQ ID NO: 127 | [4-Aminomethyl-phenylacetyl](1c)*IE(2c)WQ[Dab](2c)[Y(2- |
| | aminoethoxy)][2-Nal][2-Me-Leu]E(1c)[Dab] |
| SEQ ID NO: 128 | [4-Aminomethyl-phenylacetyl](1c)*SE(2c)WV[Dab](2c)[Y(2- |
| | aminoethoxy)][2-Nal][2-Me-Leu]E(1c)[Dab] |
| SEQ ID NO: 129 | [4-Aminomethyl-phenylacetyl](1c)*SE(2c)WH[Dab](2c)[Y(2- |
| | aminoethoxy)][2-Nal][2-Me-Leu]E(1c)[Dab] |
| SEQ ID NO: 130 | [4-Aminomethyl-phenylacetyl](1c)*SE(2c)WQ[Dab](2c)F[2-Nal][2-Me- |
| | Leu]E(1c)[Dab] |
| SEQ ID NO: 131 | [4-Aminomethyl-phenylacetyl](1c)*SE(2c)WQ[Dab](2c)V[2-Nal][2-Me- |
| | Leu]E(1c)[Dab] |
| SEQ ID NO: 132 | [4-Aminomethyl-phenylacetyl](1c)*SE(2c)WQ[Dab](2c)[F(4-Me)][2-Nal][2- |
| | Me-Leu]E(1c)[Dab] |
| SEQ ID NO: 133 | [4-Aminomethyl-phenylacetyl](1c)*SE(2c)WQ[Dab](2c)[Y(2- |
| | aminoethoxy)]H[2-Me-Leu]E(1c)[Dab] |
| SEQ ID NO: 134 | [4-Aminomethyl-phenylacetyl](1c)*SE(2c)WQ[Dab](2c)[Y(2- |
| | aminoethoxy)][F(3-F)][2-Me-Leu]E(1c)[Dab] |
| SEQ ID NO: 135 | [4-Aminomethyl-phenylacetyl](1c)*SE(2c)WQ[Dab](2c)[Y(2- |
| | aminoethoxy)][F(3,4-Me)][2-Me-Leu]E(1c)[Dab] |
| SEQ ID NO: 136 | [4-Aminomethyl-phenylacetyl](1c)*SE(2c)WQ[Dab](2c)[Y(2- |
| | aminoethoxy)][2-Nal][2-Me-Val]E(1c)[Dab] |
| SEQ ID NO: 137 | [4-Aminomethyl-phenylacetyl](1c)*SE(2c)WQ[Dab](2c)[Y(2- |
| | aminoethoxy)][2-Nal][2-Me-Leu]E(1c) |
| SEQ ID NO: 138 | [4-Aminomethyl-phenylacetyl](1c)*SE(2c)[4-F-Trp]Q[Dab](2c)[Y(2- |
| | aminoethoxy)][2-Nal][2-Me-Leu]E(1c)[Dab] |
| SEQ ID NO: 139 | [4-Aminomethyl-phenylacetyl](1c)*SE(2c)[7-F-Trp]Q[Dab](2c)[Y(2- |
| | aminoethoxy)][2-Nal][2-Me-Leu]E(1c)[Dab] |
| SEQ ID NO: 140 | [4-Methylaminomethyl-phenylacetyl](1c)*SC(2a)WQC(2a)[Y(2- |
| | aminoethoxy)][2-Nal][2-Me-Leu]E(1c)[Dab] |
| SEQ ID NO: 141 | [4-Aminomethyl-phenylacetyl](1c)*[N-Me-Ser]C(2a)WQC(2a)[Y(2- |
| | aminoethoxy)][2-Nal][2-Me-Leu]E(1c)[Dab] |
| SEQ ID NO: 142 | [4-Aminomethyl-phenylacetyl](1c)*S[N-Me-Cys](2a)WQC(2a)[Y(2- |
| | aminoethoxy)][2-Nal][2-Me-Leu]E(1c)[Dab] |
| SEQ ID NO: 143 | [4-Aminomethyl-phenylacetyl](1c)*SC(2a)[N-Me-Trp]QC(2a)[Y(2- |
| | aminoethoxy)][2-Nal][2-Me-Leu]E(1c)[Dab] |
| SEQ ID NO: 144 | [4-Aminomethyl-phenylacetyl](1c)*SC(2a)W[N-Me-Gln]C(2a)[Y(2- |
| | aminoethoxy)][2-Nal][2-Me-Leu]E(1c)[Dab] |
| SEQ ID NO: 145 | [4-Aminomethyl-phenylacetyl](1c)*[Q(pyrrolidin)]E(2c)WQ[Dab](2c)[Y(2- |
| | aminoethoxy)][2-Nal][2-Me-Leu]E(1c)[Dab] |
| SEQ ID NO: 146 | [4-Aminomethyl-phenylacetyl](1c)*SE(2c)W[Q(pyrrolidin)][Dab](2c)[Y(2- |
| | aminoethoxy)][2-Nal][2-Me-Leu]E(1c)[Dab] |
| SEQ ID NO: 147 | [4-Aminomethyl-phenylacetyl](1c)*SC(2a)WQC(2a)[Y(2-aminoethoxy)][2- |
| | Nal][2-Me-Leu]E(1c)R[3-(3-Pyridyl)-Ala] |
| SEQ ID NO: 148 | [(3-Aminomethyl)benzoyl](1c)*SC(2a)WQC(2a)[Y(2-aminoethoxy)][2- |
| | Nal][2-Me-Leu]E(1c)[Dab][3-(3-Pyridyl)-Ala] |
| SEQ ID NO: 149 | [(3-Aminomethyl)benzoyl](1c)*SC(2a)WQC(2a)][Y(2-aminoethoxy)][2- |
| | Nal][2-Me-Leu]E(1c)R[3-(3-Quinolinyl)-Ala] |
| SEQ ID NO: 150 | [(3-Aminomethyl)benzoyl](1c)*SC(2a)WQC(2a)][Y(2-aminoethoxy)][2- |
| | Nal][2-Me-Leu]E(1c)R[{d}[3-(3-Pyridyl)-Ala]] |
| SEQ ID NO: 151 | [(3-Aminomethyl)benzoyl](1c)*SC(2a)WQC(2a)][Y(2-aminoethoxy)][2- |
| | Nal][2-Me-Leu]E(1c)R[3-Amino-3-(3′-pyridyl)propionyl] |
| SEQ ID NO: 152 | [(3-Aminomethyl)benzoyl](1c)*SC(2a)WQC(2a)][Y(2-aminoethoxy)][2- |
| | Nal][2-Me-Leu]E(1c)R[F(3-F)] |
| SEQ ID NO: 153 | [(3-Aminomethyl)benzoyl](1c)*SC(2a)WQC(2a)][Y(2-aminoethoxy)][2- |
| | Nal][2-Me-Leu]E(1c)R[F(3,5-F)] |
| SEQ ID NO: 154 | [(3-Aminomethyl)benzoyl](1c)*SC(2a)WQC(2a)][Y(2-aminoethoxy)][2- |
| | Nal][2-Me-Leu]E(1c)R[4-Aminomethyl-2-pyridineacetyl] |
| SEQ ID NO: 155 | K(1c)TC(2a)WQC(2a)[Y(2-aminoethoxy)][2-Nal][2-Me-Leu]E(1c)[Dab][3- |
| | (3-Pyridyl)-Ala] |
| SEQ ID NO: 156 | [(3-Aminomethyl)benzoyl](1c)*IE(2c)WQ[Dab](2c)[Y(2-aminoethoxy)][2- |
| | Nal][2-Me-Val]E(1c)[Dab][3-(3-Pyridyl)-Ala] |
| SEQ ID NO: 157 | [4-Aminomethyl-phenylacetyl](1c)*IE(2c)WQ[Dab](2c)[Y(2- |
| | aminoethoxy)][2-Nal][2-Me-Val]E(1c)[Dab][3-(3-Pyridyl)-Ala] |
| SEQ ID NO: 158 | [4-Aminomethyl-phenylacetyl](1c)*IE(2c)WQ[Dab](2c)[Y(2- |
| | aminoethoxy)][F(3,4-Me)][2-Me-Val]E(1c)G[3-(3-Pyridyl)-Ala] |
| SEQ ID NO: 159 | [4-Aminomethyl-phenylacetyl](1c)*IE(2c)WQ[Dab](2c)[F(4-Me)][F(3,4- |
| | Me)][2-Me-Val]E(1c)G[3-(3-Pyridyl)-Ala] |
| SEQ ID NO: 160 | [4-Aminomethyl-phenylacetyl](1c)*IE(2c)WQ[Dab](2c)[F(4-Me)][F(3,4- |
| | Me)][2-Me-Val]E(1c)[GABA][3-(3-Pyridyl)-Ala] |
| SEQ ID NO: 161 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-pyridylacetyl)] |
| SEQ ID NO: 162 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-pyridylpropionyl)] |
| SEQ ID NO: 163 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-fluorobenzoyl)] |
| SEQ ID NO: 164 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-fluorophenylacetyl)] |
| SEQ ID NO: 165 | [4-Aminomethyl-phenylacetyl](1c)*IE(2c)WQ[Dab](2c)[Y(2- |
| | aminoethoxy)][F(3,4-Me)][2-Me-Val]E(1c)[Dab][2-Me-3-(3-Pyridyl)-Ala] |
| SEQ ID NO: 166 | [4-Aminomethyl-phenylacetyl](1c)*IE(2c)WQ[Dab](2c)[Y(Me)][F(3,4- |
| | Me)][2-Me-Val]E(1c)[Dab][2-Me-3-(3-Pyridyl)-Ala] |
| SEQ ID NO: 167 | [4-Aminomethyl-phenylacetyl](1c)*IE(2c)WQ[Dab](2c)[2-Me-Phe][F(3,4- |
| | Me)][2-Me-Val]E(1c)[Dab][2-Me-3-(3-Pyridyl)-Ala] |
| SEQ ID NO: 168 | [4-Aminomethyl-phenylacetyl](1c)*IE(2c)WQ[Dab](2c)[Bip][F(3,4-Me)][2- |
| | Me-Val]E(1c)[Dab][2-Me-3-(3-Pyridyl)-Ala] |
| SEQ ID NO: 169 | [4-Aminomethyl-phenylacetyl](1c)*IE(2c)WQ[Dab](2c)[2-Me-F(4-F)][F(3,4- |
| | Me)][2-Me-Val]E(1c)[Dab][2-Me-3-(3-Pyridyl)-Ala] |
| SEQ ID NO: 170 | [4-Aminomethyl-phenylacetyl](1c)*IE(2c)WQ[Dab](2c)[{d}F(4-F)][F(3,4- |
| | Me)][2-Me-Val]E(1c)[Dab][2-Me-3-(3-Pyridyl)-Ala] |
| SEQ ID NO: 171 | [4-Aminomethyl-phenylacetyl](1c)*IE(2c)WQ[Dab](2c)[F(4-Cl)][F(3,4- |
| | Me)][2-Me-Val]E(1c)[Dab][2-Me-3-(3-Pyridyl)-Ala] |
| SEQ ID NO: 172 | [4-Aminomethyl-phenylacetyl](1c)*IE(2c)WQ[Dab](2c)[alpha-Me- |
| | Trp][F(3,4-Me)][2-Me-Val]E(1c)[Dab][2-Me-3-(3-Pyridyl)-Ala] |
| SEQ ID NO: 173 | [4-Aminomethyl-phenylacetyl](1c)*IE(2c)WQ[Dab](2c)[3,3-Diphenyl- |
| | Ala][F(3,4-Me)][2-Me-Val]E(1c)[Dab][2-Me-3-(3-Pyridyl)-Ala] |
| SEQ ID NO: 174 | [4-Aminomethyl-phenylacetyl](1c)*IE(2c)WQ[Dab](2c)[Phg][F(3,4-Me)][2- |
| | Me-Val]E(1c)[Dab][2-Me-3-(3-Pyridyl)-Ala] |
| SEQ ID NO: 175 | [4-Aminomethyl-phenylacetyl](1c)*IE(2c)WQ[Dab](2c)[F(4-Me)][1-Me- |
| | Trp][2-Me-Val]E(1c)[Dab][2-Me-3-(3-Pyridyl)-Ala] |
| SEQ ID NO: 176 | [4-Aminomethyl-phenylacetyl](1c)*IE(2c)WQ[Dab](2c)[F(4-Me)][Bip][2-Me- |
| | Val]E(1c)[Dab][2-Me-3-(3-Pyridyl)-Ala] |
| SEQ ID NO: 177 | [4-Aminomethyl-phenylacetyl](1c)*IE(2c)WQ[Dab](2c)[F(4-Me)][{d}6-F- |
| | Trp][2-Me-Val]E(1c)[Dab][2-Me-3-(3-Pyridyl)-Ala] |

or a pharmaceutically acceptable salt or solvate thereof;
wherein:
*= bridge uses the peptide backbone amine or carboxylic acid at the N- or C-terminus, not the side chain amine or carboxylic acid
(1a) = [2,11] 1,3-dithio-propan-2-one bridge; (1c) = [2,11] lactam bridge; (1g) = [2,11] 1,4-disubstituted 1,2,3-triazole bridge; (1h) = [2,11] 1,5-disubstituted 1,2,3-triazole bridge; (2a) = [4,7] 1,3-dithio-propan-2-one bridge; (2c) = [4,7] lactam bridge; (2d) = [4,7] 1,2-phenylenedimethanethiol bridge; (2e) = [4,7] 1,3-phenylenedimethanethiol bridge; (2f) = [4,7] 1,4-phenylenedimethanethiol bridge; (2g) = [4,7] 1,4-disubstituted 1,2,3-triazole bridge.

In some embodiments, Z is an amino acid sequence selected from the group consisting of:

| | |
|---|---|
| SEQ ID NO: 1 | DC(1a)SC(2a)WQC(2a)WW[2-Me-Leu]C(1a)R |
| SEQ ID NO: 2 | LC(1a)SC(2a)WQC(2a)WWLC(1a)R |
| SEQ ID NO: 3 | DC(1a)SC(2a)WEC(2a)WWLC(1a)R |
| SEQ ID NO: 4 | DE(1c)SC(2a)WQC(2a)WWLK(1c)R |
| SEQ ID NO: 5 | DC(1a)SE(2c)WQK(2c)WWLC(1a)R |
| SEQ ID NO: 6 | D(1c)*SC(2a)WQC(2a)WWLR(1c)* |
| SEQ ID NO: 9 | DE(1c)SC(2a)WQC(2a)WWL[Dab](1c)R |
| SEQ ID NO: 10 | E(1c)SC(2a)WQC(2a)WWLK(1c)R |
| SEQ ID NO: 11 | DE(1c)SC(2a)WQC(2a)WWLK(1c) |
| SEQ ID NO: 12 | E(1c)SC(2a)WQC(2a)WWLK(1c) |
| SEQ ID NO: 13 | DE(1c)SC(2a)AQC(2a)WWLK(1c)R |
| SEQ ID NO: 14 | DE(1c)SC(2a)WQC(2a)AWLK(1c)R |
| SEQ ID NO: 15 | DE(1c)SC(2a)WQC(2a)WALK(1c)R |
| SEQ ID NO: 16 | DE(1c)SC(2a)[7-Me-Trp]QC(2a)WWLK(1c)R |
| SEQ ID NO: 17 | DE(1c)SC(2a)WQC(2a)W[2-Nal]LK(1c)R |
| SEQ ID NO: 18 | K(1c)SC(2a)WQC(2a)W[2-Nal]LE(1c)R |
| SEQ ID NO: 19 | DK(1c)SC(2a)WQC(2a)W[2-Nal]LE(1c)R |
| SEQ ID NO: 20 | DK(1c)SC(2d)WQC(2d)W[2-Nal]LE(1c)R |
| SEQ ID NO: 21 | DK(1c)SC(2e)WQC(2e)W[2-Nal]LE(1c)R |
| SEQ ID NO: 22 | DK(1c)SC(2f)WQC(2f)W[2-Nal]LE(1c)R |
| SEQ ID NO: 23 | DK(1c)S[K(N₃)](2g)WQ[Pra](2g)W[2-Nal]LE(1c)R |
| SEQ ID NO: 24 | DK(1c)S[Aha](2g)WQ[Pra](2g)W[2-Nal]LE(1c)R |
| SEQ ID NO: 25 | K(1c)SC(2a)[2-Nal]QC(2a)W[2-Nal]LE(1c)R |
| SEQ ID NO: 26 | K(1c)SC(2a)[Bip]QC(2a)W[2-Nal]LE(1c)R |
| SEQ ID NO: 27 | K(1c)SC(2a)[1-Me-Trp]QC(2a)W[2-Nal]LE(1c)R |
| SEQ ID NO: 28 | DK(1c)SC(2a)WQC(2a)[2-Nal][2-Nal]LE(1c)R |
| SEQ ID NO: 29 | K(1c)SC(2a)WQC(2a)[Y(2-aminoethoxy)][2-Nal]LE(1c)R |
| SEQ ID NO: 30 | K(1c)SC(2a)WQC(2a)H[2-Nal]LE(1c)R |
| SEQ ID NO: 31 | K(1c)SC(2a)WQC(2a)W[F(4-F)]LE(1c)R |
| SEQ ID NO: 32 | K(1c)SC(2a)WQC(2a)W[1-Me-Trp]LE(1c)R |
| SEQ ID NO: 33 | C(1a)TC(2a)WEC(2a)WW[2-Me-Leu]C(1a)S |
| SEQ ID NO: 34 | GK(1c)TC(2a)WEC(2c)WW[2-Me-Leu]E(1c)S |
| SEQ ID NO: 35 | GC(1a)TC(2a)WEC(2a)W[2-Nal][2-Me-Leu]C(1a)R |
| SEQ ID NO: 36 | [bAla](1c)*SC(2a)WQC(2a)W[2-Nal]LE(1c)R |
| SEQ ID NO: 37 | [(3-Aminomethyl)benzoyl](1c)*SC(2a)WQC(2a)W[2-Nal]LE(1c)R |
| SEQ ID NO: 38 | [(3-Aminomethyl)benzoyl](1c)*SC(2a)WQC(2a)[Y(2-aminoethoxy)][2- |
| | Nal][2-Me-Leu]E(1c)R-[NH2] |
| SEQ ID NO: 39 | [(3-Aminomethyl)benzoyl](1c)*SC(2a)WQC(2a)[Y(2-aminoethoxy)][2- |
| | Nal]LE(1c)R |
| SEQ ID NO: 40 | [(3-Aminomethyl)benzoyl](1c)*SC(2a)WQC(2a)W[2-Nal][2-Me-Leu]E(1c)R |
| SEQ ID NO: 41 | [(3-Aminomethyl)benzoyl](1c)*SC(2a)WQC(2a)[Y(2-aminoethoxy)][2- |
| | Nal][2-Me-Leu]E(1c)[{d}R] |
| SEQ ID NO: 42 | [(3-Aminomethyl)benzoyl](1c)*SC(2a)WQC(2a)[Y(2-aminoethoxy)][2- |
| | Nal][2-Me-Leu]E(1c)[2-Me-Arg] |
| SEQ ID NO: 43 | [(3-Aminomethyl)benzoyl](1c)*SC(2a)WQC(2a)[Y(2-aminoethoxy)][2- |
| | Nal][2-Me-Leu]E(1c)[N-Me-Arg] |
| SEQ ID NO: 44 | [(3-Aminomethyl)benzoyl](1c)*SC(2a)WQC(2a)[Y(2-aminoethoxy)][2- |
| | Nal][2-Me-Leu]E(1c)S |
| SEQ ID NO: 45 | [(3-Aminomethyl)benzoyl](1c)*SC(2a)WQC(2a)[Y(2-aminoethoxy)][2- |
| | Nal][2-Me-Leu]E(1c)[F(4-NH2)] |
| SEQ ID NO: 46 | [(3-Aminomethyl)benzoyl](1c)*SC(2a)WQC(2a)[Y(2-aminoethoxy)][2- |
| | Nal][{d}L]E(1c)Y |
| SEQ ID NO: 47 | [Ac]-K(1c)SC(2a)WQC(2a)[Y(2-aminoethoxy)][2-Nal][2-Me-Leu]E(1c)R |
| SEQ ID NO: 48 | [Ac]-K(1c)SC(2a)WQC(2a)[Y(2-aminoethoxy)][2-Nal][{d}L]E(1c)R |
| SEQ ID NO: 49 | [(4-Aminomethyl)benzoyl](1c)*SC(2a)WQC(2a)[Y(2-aminoethoxy)][2- |
| | Nal][2-Me-Leu]E(1c)R |
| SEQ ID NO: 50 | [4-(2-Aminoethyl)benzoyl](1c)*SC(2a)WQC(2a)[Y(2-aminoethoxy)][2- |
| | Nal][2-Me-Leu]E(1c)R |
| SEQ ID NO: 51 | [3-(4-Aminophenyl)propanoyl](1c)*SC(2a)WQC(2a)[Y(2-aminoethoxy)][2- |
| | Nal][2-Me-Leu]E(1c)R |
| SEQ ID NO: 52 | [4-Aminomethyl-phenylacetyl](1c)*SC(2a)WQC(2a)[Y(2-aminoethoxy)][2- |
| | Nal][2-Me-Leu]E(1c)R |
| SEQ ID NO: 53 | DC(1a)SC(2b)WQC(2b)WWLC(1a)R |
| SEQ ID NO: 54 | K(1c)SC(2b)WQC(2b)[Y(2-aminoethoxy)][2-Nal]LE(1c)R |
| SEQ ID NO: 55 | DE(1c)QC(2a)WQC(2a)YW[2-Me-Leu]K(1c)R |
| SEQ ID NO: 56 | DE(1c)QC(2a)WQC(2a)[2-Nal]W[2-Me-Leu]K(1c)R |
| SEQ ID NO: 57 | DE(1c)TC(2a)WQC(2a)W[2-Nal][2-Me-Leu]K(1c) |
| SEQ ID NO: 58 | DE(1c)SC(2a)WQC(2a)W[2-Nal][2-Me-Leu]K(1c)ENG |
| SEQ ID NO: 59 | DE(1c)SC(2a)WQC(2a)W[2-Nal][2-Me-Leu]K(1c)NG |
| SEQ ID NO: 60 | DE(1c)SC(2a)WQC(2a)W[2-Nal][2-Me-Leu]K(1c)G |
| SEQ ID NO: 61 | DE(1c)SC(2a)WQC(2a)W[2-Nal][2-Me-Lys]K(1c)ENG |
| SEQ ID NO: 62 | DC(1a)QC(2a)WQC(2a)[2-Nal]W[2-Me-Leu]C(1a)R |
| SEQ ID NO: 63 | DC(1a)QC(2a)WQC(2a)YW[2-Me-Leu]C(1a)R |
| SEQ ID NO: 64 | DC(1a)QC(2a)WQC(2a)WW[2-Me-Leu]C(1a)ENG |

or a pharmaceutically acceptable salt or solvate thereof;
wherein:
*= bridge uses the peptide backbone amine or carboxylic acid at the N- or C-terminus, not the side chain amine or carboxylic acid
(1a) = [2,11] 1,3-dithio-propan-2-one bridge; (1c) = [2,11] lactam bridge; (1g) = [2,11] 1,4-disubstituted 1,2,3-triazole bridge; (2a) = [4,7] 1,3-dithio-propan-2-one bridge; (2c) = [4,7] lactam bridge; (2d) = [4,7] 1,2-phenylenedimethanethiol bridge; (2e) = [4,7] 1,3-phenylenedimethanethiol bridge; (2f) = [4,7] 1,4-phenylenedimethanethiol bridge; (2g) = [4,7] 1,4-disubstituted 1,2,3-triazole bridge.

In some embodiments, Z is an amino acid sequence selected from the group consisting of:

| | |
|---|---|
| SEQ ID NO: 67 | [(3-Aminomethyl)benzoyl](1c)*SE(2c)WQ[Dab](2c)[Y(2-aminoethoxy)][2- |
| | Nal][2-Me-Leu]E(1c)R |
| SEQ ID NO: 68 | [(3-Aminomethyl)benzoyl](1c)*S[Orn](2c)WQE(2c)[Y(2-aminoethoxy)][2- |
| | Nal][2-Me-Leu]E(1c)R |
| SEQ ID NO: 69 | [(3-Aminomethyl)benzoyl](1c)*SE(2c)WQ[Orn](2c)[Y(2-aminoethoxy)][2- |
| | Nal][2-Me-Leu]E(1c)R |
| SEQ ID NO: 70 | [(3-Aminomethyl)benzoyl](1c)*SC(2a)[7-Me-Trp]QC(2a)[Y(2- |
| | aminoethoxy)][2-Nal][2-Me-Leu]E(1c)R |
| SEQ ID NO: 78 | [(3-Aminomethyl)benzoyl](1c)*SC(2a)WQC(2a)[Y(2-aminoethoxy)][2- |
| | Nal][2-Me-Leu]E(1c)R[3-(3-Pyridyl)-Ala] |
| SEQ ID NO: 79 | [(3-Aminomethyl)benzoyl](1c)*SC(2a)WQC(2a)[Y(2-aminoethoxy)][2- |
| | Nal][2-Me-Leu]E(1c)R[3-(4-Pyridyl)-Ala] |
| SEQ ID NO: 80 | [4-Aminomethyl-phenylacetyl](1c)*SC(2a)WQC(2a)[Y(2-aminoethoxy)][2- |
| | Nal][2-Me-Leu]E(1c)[{d}R] |
| SEQ ID NO: 81 | [4-Aminomethyl-phenylacetyl](1c)*SC(2a)WQC(2a)[Y(2-aminoethoxy)][2- |
| | Nal][2-Me-Leu]E(1c)S |
| SEQ ID NO: 82 | [4-Aminomethyl-phenylacetyl](1c)*SC(2a)WQC(2a)[Y(2-aminoethoxy)][2- |
| | Nal][2-Me-Leu]E(1c)[Dab] |
| SEQ ID NO: 84 | [Ac]-K(1c)SC(2a)WQC(2a)[Y(2-aminoethoxy)][2-Nal][2-Me-Leu]E(1c)[{d}R] |
| SEQ ID NO: 85 | [Ac]-K(1c)TC(2a)WQC(2a)[Y(2-aminoethoxy)][2-Nal][2-Me-Leu]E(1c)[{d}R] |
| SEQ ID NO: 87 | [6-Aminohexanoyl](1c)*SC(2a)WQC(2a)[Y(2-aminoethoxy)][2-Nal][2-Me- |
| | Leu]E(1c)[{d}R] |
| SEQ ID NO: 90 | [(4-(2-Aminoethyl)-piperazine-1-yl)-acetyl](1c)*SC(2a)WQC(2a)[Y(2- |
| | aminoethoxy)][2-Nal][2-Me-Leu]E(1c)[{d}R] |
| SEQ ID NO: 92 | [3-Aminomethyl-phenylacetyl](1c)*SC(2a)WQC(2a)[Y(2-aminoethoxy)][2- |
| | Nal][2-Me-Leu]E(1c)[{d}R] |

or a pharmaceutically acceptable salt or solvate thereof;
wherein:
*= bridge uses the peptide backbone amine or carboxylic acid at the N- or C-terminus, not the side chain amine or carboxylic acid
(1a) = [2,11] 1,3-dithio-propan-2-one bridge; (1c) = [2,11] lactam bridge; (1g) = [2,11] 1,4-disubstituted 1,2,3-triazole bridge; (2a) = [4,7] 1,3-dithio-propan-2-one bridge; (2c) = [4,7] lactam bridge; (2d) = [4,7] 1,2-phenylenedimethanethiol bridge; (2e) = [4,7] 1,3-phenylenedimethanethiol bridge; (2f) = [4,7] 1,4-phenylenedimethanethiol bridge; (2g) = [4,7] 1,4-disubstituted 1,2,3-triazole bridge.

In some embodiments, Z is an amino acid sequence selected from the group consisting of:

| | |
|---|---|
| SEQ ID NO: 108 | [4-Aminomethyl-phenylacetyl](1c)*SC(2a)WQC(2a)[Y(2-aminoethoxy)][2- |
| | Nal][2-Me-Leu]E(1c)[{d}R] |
| SEQ ID NO: 109 | [4-Aminomethyl-phenylacetyl](1c)*SC(2a)WQC(2a)[Y(2-aminoethoxy)][2- |
| | Nal][2-Me-Leu]E(1c)[{d}2,4-Diaminobutanoyl([2-(trimethyl-2- |
| | aminoethoxy)ethoxy]propyl)] |
| SEQ ID NO: 115 | [4-Aminomethyl-phenylacetyl](1c)*SC(2a)WQC(2a)[Y(2-aminoethoxy)][2- |
| | Nal][2-Me-Leu]E(1c)[{d}Q] |
| SEQ ID NO: 117 | [4-Aminomethyl-phenylacetyl](1c)*SC(2a)WQC(2a)[Y(2-aminoethoxy)][2- |
| | Nal][2-Me-Leu]E(1c)[{d}H] |
| SEQ ID NO: 119 | [4-Aminomethyl-phenylacetyl](1c)*SC(2a)WQC(2a)[Y(2-aminoethoxy)][2- |
| | Nal][2-Me-Leu]E(1c)[3-Aminopropanoy]] |
| SEQ ID NO: 120 | [4-Aminomethyl-phenylacetyl](1c)*SC(2a)WQC(2a)[Y(2-aminoethoxy)][2- |
| | Nal][2-Me-Leu]E(1c)[Dab][3-(3-Pyridyl)-Ala] |
| SEQ ID NO: 121 | [4-Aminomethyl-phenylacetyl](1c)*SC(2a)WQC(2a)[Y(2-aminoethoxy)][6- |
| | F-Trp][2-Me-Leu]E(1c)[Dab][3-(3-Pyridyl)-Ala] |
| SEQ ID NO: 122 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala] |
| SEQ ID NO: 123 | [4-Aminomethyl-phenylacetyl](1c)*S[Dab](2c)WQE(2c)[Y(2- |
| | aminoethoxy)][2-Nal][2-Me-Leu]E(1c)[Dab][3-(3-Pyridyl)-Ala] |
| SEQ ID NO: 124 | [4-Aminomethyl-phenylacetyl](1c)*SE(2c)WQ[Orn](2c)[Y(2- |
| | aminoethoxy)][2-Nal][2-Me-Leu]E(1c)[Dab][3-(3-Pyridyl)-Ala] |
| SEQ ID NO: 125 | [4-Aminomethyl-phenylacetyl](1c)*SE(2c)WQ[Dab](2c)[Y(2- |
| | aminoethoxy)][2-Nal][2-Me-Leu]E(1c)[Dab] |
| SEQ ID NO: 126 | [4-Aminomethyl-phenylacetyl](1c)*VE(2c)WQ[Dab](2c)[Y(2- |
| | aminoethoxy][2-Nal][2-Me-Leu]E(1c)[Dab] |
| SEQ ID NO: 130 | [4-Aminomethyl-phenylacetyl](1c)*SE(2c)WQ[Dab](2c)F[2-Nal][2-Me- |
| | Leu]E(1c)[Dab] |
| SEQ ID NO: 136 | [4-Aminomethyl-phenylacetyl](1c)*SE(2c)WQ[Dab](2c)[Y(2- |
| | aminoethoxy)][2-Nal][2-Me-Val]E(1c)[Dab] |
| SEQ ID NO: 137 | [4-Aminomethyl-phenylacetyl](1c)*SE(2c)WQ[Dab](2c)[Y(2- |
| | aminoethoxy)][2-Nal][2-Me-Leu]E(1c) |
| SEQ ID NO: 138 | [4-Aminomethyl-phenylacetyl](1c)*SE(2c)[4-F-Trp]Q[Dab](2c)[Y(2- |
| | aminoethoxy)][2-Nal][2-Me-Leu]E(1c)[Dab] |
| SEQ ID NO: 139 | [4-Aminomethyl-phenylacetyl](1c)*SE(2c)[7-F-Trp]Q[Dab](2c)[Y(2- |
| | aminoethoxy)][2-Nal][2-Me-Leu]E(1c)[Dab] |
| SEQ ID NO: 145 | [4-Aminomethyl-phenylacetyl](1c)*[Q(pyrrolidin)]E(2c)WQ[Dab](2c)[Y(2- |
| | aminoethoxy)][2-Nal][2-Me-Leu]E(1c)[Dab] |
| SEQ ID NO: 146 | [4-Aminomethyl-phenylacetyl](1c)*SE(2c)W[Q(pyrrolidin)][Dab](2c)[Y(2- |
| | aminoethoxy)][2-Nal][2-Me-Leu]E(1c)[Dab] |
| SEQ ID NO: 147 | [4-Aminomethyl-phenylacetyl](1c)*SC(2a)WQC(2a)[Y(2-aminoethoxy)][2- |
| | Nal][2-Me-Leu]E(1c)R[3-(3-Pyridyl)-Ala] |
| SEQ ID NO: 148 | [(3-Aminomethyl)benzoyl](1c)*SC(2a)WQC(2a)[Y(2-aminoethoxy)][2- |
| | Nal][2-Me-Leu]E(1c)[Dab][3-(3-Pyridyl)-Ala] |
| SEQ ID NO: 149 | [(3-Aminomethyl)benzoyl](1c)*SC(2a)WQC(2a)][Y(2-aminoethoxy)][2- |
| | Nal][2-Me-Leu]E(1c)R[3-(3-Quinolinyl)-Ala] |
| SEQ ID NO: 150 | [(3-Aminomethyl)benzoyl](1c)*SC(2a)WQC(2a)][Y(2-aminoethoxy)][2- |
| | Nal][2-Me-Leu]E(1c)R[{d}[3-(3-Pyridyl)-Ala]] |
| SEQ ID NO: 151 | [(3-Aminomethyl)benzoyl](1c)*SC(2a)WQC(2a)][Y(2-aminoethoxy)][2- |
| | Nal][2-Me-Leu]E(1c)R[3-Amino-3-(3′-pyridyl)propionyl] |
| SEQ ID NO: 155 | K(1c)TC(2a)WQC(2a)[Y(2-aminoethoxy)][2-Nal][2-Me-Leu]E(1c)[Dab][3- |
| | (3-Pyridyl)-Ala] |
| SEQ ID NO: 156 | [(3-Aminomethyl)benzoyl](1c)*IE(2c)WQ[Dab](2c)[Y(2-aminoethoxy)][2- |
| | Nal][2-Me-Val]E(1c)[Dab][3-(3-Pyridyl)-Ala] |
| SEQ ID NO: 157 | [4-Aminomethyl-phenylacetyl](1c)*IE(2c)WQ[Dab](2c)[Y(2- |
| | aminoethoxy)][2-Nal][2-Me-Val]E(1c)[Dab][3-(3-Pyridyl)-Ala] |
| SEQ ID NO: 158 | [4-Aminomethyl-phenylacetyl](1c)*IE(2c)WQ[Dab](2c)[Y(2- |
| | aminoethoxy)][F(3,4-Me)][2-Me-Val]E(1c)G[3-(3-Pyridyl)-Ala] |
| SEQ ID NO: 165 | [4-Aminomethyl-phenylacetyl](1c)*IE(2c)WQ[Dab](2c)[Y(2- |
| | aminoethoxy)][F(3,4-Me)][2-Me-Val]E(1c)[Dab][2-Me-3-(3-Pyridyl)-Ala] |
| SEQ ID NO: 166 | [4-Aminomethyl-phenylacetyl](1c)*IE(2c)WQ[Dab](2c)[Y(Me)][F(3,4- |
| | Me)][2-Me-Val]E(1c)[Dab][2-Me-3-(3-Pyridyl)-Ala] |
| SEQ ID NO: 168 | [4-Aminomethyl-phenylacetyl](1c)*IE(2c)WQ[Dab](2c)[Bip][F(3,4-Me)][2- |
| | Me-Val]E(1c)[Dab][2-Me-3-(3-Pyridyl)-Ala] |
| SEQ ID NO: 169 | [4-Aminomethyl-phenylacetyl](1c)*IE(2c)WQ[Dab](2c)[2-Me-F(4-F)][F(3,4- |
| | Me)][2-Me-Val]E(1c)[Dab][2-Me-3-(3-Pyridyl)-Ala] |
| SEQ ID NO: 170 | [4-Aminomethyl-phenylacetyl](1c)*IE(2c)WQ[Dab](2c)[{d}F(4-F)][F(3,4- |
| | Me)][2-Me-Val]E(1c)[Dab][2-Me-3-(3-Pyridyl)-Ala] |
| SEQ ID NO: 171 | [4-Aminomethyl-phenylacetyl](1c)*IE(2c)WQ[Dab](2c)[F(4-Cl)][F(3,4- |
| | Me)][2-Me-Val]E(1c)[Dab][2-Me-3-(3-Pyridyl)-Ala] |
| SEQ ID NO: 172 | [4-Aminomethyl-phenylacetyl](1c)*IE(2c)WQ[Dab](2c)[alpha-Me- |
| | Trp][F(3,4-Me)][2-Me-Val]E(1c)[Dab][2-Me-3-(3-Pyridyl)-Ala] |
| SEQ ID NO: 173 | [4-Aminomethyl-phenylacetyl](1c)*IE(2c)WQ[Dab](2c)[3,3-Diphenyl- |
| | Ala][F(3,4-Me)][2-Me-Val]E(1c)[Dab][2-Me-3-(3-Pyridyl)-Ala] |
| SEQ ID NO: 174 | [4-Aminomethyl-phenylacetyl](1c)*IE(2c)WQ[Dab](2c)[Phg][F(3,4-Me)][2- |
| | Me-Val]E(1c)[Dab][2-Me-3-(3-Pyridyl)-Ala] |
| SEQ ID NO: 175 | [4-Aminomethyl-phenylacetyl](1c)*IE(2c)WQ[Dab](2c)[F(4-Me)][1-Me- |
| | Trp][2-Me-Val]E(1c)[Dab][2-Me-3-(3-Pyridyl)-Ala] |
| SEQ ID NO: 176 | [4-Aminomethyl-phenylacetyl](1c)*IE(2c)WQ[Dab](2c)[F(4-Me)][Bip][2-Me- |
| | Val]E(1c)[Dab][2-Me-3-(3-Pyridyl)-Ala] |
| SEQ ID NO: 177 | [4-Aminomethyl-phenylacetyl](1c)*IE(2c)WQ[Dab](2c)[F(4-Me)][{d}6-F- |
| | Trp][2-Me-Val]E(1c)[Dab][2-Me-3-(3-Pyridyl)-Ala] |

or a pharmaceutically acceptable salt or solvate thereof;
wherein:
*= bridge uses the peptide backbone amine or carboxylic acid at the N- or C-terminus, not the side chain amine or carboxylic acid
(1a) = [2,11] 1,3-dithio-propan-2-one bridge; (1c) = [2,11] lactam bridge; (1g) = [2,11] 1,4-disubstituted 1,2,3-triazole bridge; (1h) = [2,11] 1,5-disubstituted 1,2,3-triazole bridge; (2a) = [4,7] 1,3-dithio-propan-2-one bridge; (2c) = [4,7] lactam bridge; (2d) = [4,7] 1,2-phenylenedimethanethiol bridge; (2e) = [4,7] 1,3-phenylenedimethanethiol bridge; (2f) = [4,7] 1,4-phenylenedimethanethiol bridge; (2g) = [4,7] 1,4-disubstituted 1,2,3-triazole bridge.

Specific compounds of the invention include:

| Compound No. | Sequence |
|---|---|
| 1 | H-DC(1a)SC(2a)WQC(2a)WW[2-Me-Leu]C(1a)R-[NH2] |
| 2 | H-LC(1a)SC(2a)WQC(2a)WWLC(1a)R-[NH2] |
| 3 | H-DC(1a)SC(2a)WEC(2a)WWLC(1a)R-[NH2] |
| 4 | H-DE(1c)SC(2a)WQC(2a)WWLK(1c)R-[NH2] |
| 5 | H-DC(1a)SE(2c)WQK(2c)WWLC(1a)R-[NH2] |
| 6 | D(1c)*SC(2a)WQC(2a)WWLR(1c)* |
| 7 | [Ac]-DK(1c)SC(2a)WQC(2a)WWLE(1c)R-[NH2] |
| 8 | [Ac]-D[Orn](1c)SC(2a)WQC(2a)WWLE(1c)R-[NH2] |
| 9 | H-DE(1c)SC(2a)WQC(2a)WWL[Dab](1c)R-[NH2] |
| 10 | [Ac]-E(1c)SC(2a)WQC(2a)WWLK(1c)R-[NH2] |
| 11 | [Ac]-DE(1c)SC(2a)WQC(2a)WWLK(1c)-[NH2] |
| 12 | [Ac]-E(1c)SC(2a)WQC(2a)WWLK(1c)-[NH2] |
| 13 | [Ac]-DE(1c)SC(2a)AQC(2a)WWLK(1c)R-[NH2] |
| 14 | [Ac]-DE(1c)SC(2a)WQC(2a)AWLK(1c)R-[NH2] |
| 15 | [Ac]-DE(1c)SC(2a)WQC(2a)WALK(1c)R-[NH2] |
| 16 | [Ac]-DE(1c)SC(2a)[7-Me-Trp]QC(2a)WWLK(1c)R-[NH2] |
| 17 | [Ac]-DE(1c)SC(2a)WQC(2a)W[2-Nal]LK(1c)R-[NH2] |
| 18 | [Ac]-K(1c)SC(2a)WQC(2a)W[2-Nal]LE(1c)R-[NH2] |
| 19 | [Ac]-DK(1c)SC(2a)WQC(2a)W[2-Nal]LE(1c)R-[NH2] |
| 20 | [Ac]-DK(1c)SC(2d)WQC(2d)W[2-Nal]LE(1c)R-[NH2] |
| 21 | [Ac]-DK(1c)SC(2e)WQC(2e)W[2-Nal]LE(1c)R-[NH2] |
| 22 | [Ac]-DK(1c)SC(2f)WQC(2f)W[2-Nal]LE(1c)R-[NH2] |
| 23 | [Ac]-DK(1c)S[K(N₃)](2g)WQ[Pra](2g)W[2-Nal]LE(1c)R-[NH2] |
| 24 | [Ac]-DK(1c)S[Aha](2g)WQ[Pra](2g)W[2-Nal]LE(1c)R-[NH2] |
| 25 | [Ac]-K(1c)SC(2a)[2-Nal]QC(2a)W[2-Nal]LE(1c)R-[NH2] |
| 26 | [Ac]-K(1c)SC(2a)[Bip]QC(2a)W[2-Nal]LE(1c)R-[NH2] |
| 27 | [Ac]-K(1c)SC(2a)[1-Me-Trp]QC(2a)W[2-Nal]LE(1c)R-[NH2] |
| 28 | [Ac]-DK(1c)SC(2a)WQC(2a)[2-Nal][2-Nal]LE(1c)R-[NH2] |
| 29 | [Ac]-K(1c)SC(2a)WQC(2a)[Y(2-aminoethoxy)][2-Nal]LE(1c)R-[NH2] |
| 30 | [Ac]-K(1c)SC(2a)WQC(2a)H[2-Nal]LE(1c)R-[NH2] |
| 31 | [Ac]-K(1c)SC(2a)WQC(2a)W[F(4-F)]LE(1c)R-[NH2] |
| 32 | [Ac]-K(1c)SC(2a)WQC(2a)W[1-Me-Trp]LE(1c)R-[NH2] |
| 33 | [Ac]-C(1a)TC(2a)WEC(2a)WW[2-Me-Leu]C(1a)S-[NH2] |
| 34 | [Ac]-GK(1c)TC(2a)WEC(2c)WW[2-Me-Leu]E(1c)S-[NH2] |
| 35 | [Ac]-GC(1a)TC(2a)WEC(2a)W[2-Nal][2-Me-Leu]C(1a)R-[NH2] |
| 36 | [bAla](1c)*SC(2a)WQC(2a)W[2-Nal]LE(1c)R-[NH2] |
| 37 | [(3-Aminomethyl)benzoyl](1c)*SC(2a)WQC(2a)W[2-Nal]LE(1c)R-[NH2] |
| 38 | [(3-Aminomethyl)benzoyl](1c)*SC(2a)WQC(2a)[Y(2-aminoethoxy)][2- |
| | Nal][2-Me-Leu]E(1c)R-[NH2] |
| 39 | [(3-Aminomethyl)benzoyl](1c)*SC(2a)WQC(2a)[Y(2-aminoethoxy)][2- |
| | Nal]LE(1c)R-[NH2] |
| 40 | [(3-Aminomethyl)benzoyl](1c)*SC(2a)WQC(2a)W[2-Nal][2-Me- |
| | Leu]E(1c)R-[NH2] |
| 41 | [(3-Aminomethyl)benzoyl](1c)*SC(2a)WQC(2a)[Y(2-aminoethoxy)][2- |
| | Nal][2-Me-Leu]E(1c)[{d}R]-[NH2] |
| 42 | [(3-Aminomethyl)benzoyl](1c)*SC(2a)WQC(2a)[Y(2-aminoethoxy)][2- |
| | Nal][2-Me-Leu]E(1c)[2-Me-Arg]-[NH2] |
| 43 | [(3-Aminomethyl)benzoyl](1c)*SC(2a)WQC(2a)[Y(2-aminoethoxy)][2- |
| | Nal][2-Me-Leu]E(1c)[N-Me-Arg]-[NH2] |
| 44 | [(3-Aminomethyl)benzoyl](1c)*SC(2a)WQC(2a)[Y(2-aminoethoxy)][2- |
| | Nal][2-Me-Leu]E(1c)S-[NH2] |
| 45 | [(3-Aminomethyl)benzoyl](1c)*SC(2a)WQC(2a)[Y(2-aminoethoxy)][2- |
| | Nal][2-Me-Leu]E(1c)[F(4-NH₂)]-[NH2] |
| 46 | [(3-Aminomethyl)benzoyl](1c)*SC(2a)WQC(2a)[Y(2-aminoethoxy)][2- |
| | Nal][{d}L]E(1c)Y-[NH2] |
| 47 | [Ac]-K(1c)SC(2a)WQC(2a)[Y(2-aminoethoxy)][2-Nal][2-Me-Leu]E(1c)R- |
| | [NH2] |
| 48 | [Ac]-K(1c)SC(2a)WQC(2a)[Y(2-aminoethoxy)][2-Nal][{d}L]E(1c)R-[NH2] |
| 49 | [(4-Aminomethyl)benzoyl](1c)*SC(2a)WQC(2a)[Y(2-aminoethoxy)][2- |
| | Nal][2-Me-Leu]E(1c)R-[NH2] |
| 50 | [4-(2-Aminoethyl)benzoyl](1c)*SC(2a)WQC(2a)[Y(2-aminoethoxy)][2- |
| | Nal][2-Me-Leu]E(1c)R-[NH2] |
| 51 | [3-(4-Aminophenyl)propanoyl](1c)*SC(2a)WQC(2a)[Y(2-aminoethoxy)][2- |
| | Nal][2-Me-Leu]E(1c)R-[NH2] |
| 52 | [4-Aminomethyl-phenylacetyl](1c)*SC(2a)WQC(2a)[Y(2-aminoethoxy)][2- |
| | Nal][2-Me-Leu]E(1c)R-[NH2] |
| 53 | H-DC(1a)SC(2b)WQC(2b)WWLC(1a)R-[NH2] |
| 54 | [Ac]-K(1c)SC(2b)WQC(2b)[Y(2-aminoethoxy)][2-Nal]LE(1c)R-[NH2] |
| 55 | [Ac]-DE(1c)QC(2a)WQC(2a)YW[2-Me-Leu]K(1c)R-[NH2] |
| 56 | [Ac]-DE(1c)QC(2a)WQC(2a)[2-Nal]W[2-Me-Leu]K(1c)R-[NH2] |
| 57 | [Ac]-DE(1c)TC(2a)WQC(2a)W[2-Nal][2-Me-Leu]K(1c)R-[NH2] |
| 58 | [Ac]-DE(1c)SC(2a)WQC(2a)W[2-Nal][2-Me-Leu]K(1c)ENG-[NH2] |
| 59 | [Ac]-DE(1c)SC(2a)WQC(2a)W[2-Nal][2-Me-Leu]K(1c)NG-[NH2] |
| 60 | [Ac]-DE(1c)SC(2a)WQC(2a)W[2-Nal][2-Me-Leu]K(1c)G-[NH2] |
| 61 | [Ac]-DE(1c)SC(2a)WQC(2a)W[2-Nal][2-Me-Lys]K(1c)ENG-[NH2] |
| 62 | [Ac]-DC(1a)QC(2a)WQC(2a)[2-Nal]W[2-Me-Leu]C(1a)R-[NH2] |
| 63 | [Ac]-DC(1a)QC(2a)WQC(2a)YW[2-Me-Leu]C(1a)R-[NH2] |
| 64 | [Ac]-DC(1a)QC(2a)WQC(2a)WW[2-Me-Leu]C(1a)ENG-[NH2] |
| 65 | [(3-Aminomethyl)benzoyl](1c)*S[Dpr](2c)WQE(2c)[Y(2-aminoethoxy)][2- |
| | Nal][2-Me-Leu]E(1c)R-[NH2] |
| 66 | [(3-Aminomethyl)benzoyl](1c)*SE(2c)WQ[Dpr](2c)[Y(2-aminoethoxy)][2- |
| | Nal][2-Me-Leu]E(1c)R-[NH2] |
| 67 | [(3-Aminomethyl)benzoyl](1c)*SE(2c)WQ[Dab](2c)[Y(2-aminoethoxy)][2- |
| | Nal][2-Me-Leu]E(1c)R-[NH2] |
| 68 | [(3-Aminomethyl)benzoyl](1c)*S[Orn](2c)WQE(2c)[Y(2-aminoethoxy)][2- |
| | Nal][2-Me-Leu]E(1c)R-[NH2] |
| 69 | [(3-Aminomethyl)benzoyl](1c)*SE(2c)WQ[Orn](2c)[Y(2-aminoethoxy)][2- |
| | Nal][2-Me-Leu]E(1c)R-[NH2] |
| 70 | [(3-Aminomethyl)benzoyl](1c)*SC(2a)[7-Me-Trp]QC(2a)[Y(2- |
| | aminoethoxy)][2-Nal][2-Me-Leu]E(1c)R-[NH2] |
| 71 | [(3-Aminomethyl)benzoyl](1c)*SC(2a)[7-Ph-Trp]QC(2a)[Y(2- |
| | aminoethoxy)][2-Nal][2-Me-Leu]E(1c)R-[NH2] |
| 72 | [(3-Aminomethyl)benzoyl](1c)*SC(2a)[7-(Naphth-2-yl)-Trp]QC(2a)[Y(2- |
| | aminoethoxy)][2-Nal][2-Me-Leu]E(1c)R-[NH2] |
| 73 | [(3-Aminomethyl)benzoyl](1c)*SC(2a)WQC(2a)[Y(2-aminoethoxy)][3-(3- |
| | Quinolinyl)-Ala][2-Me-Leu]E(1c)R-[NH2] |
| 74 | [(3-Aminomethyl)benzoyl](1c)*SC(2a)WQC(2a)[Y(2-aminoethoxy)][6-Cl- |
| | Trp][2-Me-Leu]E(1c)R-[NH2] |
| 75 | [(3-Aminomethyl)benzoyl](1c)*SC(2a)WQC(2a)[Y(2-aminoethoxy)][2- |
| | Nal][4-aminotetrahydro-2H-pyran-4-acetyl]E(1c)R-[NH2] |
| 76 | [(3-Aminomethyl)benzoyl](1c)*SC(2a)WQC(2a)[Y(2-aminoethoxy)][2- |
| | Nal][2-Me-Leu]E(1c)[3-(3-Pyridyl)-Ala]-[NH2] |
| 77 | [(3-Aminomethyl)benzoyl](1c)*SC(2a)WQC(2a)[Y(2-aminoethoxy)][2- |
| | Nal][2-Me-Leu]E(1c)[3-(4-Pyridyl)-Ala]-[NH2] |
| 78 | [(3-Aminomethyl)benzoyl](1c)*SC(2a)WQC(2a)[Y(2-aminoethoxy)][2- |
| | Nal][2-Me-Leu]E(1c)R[3-(3-Pyridyl)-Ala]-[NH2] |
| 79 | [(3-Aminomethyl)benzoyl](1c)*SC(2a)WQC(2a)[Y(2-aminoethoxy)][2- |
| | Nal][2-Me-Leu]E(1c)R[3-(4-Pyridyl)-Ala]-[NH2] |
| 80 | [4-Aminomethyl-phenylacetyl](1c)*SC(2a)WQC(2a)[Y(2-aminoethoxy)][2- |
| | Nal][2-Me-Leu]E(1c)[{d}R]-[NH2] |
| 81 | [4-Aminomethyl-phenylacetyl](1c)*SC(2a)WQC(2a)[Y(2-aminoethoxy)][2- |
| | Nal][2-Me-Leu]E(1c)S-[NH2] |
| 82 | [4-Aminomethyl-phenylacetyl](1c)*SC(2a)WQC(2a)[Y(2-aminoethoxy)][2- |
| | Nal][2-Me-Leu]E(1c)[Dab]-[NH2] |
| 83 | [4-Aminomethyl-phenylacetyl](1c)*TC(2a)WQC(2a)[Y(2-aminoethoxy)][2- |
| | Nal][2-Me-Leu]E(1c)[{d}R]-[NH2] |
| 84 | [Ac]-K(1c)SC(2a)WQC(2a)[Y(2-aminoethoxy)][2-Nal][2-Me- |
| | Leu]E(1c)[{d}R]-[NH2] |
| 85 | [Ac]-K(1c)TC(2a)WQC(2a)[Y(2-aminoethoxy)][2-Nal][2-Me- |
| | Leu]E(1c)[{d}R]-[NH2] |
| 86 | [Ac]-K(1c)SC(2a)WQC(2a)[Y(2-aminoethoxy)][2-Nal][2-Me-Leu]E(1c)S-[NH2] |
| 87 | [6-Aminohexanoyl](1c)*SC(2a)WQC(2a)[Y(2-aminoethoxy)][2-Nal][2-Me- |
| | Leu]E(1c)[{d}R]-[NH2] |
| 88 | [6-Amino-4-oxahexanoyl](1c)*SC(2a)WQC(2a)[Y(2-aminoethoxy)][2- |
| | Nal][2-Me-Leu]E(1c)[{d}R]-[NH2] |
| 89 | [trans-4-Aminomethyl-cyclohexyl-1-carbonyl](1c)*[beta-homo- |
| | Ser]C(2a)WQC(2a)[Y(2-aminoethoxy)][2-Nal][2-Me-Leu]E(1c)[{d}R]-[NH2] |
| 90 | [(4-(2-Aminoethyl)-piperazine-1-yl)-acetyl](1c)*SC(2a)WQC(2a)[Y(2- |
| | aminoethoxy)][2-Nal][2-Me-Leu]E(1c)[{d}R]-[NH2] |
| 91 | [(4-(2-Aminoethyl)-piperazine-1-yl)-acetyl](1c)*SC(2a)WQC(2a)[Y(2- |
| | aminoethoxy)][2-Nal][2-Me-Leu]D(1c)[{d}R]-[NH2] |
| 92 | [3-Aminomethyl-phenylacetyl](1c)*SC(2a)WQC(2a)[Y(2-aminoethoxy)][2- |
| | Nal][2-Me-Leu]E(1c)[{d}R]-[NH2] |
| 93 | [2-Aminomethyl-phenylacetyl](1c)*SC(2a)WQC(2a)[Y(2-aminoethoxy)][2- |
| | Nal][2-Me-Leu]E(1c)[{d}R]-[NH2] |
| 94 | [Ac]-[K(N₃)](1g)SC(2a)WQC(2a)[Y(2-aminoethoxy)][2-Nal][2-Me- |
| | Leu][Pra](1g)[{d}R]-[NH2] |
| 95 | [Azidoacetic acid](1g)*SC(2a)WQC(2a)[Y(2-aminoethoxy)][2-Nal][2-Me- |
| | Leu][Glu(propargylamine)](1g)[{d}R]-[NH2] |
| 96 | [Azidoacetic acid](1g)*SC(2a)WQC(2a)[Y(2-aminoethoxy)][2-Nal][2-Me- |
| | Leu][Dab(3-butynoic acid)](1g)[{d}R]-[NH2] |
| 97 | [but-3-ynoic acid](1g)*SC(2a)WQC(2a)[Y(2-aminoethoxy)][2-Nal][2-Me- |
| | Leu][Dab(azidoacetic acid)](1g)[{d}R]-[NH2] |
| 98 | [(N₃)-Ala](1g)*SC(2a)WQC(2a)[Y(2-aminoethoxy)][2-Nal][2-Me- |
| | Leu][Glu(propargylamine)](1g)[{d}R]-[NH2] |
| 99 | [(N₃)-Ala](1g)*SC(2a)WQC(2a)[Y(2-aminoethoxy)][2-Nal][2-Me- |
| | Leu][Dab(3-butynoic acid)](1g)[{d}R]-[NH2] |
| 100 | [but-3-ynoic acid](1g)*SC(2a)WQC(2a)[Y(2-aminoethoxy)][2-Nal][2-Me- |
| | Leu][Dab((N₃)-Ala))](1g)[{d}R]-[NH2] |
| 101 | [4-Aminomethyl-2-pyridineacetyl](1c)*SC(2a)WQC(2a)[Y(2- |
| | aminoethoxy)][2-Nal][2-Me-Leu]E(1c)[{d}R]-[NH2] |
| 102 | [4-Aminomethyl-3-pyridineacetyl](1c)*SC(2a)WQC(2a)[Y(2- |
| | aminoethoxy)][2-Nal][2-Me-Leu]E(1c)[{d}R]-[NH2] |
| 103 | [4-Aminomethyl-2-fluoro-phenylacetyl](1c)*SC(2a)WQC(2a)[Y(2- |
| | aminoethoxy)][2-Nal][2-Me-Leu]E(1c)[{d}R]-[NH2] |
| 104 | [4-Aminomethyl-3-methyl-phenylacetyl](1c)*SC(2a)WQC(2a)[Y(2- |
| | aminoethoxy)][2-Nal][2-Me-Leu]E(1c)[{d}R]-[NH2] |
| 105 | [4-Aminomethyl-3-methoxy-phenylacetyl](1c)*SC(2a)WQC(2a)[Y(2- |
| | aminoethoxy)][2-Nal][2-Me-Leu]E(1c)[{d}R]-[NH2] |

and pharmaceutically acceptable salts and solvates thereof;
wherein:
*= bridge uses the peptide backbone amine or carboxylic acid at the N- or C-terminus, not the side chain amine or carboxylic acid
(1a) = [2,11] 1,3-dithio-propan-2-one bridge; (1c) = [2,11] lactam bridge; (1g) = [2,11] 1,4-disubstituted 1,2,3-triazole bridge; (2a) = [4,7] 1,3-dithio-propan-2-one bridge; (2c) = [4,7] lactam bridge; (2d) = [4,7] 1,2-phenylenedimethanethiol bridge; (2e) = [4,7] 1,3-phenylenedimethanethiol bridge; (2f) = [4,7] 1,4-phenylenedimethanethiol bridge; (2g) = [4,7] 1,4-disubstituted 1,2,3-triazole bridge.

Other specific compounds of the invention include:

| Compound No. | Sequence |
|---|---|
| 106 | [but-3-ynoic acid](1h)*SC(2a)WQC(2a)[Y(2-aminoethoxy)][2-Nal][2-Me- |
| | Leu][Dab(azidoacetic acid)](1h)[{d}R]-[NH2] |
| 107 | [but-3-ynoic acid](1h)*SC(2a)WQC(2a)[Y(2-aminoethoxy)][2-Nal][2-Me-Leu][Dab((N₃)- |
| | Ala))](1h)[{d}R]-[NH2] |
| 108 | [4-Aminomethyl-phenylacetyl](1c)*SC(2a)WQC(2a)[Y(2-aminoethoxy)][2-Nal][2-Me- |
| | Leu]E(1c)[{d}R]-[OH] |
| 109 | [4-Aminomethyl-phenylacetyl](1c)*SC(2a)WQC(2a)[Y(2-aminoethoxy)][2-Nal][2-Me- |
| | Leu]E(1c)[{d}2,4-Diaminobutanoyl([2-(trimethyl-2-aminoethoxy)ethoxy]propyl)]-[NH2] |
| 110 | [4-Aminomethyl-phenylacetyl](1c)*SC(2a)WQC(2a)[Dab][2-Nal][2-Me- |
| | Leu]E(1c)[{d}R]-[NH2] |
| 111 | [4-Aminomethyl-phenylacetyl](1c)*SC(2a)WQC(2a)[2,4-Diaminobutanoyl([2- |
| | (trimethyl-2-aminoethoxy)ethoxy]propyl)][2-Nal][2-Me-Leu]E(1c)[{d}R]-[NH2] |
| 112 | [Dab](1c)*SC(2a)WQC(2a)[Y(2-aminoethoxy)][2-Nal][2-Me-Leu]E(1c)[{d}R]-[NH2] |
| 113 | [2,4-Diaminobutanoyl([2-(trimethyl-2- |
| | aminoethoxy)ethoxy]propyl)](1c)*SC(2a)WQC(2a)[Y(2-aminoethoxy)][2-Nal][2-Me- |
| | Leu]E(1c)[{d}R]-[NH2] |
| 114 | [4-Aminomethyl-phenylacetyl](1c)*SC(2a)WQC(2a)[F(4-NH2-(2-(trimethyl-2- |
| | aminoethoxy)ethoxy)propyl)][2-Nal][2-Me-Leu]E(1c)[{d}R]-[NH2] |
| 115 | [4-Aminomethyl-phenylacetyl](1c)*SC(2a)WQC(2a)[Y(2-aminoethoxy)][2-Nal][2-Me- |
| | Leu]E(1c)[{d}Q]-[NH2] |
| 116 | [4-Aminomethyl-phenylacetyl](1c)*SC(2a)WQC(2a)[Y(2-aminoethoxy)][2-Nal][2-Me- |
| | Leu]E(1c)[{d}E]-[NH2] |
| 117 | [4-Aminomethyl-phenylacetyl](1c)*SC(2a)WQC(2a)[Y(2-aminoethoxy)][2-Nal][2-Me- |
| | Leu]E(1c)[{d}H]-[NH2] |
| 118 | [4-Aminomethyl-phenylacetyl](1c)*SC(2a)WQC(2a)[Y(2-aminoethoxy)][2-Nal][2-Me- |
| | Leu]E(1c)[3-Aminopropanoy[]-[OH] |
| 119 | [4-Aminomethyl-phenylacetyl](1c)*SC(2a)WQC(2a)[Y(2-aminoethoxy)][2-Nal][2-Me- |
| | Leu]E(1c)[3-Aminopropanoyl]-[NH2] |
| 120 | [4-Aminomethyl-phenylacetyl](1c)*SC(2a)WQC(2a)[Y(2-aminoethoxy)][2-Nal][2-Me- |
| | Leu]E(1c)[Dab][3-(3-Pyridyl)-Ala]-[NH2] |
| 121 | [4-Aminomethyl-phenylacetyl](1c)*SC(2a)WQC(2a)[Y(2-aminoethoxy)][6-F-Trp][2-Me- |
| | Leu]E(1c)[Dab][3-(3-Pyridyl)-Ala]-[NH2] |
| 122 | [4-Aminomethyl-phenylacetyl](1c)*SE(2c)WQ[Dab](2c)[Y(2-aminoethoxy)][2-Nal][2- |
| | Me-Leu]E(1c)[Dab][3-(3-Pyridyl)-Ala]-[NH2] |
| 123 | [4-Aminomethyl-phenylacetyl](1c)*S[Dab](2c)WQE(2c)[Y(2-aminoethoxy)][2-Nal][2- |
| | Me-Leu]E(1c)[Dab][3-(3-Pyridyl)-Ala]-[NH2] |
| 124 | [4-Aminomethyl-phenylacetyl](1c)*SE(2c)WQ[Orn](2c)[Y(2-aminoethoxy)][2-Nal][2- |
| | Me-Leu]E(1c)[Dab][3-(3-Pyridyl)-Ala]-[NH2] |
| 125 | [4-Aminomethyl-phenylacetyl](1c)*SE(2c)WQ[Dab](2c)[Y(2-aminoethoxy)][2-Nal][2- |
| | Me-Leu]E(1c)[Dab]-[NH2] |
| 126 | [4-Aminomethyl-phenylacetyl](1c)*VE(2c)WQ[Dab](2c)[Y(2-aminoethoxy][2-Nal][2- |
| | Me-Leu]E(1c)[Dab]-[NH2] |
| 127 | [4-Aminomethyl-phenylacetyl](1c)*IE(2c)WQ[Dab](2c)[Y(2-aminoethoxy)][2-Nal][2- |
| | Me-Leu]E(1c)[Dab]-[NH2] |
| 128 | [4-Aminomethyl-phenylacetyl](1c)*SE(2c)WV[Dab](2c)[Y(2-aminoethoxy)][2-Nal][2- |
| | Me-Leu]E(1c)[Dab]-[NH2] |
| 129 | [4-Aminomethyl-phenylacetyl](1c)*SE(2c)WH[Dab](2c)[Y(2-aminoethoxy)][2-Nal][2- |
| | Me-Leu]E(1c)[Dab]-[NH2] |
| 130 | [4-Aminomethyl-phenylacetyl](1c)*SE(2c)WQ[Dab](2c)F[2-Nal][2-Me-Leu]E(1c)[Dab]-[NH2] |
| 131 | [4-Aminomethyl-phenylacetyl](1c)*SE(2c)WQ[Dab](2c)V[2-Nal][2-Me-Leu]E(1c)[Dab]-[NH2] |
| 132 | [4-Aminomethyl-phenylacetyl](1c)*SE(2c)WQ[Dab](2c)[F(4-Me)][2-Nal][2-Me- |
| | Leu]E(1c)[Dab]-[NH2] |
| 133 | [4-Aminomethyl-phenylacetyl](1c)*SE(2c)WQ[Dab](2c)[Y(2-aminoethoxy)]H[2-Me- |
| | Leu]E(1c)[Dab]-[NH2] |
| 134 | [4-Aminomethyl-phenylacetyl](1c)*SE(2c)WQ[Dab](2c)[Y(2-aminoethoxy)][F(3-F)][2- |
| | Me-Leu]E(1c)[Dab]-[NH2] |
| 135 | [4-Aminomethyl-phenylacetyl](1c)*SE(2c)WQ[Dab](2c)[Y(2-aminoethoxy)][F(3,4- |
| | Me)][2-Me-Leu]E(1c)[Dab]-[NH2] |
| 136 | [4-Aminomethyl-phenylacetyl](1c)*SE(2c)WQ[Dab](2c)[Y(2-aminoethoxy)][2-Nal][2- |
| | Me-Val]E(1c)[Dab]-[NH2] |
| 137 | [4-Aminomethyl-phenylacetyl](1c)*SE(2c)WQ[Dab](2c)[Y(2-aminoethoxy)][2-Nal][2- |
| | Me-Leu]E(1c)-[NH-3-aminopropanoyl] |
| 138 | [4-Aminomethyl-phenylacetyl](1c)*SE(2c)[4-F-Trp]Q[Dab](2c)[Y(2-aminoethoxy)][2- |
| | Nal][2-Me-Leu]E(1c)[Dab]-[NH2] |
| 139 | [4-Aminomethyl-phenylacetyl](1c)*SE(2c)[7-F-Trp]Q[Dab](2c)[Y(2-aminoethoxy)][2- |
| | Nal][2-Me-Leu]E(1c)[Dab]-[NH2] |
| 140 | [4-Methylaminomethyl-phenylacetyl](1c)*SC(2a)WQC(2a)[Y(2-aminoethoxy)][2- |
| | Nal][2-Me-Leu]E(1c)[Dab]-[NH2] |
| 141 | [4-Aminomethyl-phenylacetyl](1c)*[N-Me-Ser]C(2a)WQC(2a)[Y(2-aminoethoxy)][2- |
| | Nal][2-Me-Leu]E(1c)[Dab]-[NH2] |
| 142 | [4-Aminomethyl-phenylacetyl](1c)*S[N-Me-Cys](2a)WQC(2a)[Y(2-aminoethoxy)][2- |
| | Nal][2-Me-Leu]E(1c)[Dab]-[NH2] |
| 143 | [4-Aminomethyl-phenylacetyl](1c)*SC(2a)[N-Me-Trp]QC(2a)[Y(2-aminoethoxy)][2- |
| | Nal][2-Me-Leu]E(1c)[Dab]-[NH2] |
| 144 | [4-Aminomethyl-phenylacetyl](1c)*SC(2a)W[N-Me-Gln]C(2a)[Y(2-aminoethoxy)][2- |
| | Nal][2-Me-Leu]E(1c)[Dab]-[NH2] |
| 145 | [4-Aminomethyl-phenylacetyl](1c)*[Q(pyrrolidin)]E(2c)WQ[Dab](2c)[Y(2- |
| | aminoethoxy)][2-Nal][2-Me-Leu]E(1c)[Dab]-[NH2] |
| 146 | [4-Aminomethyl-phenylacetyl](1c)*SE(2c)W[Q(pyrrolidin)][Dab](2c)[Y(2- |
| | aminoethoxy)][2-Nal][2-Me-Leu]E(1c)[Dab]-[NH2] |
| 147 | [4-Aminomethyl-phenylacetyl](1c)*SC(2a)WQC(2a)[Y(2-aminoethoxy)][2-Nal][2-Me- |
| | Leu]E(1c)R[3-(3-Pyridyl)-Ala]-[NH2] |
| 148 | [(3-Aminomethyl)benzoyl](1c)*SC(2a)WQC(2a)[Y(2-aminoethoxy)][2-Nal][2-Me- |
| | Leu]E(1c)[Dab][3-(3-Pyridyl)-Ala]-[NH2] |
| 149 | [(3-Aminomethyl)benzoyl](1c)*SC(2a)WQC(2a)][Y(2-aminoethoxy)][2-Nal][2-Me- |
| | Leu]E(1c)R[3-(3-Quinolinyl)-Ala]-[NH2] |
| 150 | [(3-Aminomethyl)benzoyl](1c)*SC(2a)WQC(2a)][Y(2-aminoethoxy)][2-Nal][2-Me- |
| | Leu]E(1c)R[{d}[3-(3-Pyridyl)-Ala]]-[NH2] |
| 151 | [(3-Aminomethyl)benzoyl](1c)*SC(2a)WQC(2a)][Y(2-aminoethoxy)][2-Nal][2-Me- |
| | Leu]E(1c)R[3-Amino-3-(3′-pyridyl)propionyl]-[NH2] |
| 152 | [(3-Aminomethyl)benzoyl](1c)*SC(2a)WQC(2a)][Y(2-aminoethoxy)][2-Nal][2-Me- |
| | Leu]E(1c)R[F(3-F)]-[NH2] |
| 153 | [(3-Aminomethyl)benzoyl](1c)*SC(2a)WQC(2a)][Y(2-aminoethoxy)][2-Nal][2-Me- |
| | Leu]E(1c)R[F(3,5-F)]-[NH2] |
| 154 | [(3-Aminomethyl)benzoyl](1c)*SC(2a)WQC(2a)][Y(2-aminoethoxy)][2-Nal][2-Me- |
| | Leu]E(1c)R[4-Aminomethyl-2-pyridineacetyl]-[NH2] |
| 155 | [Ac]-K(1c)TC(2a)WQC(2a)[Y(2-aminoethoxy)][2-Nal][2-Me-Leu]E(1c)[Dab][3-(3- |
| | Pyridyl)-Ala]-[NH2] |
| 156 | [(3-Aminomethyl)benzoyl](1c)*IE(2c)WQ[Dab](2c)[Y(2-aminoethoxy)][2-Nal][2-Me- |
| | Val]E(1c)[Dab][3-(3-Pyridyl)-Ala]-[NH2] |
| 157 | [4-Aminomethyl-phenylacetyl](1c)*IE(2c)WQ[Dab](2c)[Y(2-aminoethoxy)][2-Nal][2- |
| | Me-Val]E(1c)[Dab][3-(3-Pyridyl)-Ala]-[NH2] |
| 158 | [4-Aminomethyl-phenylacetyl](1c)*IE(2c)WQ[Dab](2c)[Y(2-aminoethoxy)][F(3,4- |
| | Me)][2-Me-Val]E(1c)G[3-(3-Pyridyl)-Ala]-[NH2] |
| 159 | [4-Aminomethyl-phenylacetyl](1c)*IE(2c)WQ[Dab](2c)[F(4-Me)][F(3,4-Me)][2-Me- |
| | Val]E(1c)G[3-(3-Pyridyl)-Ala]-[NH2] |
| 160 | [4-Aminomethyl-phenylacetyl](1c)*IE(2c)WQ[Dab](2c)[F(4-Me)][F(3,4-Me)][2-Me- |
| | Val]E(1c)[GABA][3-(3-Pyridyl)-Ala]-[NH2] |
| 161 | [4-Aminomethyl-phenylacetyl](1c)*IE(2c)WQ[Dab](2c)[F(4-Me)][F(3,4-Me)][2-Me- |
| | Val]E(1c)G[2,3-Diaminopropanoyl(3-pyridylacetyl)]-[NH2] |
| 162 | [4-Aminomethyl-phenylacetyl](1c)*IE(2c)WQ[Dab](2c)[F(4-Me)][F(3,4-Me)][2-Me- |
| | Val]E(1c)G[2,3-Diaminopropanoyl(3-pyridylpropionyl)]-[NH2] |
| 163 | [4-Aminomethyl-phenylacetyl](1c)*IE(2c)WQ[Dab](2c)[F(4-Me)][F(3,4-Me)][2-Me- |
| | Val]E(1c)G[2,3-Diaminopropanoyl(3-fluorobenzoyl)]-[NH2] |
| 164 | [4-Aminomethyl-phenylacetyl](1c)*IE(2c)WQ[Dab](2c)[F(4-Me)][F(3,4-Me)][2-Me- |
| | Val]E(1c)G[2,3-Diaminopropanoyl(3-fluorophenylacetyl)]-[NH2] |
| 165 | [4-Aminomethyl-phenylacetyl](1c)*IE(2c)WQ[Dab](2c)[Y(2-aminoethoxy)][F(3,4- |
| | Me)][2-Me-Val]E(1c)[Dab][2-Me-3-(3-Pyridyl)-Ala]-[NH2] |
| 166 | [4-Aminomethyl-phenylacetyl](1c)*IE(2c)WQ[Dab](2c)[Y(Me)][F(3,4-Me)][2-Me- |
| | Val]E(1c)[Dab][2-Me-3-(3-Pyridyl)-Ala]-[NH2] |
| 167 | [4-Aminomethyl-phenylacetyl](1c)*IE(2c)WQ[Dab](2c)[2-Me-Phe][F(3,4-Me)][2-Me- |
| | Val]E(1c)[Dab][2-Me-3-(3-Pyridyl)-Ala]-[NH2] |
| 168 | [4-Aminomethyl-phenylacetyl](1c)*IE(2c)WQ[Dab](2c)[Bip][F(3,4-Me)][2-Me- |
| | Val]E(1c)[Dab][2-Me-3-(3-Pyridyl)-Ala]-[NH2] |
| 169 | [4-Aminomethyl-phenylacetyl](1c)*IE(2c)WQ[Dab](2c)[2-Me-F(4-F)][F(3,4-Me)][2-Me- |
| | Val]E(1c)[Dab][2-Me-3-(3-Pyridyl)-Ala]-[NH2] |
| 170 | [4-Aminomethyl-phenylacetyl](1c)*IE(2c)WQ[Dab](2c)[{d}F(4-F)][F(3,4-Me)][2-Me- |
| | Val]E(1c)[Dab][2-Me-3-(3-Pyridyl)-Ala]-[NH2] |
| 171 | [4-Aminomethyl-phenylacetyl](1c)*IE(2c)WQ[Dab](2c)[F(4-Cl)][F(3,4-Me)][2-Me- |
| | Val]E(1c)[Dab][2-Me-3-(3-Pyridyl)-Ala]-[NH2] |
| 172 | [4-Aminomethyl-phenylacetyl](1c)*IE(2c)WQ[Dab](2c)[alpha-Me-Trp][F(3,4-Me)][2- |
| | Me-Val]E(1c)[Dab][2-Me-3-(3-Pyridyl)-Ala]-[NH2] |
| 173 | [4-Aminomethyl-phenylacetyl](1c)*IE(2c)WQ[Dab](2c)[3,3-Diphenyl-Ala][F(3,4-Me)][2- |
| | Me-Val]E(1c)[Dab][2-Me-3-(3-Pyridyl)-Ala]-[NH2] |
| 174 | [4-Aminomethyl-phenylacetyl](1c)*IE(2c)WQ[Dab](2c)[Phg][F(3,4-Me)][2-Me- |
| | Val]E(1c)[Dab][2-Me-3-(3-Pyridyl)-Ala]-[NH2] |
| 175 | [4-Aminomethyl-phenylacetyl](1c)*IE(2c)WQ[Dab](2c)[F(4-Me)][1-Me-Trp][2-Me- |
| | Val]E(1c)[Dab][2-Me-3-(3-Pyridyl)-Ala]-[NH2] |
| 176 | [4-Aminomethyl-phenylacetyl](1c)*IE(2c)WQ[Dab](2c)[F(4-Me)][Bip][2-Me- |
| | Val]E(1c)[Dab][2-Me-3-(3-Pyridyl)-Ala]-[NH2] |
| 177 | [4-Aminomethyl-phenylacetyl](1c)*IE(2c)WQ[Dab](2c)[F(4-Me)][{d}6-F-Trp][2-Me- |
| | Val]E(1c)[Dab][2-Me-3-(3-Pyridyl)-Ala]-[NH2] |

and pharmaceutically acceptable salts and solvates thereof;
*= bridge uses the peptide backbone amine or carboxylic acid at the N- or C-terminus, not the side chain amine or carboxylic acid
(1a) = [2,11] 1,3-dithio-propan-2-one bridge; (1c) = [2,11] lactam bridge; (1g) = [2,11] 1,4-disubstituted 1,2,3-triazole bridge; (1h) = [2,11] 1,5-disubstituted 1,2,3-triazole bridge; (2a) = [4,7] 1,3-dithio-propan-2-one bridge; (2c) = [4,7] lactam bridge; (2d) = [4,7] 1,2-phenylenedimethanethiol bridge; (2e) = [4,7] 1,3-phenylenedimethanethiol bridge; (2f) = [4,7] 1,4-phenylenedimethanethiol bridge; (2g) = [4,7] 1,4-disubstituted 1,2,3-triazole bridge.

In some embodiments, specific compounds of the invention include:

| Compound No. | Sequence |
|---|---|
| 1 | H-DC(1a)SC(2a)WQC(2a)WW[2-Me-Leu]C(1a)R-[NH2] |
| 2 | H-LC(1a)SC(2a)WQC(2a)WWLC(1a)R-[NH2] |
| 3 | H-DC(1a)SC(2a)WEC(2a)WWLC(1a)R-[NH2] |
| 4 | H-DE(1c)SC(2a)WQC(2a)WWLK(1c)R-[NH2] |
| 5 | H-DC(1a)SE(2c)WQK(2c)WWLC(1a)R-[NH2] |
| 6 | D(1c)*SC(2a)WQC(2a)WWLR(1c)* |
| 9 | H-DE(1c)SC(2a)WQC(2a)WWL[Dab](1c)R-[NH2] |
| 10 | [Ac]-E(1c)SC(2a)WQC(2a)WWLK(1c)R-[NH2] |
| 11 | [Ac]-DE(1c)SC(2a)WQC(2a)WWLK(1c)-[NH2] |
| 12 | [Ac]-E(1c)SC(2a)WQC(2a)WWLK(1c)-[NH2] |
| 13 | [Ac]-DE(1c)SC(2a)AQC(2a)WWLK(1c)R-[NH2] |
| 14 | [Ac]-DE(1c)SC(2a)WQC(2a)AWLK(1c)R-[NH2] |
| 15 | [Ac]-DE(1c)SC(2a)WQC(2a)WALK(1c)R-[NH2] |
| 16 | [Ac]-DE(1c)SC(2a)[7-Me-Trp]QC(2a)WWLK(1c)R-[NH2] |
| 17 | [Ac]-DE(1c)SC(2a)WQC(2a)W[2-Nal]LK(1c)R-[NH2] |
| 18 | [Ac]-K(1c)SC(2a)WQC(2a)W[2-Nal]LE(1c)R-[NH2] |
| 19 | [Ac]-DK(1c)SC(2a)WQC(2a)W[2-Nal]LE(1c)R-[NH2] |
| 20 | [Ac]-DK(1c)SC(2d)WQC(2d)W[2-Nal]LE(1c)R-[NH2] |
| 21 | [Ac]-DK(1c)SC(2e)WQC(2e)W[2-Nal]LE(1c)R-[NH2] |
| 22 | [Ac]-DK(1c)SC(2f)WQC(2f)W[2-Nal]LE(1c)R-[NH2] |
| 23 | [Ac]-DK(1c)S[K(N₃)](2g)WQ[Pra](2g)W[2-Nal]LE(1c)R-[NH2] |
| 24 | [Ac]-DK(1c)S[Aha](2g)WQ[Pra](2g)W[2-Nal]LE(1c)R-[NH2] |
| 25 | [Ac]-K(1c)SC(2a)[2-Nal]QC(2a)W[2-Nal]LE(1c)R-[NH2] |
| 26 | [Ac]-K(1c)SC(2a)[Bip]QC(2a)W[2-Nal]LE(1c)R-[NH2] |
| 27 | [Ac]-K(1c)SC(2a)[1-Me-Trp]QC(2a)W[2-Nal]LE(1c)R-[NH2] |
| 28 | [Ac]-DK(1c)SC(2a)WQC(2a)[2-Nal][2-Nal]LE(1c)R-[NH2] |
| 29 | [Ac]-K(1c)SC(2a)WQC(2a)[Y(2-aminoethoxy)][2-Nal]LE(1c)R-[NH2] |
| 30 | [Ac]-K(1c)SC(2a)WQC(2a)H[2-Nal]LE(1c)R-[NH2] |
| 31 | [Ac]-K(1c)SC(2a)WQC(2a)W[F(4-F)]LE(1c)R-[NH2] |
| 32 | [Ac]-K(1c)SC(2a)WQC(2a)W[1-Me-Trp]LE(1c)R-[NH2] |
| 33 | [Ac]-C(1a)TC(2a)WEC(2a)WW[2-Me-Leu]C(1a)S-[NH2] |
| 34 | [Ac]-GK(1c)TC(2a)WEC(2c)WW[2-Me-Leu]E(1c)S-[NH2] |
| 35 | [Ac]-GC(1a)TC(2a)WEC(2a)W[2-Nal][2-Me-Leu]C(1a)R-[NH2] |
| 36 | [bAla](1c)*SC(2a)WQC(2a)W[2-Nal]LE(1c)R-[NH2] |
| 37 | [(3-Aminomethyl)benzoyl](1c)*SC(2a)WQC(2a)W[2-Nal]LE(1c)R-[NH2] |
| 38 | [(3-Aminomethyl)benzoyl](1c)*SC(2a)WQC(2a)[Y(2-aminoethoxy)][2- |
| | Nal][2-Me-Leu]E(1c)R-[NH2] |
| 39 | [(3-Aminomethyl)benzoyl](1c)*SC(2a)WQC(2a)[Y(2-aminoethoxy)][2- |
| | Nal]LE(1c)R-[NH2] |
| 40 | [(3-Aminomethyl)benzoyl](1c)*SC(2a)WQC(2a)W[2-Nal][2-Me- |
| | Leu]E(1c)R-[NH2] |
| 41 | [(3-Aminomethyl)benzoyl](1c)*SC(2a)WQC(2a)[Y(2-aminoethoxy)][2- |
| | Nal][2-Me-Leu]E(1c)[{d}R]-[NH2] |
| 42 | [(3-Aminomethyl)benzoyl](1c)*SC(2a)WQC(2a)[Y(2-aminoethoxy)][2- |
| | Nal][2-Me-Leu]E(1c)[2-Me-Arg]-[NH2] |
| 43 | [(3-Aminomethyl)benzoyl](1c)*SC(2a)WQC(2a)[Y(2-aminoethoxy)][2- |
| | Nal][2-Me-Leu]E(1c)[N-Me-Arg]-[NH2] |
| 44 | [(3-Aminomethyl)benzoyl](1c)*SC(2a)WQC(2a)[Y(2-aminoethoxy)][2- |
| | Nal][2-Me-Leu]E(1c)S-[NH2] |
| 45 | [(3-Aminomethyl)benzoyl](1c)*SC(2a)WQC(2a)[Y(2-aminoethoxy)][2- |
| | Nal][2-Me-Leu]E(1c)[F(4-NH₂)]-[NH2] |
| 46 | [(3-Aminomethyl)benzoyl](1c)*SC(2a)WQC(2a)[Y(2-aminoethoxy)][2- |
| | Nal][{d}L]E(1c)Y-[NH2] |
| 47 | [Ac]-K(1c)SC(2a)WQC(2a)[Y(2-aminoethoxy)][2-Nal][2-Me-Leu]E(1c)R-[NH2] |
| 48 | [Ac]-K(1c)SC(2a)WQC(2a)[Y(2-aminoethoxy)][2-Nal][{d}L]E(1c)R-[NH2] |
| 49 | [(4-Aminomethyl)benzoyl](1c)*SC(2a)WQC(2a)[Y(2-aminoethoxy)][2- |
| | Nal][2-Me-Leu]E(1c)R-[NH2] |
| 50 | [4-(2-Aminoethyl)benzoyl](1c)*SC(2a)WQC(2a)[Y(2-aminoethoxy)][2- |
| | Nal][2-Me-Leu]E(1c)R-[NH2] |
| 51 | [3-(4-Aminophenyl)propanoyl](1c)*SC(2a)WQC(2a)[Y(2-aminoethoxy)][2- |
| | Nal][2-Me-Leu]E(1c)R-[NH2] |
| 52 | [4-Aminomethyl-phenylacetyl](1c)*SC(2a)WQC(2a)[Y(2-aminoethoxy)][2- |
| | Nal][2-Me-Leu]E(1c)R-[NH2] |
| 53 | H-DC(1a)SC(2b)WQC(2b)WWLC(1a)R-[NH2] |
| 54 | [Ac]-K(1c)SC(2b)WQC(2b)[Y(2-aminoethoxy)][2-Nal]LE(1c)R-[NH2] |
| 55 | [Ac]-DE(1c)QC(2a)WQC(2a)YW[2-Me-Leu]K(1c)R-[NH2] |
| 56 | [Ac]-DE(1c)QC(2a)WQC(2a)[2-Nal]W[2-Me-Leu]K(1c)R-[NH2] |
| 57 | [Ac]-DE(1c)TC(2a)WQC(2a)W[2-Nal][2-Me-Leu]K(1c)R-[NH2] |
| 58 | [Ac]-DE(1c)SC(2a)WQC(2a)W[2-Nal][2-Me-Leu]K(1c)ENG-[NH2] |
| 59 | [Ac]-DE(1c)SC(2a)WQC(2a)W[2-Nal][2-Me-Leu]K(1c)NG-[NH2] |
| 60 | [Ac]-DE(1c)SC(2a)WQC(2a)W[2-Nal][2-Me-Leu]K(1c)G-[NH2] |
| 61 | [Ac]-DE(1c)SC(2a)WQC(2a)W[2-Nal][2-Me-Lys]K(1c)ENG-[NH2] |
| 62 | [Ac]-DC(1a)QC(2a)WQC(2a)[2-Nal]W[2-Me-Leu]C(1a)R-[NH2] |
| 63 | [Ac]-DC(1a)QC(2a)WQC(2a)YW[2-Me-Leu]C(1a)R-[NH2] |
| 64 | [Ac]-DC(1a)QC(2a)WQC(2a)WW[2-Me-Leu]C(1a)ENG-[NH2] |

and pharmaceutically acceptable salts and solvates thereof;
wherein:
*= bridge uses the peptide backbone amine or carboxylic acid at the N- or C-terminus, not the side chain amine or carboxylic acid
(1a) = [2,11] 1,3-dithio-propan-2-one bridge; (1c) = [2,11] lactam bridge; (1g) = [2,11] 1,4-disubstituted 1,2,3-triazole bridge; (2a) = [4,7] 1,3-dithio-propan-2-one bridge; (2c) = [4,7] lactam bridge; (2d) = [4,7] 1,2-phenylenedimethanethiol bridge; (2e) = [4,7] 1,3-phenylenedimethanethiol bridge; (2f) = [4,7] 1,4-phenylenedimethanethiol bridge; (2g) = [4,7] 1,4-disubstituted 1,2,3-triazole bridge.

In some embodiments, specific compounds of the invention include:

| Compound | |
|---|---|
| No. | Sequence |
| 67 | [(3-Aminomethyl)benzoyl](1c)*SE(2c)WQ[Dab](2c)[Y(2-aminoethoxy)][2- |
| | Nal][2-Me-Leu]E(1c)R-[NH2] |
| 68 | [(3-Aminomethyl)benzoyl](1c)*S[Orn](2c)WQE(2c)[Y (2-aminoethoxy)][2- |
| | Nal][2-Me-Leu]E(1c)R-[NH2] |
| 69 | [(3-Aminomethyl)benzoyl](1c)*SE(2c)WQ[Orn](2c)[Y(2-aminoethoxy)][2- |
| | Nal][2-Me-Leu]E(1c)R-[NH2] |
| 70 | [(3-Aminomethyl)benzoyl](1c)*SC(2a)[7-Me-Trp]QC(2a)[Y(2- |
| | aminoethoxy)][2-Nal][2-Me-Leu]E(1c)R-[NH2] |
| 78 | [(3-Aminomethyl)benzoyl](1c)*SC(2a)WQC(2a)[Y(2-aminoethoxy)][2- |
| | Nal][2-Me-Leu]E(1c)R[3-(3-Pyridyl)-Ala]-[NH2] |
| 79 | [(3-Aminomethyl)benzoyl](1c)*SC(2a)WQC(2a)[Y(2-aminoethoxy)][2- |
| | Nal][2-Me-Leu]E(1c)R[3-(4-Pyridyl)-Ala]-[NH2] |
| 80 | [4-Aminomethyl-phenylacetyl](1c)*SC(2a)WQC(2a)[Y(2-aminoethoxy)][2- |
| | Nal][2-Me-Leu]E(1c)[{d}R]-[NH2] |
| 81 | [4-Aminomethyl-phenylacetyl](1c)*SC(2a)WQC(2a)[Y(2-aminoethoxy)][2- |
| | Nal][2-Me-Leu]E(1c)S-[NH2] |
| 82 | [4-Aminomethyl-phenylacetyl](1c)*SC(2a)WQC(2a)[Y(2-aminoethoxy)][2- |
| | Nal][2-Me-Leu]E(1c)[Dab]-[NH2] |
| 84 | [Ac]-K(1c)SC(2a)WQC(2a)[Y(2-aminoethoxy)][2-Nal][2-Me- |
| | Leu]E(1c)[{d}R]-[NH2] |
| 85 | [Ac]-K(1c) TC(2a)WQC(2a)[Y(2-aminoethoxy)][2-Nal][2-Me- |
| | Leu]E(1c)[{d}R]-[NH2] |
| 87 | [6-Aminohexanoyl](1c)*SC(2a)WQC(2a)[Y(2-aminoethoxy)][2-Nal][2-Me- |
| | Leu]E(1c)[{d}R]-[NH2] |
| 90 | [(4-(2-Aminoethyl)-piperazine-1-yl)-acetyl](1c)*SC(2a)WQC(2a)[Y(2- |
| | aminoethoxy)][2-Nal][2-Me-Leu]E(1c)[{d}R]-[NH2] |
| 92 | [3-Aminomethyl-phenylacetyl](1c)*SC(2a)WQC(2a)[Y(2-aminoethoxy)][2- |
| | Nal][2-Me-Leu]E(1c)[{d}R]-[NH2] |

and pharmaceutically acceptable salts and solvates thereof;
wherein:
*= bridge uses the peptide backbone amine or carboxylic acid at the N- or C-terminus, not the side chain amine or carboxylic acid
(1a) = [2,11] 1,3-dithio-propan-2-one bridge; (1c) = [2,11] lactam bridge; (1g) = [2,11] 1,4-disubstituted 1,2,3-triazole bridge; (2a) = [4,7] 1,3-dithio-propan-2-one bridge; (2c) = [4,7] lactam bridge; (2d) = [4,7] 1,2-phenylenedimethanethiol bridge; (2e) = [4,7] 1,3-phenylenedimethanethiol bridge; (2f) = [4,7] 1,4-phenylenedimethanethiol bridge; (2g) = [4,7] 1,4-disubstituted 1,2,3-triazole bridge.

In some embodiments, specific compounds of the invention include:

| Compound | |
|---|---|
| No. | Sequence |
| 108 | [4-Aminomethyl-phenylacetyl](1c)*SC(2a)WQC(2a)[Y(2-aminoethoxy)][2- |
| | Nal][2-Me-Leu]E(1c)[{d}R]-[OH] |
| 109 | [4-Aminomethyl-phenylacetyl](1c)*SC(2a)WQC(2a)[Y(2-aminoethoxy)][2- |
| | Nal][2-Me-Leu]E(1c)[{d}2,4-Diaminobutanoyl([2-(trimethyl-2- |
| | aminoethoxy)ethoxy]propyl)]-[NH2] |
| 115 | [4-Aminomethyl-phenylacetyl](1c)*SC(2a)WQC(2a)[Y(2-aminoethoxy)][2- |
| | Nal][2-Me-Leu]E(1c)[{d}Q]-[NH2] |
| 117 | [4-Aminomethyl-phenylacetyl](1c)*SC(2a)WQC(2a)[Y(2-aminoethoxy)][2- |
| | Nal][2-Me-Leu]E(1c)[{d}H]-[NH2] |
| 119 | [4-Aminomethyl-phenylacetyl](1c)*SC(2a)WQC(2a)[Y(2-aminoethoxy)][2- |
| | Nal][2-Me-Leu]E(1c)[3-Aminopropanoyl]-[NH2] |
| 120 | [4-Aminomethyl-phenylacetyl](1c)*SC(2a)WQC(2a)[Y(2-aminoethoxy)][2- |
| | Nal][2-Me-Leu]E(1c)[Dab][3-(3-Pyridyl)-Ala]-[NH2] |
| 121 | [4-Aminomethyl-phenylacetyl](1c)*SC(2a)WQC(2a)[Y(2-aminoethoxy)][6- |
| | F-Trp][2-Me-Leu]E(1c)[Dab][3-(3-Pyridyl)-Ala]-[NH2] |
| 122 | [4-Aminomethyl-phenylacetyl](1c)*SE(2c)WQ[Dab](2c)[Y(2- |
| | aminoethoxy) ][2-Nal][2-Me-Leu]E(1c)[Dab][3-(3-Pyridyl)-Ala]-[NH2] |
| 123 | [4-Aminomethyl-phenylacetyl](1c)*S[Dab](2c)WQE(2c)[Y(2- |
| | aminoethoxy) ][2-Nal][2-Me-Leu]E(1c)[Dab][3-(3-Pyridyl)-Ala]-[NH2] |
| 124 | [4-Aminomethyl-phenylacetyl](1c)*SE(2c)WQ[Orn](2c)[Y(2- |
| | aminoethoxy)][2-Nal][2-Me-Leu]E(1c)[Dab][3-(3-Pyridyl)-Ala]-[NH2] |
| 125 | [4-Aminomethyl-phenylacetyl](1c)*SE(2c)WQ[Dab](2c)[Y(2- |
| | aminoethoxy)][2-Nal][2-Me-Leu]E(1c)[Dab]-[NH2] |
| 126 | [4-Aminomethyl-phenylacetyl](1c)*VE(2c)WQ[Dab](2c)[Y(2- |
| | aminoethoxy][2-Nal][2-Me-Leu]E(1c)[Dab]-[NH2] |
| 130 | [4-Aminomethyl-phenylacetyl](1c)*SE(2c)WQ[Dab](2c)F[2-Nal][2-Me- |
| | Leu]E(1c)[Dab]-[NH2] |
| 136 | [4-Aminomethyl-phenylacetyl](1c)*SE(2c)WQ[Dab](2c)[Y(2- |
| | aminoethoxy)][2-Nal][2-Me-Val]E(1c)[Dab]-[NH2] |
| 137 | [4-Aminomethyl-phenylacetyl](1c)*SE(2c)WQ[Dab](2c)[Y(2- |
| | aminoethoxy)][2-Nal][2-Me-Leu]E(1c)-[NH-3-aminopropanoyl] |
| 138 | [4-Aminomethyl-phenylacetyl](1c)*SE(2c)[4-F-Trp]Q[Dab](2c)[Y(2- |
| | aminoethoxy)][2-Nal][2-Me-Leu]E(1c)[Dab]-[NH2] |
| 139 | [4-Aminomethyl-phenylacetyl](1c)*SE(2c)[7-F-Trp]Q[Dab](2c)[Y(2- |
| | aminoethoxy)][2-Nal][2-Me-Leu]E(1c)[Dab]-[NH2] |
| 145 | [4-Aminomethyl-phenylacetyl](1c)*[Q(pyrrolidin)]E(2c)WQ[Dab](2c)[Y(2- |
| | aminoethoxy)][2-Nal][2-Me-Leu]E(1c)[Dab]-[NH2] |
| 146 | [4-Aminomethyl-phenylacetyl](1c)*SE(2c)W[Q(pyrrolidin)][Dab](2c)[Y (2- |
| | aminoethoxy)][2-Nal][2-Me-Leu]E(1c)[Dab]-[NH2] |
| 147 | [4-Aminomethyl-phenylacetyl](1c)*SC(2a)WQC(2a)[Y(2-aminoethoxy)][2- |
| | Nal][2-Me-Leu]E(1c)R[3-(3-Pyridyl)-Ala]-[NH2] |
| 148 | [(3-Aminomethyl)benzoyl](1c)*SC(2a)WQC(2a)[Y(2-aminoethoxy)][2- |
| | Nal][2-Me-Leu]E(1c)[Dab][3-(3-Pyridyl)-Ala]-[NH2] |
| 149 | [(3-Aminomethyl)benzoyl](1c)*SC(2a)WQC(2a)][Y (2-aminoethoxy)][2- |
| | Nal][2-Me-Leu]E(1c)R[3-(3-Quinolinyl)-Ala]-[NH2] |
| 150 | [(3-Aminomethyl)benzoyl](1c)*SC(2a)WQC(2a)][Y(2-aminoethoxy)][2- |
| | Nal][2-Me-Leu]E(1c)R[{d}[3-(3-Pyridyl)-Ala]]-[NH2] |
| 151 | [(3-Aminomethyl)benzoyl](1c)*SC(2a)WQC(2a)][Y(2-aminoethoxy)][2- |
| | Nal][2-Me-Leu]E(1c)R[3-Amino-3-(3′-pyridyl)propionyl]-[NH2] |
| 155 | [Ac]-K(1c) TC(2a)WQC(2a)[Y(2-aminoethoxy)][2-Nal][2-Me- |
| | Leu]E(1c)[Dab][3-(3-Pyridyl)-Ala]-[NH2] |
| 156 | [(3-Aminomethyl)benzoyl](1c)*IE(2c)WQ[Dab](2c)[Y(2-aminoethoxy)][2- |
| | Nal][2-Me-Val]E(1c)[Dab][3-(3-Pyridyl)-Ala]-[NH2] |
| 157 | [4-Aminomethyl-phenylacetyl](1c)*IE(2c) WQ[Dab](2c)[Y(2- |
| | aminoethoxy) ][2-Nal][2-Me-Val]E(1c)[Dab][3-(3-Pyridyl)-Ala]-[NH2] |
| 158 | [4-Aminomethyl-phenylacetyl](1c)*IE(2c)WQ[Dab](2c)[Y(2- |
| | aminoethoxy)][F(3,4-Me)][2-Me-Val]E(1c)G[3-(3-Pyridyl)-Ala]-[NH2] |
| 165 | [4-Aminomethyl-phenylacetyl](1c)*IE(2c)WQ[Dab](2c)[Y(2- |
| | aminoethoxy)][F(3,4-Me)][2-Me-Val]E(1c)[Dab][2-Me-3-(3-Pyridyl)-Ala]- |
| | [NH2] |
| 166 | [4-Aminomethyl-phenylacetyl](1c)*IE(2c)WQ[Dab](2c)[Y(Me)][F(3,4- |
| | Me)][2-Me-Val]E(1c)[Dab][2-Me-3-(3-Pyridyl)-Ala]-[NH2] |
| 167 | [4-Aminomethyl-phenylacetyl](1c)*IE(2c)WQ[Dab](2c)[2-Me-Phe][F(3,4- |
| | Me)][2-Me-Val]E(1c)[Dab][2-Me-3-(3-Pyridyl)-Ala]-[NH2] |
| 168 | [4-Aminomethyl-phenylacetyl](1c)*IE(2c)WQ[Dab](2c)[Bip][F(3,4-Me)][2- |
| | Me-Val]E(1c)[Dab][2-Me-3-(3-Pyridyl)-Ala]-[NH2] |
| 169 | [4-Aminomethyl-phenylacetyl](1c)*IE(2c)WQ[Dab](2c)[2-Me-F(4-F)][F(3,4- |
| | Me)][2-Me-Val]E(1c)[Dab][2-Me-3-(3-Pyridyl)-Ala]-[NH2] |
| 170 | [4-Aminomethyl-phenylacetyl](1c)*IE(2c)WQ[Dab](2c)[{d}F(4-F)][F(3,4- |
| | Me)][2-Me-Val]E(1c)[Dab][2-Me-3-(3-Pyridyl)-Ala]-[NH2] |
| 171 | [4-Aminomethyl-phenylacetyl](1c)*IE(2c)WQ[Dab](2c)[F(4-CI)][F(3,4- |
| | Me)][2-Me-Val]E(1c)[Dab][2-Me-3-(3-Pyridyl)-Ala]-[NH2] |
| 172 | [4-Aminomethyl-phenylacetyl](1c)*IE(2c)WQ[Dab](2c)[alpha-Me- |
| | Trp][F(3,4-Me)][2-Me-Val]E(1c)[Dab][2-Me-3-(3-Pyridyl)-Ala]-[NH2] |
| 173 | [4-Aminomethyl-phenylacetyl](1c)*IE(2c)WQ[Dab](2c)[3,3-Diphenyl- |
| | Ala][F(3,4-Me)][2-Me-Val]E(1c)[Dab][2-Me-3-(3-Pyridyl)-Ala]-[NH2] |
| 174 | [4-Aminomethyl-phenylacetyl](1c)*IE(2c)WQ[Dab](2c)[Phg][F(3,4-Me)][2- |
| | Me-Val]E(1c)[Dab][2-Me-3-(3-Pyridyl)-Ala]-[NH2] |
| 175 | [4-Aminomethyl-phenylacetyl](1c)*IE(2c)WQ[Dab](2c)[F(4-Me)][1-Me- |
| | Trp][2-Me-Val]E(1c)[Dab][2-Me-3-(3-Pyridyl)-Ala]-[NH2] |
| 176 | [4-Aminomethyl-phenylacetyl](1c)*IE(2c)WQ[Dab](2c)[F(4-Me)][Bip][2-Me- |
| | Val]E(1c)[Dab][2-Me-3-(3-Pyridyl)-Ala]-[NH2] |
| 177 | [4-Aminomethyl-phenylacetyl](1c)*IE(2c)WQ[Dab](2c)[F(4-Me)][{d}6-F- |
| | Trp][2-Me-Val]E(1c)[Dab][2-Me-3-(3-Pyridyl)-Ala]-[NH2] |

and pharmaceutically acceptable salts and solvates thereof;
wherein:
*= bridge uses the peptide backbone amine or carboxylic acid at the N- or C-terminus, not the side chain amine or carboxylic acid
(1a) = [2,11] 1,3-dithio-propan-2-one bridge; (1c) = [2,11] lactam bridge; (1g) = [2,11] 1,4-disubstituted 1,2,3-triazole bridge; (1h) = [2,11] 1,5-disubstituted 1,2,3-triazole bridge; (2a) = [4,7] 1,3-dithio-propan-2-one bridge; (2c) = [4,7] lactam bridge; (2d) = [4,7] 1,2-phenylenedimethanethiol bridge; (2e) = [4,7] 1,3-phenylenedimethanethiol bridge; (2f) = [4,7] 1,4-phenylenedimethanethiol bridge; (2g) = [4,7] 1,4-disubstituted 1,2,3-triazole bridge.

The invention further provides a composition comprising a compound as described above. The composition may be a pharmaceutical composition, and may comprise a pharmaceutically acceptable carrier, excipient or vehicle.

The invention further provides a method for the synthesis of a compound as described above. The method may comprise the steps of synthesising the peptide by solid-phase or liquid-phase methodology, and optionally isolating and/or purifying the final product, and optionally further comprising the step of forming an amide bond, forming two thioether bonds with a linker, or forming a triazole between the amino acid residues at positions X2 and X11, and optionally further comprising the step of forming an amide bond, forming two thioether bonds with a linker, or forming a triazole between the amino acid residues at positions X4 and X7.

The invention further provides a compound of the invention, or a pharmaceutical compositions comprising said compound, for use in a method of medical treatment.

The invention also provides a compound of the invention, or a pharmaceutical composition comprising said compound, for use in a method of prevention or treatment of Inflammatory Bowel Disease (IBD), ulcerative colitis, Crohn's disease, Celiac disease (nontropical Sprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-I, chronic granulomatous disease, glycogen storage disease type 1b, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, ankylosing spondylitis, or graft versus host disease in a subject, and combinations thereof.

In some embodiments, the compound of the invention, or the pharmaceutical composition comprising said compound, for use in a method of prevention or treatment of inflammatory bowel (IBD), Crohn's Disease, ulcerative colitis, and psoriasis.

The invention also provides use of a compound of the invention, or the pharmaceutical composition comprising said compound, in the manufacture of a medicament for the prevention or treatment of Inflammatory Bowel Disease (IBD), ulcerative colitis, Crohn's disease, Celiac disease (nontropical Sprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-I, chronic granulomatous disease, glycogen storage disease type 1b, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, ankylosing spondylitis, or graft versus host disease in a subject, and combinations thereof.

In some embodiments, the use of the compound of the invention, or the pharmaceutical composition comprising said compound, in the manufacture of a medicament for the prevention or treatment of inflammatory bowel (IBD), Crohn's Disease, ulcerative colitis, and psoriasis.

The invention also provides a method of prevention or treatment of Inflammatory Bowel Disease (IBD), ulcerative colitis, Crohn's disease, Celiac disease (nontropical Sprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-I, chronic granulomatous disease, glycogen storage disease type 1b, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, ankylosing spondylitis, or graft versus host disease in a subject, and combinations thereof; which comprises administering to a subject an effective amount of a compound of the invention, or a pharmaceutical composition comprising said compound.

In some embodiments, the method of prevention or treatment of inflammatory bowel (IBD), Crohn's Disease, ulcerative colitis, and psoriasis; which comprises administering to the subject an effective amount of the compound of the invention, or the pharmaceutical composition comprising said compound.

Further aspects and embodiments of the present invention will become apparent from the disclosure below.

### DETAILED DESCRIPTION OF THE INVENTION

### Definitions

Unless otherwise defined herein, scientific and technical terms used herein shall have the meanings that are commonly understood by those of ordinary skill in the art. Generally, nomenclature employed herein in connection with techniques of chemistry, molecular biology, cell and cancer biology, immunology, microbiology, pharmacology, and protein and nucleic acid chemistry, described herein, is that well known and commonly used in the art.

All publications, patents and published patent applications referred to in this application are specifically incorporated by reference herein. In case of conflict, the present specification, including its specific definitions, will control.

Throughout this specification, the word “comprise” or variations such as “comprises” or “comprising” will be understood to imply the inclusion of a stated integer or component, or of a stated group of integers or components, but not the exclusion of any other integer or component or group of integers or components.

The singular forms “a”, “an”, and “the” include the plurals unless the context clearly dictates otherwise.

The term “including” is used to mean “including but not limited to”. “Including” and “including but not limited to” are used interchangeably.

The terms “patient”, “subject”, and “individual” may be used interchangeably and may refer to either a human or a non-human animal. Subjects are typically mammals, including humans, non-human primates (including great apes, Old World monkeys and New World monkeys), livestock animals (e.g., bovines, porcines), companion animals (e.g., canines, felines) and rodents (e.g., mice and rats).

As used herein, the term “pharmaceutically acceptable salt” is intended to indicate a salt which is not harmful to a patient or subject to which the salt in question is administered. It may suitably be a salt chosen, e.g., among acid addition salts and basic salts. Examples of acid addition salts include chloride salts, citrate salts and acetate salts. Examples of basic salts include salts where the cation is selected among alkali metal cations, such as sodium or potassium ions, alkaline earth metal cations, such as calcium or magnesium ions, as well as substituted ammonium ions, such as ions of the type N(R¹)(R²)(R³)(R⁴)⁺, where R¹, R², R³ and R⁴ independently will typically designate hydrogen, optionally substituted C₁₋₆-alkyl or optionally substituted C₂₋₆-alkenyl. Examples of relevant C₁₋₆-alkyl groups include methyl, ethyl, 1-propyl and 2-propyl groups. Examples of C₂₋₆-alkenyl groups of possible relevance include ethenyl, 1-propenyl and 2-propenyl. Other examples of pharmaceutically acceptable salts are described in “Remington's Pharmaceutical Sciences”, 17^{th} edition, Alfonso R. Gennaro (Ed.), Mark Publishing Company, Easton, PA, USA, 1985 (and more recent editions thereof), in the “Encyclopaedia of Pharmaceutical Technology”, 3^{rd} edition, James Swarbrick (Ed.), Informa Healthcare USA (Inc.), NY, USA, 2007, and in J. Pharm. Sci. 66: 2 (1977).

The term “solvate” in the context of the present invention refers to a complex of defined stoichiometry formed between a solute (in casu, a peptide or pharmaceutically acceptable salt thereof according to the invention) and a solvent. The solvent in this connection may, for example, be water, ethanol or another pharmaceutically acceptable—typically small-molecular—organic species, such as, but not limited to, acetic acid or lactic acid. When the solvent in question is water, such a solvate is normally referred to as a hydrate.

The term “antagonist” as employed in the context of the invention refers to a substance that inhibits the receptor type in question, typically by binding to it (i.e. as a ligand) and blocking it.

Each embodiment of the invention described herein may be taken alone or in combination with one or more other embodiments of the invention.

The term “therapeutically effective amount” as used herein in the context of the above-described methods of treatment or other therapeutic interventions according to the invention refers to an amount that is sufficient to cure, ameliorate, alleviate or partially arrest the clinical manifestations of the particular disease, disorder or condition that is the object of the treatment or other therapeutic intervention in question e.g. as measured by established clinical endpoints or other biomarkers (established or experimental). A therapeutically relevant amount may be determined empirically by one skilled in the art based on the indication being treated or prevented and the subject to whom the therapeutically relevant amount is being administered. For example, the skilled worker may measure one or more of the clinically relevant indicators of bioactivity described herein, e.g. myeloperoxidase (MPO), interleukin-1β (IL-1β), interleukin-6 (IL-6), interleukin-22 (IL-22), interleukin-17A (IL-17A), interleukin-17F (IL-17F), lipocalin 2 (LCN2), matrix metallopeptidase 9 (MMP9), S100 calcium-binding protein A8 (S100A8), microRNA-223-3p (miR223-3p), Claudin 8 (CLDN8), and phosphorylated signal transducer and activator of transcription 3 (pSTAT3) proteins, polynucleotides encoding any of the proteins, and polynucleotides comprising a region complementary to microRNA-223-3p or any of the polynucleotides that encode any of the proteins, as described in WO 2018/089693. The skilled worker may determine a clinically relevant amount through in vitro or in vivo measurements.

An amount adequate to accomplish any or all of these effects is defined as a therapeutically effective amount. The administered amount and the method of administration can be tailored to achieve optimal efficacy. An amount effective for a given purpose will depend, inter alia, on the severity of the disease, disorder or condition that is the object of the particular treatment or other therapeutic intervention, on the body weight and general condition of the subject in question, on diet, on possible concurrent medication, and on other factors well known to those skilled in the medical arts. Determination of an appropriate dosage size and dosing regimen most appropriate for administration of a peptide or pharmaceutically acceptable salt or solvate thereof according to the invention to a human may be guided by the results obtained by the present invention, and may be confirmed in properly designed clinical trials. An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are well known to the skilled person.

The terms “treatment” and grammatical variants thereof (e.g. “treated”, “treating”, “treat”) as employed in the present context refer to an approach for obtaining beneficial or desired clinical results. For the purposes of this invention, beneficial or desired clinical results include, but are not limited to, alleviation of symptoms, diminishment of extent of disease, stabilization (i.e. not worsening) of state of disease, delay or slowing of disease progression, amelioration or palliation of the disease state, and remission (whether partial or total), whether detectable or undetectable. “Treatment” can also mean prolonging survival relative to expected survival time if not receiving treatment. A subject (e.g. a human) in need of treatment may thus be a subject already afflicted with the disease or disorder in question. The term “treatment” includes inhibition or reduction of an increase in severity of a pathological state or symptoms (e.g. inflammation) relative to the absence of treatment, and is not necessarily meant to imply complete cessation of the relevant disease, disorder or condition.

The terms “prevention” and grammatical variants thereof (e.g., “prevented”, “preventing”, “prevent”) as employed in the present context refer to an approach for hindering or preventing the development of, or altering the pathology of, a condition, disease or disorder. Accordingly, “prevention” may refer to prophylactic or preventive measures. For the purposes of this invention, beneficial or desired clinical results include, but are not limited to, prevention or slowing of symptoms, progression or development of a disease, whether detectable or undetectable. A subject (e.g. a human) in need of “prevention” may thus be a subject not yet afflicted with the disease or disorder in question. The term “prevention” thus includes inhibiting or slowing the onset of disease relative to the absence of treatment, and is not necessarily meant to imply permanent prevention of the relevant disease, disorder or condition.

### Amino Acids Nomenclature

The term “amino acid” is an organic compound that contains an amino or amine group (—NH₂ or —NHR) and a carboxylic acid (—COOH) group. The amine group of the amino acid may be further functionalised, such as an azide group (—N₃), for example in azidoacetic acid or (N₃)-Ala, or such as a propargyl group (—CH₂C≡CH), for example in but-3-ynoic acid.

Some amino acids described herein have the amine and carboxylic acid groups attached to the same carbon, called alpha (a) amino acids. Some amino acids described herein have the amine and carboxylic acid groups 1, 2, 3, 4, 5, or 6 carbon atoms away. For example, beta-alanine (bAla) has the amine and the carboxylic acid groups are 1 carbon away, such that the carbon connected to the amine group and the carbon connected to the carboxylic acid group are adjacent to one another.

Some amino acids described herein have a side chain specific to each amino acid. The side chain may also be further functionalised. For example, a side chain amine group may be functionalised into an azide group (—N₃), such as Lys(N₃). Another example may be where a side chain amine may be functionalised into an amide with a pendant azide group (—N₃), such as in Dab(azidoacetic acid) and Dab((N₃)-Ala)). Another example may be where a side chain amine may be functionalised into an amide with a pendant alkyne group, such as in Dab(3-butynoic acid). Another example may be where the side chain carboxylic acid may be functionalised into an amide with a pendant alkyne group, such as in Glu(propargylamine).

Throughout the present specification, unless naturally occurring amino acids are referred to by their full name (e.g. alanine, arginine, etc.), they are designated by their conventional three-letter or single-letter abbreviations (e.g. Ala or A for alanine, Arg or R for arginine, etc.). In the case of certain less common or non-naturally occurring amino acids (i.e. amino acids other than the 20 encoded by the standard mammalian genetic code), unless they are referred to by their full name (e.g. omithine, etc.), frequently employed three- or four-character codes are employed for residues thereof, including 2-Nal (3-(2-naphthyl)-alanine).

Unless otherwise indicated, reference is made to the L-isomeric forms of the amino acids in question.
Additional abbreviations include the following:
2-Nal 3-(2-naphthyl)-L-alanine
{d}R or D-Arg D-arginine
{d}L or D-Leu D-leucine
{d}C or D-Cys D-cysteine
{d}Q or D-Gln D-glutamine
{d}E or D-Glu D-glutamic acid
{d}H or D-His D-histidine
alpha-Me-Trp α-methyl-L-tryptophan
1-Me-Trp 1-methyl-L-tryptophan
4-F-Trp 4-fluoro-L-tryptophan
6-F-Trp 6-fluoro-L-tryptophan
{d}6-F-Trp 6-fluoro-D-tryptophan
6-Cl-Trp 6-chloro-L-tryptophan
7-Me-Trp 7-methyl-L-tryptophan
7-F-Trp 7-fluoro-L-tryptophan
7-Ph-Trp 7-phenyl-L-tryptophan
7-(Naphth-2-yl)-Trp 7-(naphth-2-yl)-L-tryptophan
3-(3-Pyridyl)-Ala 3-(3-pyridyl)-L-alanine
3-(4-Pyridyl)-Ala 3-(4-pyridyl)-L-alanine
{d}[3-(3-Pyridyl)-Ala]3-(3-pyridyl)-D-alanine
2-Me-3-(3-Pyridyl)-Ala (2S)-2-amino-2-methyl-3-(3-pyridyl)propanoic acid
3-(3-Quinolinyl)-Ala 3-(3-quinolinyl)-L-alanine; also known as (2S)-2-amino-3-(3-quinolyl)propanoic acid
F(3-F) or 3-F-Phe 3-fluoro-L-phenylalanine
F(4-F) or 4-F-Phe 4-fluoro-L-phenylalanine
{d}F(4-F) 4-fluoro-D-phenylalanine
F(4-Cl) or 4-Cl-Phe 4-chloro-L-phenylalanine
F(4-NH₂) or 4-NH₂-Phe 4-amino-L-phenylalanine
F(4-Me) or 4-Me-Phe 4-methyl-L-phenylalanine
F(3,4-Me) or 3,4-Me-Phe 3,4-dimethyl-L-phenylalanine
F(3,5-F) or 3,5-F-Phe 3,5-difluoro-L-phenylalanine
2-Me-F(4-F) (2S)-2-amino-3-(4-fluorophenyl)-2-methyl-propanoic acid
2-Me-Leu 2-methyl-L-leucine
2-Me-Lys 2-methyl-L-lysine
2-Me-Arg 2-methyl-L-arginine
2-Me-Val 2-methyl-L-valine
2-Me-Phe alpha-methyl-L-phenylalanine; also known as
(2S)-2-amino-2-methyl-3-phenyl-propanoic acid
N-Me-Arg N₂-methyl-L-arginine
N-Me-Ser N-methyl-L-serine
N-Me-Cys N-methyl-L-cysteine
N-Me-Trp Na-methyl-L-tryptophan; also known as L-abrine
N-Me-Gln N₂-methyl-L-glutamine
Bip biphenyl-L-alanine
3,3-Diphenyl-Ala 3,3-diphenyl-L-alanine; also known as β-phenyl-L-phenylalanine, or (S)-2-amino-3,3-diphenylpropionic acid
Aad (2S)-2-aminoadipic acid, also known as (2S)-2-aminohexanedioic acid, or L-homoglutamic acid
Apm (2S)-2-aminopimelic acid, also known as (2S)-2-aminoheptanedioic acid, or L-bishomoglutamic acid
Dab (2S)-2,4-diaminobutanoic acid
Orn L-omithine, also known as 2,5-diaminopentanoic acid
hLys (2S)-2-amino-7-amino-heptanoic acid, also known as L-homolysine
bAla 3-aminopropionic acid, also known as beta-alanine or β-alanine
beta-homo-Ser L-β-homoserine
Ala(N₃) 3-azido-L-alanine
Aha azidohomo-L-alanine or 4-azido-L-homoalanine
Orn(N₃) azido-L-omithine
K(N₃) or Lys(N₃) azido-L-lysine
Pra L-propargylglycine
Hpg L-homopropargylglycine
Bpg L-bishomopropargylglycine
Dpr (2S)-2,3-diaminopropanoic acid, also known as 3-amino-L-alanine
Glu(propargylamine) (2S)-2-amino-5-oxo-5-(prop-2-ynylamino)pentanoic acid
Dab(3-butynoic acid) (2S)-2-amino-4-(but-3-ynoylamino)butanoic acid
Dab(azidoacetic acid) (2S)-2-amino-4-[(2-azidoacetyl)amino]butanoic acid
(N₃)-Ala (2S)-2-azidopropanoic acid
Dab((N₃)-Ala)) (2S)-2-amino-4-[[(2S)-2-azidopropanoyl]amino]butanoic acid
Abu (2S)-2-aminobutyric acid
Y(2-aminoethoxy) (2S)-2-amino-3-[4-(2-aminoethoxy)phenyl]propanoic acid
Y(Me) (2S)-2-amino-3-(4-methoxyphenyl)propanoic acid
Q(pyrrolidin) (2S)-2-amino-5-oxo-5-pyrrolidin-1-yl-pentanoic acid
GABA gamma aminobutyric acid; also known as 4-aminobutanoic acid
Phg L-2-phenylglycine
F(4-NH₂-(2-(trimethyl-2-aminoethoxy)ethoxy)propyl) 2-[2-[3-[4-[(2S)-2-amino-2-carboxy-ethyl]anilino]-3-oxo-propoxy]ethoxy]ethyl-trimethyl-ammonium

| Residue | Structure |
|---|---|
| F(4-NH₂-(2-(trimethyl-2- aminoethoxy)ethoxy)propyl) | 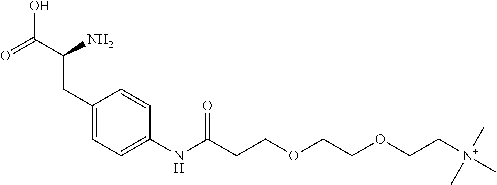 [SMILES: C[N+](C)(C)CCOCCOCCC(=O)Nc1ccc(C[C@H](N)C(=O)O)cc1] |
| 2,4-diaminobutanoyl([2-(trimethyl-2- aminoethoxy)ethoxy]propyl) | 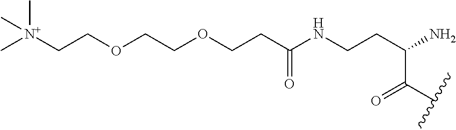 [SMILES: *C(=O)[C@@H](N)CCNC(=O)CCOCCOCC[N+](C)(C)C] |
| 4-aminotetrahydro-2H-pyran-4-acetyl | 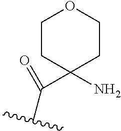 [SMILES: *C(=O)C1(N)CCOCC1] |
| {d}2,4-diaminobutanoyl([2-(trimethyl-2- aminoethoxy)ethoxy]propyl) | 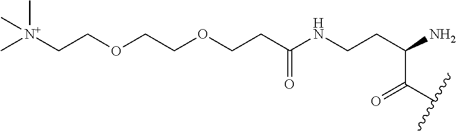 [SMILES: *C(=O)[C@H](N)CCNC(=O)CCOCCOCC[N+](C)(C)C] |
| 3-aminopropanoyl | 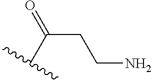 [SMILES: *C(=O)CCN] |
| 3-amino-3-(3′-pyridyl)propionyl | 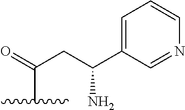 [SMILES: *C(=O)C[C@@H](N)c1cccnc1] |
| 4-aminomethyl-2-pyridineacetyl | 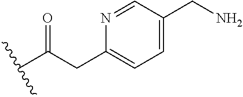 [SMILES: *C(=O)Cc1ccc(CN)cn1] |
| 2,3-diaminopropanoyl(3-pyridylacetyl) | 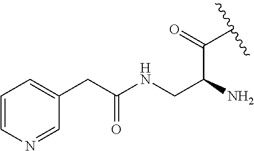 [SMILES: *C(=O)[C@@H](N)CNC(=O)Cc1cccnc1] |
| 2,3-diaminopropanoyl(3-pyridylpropionyl) | 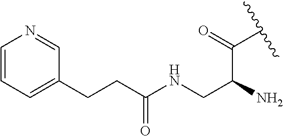 [SMILES: *C(=O)[C@@H](N)CNC(=O)CCc1cccnc1] |
| 2,3-diaminopropanoyl(3-fluorobenzoyl) | 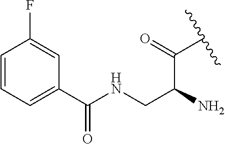 [SMILES: *C(=O)[C@@H](N)CNC(=O)c1cccc(F)c1] |
| 2,3-diaminopropanoyl(3- fluorophenylacetyl) | 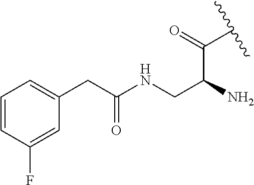 [SMILES: *C(=O)[C@@H](N)CNC(=O)Cc1cccc(F)c1] |
| 3-(4-aminophenyl)propanoyl | 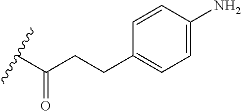 [SMILES: *C(=O)CCc1ccc(N)cc1] |
| (3-aminomethyl)benzoyl | 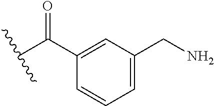 [SMILES: *C(=O)c1cccc(CN)c1] |
| (4-aminomethyl)benzoyl | 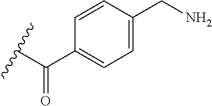 [SMILES: *C(=O)c1ccc(CN)cc1] |
| 4-(2-aminoethyl)benzoyl | 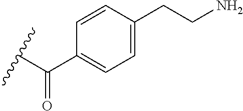 [SMILES: *C(=O)c1ccc(CCN)cc1] |
| 2-aminomethyl-phenylacetyl | 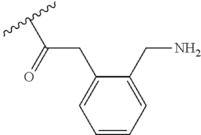 [SMILES: *C(=O)Cc1ccccc1CN] |
| 3-aminomethyl-phenylacetyl | 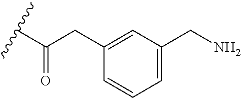 [SMILES: *C(=O)Cc1cccc(CN)c1] |
| 4-aminomethyl-phenylacetyl | 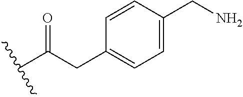 [SMILES: *C(=O)Cc1ccc(CN)cc1] |
| 4-aminomethyl-2-pyridineacetyl | 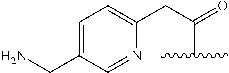 [SMILES: *C(=O)Cc1ccc(CN)cn1] |
| 4-aminomethyl-3-pyridineacetyl | 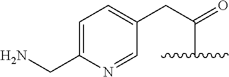 [SMILES: *C(=O)Cc1ccc(CN)nc1] |
| 4-aminomethyl-2-fluoro-phenylacetyl | 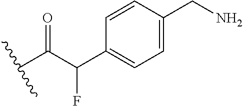 [SMILES: *C(=O)C(F)c1ccc(CN)cc1] |
| 4-aminomethyl-3-fluoro-phenylacetyl | 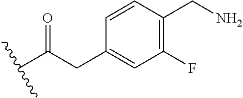 [SMILES: *C(=O)Cc1ccc(CN)c(F)c1] |
| 4-aminomethyl-2-methyl-phenylacetyl | 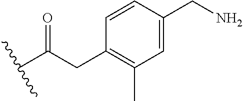 [SMILES: *C(=O)Cc1ccc(CN)cc1C] |
| 4-aminomethyl-3-methyl-phenylacetyl | 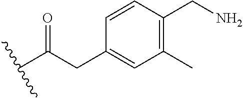 [SMILES: *C(=O)Cc1ccc(CN)c(C)c1] |
| 4-aminomethyl-2-methoxy-phenylacetyl | 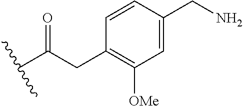 [SMILES: *C(=O)Cc1ccc(CN)cc1OC] |
| 4-aminomethyl-3-methoxy-phenylacetyl | 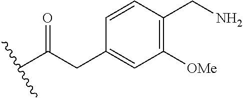 [SMILES: *C(=O)Cc1ccc(CN)c(OC)c1] |
| 6-aminohexanoyl | 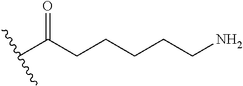 [SMILES: *C(=O)CCCCCN] |
| 6-amino-4-oxahexanoyl | 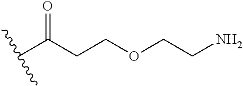 [SMILES: *C(=O)CCOCCN] |
| trans-4-aminomethyl-cyclohexyl-1- carbonyl | 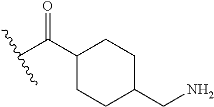 [SMILES: *C(=O)C1CCC(CN)CC1] |
| (4-(2-aminoethyl)-piperazine-1-yl)-acetyl | 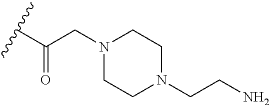 [SMILES: *C(=O)CN1CCN(CCN)CC1] |
| 4-methylaminomethyl-phenylacetyl | 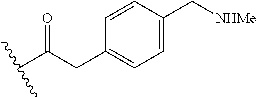 [SMILES: *C(=O)Cc1ccc(CNC)cc1] |

Amino acid residues are amino acid moieties within a peptide chain. Unnatural amino acid residues may be identified as the fragment of the unnatural amino acid defined in a peptide chain (for example, the unnatural amino acid 3-aminomethylbenzoic acid may be identified as the unnatural amino acid residue (3-aminomethyl)benzoyl in a peptide chain).

Linear peptides are written from N-terminus to C-terminus, left to right.

Unnatural (or non-naturally occurring) amino acids and unnatural (or non-naturally occurring) amino acid residues are amino acids and amino acid residues that do not naturally occur in peptide chains. Unnatural amino acids may be formed as secondary metabolites in bacteria, fungi, plants, or marine organisms, or they can be synthesised chemically.

### Lactam

A lactam is cyclic amide of formula cyclo(R—NH—C(═O)—R) wherein each R may be any other suitable functional group that joins to the other R. Each R may the the same or different.

### Dithioether

A thioether is a functional group of the formula R—S—R, wherein R may be any other suitable functional group. A dithioether is a functional group comprising two thioether groups linked together by a linker, such as R—S—L—Y—L—S—R wherein the linker is —L—Y—L—.

### Triazole

A triazole (or triazole ring) is a heterocyclic compound with molecular formula C₂H₃N₃, having a five-membered ring of two carbons and three nitrogen atoms. There are two sets of isomers for triazole based upon the relative positions of the three nitrogen atoms: 1,2,3-triazoles and 1,2,4-triazoles. Preferably, the triazole in the bridge containing a triazole ring is a 1,2,3-triazole.

### Head-to-Tail Cyclisation

The term “head-to-tail cyclisation” is cyclisation of the N-terminal amine (or derivative thereof) and the C-terminal carboxylic acid to form a cyclic peptide. Typically, this cyclisation forms an amide bond.

### C₁₋₄ Acyl Groups

C₁₋₄ acyl groups that may be present as a group R¹ in the context of compounds of the present invention include formyl (i.e. methanoyl), acetyl (i.e. ethanoyl or —C(═O)CH₃), propanoyl, 1-butanoyl and 2-methylpropanoyl groups.

### C₁₋₄ Alkyl Groups

C₁₋₄ alkyl groups that may be present as a group R¹ in the context of compounds of the present invention include, but are not limited to, C₁₋₃ alkyl groups, such as methyl(Me or —CH₃), ethyl (—CH₂CH₃), 1-propyl (—CH₂CH₂CH₃), or 2-propyl (—CH(CH₃)₂).

### C₁₋₃ Alkyl Groups

C₁₋₃ alkyl groups that may be present as a group R² and X8 in the context of compounds of the present invention include methyl(Me or —CH₃), ethyl (—CH₂CH₃), 1-propyl (—CH₂CH₂CH₃), and 2-propyl (—CH(CH₃)₂).

For X8, the C₁₋₃ alkyl group may be optionally substituted with NH₂, such as —CH₂CH₂NH₂.

### C₁₋₄ Alkylene Groups

C₁₋₄ alkylene groups that may be present as a group L of the dithioether bridge in the context of compounds of the present invention include, but are not limited to, C₁₋₂ alkylene groups, such as methylene (—CH₂—) and ethylene (—CH₂CH₂—).

### Arylene Groups

Arylene groups that may be present as a group Y of the dithioether bridge in the context of compounds of the present invention include, but are not limited to, phenylene (such as 1,2-phenylene, 1,3-phenylene, and 1,4-phenylene).

### Bridging Moieties

The sequences disclosed herein containing bridging moieties noted in the rounded brackets (e.g., (1a), (2a), etc.). These represent chemical bridges between the specific residue pairs. Each rounded bracket will appear twice in the sequence as a pair to indicate a single bridging moiety. Most of the sequences have two bridging moieties, indicated by 4 sets of rounded brackets meaning two bridging moiety pairs.

The number in the rounded bracket indicates a specific bridging moiety pair (e.g. “1” indicates a bridge between amino acid residues at positions 2 and I1, which is also indicated by the square bracket notation defining the specific chemical bridge). The letter indicates the type of chemical bridge (e.g. “a” indicates a 1,3-dithio-propan-2-one bridge).

The specific chemical bridge is defined at the end of the tables, using a square bracket (e.g. [2,11], [4,7], etc.) to indicate the amino acid residues using in the bridging moiety as compared to the original starting peptide (the I3 peptide (isomer 3) as described in Example 2), so these may not line-up specifically with the actual amino acid numbering of the SEQ ID NO: (as in some of these sequences for example, the first amino acid residue had been deleted as compared to the original starting peptide).

The residue directly preceding the rounded bracket notation indicates that specific residue is used in the bridging moiety.

The “*” notation directly after the rounded bracket notation indicates that the terminal —NH₂ (if at the start of the sequence i.e. the N-terminus) or —COOH (if at the end of the sequence i.e. the C-terminus) is used to form the bridge. When the bridging moiety is a triazole and the “*” notation is used on the N-terminus, the terminal —NH₂ has been converted to an azide (—N₃) of the N-terminal amino acid residue.

### Compounds

The invention provides compounds which are peptide inhibitors of IL-23R.

The invention provides a compound of the formula:

R¹—Z—R²
wherein
R¹ is H, C₁₋₄ acyl, benzoyl, C₁₋₄ alkyl, or is absent;
R² is NHR³, OH, or is absent, wherein R³ is hydrogen or C₁₋₃ alkyl optionally substituted with NH₂; and
Z is an amino acid sequence of formula I:

X1-X2-X3-X4-X5-X6-X7-X8-X9-X10-X11-X12-X13-X14 (1)
wherein
X1 is absent or is selected from the group consisting of Asp, Gly, Leu, Glu, Ser, Cys, and Lys;
X3 is selected from the group consisting of Ser, beta-homo-Ser, Thr, Leu, Cys, Gln, Val, Ile, N-Me-Ser, and Q(pyrrolidin);
X5 is selected from the group consisting of Trp, Tyr, Ala, 1-Me-Trp, 7-Me-Trp, 7-Ph-Trp, 7-(Naphth-2-yl)-Trp, 2-Nal, Bip, 4-F-Trp, 7-F-Trp, and N-Me-Trp;
X6 is selected from the group consisting of Gln, Glu, Tyr, Cys, Val, His, N-Me-Gln, and Q(pyrrolidin);
X8 is selected from the group consisting of Trp, Tyr, Asn, Ala, His, 2-Nal, Dab, 2,4-diaminobutanoyl([2-(trimethyl-2-aminoethoxy)ethoxy]propyl), F(4-NH₂-(2-(trimethyl-2-aminoethoxy)ethoxy)propyl), Phe, Val, 4-Me-Phe, 2-Me-Phe, Bip, 2-Me-F(4-F), {d}F(4-F), 4-Cl-Phe, alpha-Me-Trp, 3,3-Diphenyl-Ala, and Phg, wherein the hydroxyl group of Tyr is optionally substituted with C₁₋₃ alkyl optionally substituted with NH₂;
X9 is selected from the group consisting of 2-Nal, Trp, 1-Me-Trp, 6-Cl-Trp, 3-(3-Quinolinyl)-Ala, Phe, 4-F-Phe, Glu, Cys, Ala, 6-F-Trp, His, 3-F-Phe, 3,4-Me-Phe, Bip, and {d}6-F-Trp;
X10 is selected from the group consisting of Leu, D-Leu, 2-Me-Leu, 2-Me-Lys, Trp, Asn, Cys, 4-aminotetrahydro-2H-pyran-4-acetyl, and 2-Me-Val;
X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, 3-(4-Pyridyl)-Ala, {d}2,4-diaminobutanoyl([2-(trimethyl-2-aminoethoxy)ethoxy]propyl), D-Gln, D-Glu, D-His, 3-aminopropanoyl, and GABA, or is absent;
X13 is absent or is selected from the group consisting of Asn, Gly, 3-(3-Pyridyl)-Ala, 3-(4-Pyridyl)-Ala, 3-(3-Quinolinyl)-Ala, {d}[3-(3-Pyridyl)-Ala], 3-amino-3-(3′-pyridyl)propionyl, 3-F-Phe, 3,5-F-Phe, 4-aminomethyl-2-pyridineacetyl, 2,3-diaminopropanoyl(3-pyridylacetyl), 2,3-diaminopropanoyl(3-pyridylpropionyl), 2,3-diaminopropanoyl(3-fluorobenzoyl), 2,3-diaminopropanoyl(3-fluorophenylacetyl), and 2-Me-3-(3-Pyridyl)-Ala;
X14 is absent or is Gly;
X2 and X11 are amino acid residues who together form a lactam bridge, a dithioether bridge, or a bridge containing a triazole ring; and
X4 and X7 are amino acid residues who together form a lactam bridge, a dithioether bridge, or a bridge containing a triazole ring;
or a pharmaceutically acceptable salt or solvate thereof;
wherein the compound is not:

I3(isomer 3)H-DC(1a)SC(2a)WQC(2a)WWLC(1a)R-[NH₂];
wherein (1a) is a [2,11]1,3-dithio-propan-2-one bridge and (2a) is a [4,7]1,3-dithio-propan-2-one bridge.

In some embodiments, the invention provides a compound of the formula:
R¹—Z—R²
wherein
R¹ is H, C₁₋₄ acyl, benzoyl, C₁₋₄ alkyl, or is absent;
R² is NHR³, OH, or is absent, wherein R³ is hydrogen or C₁₋₃ alkyl; and
Z is an amino acid sequence of formula Ia:

X1-X2-X3-X4-X5-X6-X7-X8-X9-X10-X11-X12-X13-X14 (Ia)
wherein
X1 is absent or is selected from the group consisting of Asp, Glu, Ser, Cys, and Lys;
X3 is selected from the group consisting of Ser, beta-homo-Ser, Thr, Leu, Cys, and Gln;
X5 is selected from the group consisting of Trp, Tyr, Ala, 1-Me-Trp, 7-Me-Trp, 7-Ph-Trp, 7-(Naphth-2-yl)-Trp, 2-Nal, and Bip;
X6 is selected from the group consisting of Gln, Glu, Tyr and Cys;
X8 is selected from the group consisting of Trp, Tyr, Asn, Ala, His, and 2-Nal, wherein the hydroxyl group of Tyr is optionally substituted with C₁₋₃ alkyl optionally substituted with NH₂;
X9 is selected from the group consisting of 2-Nal, Trp, 1-Me-Trp, 6-Cl-Trp, 3-(3-Quinolinyl)-Ala, Phe, 4-F-Phe, Glu, Cys, and Ala;
X10 is selected from the group consisting of Leu, D-Leu, 2-Me-Leu, 2-Me-Lys, Trp, Asn, Cys, and 4-aminotetrahydro-2H-pyran-4-acetyl;
X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent;
X13 is absent or is selected from the group consisting of Asn, Gly, 3-(3-Pyridyl)-Ala, and 3-(4-Pyridyl)-Ala;
X14 is absent or is Gly;
X2 and X11 are amino acid residues who together form a lactam bridge, a dithioether bridge, or a bridge containing a triazole ring; and
X4 and X7 are amino acid residues who together form a lactam bridge, a dithioether bridge, or a bridge containing a triazole ring;
or a pharmaceutically acceptable salt or solvate thereof;
wherein the compound is not:

I3(isomer 3)H-DC(1a)SC(2a)WQC(2a)WWLC(1a)R-[NH₂];
wherein (1a) is a [2,11]1,3-dithio-propan-2-one bridge and (2a) is a [4,7]1,3-dithio-propan-2-one bridge.

In some embodiments, the invention provides a compound of the formula:

R¹—Z—R²
wherein
R¹ is H, C₁₋₄ acyl, benzoyl, C₁₋₄ alkyl, or is absent;
R² is NHR³, OH, or is absent, wherein R³ is hydrogen or C₁₋₃ alkyl optionally substituted with NH₂; and
Z is an amino acid sequence of formula II:

X1-X2-X3-X4-X5-X6-X7-X8-X9-X10-X11-X12-X13-X14 (II)

wherein
X1 is absent or Asp;
X3 is Ser or lie;
X5 is Trp or 7-Me-Trp;
X6 is Gln or Glu;
X8 is Trp, Tyr, or 4-Me-Phe; wherein the hydroxyl group of Tyr is optionally substituted with —CH₂CH₂NH₂;
X9 is 2-Nal, Trp, or 3,4-Me-Phe;
X10 is Leu, 2-Me-Leu, or 2-Me-Val;
X12 is Arg, D-Arg, and Dab;
X13 is absent, 3-(3-Pyridyl)-Ala, or 2-Me-3-(3-Pyridyl)-Ala;
X14 is absent;
X2 and X11 are amino acid residues who together form a lactam bridge, a dithioether bridge, or a bridge containing a triazole ring; and
X4 and X7 are amino acid residues who together form a lactam bridge, a dithioether bridge, or a bridge containing a triazole ring;
or a pharmaceutically acceptable salt or solvate thereof;
wherein the compound is not:

I3(isomer 3)H-DC(1a)SC(2a)WQC(2a)WWLC(1a)R-[NH₂];
wherein (1a) is a [2,11]1,3-dithio-propan-2-one bridge and (2a) is a [4,7]1,3-dithio-propan-2-one bridge.

In some embodiments of formulae I, Ia, or II, X2 and X11 are amino acid residues who together form a lactam bridge or a dithioether bridge. In some embodiments of formulae I, Ia, or II, X2 and X11 are amino acid residues who together form a lactam bridge. In some such embodiments, X2 is (3-aminomethyl)benzoyl and X11 is Glu; X2 is 4-aminomethyl-phenylacetyl and X11 is Glu; X2 is Glu and X11 is Lys; or X2 is Lys and X11 is Glu.

In some embodiments of formulae I, Ia, or II, X4 and X7 are amino acid residues who together form a lactam bridge or a dithioether bridge.

In some embodiments of formulae I, Ia, or II, X4 and X7 are amino acid residues who together form a dithioether bridge. In some such embodiments, the dithioether bridge is of formula —S—L—Y—L—S—, such as —SCH₂C(═O)CH₂S—. In some such embodiments, X4 is Cys and X7 is Cys.

In some embodiments of formulae I, Ia, or II, X4 and X7 are amino acid residues who together form a lactam bridge. In some such embodiments, X4 is Glu and X7 is Dab.

In some embodiments of formulae I, Ia, or II, X2 and X11 are amino acid residues who together form a lactam bridge; and X4 and X7 are amino acid residues who together form a lactam bridge or a dithioether bridge. In some embodiments of formulae I, Ia, or II, X2 and X11 are amino acid residues who together form a lactam bridge; and X4 and X7 are amino acid residues who together form a dithioether bridge. In some embodiments of formulae I, Ia, or II, X2 and X11 are amino acid residues who together form a lactam bridge; and X4 and X7 are amino acid residues who together form a lactam bridge.

Any internal truncation, that is deletion of amino acid residues, between X2 and X11 leads to inactive compounds (see reference compounds Ref 5, Ref 6, and Ref 7 in the Examples below).

It will be understood that the invention encompasses salts and solvates of the compounds. Suitable salts and solvates of peptides are known in the art.

It will also be understood any of the following references to embodiments may be applicable and are combinable with any of the formulae described herein.

### R¹

R¹ is H, C₁₋₄ acyl, benzoyl, C₁₋₄ alkyl, or is absent.

In some embodiments, R¹ is H, C₁₋₂ acyl, or absent. In some embodiments, R¹ is H, —C(═O)CH₃, or absent. In some embodiments, R¹ is H or C₁₋₂ acyl. In some embodiments, R¹ is H. In some embodiments, R¹ is —C(═O)CH₃. In some embodiments,
R¹ is absent.

In some embodiments, R¹ is absent when X¹ is absent.

In some embodiments, X1 is absent, R¹ is absent, and X2 and X11 are amino acid residues that together form a lactam bridge or a bridge containing a triazole ring via the N-terminus of X2.

In some embodiments, X1 and X12 to X14 are absent, R¹ and R² are absent, and X2 and X11 are amino acid residues that together form a head-to-tail cyclised lactam bridge via the N-terminus of X2 and the C-terminus of X11.

### R²

R² is NHR³, OH, or is absent, wherein R³ is hydrogen or C₁₋₃ alkyl optionally substituted with NH₂.

In some embodiments, R² is NHR³, OH, or is absent, wherein R³ is hydrogen or C₁₋₃ alkyl.

In some embodiments, R² is NH₂, NH—(CH₂)₃—NH₂ (that is NH-3-aminopropanoyl), OH, or is absent.

In some embodiments, R² is NHR³. In some embodiments, R² is NH—(CH₂)—NH₂, that is NH-3-aminopropanoyl. In some embodiments, R² is NH₂. In some embodiments, R¹ is OH. In some embodiments, R² is absent.

Preferably, R² is NH₂.

In some embodiments, X1 and X12 to X14 are absent, R¹ and R² are absent, and X2 and X11 are amino acid residues that together form a head-to-tail cyclised lactam bridge via the N-terminus of X2 and the C-terminus of X11.

### Z

Z is an amino acid sequence of formula I:

X1-X2-X3-X4-X5-X6-X7-X8-X9-X10-X11-X12-X13-X14 (I)
X1 is absent or is selected from the group consisting of Asp, Gly, Leu, Glu, Ser, Cys, and Lys;
X3 is selected from the group consisting of Ser, beta-homo-Ser, Thr, Leu, Cys, Gln, Val, Ile, N-Me-Ser, and Q(pyrrolidin);
X5 is selected from the group consisting of Trp, Tyr, Ala, 1-Me-Trp, 7-Me-Trp, 7-Ph-Trp, 7-(Naphth-2-yl)-Trp, 2-Nal, Bip, 4-F-Trp, 7-F-Trp, and N-Me-Trp;
X6 is selected from the group consisting of Gln, Glu, Tyr, Cys, Val, His, N-Me-Gln, and Q(pyrrolidin);
X8 is selected from the group consisting of Trp, Tyr, Asn, Ala, His, 2-Nal, Dab, 2,4-diaminobutanoyl([2-(trimethyl-2-aminoethoxy)ethoxy]propyl), F(4-NH₂-(2-(trimethyl-2-aminoethoxy)ethoxy)propyl), Phe, Val, 4-Me-Phe, 2-Me-Phe, Bip, 2-Me-F(4-F), {d}F(4-F), 4-Cl-Phe, alpha-Me-Trp, 3,3-Diphenyl-Ala, and Phg, wherein the hydroxyl group of Tyr is optionally substituted with C₁₋₃ alkyl optionally substituted with NH₂;
X9 is selected from the group consisting of 2-Nal, Trp, 1-Me-Trp, 6-Cl-Trp, 3-(3-Quinolinyl)-Ala, Phe, 4-F-Phe, Glu, Cys, Ala, 6-F-Trp, His, 3-F-Phe, 3,4-Me-Phe, Bip, and {d}6-F-Trp;
X10 is selected from the group consisting of Leu, D-Leu, 2-Me-Leu, 2-Me-Lys, Trp, Asn, Cys, 4-aminotetrahydro-2H-pyran-4-acetyl, and 2-Me-Val;
X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, 3-(4-Pyridyl)-Ala, {d}2,4-diaminobutanoyl([2-(trimethyl-2-aminoethoxy)ethoxy]propyl), D-Gln, D-Glu, D-His, 3-aminopropanoyl, and GABA, or is absent;
X13 is absent or is selected from the group consisting of Asn, Gly, 3-(3-Pyridyl)-Ala, 3-(4-Pyridyl)-Ala, 3-(3-Quinolinyl)-Ala, {d}[3-(3-Pyridyl)-Ala], 3-amino-3-(3′-pyridyl)propionyl, 3-F-Phe, 3,5-F-Phe, 4-aminomethyl-2-pyridineacetyl, 2,3-diaminopropanoyl(3-pyridylacetyl), 2,3-diaminopropanoyl(3-pyridylpropionyl), 2,3-diaminopropanoyl(3-fluorobenzoyl), 2,3-diaminopropanoyl(3-fluorophenylacetyl), and 2-Me-3-(3-Pyridyl)-Ala;
X14 is absent or is Gly;
X2 and X11 are amino acid residues who together form a lactam bridge, a dithioether bridge, or a bridge containing a triazole ring; and
X4 and X7 are amino acid residues who together form a lactam bridge, a dithioether bridge, or a bridge containing a triazole ring.

In some embodiments, Z is an amino acid sequence of formula Ia:

X1-X2-X3-X4-X5-X6-X7-X8-X9-X10-X11-X12-X13-X14 (Ia)
wherein
X1 is absent or is selected from the group consisting of Asp, Glu, Ser, Cys, and Lys;
X3 is selected from the group consisting of Ser, beta-homo-Ser, Thr, Leu, Cys, and Gln;
X5 is selected from the group consisting of Trp, Tyr, Ala, 1-Me-Trp, 7-Me-Trp, 7-Ph-Trp, 7-(Naphth-2-yl)-Trp, 2-Nal, and Bip;
X6 is selected from the group consisting of Gln, Glu, Tyr and Cys;
X8 is selected from the group consisting of Trp, Tyr, Asn, Ala, His, and 2-Nal, wherein the hydroxyl group of Tyr is optionally substituted with C₁₋₃ alkyl optionally substituted with NH₂;
X9 is selected from the group consisting of 2-Nal, Trp, 1-Me-Trp, 6-Cl-Trp, 3-(3-Quinolinyl)-Ala, Phe, 4-F-Phe, Glu, Cys, and Ala;
X10 is selected from the group consisting of Leu, D-Leu, 2-Me-Leu, 2-Me-Lys, Trp, Asn, Cys, and 4-aminotetrahydro-2H-pyran-4-acetyl;
X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent;
X13 is absent or is selected from the group consisting of Asn, Gly, 3-(3-Pyridyl)-Ala, and 3-(4-Pyridyl)-Ala;
X14 is absent or is Gly;
X2 and X11 are amino acid residues who together form a lactam bridge, a dithioether bridge, or a bridge containing a triazole ring; and
X4 and X7 are amino acid residues who together form a lactam bridge, a dithioether bridge, or a bridge containing a triazole ring.

In some embodiments, Z is an amino acid sequence of formula II:

X1-X2-X3-X4-X5-X6-X7-X8-X9-X10-X11-X12-X13-X14 (II)
wherein
X1 is absent or Asp;
X3 is Ser or lie;
X5 is Trp or 7-Me-Trp;
X6 is Gln or Glu;
X8 is Trp, Tyr, or 4-Me-Phe; wherein the hydroxyl group of Tyr is optionally substituted with —CH₂CH₂NH₂;
X9 is 2-Nal, Trp, or 3,4-Me-Phe;
X10 is Leu, 2-Me-Leu, or 2-Me-Val;
X12 is Arg, D-Arg, and Dab;
X13 is absent, 3-(3-Pyridyl)-Ala, or 2-Me-3-(3-Pyridyl)-Ala;
X14 is absent;
and X2, X4, X7, and X11 are defined as described above for formula I.

In some embodiments, Z is an amino acid sequence of formula III:

X1-X2-X3-X4-X5-Gln-X7-X8-X9-X10-X11-X12 (III)
wherein X1, X2, X3, X4, X5, X7, X8, X9, X10, X11, and X12 are defined as described above for formulae I, Ia, or II.

In some embodiments, Z is an amino acid sequence of formula IV:

X2-Ser-X4-Trp-Gln-X7-X8-(2-Nal)-Leu-X11-Arg (IV)

wherein X2, X4, X7, X8, and X11 are defined as described above for formula I, Ia, or II.

In some embodiments, Z is an amino acid sequence of formula V:

X2-Ser-X4-Trp-Gln-X7-X8-(2-Nal)-Leu-X11-Arg (V)
wherein
X8 is selected from the group consisting of Trp and Tyr, wherein the hydroxyl group of Tyr is optionally substituted with C₁₋₃ alkyl optionally substituted with NH₂;
X2 is Lys and X11 is Gin, who together form a lactam bridge;
X4 and X7 are both Cys who together form a dithioether bridge.

In some embodiments, Z is an amino acid sequence selected from the group consisting of:

| | |
|---|---|
| SEQ ID NO: 1 | DC(1a)SC(2a)WQC(2a)WW[2-Me-Leu]C(1a)R |
| SEQ ID NO: 2 | LC(1a)SC(2a)WQC(2a)WWLC(1a)R |
| SEQ ID NO: 3 | DC(1a)SC(2a)WEC(2a)WWLC(1a)R |
| SEQ ID NO: 4 | DE(1c)SC(2a)WQC(2a)WWLK(1c)R |
| SEQ ID NO: 5 | DC(1a)SE(2c)WQK(2c)WWLC(1a)R |
| SEQ ID NO: 6 | D(1c)*SC(2a)WQC(2a)WWLR(1c)* |
| SEQ ID NO: 7 | DK(1c)SC(2a)WQC(2a)WWLE(1c)R |
| SEQ ID NO: 8 | D[Orn](1c)SC(2a)WQC(2a)WWLE(1c)R |
| SEQ ID NO: 9 | DE(1c)SC(2a)WQC(2a)WWL[Dab](1c)R |
| SEQ ID NO: 10 | E(1c)SC(2a)WQC(2a)WWLK(1c)R |
| SEQ ID NO: 11 | DE(1c)SC(2a)WQC(2a)WWLK(1c) |
| SEQ ID NO: 12 | E(1c)SC(2a)WQC(2a)WWLK(1c) |
| SEQ ID NO: 13 | DE(1c)SC(2a)AQC(2a)WWLK(1c)R |
| SEQ ID NO: 14 | DE(1c)SC(2a)WQC(2a)AWLK(1c)R |
| SEQ ID NO: 15 | DE(1c)SC(2a)WQC(2a)WALK(1c)R |
| SEQ ID NO: 16 | DE(1c)SC(2a)[7-Me-Trp]QC(2a)WWLK(1c)R |
| SEQ ID NO: 17 | DE(1c)SC(2a)WQC(2a)W[2-Nal]LK(1c)R |
| SEQ ID NO: 18 | K(1c)SC(2a)WQC(2a)W[2-Nal]LE(1c)R |
| SEQ ID NO: 19 | DK(1c)SC(2a)WQC(2a)W[2-Nal]LE(1c)R |
| SEQ ID NO: 20 | DK(1c)SC(2d)WQC(2d)W[2-Nal]LE(1c)R |
| SEQ ID NO: 21 | DK(1c)SC(2e)WQC(2e)W[2-Nal]LE(1c)R |
| SEQ ID NO: 22 | DK(1c)SC(2f)WQC(2f)W[2-Nal]LE(1c)R |
| SEQ ID NO: 23 | DK(1c)S[K(N3)](2g)WQ[Pra](2g)W[2-Nal]LE(1c)R |
| SEQ ID NO: 24 | DK(1c)S[Aha](2g)WQ[Pra](2g)W[2-Nal]LE(1c)R |
| SEQ ID NO: 25 | K(1c)SC(2a)[2-Nal]QC(2a)W[2-Nal]LE(1c)R |
| SEQ ID NO: 26 | K(1c)SC(2a)[Bip]QC(2a)W[2-Nal]LE(1c)R |
| SEQ ID NO: 27 | K(1c)SC(2a)[1-Me-Trp]QC(2a)W[2-Nal]LE(1c)R |
| SEQ ID NO: 28 | DK(1c)SC(2a)WQC(2a)[2-Nal][2-Nal]LE(1c)R |
| SEQ ID NO: 29 | K(1c)SC(2a)WQC(2a)[Y(2-aminoethoxy)][2-Nal]LE(1c)R |
| SEQ ID NO: 30 | K(1c)SC(2a)WQC(2a)H[2-Nal]LE(1c)R |
| SEQ ID NO: 31 | K(1c)SC(2a)WQC(2a)W[F(4-F)]LE(1c)R |
| SEQ ID NO: 32 | K(1c)SC(2a)WQC(2a)W[1-Me-Trp]LE(1c)R |
| SEQ ID NO: 33 | C(1a)TC(2a)WEC(2a)WW[2-Me-Leu]C(1a)S |
| SEQ ID NO: 34 | GK(1c)TC(2a)WEC(2c)WW[2-Me-Leu]E(1c)S |
| SEQ ID NO: 35 | GC(1a)TC(2a)WEC(2a)W[2-Nal][2-Me-Leu]C(1a)R |
| SEQ ID NO: 36 | [bAla](1c)*SC(2a)WQC(2a)W[2-Nal]LE(1c)R |
| SEQ ID NO: 37 | [(3-Aminomethyl)benzoyl](1c)*SC(2a)WQC(2a)W[2-Nal]LE(1c)R |
| SEQ ID NO: 38 | [(3-Aminomethyl)benzoyl](1c)*SC(2a)WQC(2a)[Y(2-aminoethoxy)][2- |
| | Nal][2-Me-Leu]E(1c)R-[NH2] |
| SEQ ID NO: 39 | [(3-Aminomethyl)benzoyl](1c)*SC(2a)WQC(2a)[Y(2-aminoethoxy)][2- |
| | Nal]LE(1c)R |
| SEQ ID NO: 40 | [(3-Aminomethyl)benzoyl](1c)*SC(2a)WQC(2a)W[2-Nal][2-Me-Leu]E(1c)R |
| SEQ ID NO: 41 | [(3-Aminomethyl)benzoyl](1c)*SC(2a)WQC(2a)[Y(2-aminoethoxy)][2- |
| | Nal][2-Me-Leu]E(1c)[{d}R] |
| SEQ ID NO: 42 | [(3-Aminomethyl)benzoyl](1c)*SC(2a)WQC(2a)[Y(2-aminoethoxy)][2- |
| | Nal][2-Me-Leu]E(1c)[2-Me-Arg] |
| SEQ ID NO: 43 | [(3-Aminomethyl)benzoyl](1c)*SC(2a)WQC(2a)[Y(2-aminoethoxy)][2- |
| | Nal][2-Me-Leu]E(1c)[N-Me-Arg] |
| SEQ ID NO: 44 | [(3-Aminomethyl)benzoyl](1c)*SC(2a)WQC(2a)[Y(2-aminoethoxy)][2- |
| | Nal][2-Me-Leu]E(1c)S |
| SEQ ID NO: 45 | [(3-Aminomethyl)benzoyl](1c)*SC(2a)WQC(2a)[Y(2-aminoethoxy)][2- |
| | Nal][2-Me-Leu]E(1c)[F(4-NH2)] |
| SEQ ID NO: 46 | [(3-Aminomethyl)benzoyl](1c)*SC(2a)WQC(2a)[Y(2-aminoethoxy)][2- |
| | Nal][{d}LJE(1c)Y |
| SEQ ID NO: 47 | [Ac]-K(1c)SC(2a)WQC(2a)[Y(2-aminoethoxy)][2-Nal][2-Me-Leu]E(1c)R |
| SEQ ID NO: 48 | [Ac]-K(1c)SC(2a)WQC(2a)[Y(2-aminoethoxy)][2-Nal][{d}L]E(1c)R |
| SEQ ID NO: 49 | [(4-Aminomethyl)benzoyl](1c)*SC(2a)WQC(2a)[Y(2-aminoethoxy)][2- |
| | Nal][2-Me-Leu]E(1c)R |
| SEQ ID NO: 50 | [4-(2-Aminoethyl)benzoyl](1c)*SC(2a)WQC(2a)[Y(2-aminoethoxy)][2- |
| | Nal][2-Me-Leu]E(1c)R |
| SEQ ID NO: 51 | [3-(4-Aminophenyl)propanoyl](1c)*SC(2a)WQC(2a)[Y(2-aminoethoxy)][2- |
| | Nal][2-Me-Leu]E(1c)R |
| SEQ ID NO: 52 | [4-Aminomethyl-phenylacetyl](1c)*SC(2a)WQC(2a)[Y(2-aminoethoxy)][2- |
| | Nal][2-Me-Leu]E(1c)R |
| SEQ ID NO: 53 | DC(1a)SC(2b)WQC(2b)WWLC(1a)R |
| SEQ ID NO: 54 | K(1c)SC(2b)WQC(2b)[Y(2-aminoethoxy)][2-Nal]LE(1c)R |
| SEQ ID NO: 55 | DE(1c)QC(2a)WQC(2a)YW[2-Me-Leu]K(1c)R |
| SEQ ID NO: 56 | DE(1c)QC(2a)WQC(2a)[2-Nal]W[2-Me-Leu]K(1c)R |
| SEQ ID NO: 57 | DE(1c)TC(2a)WQC(2a)W[2-Nal][2-Me-Leu]K(1c) |
| SEQ ID NO: 58 | DE(1c)SC(2a)WQC(2a)W[2-Nal][2-Me-Leu]K(1c)ENG |
| SEQ ID NO: 59 | DE(1c)SC(2a)WQC(2a)W[2-Nal][2-Me-Leu]K(1c)NG |
| SEQ ID NO: 60 | DE(1c)SC(2a)WQC(2a)W[2-Nal][2-Me-Leu]K(1c)G |
| SEQ ID NO: 61 | DE(1c)SC(2a)WQC(2a)W[2-Nal][2-Me-Lys]K(1c)ENG |
| SEQ ID NO: 62 | DC(1a)QC(2a)WQC(2a)[2-Nal]W[2-Me-Leu]C(1a)R |
| SEQ ID NO: 63 | DC(1a)QC(2a)WQC(2a)YW[2-Me-Leu]C(1a)R |
| SEQ ID NO: 64 | DC(1a)QC(2a)WQC(2a)WW[2-Me-Leu]C(1a)ENG |
| SEQ ID NO: 65 | [(3-Aminomethyl)benzoyl](1c)*S[Dpr](2c)WQE(2c)[Y(2-aminoethoxy)][2- |
| | Nal][2-Me-Leu]E(1c)R |
| SEQ ID NO: 66 | [(3-Aminomethyl)benzoyl](1c)*SE(2c)WQ[Dpr](2c)[Y(2-aminoethoxy)][2- |
| | Nal][2-Me-Leu]E(1c)R |
| SEQ ID NO: 67 | [(3-Aminomethyl)benzoyl](1c)*SE(2c)WQ[Dab](2c)[Y (2-aminoethoxy)][2- |
| | Nal][2-Me-Leu]E(1c)R |
| SEQ ID NO: 68 | [(3-Aminomethyl)benzoyl](1c)*S[Orn](2c)WQE(2c)[Y(2-aminoethoxy)][2- |
| | Nal][2-Me-Leu]E(1c)R |
| SEQ ID NO: 69 | [(3-Aminomethyl)benzoyl](1c)*SE(2c)WQ[Orn](2c)[Y(2-aminoethoxy)][2- |
| | Nal][2-Me-Leu]E(1c)R |
| SEQ ID NO: 70 | [(3-Aminomethyl)benzoyl](1c)*SC(2a)[7-Me-Trp]QC(2a)[Y(2- |
| | aminoethoxy)][2-Nal][2-Me-Leu]E(1c)R |
| SEQ ID NO: 71 | [(3-Aminomethyl)benzoyl](1c)*SC(2a)[7-Ph-Trp]QC(2a)[Y(2- |
| | aminoethoxy)][2-Nal][2-Me-Leu]E(1c)R |
| SEQ ID NO: 72 | [(3-Aminomethyl)benzoyl](1c)*SC(2a)[7-(Naphth-2-yl)-Trp]QC(2a)[Y(2- |
| | aminoethoxy)][2-Nal][2-Me-Leu]E(1c)R |
| SEQ ID NO: 73 | [(3-Aminomethyl)benzoyl](1c)*SC(2a)WQC(2a)[Y(2-aminoethoxy)][3-(3- |
| | Quinolinyl)-Ala][2-Me-Leu]E(1c)R |
| SEQ ID NO: 74 | [(3-Aminomethyl)benzoyl](1c)*SC(2a)WQC(2a)[Y(2-aminoethoxy)][6-CI- |
| | Trp][2-Me-Leu]E(1c)R |
| SEQ ID NO: 75 | [(3-Aminomethyl)benzoyl](1c)*SC(2a)WQC(2a)[Y(2-aminoethoxy)][2- |
| | Nal][4-aminotetrahydro-2H-pyran-4-acetyl]E(1c)R |
| SEQ ID NO: 76 | [(3-Aminomethyl)benzoyl](1c)*SC(2a)WQC(2a)[Y(2-aminoethoxy)][2- |
| | Nal][2-Me-Leu]E(1c)[3-(3-Pyridyl)-Ala] |
| SEQ ID NO: 77 | [(3-Aminomethyl)benzoyl](1c)*SC(2a)WQC(2a)[Y(2-aminoethoxy)][2- |
| | Nal][2-Me-Leu]E(1c)[3-(4-Pyridyl)-Ala] |
| SEQ ID NO: 78 | [(3-Aminomethyl)benzoyl](1c)*SC(2a)WQC(2a)[Y(2-aminoethoxy)][2- |
| | Nal][2-Me-Leu]E(1c)R[3-(3-Pyridyl)-Ala] |
| SEQ ID NO: 79 | [(3-Aminomethyl)benzoyl](1c)*SC(2a)WQC(2a)[Y(2-aminoethoxy)][2- |
| | Nal][2-Me-Leu]E(1c)R[3-(4-Pyridyl)-Ala] |
| SEQ ID NO: 80 | [4-Aminomethyl-phenylacetyl](1c)*SC(2a)WQC(2a)[Y(2-aminoethoxy)][2- |
| | Nal][2-Me-Leu]E(1c)[{d}R] |
| SEQ ID NO: 81 | [4-Aminomethyl-phenylacetyl](1c)*SC(2a)WQC(2a)[Y(2-aminoethoxy)][2- |
| | Nal][2-Me-Leu]E(1c)S |
| SEQ ID NO: 82 | [4-Aminomethyl-phenylacetyl](1c)*SC(2a)WQC(2a)[Y(2-aminoethoxy)][2- |
| | Nal][2-Me-Leu]E(1c)[Dab] |
| SEQ ID NO: 83 | [4-Aminomethyl-phenylacetyl](1c)*TC(2a)WQC(2a)[Y(2-aminoethoxy)][2- |
| | Nal][2-Me-Leu]E(1c)[{d}R] |
| SEQ ID NO: 84 | K(1c)SC(2a)WQC(2a)[Y(2-aminoethoxy)][2-Nal][2-Me-Leu]E(1c)[{d}R] |
| SEQ ID NO: 85 | K(1c) TC(2a)WQC(2a)[Y(2-aminoethoxy)][2-Nal][2-Me-Leu]E(1c)[{d}R] |
| SEQ ID NO: 86 | K(1c)SC(2a)WQC(2a)[Y(2-aminoethoxy)][2-Nal][2-Me-Leu]E(1c)S |
| SEQ ID NO: 87 | [6-Aminohexanoyl](1c)*SC(2a)WQC(2a)[Y(2-aminoethoxy)][2-Nal][2-Me- |
| | Leu]E(1c)[{d}R] |
| SEQ ID NO: 88 | [6-Amino-4-oxahexanoyl](1c)*SC(2a)WQC(2a)[Y(2-aminoethoxy)][2- |
| | Nal][2-Me-Leu]E(1c)[{d}R] |
| SEQ ID NO: 89 | [trans-4-Aminomethyl-cyclohexyl-1-carbonyl](1c)*[beta-homo- |
| | Ser]C(2a)WQC(2a)[Y(2-aminoethoxy)][2-Nal][2-Me-Leu]E(1c)[{d}R] |
| SEQ ID NO: 90 | [(4-(2-Aminoethyl)-piperazine-1-yl)-acetyl](1c)*SC(2a)WQC(2a)[Y(2- |
| | aminoethoxy)][2-Nal][2-Me-Leu]E(1c)[{d}R] |
| SEQ ID NO: 91 | [(4-(2-Aminoethyl)-piperazine-1-yl)-acetyl](1c)*SC(2a)WQC(2a)[Y(2- |
| | aminoethoxy)][2-Nal][2-Me-Leu]D(1c)[{d}R] |
| SEQ ID NO: 92 | [3-Aminomethyl-phenylacetyl](1c)*SC(2a)WQC(2a)[Y(2-aminoethoxy)][2- |
| | Nal][2-Me-Leu]E(1c)[{d}R] |
| SEQ ID NO: 93 | [2-Aminomethyl-phenylacetyl](1c)*SC(2a)WQC(2a)[Y(2-aminoethoxy)][2- |
| | Nal][2-Me-Leu]E(1c)[{d}R] |
| SEQ ID NO: 94 | [K(N3)](1g)SC(2a)WQC(2a)[Y(2-aminoethoxy)][2-Nal][2-Me- |
| | Leu][Pra](1g)[{d}R] |
| SEQ ID NO: 95 | [Azidoacetic acid](1g)*SC(2a)WQC(2a)[Y(2-aminoethoxy)][2-Nal][2-Me- |
| | Leu][Glu(propargylamine)] (1g)[{d}R] |
| SEQ ID NO: 96 | [Azidoacetic acid](1g)*SC(2a)WQC(2a)[Y(2-aminoethoxy)][2-Nal][2-Me- |
| | Leu][Dab(3-butynoic acid)](1g)[{d}R] |
| SEQ ID NO: 97 | [but-3-ynoic acid](1g)*SC(2a)WQC(2a)[Y(2-aminoethoxy)][2-Nal][2-Me- |
| | Leu][Dab(azidoacetic acid)](1g)[{d}R] |
| SEQ ID NO: 98 | [(N3)-Ala](1g)*SC(2a)WQC(2a)[Y(2-aminoethoxy)][2-Nal][2-Me- |
| | Leu][Glu(propargylamine)](1g)[{d}R] |
| SEQ ID NO: 99 | [(N3)-Ala](1g)*SC(2a)WQC(2a)[Y(2-aminoethoxy)][2-Nal][2-Me- |
| | Leu][Dab(3-butynoic acid)](1g)[{d}R] |
| SEQ ID NO: 100 | [but-3-ynoic acid](1g)*SC(2a)WQC(2a)[Y(2-aminoethoxy)][2-Nal][2-Me- |
| | Leu][Dab((N3)-Ala))](1g)[{d}R] |
| SEQ ID NO: 101 | [4-Aminomethyl-2-pyridineacetyl](1c)*SC(2a)WQC(2a)[Y(2- |
| | aminoethoxy)][2-Nal][2-Me-Leu]E(1c)[{d}R] |
| SEQ ID NO: 102 | [4-Aminomethyl-3-pyridineacetyl](1c)*SC(2a)WQC(2a)[Y(2- |
| | aminoethoxy)][2-Nal][2-Me-Leu]E(1c)[{d}R] |
| SEQ ID NO: 103 | [4-Aminomethyl-2-fluoro-phenylacetyl](1c)*SC(2a)WQC(2a)[Y(2- |
| | aminoethoxy)][2-Nal][2-Me-Leu]E(1c)[{d}R] |
| SEQ ID NO: 104 | [4-Aminomethyl-3-methyl-phenylacetyl](1c)*SC(2a)WQC(2a)[Y(2- |
| | aminoethoxy)][2-Nal][2-Me-Leu]E(1c)[{d}R] |
| SEQ ID NO: 105 | [4-Aminomethyl-3-methoxy-phenylacetyl](1c)*SC(2a)WQC(2a)[Y(2- |

wherein:
*= bridge uses the peptide backbone amine or carboxylic acid at the N- or C-terminus, not the side chain amine or carboxylic acid
(1a) = [2,11] 1,3-dithio-propan-2-one bridge; (1c) = [2,11] lactam bridge; (1g) = [2,11] 1,4-disubstituted 1,2,3-triazole bridge; (2a) = [4,7] 1,3-dithio-propan-2-one bridge; (2c) = [4,7] lactam bridge; (2d) = [4,7] 1,2-phenylenedimethanethiol bridge; (2e) = [4,7] 1,3-phenylenedimethanethiol bridge; (2f) = [4,7] 1,4-phenylenedimethanethiol bridge; (2g) =
[4,7] 1,4-disubstituted 1,2,3-triazole bridge.

In some embodiments, Z is an amino acid sequence selected from the group consisting of:

| | |
|---|---|
| SEQ ID NO: 106 | [but-3-ynoic acid] (1h)*SC(2a)WQC(2a)[Y(2-aminoethoxy)][2-Nal][2-Me- |
| | Leu][Dab(azidoacetic acid)](1h)[{d}R] |
| SEQ ID NO: 107 | [but-3-ynoic acid](1h)*SC(2a)WQC(2a)[Y(2-aminoethoxy)][2-Nal][2-Me- |
| | Leu][Dab((N3)-Ala)](1h)[{d}R] |
| SEQ ID NO: 108 | [4-Aminomethyl-phenylacetyl](1c)*SC(2a)WQC(2a)[Y(2-aminoethoxy)][2- |
| | Nal][2-Me-Leu]E(1c)[{d}R] |
| SEQ ID NO: 109 | [4-Aminomethyl-phenylacetyl](1c)*SC(2a)WQC(2a)[Y(2-aminoethoxy)][2- |
| | Nal][2-Me-Leu]E(1c)[{d}2,4-Diaminobutanoyl([2-(trimethyl-2- |
| | aminoethoxy)ethoxy]propyl)] |
| SEQ ID NO: 110 | [4-Aminomethyl-phenylacetyl](1c)*SC(2a)WQC(2a)[Dab][2-Nal][2-Me- |
| | Leu]E(1c)[{d}R] |
| SEQ ID NO: 111 | [4-Aminomethyl-phenylacetyl](1c)*SC(2a)WQC(2a)[2,4- |
| | Diaminobutanoyl([2-(trimethyl-2-aminoethoxy)ethoxy]propyl)][2-Nal][2-Me- |
| | Leu]E(1c)[{d}R] |
| SEQ ID NO: 112 | [Dab](1c)*SC(2a)WQC(2a)[Y(2-aminoethoxy)][2-Nal][2-Me- |
| | Leu]E(1c)[{d}R] |
| SEQ ID NO: 113 | [2,4-Diaminobutanoyl([2-(trimethyl-2- |
| | aminoethoxy)ethoxy]propyl)](1c)*SC(2a)WQC(2a)[Y(2-aminoethoxy)][2- |
| | Nal][2-Me-Leu]E(1c)[{d}R] |
| SEQ ID NO: 114 | [4-Aminomethyl-phenylacetyl](1c)*SC(2a)WQC(2a)[F(4-NH2-(2-(trimethyl- |
| | 2-aminoethoxy)ethoxy)propyl)][2-Nal][2-Me-Leu]E(1c)[{d}R] |
| SEQ ID NO: 115 | [4-Aminomethyl-phenylacetyl](1c)*SC(2a)WQC(2a)[Y(2-aminoethoxy)][2- |
| | Nal][2-Me-Leu]E(1c)[{d}Q] |
| SEQ ID NO: 116 | [4-Aminomethyl-phenylacetyl](1c)*SC(2a)WQC(2a)[Y(2-aminoethoxy)][2- |
| | Nal][2-Me-Leu]E(1c)[{d}E] |
| SEQ ID NO: 117 | [4-Aminomethyl-phenylacetyl](1c)*SC(2a)WQC(2a)[Y(2-aminoethoxy)][2- |
| | Nal][2-Me-Leu]E(1c)[{d}H] |
| SEQ ID NO: 118 | [4-Aminomethyl-phenylacetyl](1c)*SC(2a)WQC(2a)[Y(2-aminoethoxy)][2- |
| | Nal][2-Me-Leu]E(1c)[3-Aminopropanoy] |
| SEQ ID NO: 119 | [4-Aminomethyl-phenylacetyl](1c)*SC(2a)WQC(2a)[Y(2-aminoethoxy)][2- |
| | Nal][2-Me-Leu]E(1c)[3-Aminopropanoy]] |
| SEQ ID NO: 120 | [4-Aminomethyl-phenylacetyl](1c)*SC(2a)WQC(2a)[Y(2-aminoethoxy)][2- |
| | Nal][2-Me-Leu]E(1c)[Dab][3-(3-Pyridyl)-Ala] |
| SEQ ID NO: 121 | [4-Aminomethyl-phenylacetyl](1c)*SC(2a)WQC(2a)[Y(2-aminoethoxy)][6- |
| | F-Trp][2-Me-Leu]E(1c)[Dab][3-(3-Pyridyl)-Ala] |
| SEQ ID NO: 122 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala] |
| SEQ ID NO: 123 | [4-Aminomethyl-phenylacetyl](1c)*S[Dab](2c)WQE(2c)[Y(2- |
| | aminoethoxy)][2-Nal][2-Me-Leu]E(1c)[Dab][3-(3-Pyridyl)-Ala] |
| SEQ ID NO: 124 | [4-Aminomethyl-phenylacetyl](1c)*SE(2c)WQ[Orn](2c)[Y(2- |
| | aminoethoxy)][2-Nal][2-Me-Leu]E(1c)[Dab][3-(3-Pyridyl)-Ala] |
| SEQ ID NO: 125 | [4-Aminomethyl-phenylacetyl](1c)*SE(2c)WQ[Dab](2c)[Y(2- |
| | aminoethoxy)][2-Nal][2-Me-Leu]E(1c)[Dab] |
| SEQ ID NO: 126 | [4-Aminomethyl-phenylacetyl](1c)*VE(2c)WQ[Dab](2c)[Y(2- |
| | aminoethoxy][2-Nal][2-Me-Leu]E(1c)[Dab] |
| SEQ ID NO: 127 | [4-Aminomethyl-phenylacetyl](1c)*IE(2c) WQ[Dab](2c)[Y(2- |
| | aminoethoxy)][2-Nal][2-Me-Leu]E(1c)[Dab] |
| SEQ ID NO: 128 | [4-Aminomethyl-phenylacetyl](1c)*SE(2c)WV[Dab](2c)[Y(2- |
| | aminoethoxy)][2-Nal][2-Me-Leu]E(1c)[Dab] |
| SEQ ID NO: 129 | [4-Aminomethyl-phenylacetyl](1c)*SE(2c)WH[Dab](2c)[Y(2- |
| | aminoethoxy)][2-Nal][2-Me-Leu]E(1c)[Dab] |
| SEQ ID NO: 130 | [4-Aminomethyl-phenylacetyl](1c)*SE(2c)WQ[Dab](2c)F[2-Nal][2-Me- |
| | Leu]E(1c)[Dab] |
| SEQ ID NO: 131 | [4-Aminomethyl-phenylacetyl](1c)*SE(2c)WQ[Dab](2c)V[2-Nal][2-Me- |
| | Leu]E(1c)[Dab] |
| SEQ ID NO: 132 | [4-Aminomethyl-phenylacetyl](1c)*SE(2c)WQ[Dab](2c)[F(4-Me)][2-Nal][2- |
| | Me-Leu]E(1c)[Dab] |
| SEQ ID NO: 133 | [4-Aminomethyl-phenylacetyl](1c)*SE(2c)WQ[Dab](2c)[Y(2- |
| | aminoethoxy)]H[2-Me-Leu]E(1c)[Dab] |
| SEQ ID NO: 134 | [4-Aminomethyl-phenylacetyl](1c)*SE(2c)WQ[Dab](2c)[Y(2- |
| | aminoethoxy)][F(3-F)][2-Me-Leu]E(1c)[Dab] |
| SEQ ID NO: 135 | [4-Aminomethyl-phenylacetyl](1c)*SE(2c)WQ[Dab](2c)[Y(2- |
| | aminoethoxy)][F(3,4-Me)][2-Me-Leu]E(1c)[Dab] |
| SEQ ID NO: 136 | [4-Aminomethyl-phenylacetyl](1c)*SE(2c)WQ[Dab](2c)[Y(2- |
| | aminoethoxy)][2-Nal][2-Me-Val]E(1c)[Dab] |
| SEQ ID NO: 137 | [4-Aminomethyl-phenylacetyl](1c)*SE(2c)WQ[Dab](2c)[Y(2- |
| | aminoethoxy)][2-Nal][2-Me-Leu]E(1c) |
| SEQ ID NO: 138 | [4-Aminomethyl-phenylacetyl](1c)*SE(2c)[4-F-Trp]Q[Dab](2c)[Y (2- |
| | aminoethoxy)][2-Nal][2-Me-Leu]E(1c)[Dab] |
| SEQ ID NO: 139 | [4-Aminomethyl-phenylacetyl](1c)*SE(2c)[7-F-Trp]Q[Dab](2c)[Y (2- |
| | aminoethoxy)][2-Nal][2-Me-Leu]E(1c)[Dab] |
| SEQ ID NO: 140 | [4-Methylaminomethyl-phenylacetyl](1c)*SC(2a)WQC(2a)[Y(2- |
| | aminoethoxy)][2-Nal][2-Me-Leu]E(1c)[Dab] |
| SEQ ID NO: 141 | [4-Aminomethyl-phenylacetyl](1c)*[N-Me-Ser]C(2a)WQC(2a)[Y(2- |
| | aminoethoxy)][2-Nal][2-Me-Leu]E(1c)[Dab] |
| SEQ ID NO: 142 | [4-Aminomethyl-phenylacetyl](1c)*S[N-Me-Cys](2a)WQC(2a)[Y(2- |
| | aminoethoxy)][2-Nal][2-Me-Leu]E(1c)[Dab] |
| SEQ ID NO: 143 | [4-Aminomethyl-phenylacetyl](1c)*SC(2a)[N-Me-Trp]QC(2a)[Y (2- |
| | aminoethoxy)][2-Nal][2-Me-Leu]E(1c)[Dab] |
| SEQ ID NO: 144 | [4-Aminomethyl-phenylacetyl](1c)*SC(2a)W[N-Me-Gln]C(2a)[Y (2- |
| | aminoethoxy)][2-Nal][2-Me-Leu]E(1c)[Dab] |
| SEQ ID NO: 145 | [4-Aminomethyl-phenylacetyl](1c)*[Q(pyrrolidin)]E(2c)WQ[Dab](2c)[Y (2- |
| | aminoethoxy)][2-Nal][2-Me-Leu]E(1c)[Dab] |
| SEQ ID NO: 146 | [4-Aminomethyl-phenylacetyl](1c)*SE(2c)W[Q(pyrrolidin)] [Dab](2c)[Y(2- |
| | aminoethoxy)][2-Nal][2-Me-Leu]E(1c)[Dab] |
| SEQ ID NO: 147 | [4-Aminomethyl-phenylacetyl](1c)*SC(2a)WQC(2a)[Y(2-aminoethoxy)][2- |
| | Nal][2-Me-Leu]E(1c)R[3-(3-Pyridyl)-Ala] |
| SEQ ID NO: 148 | [(3-Aminomethyl)benzoyl](1c)*SC(2a)WQC(2a)[Y(2-aminoethoxy)][2- |
| | Nal][2-Me-Leu]E(1c)[Dab][3-(3-Pyridyl)-Ala] |
| SEQ ID NO: 149 | [(3-Aminomethyl)benzoyl](1c)*SC(2a)WQC(2a)][Y(2-aminoethoxy)][2- |
| | Nal][2-Me-Leu]E(1c)R[3-(3-Quinolinyl)-Ala] |
| SEQ ID NO: 150 | [(3-Aminomethyl)benzoyl](1c)*SC(2a)WQC(2a)][Y(2-aminoethoxy)][2- |
| | Nal][2-Me-Leu]E(1c)R[{d}[3-(3-Pyridyl)-Ala]] |
| SEQ ID NO: 151 | [(3-Aminomethyl)benzoyl](1c)*SC(2a)WQC(2a)][Y(2-aminoethoxy)][2- |
| | Nal][2-Me-Leu]E(1c)R[3-Amino-3-(3′-pyridyl)propionyl] |
| SEQ ID NO: 152 | [(3-Aminomethyl)benzoyl](1c)*SC(2a)WQC(2a)][Y(2-aminoethoxy)][2- |
| | Nal][2-Me-Leu]E(1c)R[F(3-F)] |
| SEQ ID NO: 153 | [(3-Aminomethyl)benzoyl](1c)*SC(2a)WQC(2a)][Y(2-aminoethoxy)][2- |
| | Nal][2-Me-Leu]E(1c)R[F(3,5-F)] |
| SEQ ID NO: 154 | [(3-Aminomethyl)benzoyl](1c)*SC(2a)WQC(2a)][Y(2-aminoethoxy)][2- |
| | Nal][2-Me-Leu]E(1c)R[4-Aminomethyl-2-pyridineacetyl] |
| SEQ ID NO: 155 | K(1c)TC(2a)WQC(2a)[Y(2-aminoethoxy)][2-Nal][2-Me-Leu]E(1c)[Dab][3- |
| | (3-Pyridyl)-Ala] |
| SEQ ID NO: 156 | [(3-Aminomethyl)benzoyl](1c)*IE(2c)WQ[Dab](2c)[Y (2-aminoethoxy)][2- |
| | Nal][2-Me-Val]E(1c)[Dab][3-(3-Pyridyl)-Ala] |
| SEQ ID NO: 157 | [4-Aminomethyl-phenylacetyl](1c)*IE(2c)WQ[Dab](2c)[Y(2- |
| | aminoethoxy)][2-Nal][2-Me-Val]E(1c)[Dab][3-(3-Pyridyl)-Ala] |
| SEQ ID NO: 158 | [4-Aminomethyl-phenylacetyl](1c)*IE(2c)WQ[Dab](2c)[Y(2- |
| | aminoethoxy)][F(3,4-Me)][2-Me-Val]E(1c)G[3-(3-Pyridyl)-Ala] |
| SEQ ID NO: 159 | [4-Aminomethyl-phenylacetyl](1c)*IE(2c)WQ[Dab](2c)[F(4-Me)][F(3,4- |
| | Me)][2-Me-Val]E(1c)G[3-(3-Pyridyl)-Ala] |
| SEQ ID NO: 160 | [4-Aminomethyl-phenylacetyl](1c)*IE(2c)WQ[Dab](2c)[F(4-Me)][F(3,4- |
| | Me)][2-Me-Val]E(1c)[GABA][3-(3-Pyridyl)-Ala] |
| SEQ ID NO: 161 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-pyridylacetyl)] |
| SEQ ID NO: 162 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-pyridylpropionyl)] |
| SEQ ID NO: 163 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-fluorobenzoyl)] |
| SEQ ID NO: 164 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-fluorophenylacetyl)] |
| SEQ ID NO: 165 | [4-Aminomethyl-phenylacetyl](1c)*IE(2c)WQ[Dab](2c)[Y(2- |
| | aminoethoxy)][F(3,4-Me)][2-Me-Val]E(1c)[Dab][2-Me-3-(3-Pyridyl)-Ala] |
| SEQ ID NO: 166 | [4-Aminomethyl-phenylacetyl](1c)*IE(2c)WQ[Dab](2c)[Y(Me)][F(3,4- |
| | Me)][2-Me-Val]E(1c)[Dab][2-Me-3-(3-Pyridyl)-Ala] |
| SEQ ID NO: 167 | [4-Aminomethyl-phenylacetyl](1c)*IE(2c)WQ[Dab](2c)[2-Me-Phe][F(3,4- |
| | Me)][2-Me-Val]E(1c)[Dab][2-Me-3-(3-Pyridyl)-Ala] |
| SEQ ID NO: 168 | [4-Aminomethyl-phenylacetyl](1c)*IE(2c)WQ[Dab](2c)[Bip][F(3,4-Me)][2- |
| | Me-Val]E(1c)[Dab][2-Me-3-(3-Pyridyl)-Ala] |
| SEQ ID NO: 169 | [4-Aminomethyl-phenylacetyl](1c)*IE(2c)WQ[Dab](2c)[2-Me-F(4-F)] [F(3,4- |
| | Me)][2-Me-Val]E(1c)[Dab][2-Me-3-(3-Pyridyl)-Ala] |
| SEQ ID NO: 170 | [4-Aminomethyl-phenylacetyl](1c)*IE(2c)WQ[Dab](2c)[{d}F(4-F)][F(3,4- |
| | Me)][2-Me-Val]E(1c)[Dab][2-Me-3-(3-Pyridyl)-Ala] |
| SEQ ID NO: 171 | [4-Aminomethyl-phenylacetyl](1c)*IE(2c)WQ[Dab](2c)[F(4-CI)] [F(3,4- |
| | Me)][2-Me-Val]E(1c)[Dab][2-Me-3-(3-Pyridyl)-Ala] |
| SEQ ID NO: 172 | [4-Aminomethyl-phenylacetyl](1c)*IE(2c)WQ[Dab](2c)[alpha-Me- |
| | Trp][F(3,4-Me)][2-Me-Val]E(1c)[Dab][2-Me-3-(3-Pyridyl)-Ala] |
| SEQ ID NO: 173 | [4-Aminomethyl-phenylacetyl](1c)*IE(2c)WQ[Dab](2c)[3,3-Diphenyl- |
| | Ala][F(3,4-Me)][2-Me-Val]E(1c)[Dab][2-Me-3-(3-Pyridyl)-Ala] |
| SEQ ID NO: 174 | [4-Aminomethyl-phenylacetyl](1c)*IE(2c)WQ[Dab](2c)[Phg][F(3,4-Me)][2- |
| | Me-Val]E(1c)[Dab][2-Me-3-(3-Pyridyl)-Ala] |
| SEQ ID NO: 175 | [4-Aminomethyl-phenylacetyl](1c)*IE(2c)WQ[Dab](2c)[F(4-Me)][1-Me- |
| | Trp][2-Me-Val]E(1c)[Dab][2-Me-3-(3-Pyridyl)-Ala] |
| SEQ ID NO: 176 | [4-Aminomethyl-phenylacetyl](1c)*IE(2c)WQ[Dab](2c)[F(4-Me)][Bip][2-Me- |
| | Val]E(1c)[Dab][2-Me-3-(3-Pyridyl)-Ala] |
| SEQ ID NO: 177 | [4-Aminomethyl-phenylacetyl](1c)*IE(2c)WQ[Dab](2c)[F(4-Me)][{d}6-F- |
| | Trp][2-Me-Val]E(1c)[Dab][2-Me-3-(3-Pyridyl)-Ala] |

wherein:
*= bridge uses the peptide backbone amine or carboxylic acid at the N- or C-terminus, not the side chain amine or carboxylic acid
(1a) = [2,11] 1,3-dithio-propan-2-one bridge; (1c) = [2,11] lactam bridge; (1g) = [2,11] 1,4-disubstituted 1,2,3-triazole bridge; (1h) = [2,11] 1,5-disubstituted 1,2,3-triazole bridge; (2a) = [4,7] 1,3-dithio-propan-2-one bridge; (2c) = [4,7] lactam bridge; (2d) = [4,7] 1,2-phenylenedimethanethiol bridge; (2e) = [4,7] 1,3-phenylenedimethanethiol bridge; (2f) = [4,7] 1,4-phenylenedimethanethiol bridge; (2g) = [4,7] 1,4-disubstituted 1,2,3-triazole bridge.

### X1

X1 is absent or is selected from the group consisting of Asp, Gly, Leu, Glu, Ser, Cys, and Lys.

In some embodiments, X1 is absent or is selected from the group consisting of Asp, Glu, Ser, Cys, and Lys.

In some embodiments, X1 may be absent or is selected from the group consisting of Asp, Gly, and Leu.

In some embodiments, X1 may be absent or Asp.

In some embodiments, X1 is absent, R¹ is absent, and X2 and X11 are amino acid residues that together form a lactam bridge or a bridge containing a triazole ring via the N-terminus of X2.

In some embodiments, X1 and X12 to X14 are absent, R¹ and R² are absent, and X2 and X11 are amino acid residues that together form a head-to-tail cyclised lactam bridge via the N-terminus of X2 and the C-terminus of X11.

Preferably, X1 is absent. As shown in Example 2, truncating the compound by removing the N-terminal amino acid residue (i.e. X1) can retain or increase potency of the peptide.

### X3

X3 is selected from the group consisting of Ser, beta-homo-Ser, Thr, Leu, Cys, Gln, Val, Ile, N-Me-Ser, and Q(pyrrolidin).

In some embodiments, X3 is selected from the group consisting of Ser, beta-homo-Ser, Thr, Gln, Val, Ile, N-Me-Ser, and Q(pyrrolidin).

In some embodiments, X3 is selected from the group consisting of Ser, beta-homo-Ser, Thr, Leu, Cys, and Gln.

In some embodiments, X3 is Ser or lie.

In some embodiments, X3 is Ser.

X5
X5 is selected from the group consisting of Trp, Tyr, Ala, 1-Me-Trp, 7-Me-Trp, 7-Ph-Trp, 7-(Naphth-2-yl)-Trp, 2-Nal, Bip, 4-F-Trp, 7-F-Trp, and N-Me-Trp.

In some embodiments, X5 is selected from the group consisting of Trp, Tyr, Ala, 1-Me-Trp, 7-Me-Trp, 7-Ph-Trp, 7-(Naphth-2-yl)-Trp, 2-Nal, and Bip.

In some embodiments, X5 is selected from the group consisting of Trp, Ala, 1-Me-Trp, 7-Me-Trp, 7-Ph-Trp, 7-(Naphth-2-yl)-Trp, 2-Nal, Bip, 4-F-Trp, 7-F-Trp, and N-Me-Trp.

In some embodiments, X5 is Trp or 7-Me-Trp. In some embodiments, X5 is Trp.

X6 is selected from the group consisting of Gln, Glu, Tyr, Cys, Val, His, N-Me-Gln, and Q(pyrrolidin).

In some embodiments, X6 is selected from the group consisting of Gln, Glu, Val, His, N-Me-Gln, and Q(pyrrolidin).

In some embodiments, X6 is selected from the group consisting of Gln, Glu, Tyr and Cys.

In some embodiments, X6 is Gln or Glu. In some embodiments, X6 is Gln.

### X8

X8 is selected from the group consisting of Trp, Tyr, Asn, Ala, His, 2-Nal, Dab, 2,4-diaminobutanoyl([2-(trimethyl-2-aminoethoxy)ethoxy]propyl), F(4-NH₂-(2-(trimethyl-2-aminoethoxy)ethoxy)propyl), Phe, Val, 4-Me-Phe, 2-Me-Phe, Bip, 2-Me-F(4-F), {d}F(4-F), 4-Cl-Phe, alpha-Me-Trp, 3,3-Diphenyl-Ala, and Phg, wherein the hydroxyl group of Tyr is optionally substituted with C₁₋₃ alkyl optionally substituted with NH₂.

In some embodiments, X8 is selected from the group consisting of Trp, Tyr, Ala, His, 2-Nal, Dab, 2,4-diaminobutanoyl([2-(trimethyl-2-aminoethoxy)ethoxy]propyl), F(4-NH₂(2-(trimethyl-2-aminoethoxy)ethoxy)propyl), Phe, Val, 4-Me-Phe, 2-Me-Phe, Bip, 2-Me-F(4-F), {d}F(4-F), 4-Cl-Phe, alpha-Me-Trp, 3,3-Diphenyl-Ala, and Phg, wherein the hydroxyl group of Tyr is optionally substituted with C₁₋₃ alkyl optionally substituted with NH₂.

In some embodiments, X8 is selected from the group consisting of Trp, Tyr, Ala, His, 2-Nal, Dab, 2,4-diaminobutanoyl([2-(trimethyl-2-aminoethoxy)ethoxy]propyl), F(4-NH₂-(2-(trimethyl-2-aminoethoxy)ethoxy)propyl), Phe, Val, 4-Me-Phe, 2-Me-Phe, Bip, 2-Me-F(4-F), {d}F(4-F), 4-Cl-Phe, alpha-Me-Trp, 3,3-Diphenyl-Ala, Phg, Y(2-aminoethoxy), and Y(Me).

In some embodiments, X8 is selected from the group consisting of Trp, Tyr, Asn, Ala, His, and 2-Nal, wherein the hydroxyl group of Tyr is optionally substituted with C₁₋₃ alkyl optionally substituted with NH₂.

In some embodiments, X8 is selected from the group consisting of Trp, Tyr, and 4-Me-Phe, wherein the hydroxyl group of Tyr is substituted with C₁₋₃ alkyl optionally substituted with NH₂. In some embodiments, X8 is selected from the group consisting of Trp, Tyr, and 4-Me-Phe, wherein the hydroxyl group of Tyr is substituted with —CH₂CH₂NH₂.

In some embodiments, X8 is Trp or Tyr, wherein the hydroxyl group of Tyr is substituted with C₁₋₃ alkyl optionally substituted with NH₂. In some embodiments, X8 is Trp or Tyr, wherein the hydroxyl group of Tyr is substituted with —CH₂CH₂NH₂.

In some embodiments, X8 is Trp.

In some embodiments, X8 is Tyr wherein the hydroxyl group of Tyr is optionally substituted with C₁₋₃ alkyl optionally substituted with NH₂. In some embodiments, X8 is Tyr wherein the hydroxyl group of Tyr is optionally substituted with —CH₂CH₂NH₂ or —CH₃.

In some embodiments, X8 is Tyr wherein the hydroxyl group of Tyr is optionally substituted with —CH₂CH₂NH₂. In some embodiments, X8 is Tyr wherein the hydroxyl group of Tyr is optionally substituted with —CH₃.

In some embodiments, X8 is Tyr.

In some embodiments, X8 is Tyr wherein the hydroxyl group of Tyr is substituted with C₁-alkyl optionally substituted with NH₂. In some embodiments, X8 is Tyr wherein the hydroxyl group of Tyr is substituted with C₁₋₃ alkyl substituted with NH₂. In some embodiments, X8 is Tyr wherein the hydroxyl group of Tyr is substituted with —CH₂CH₂NH₂, that is Y(2-aminoethoxy).

In some embodiments, X8 is Tyr wherein the hydroxyl group of Tyr is substituted with C₁-alkyl. In some embodiments, X8 is Tyr wherein the hydroxyl group of Tyr is optionally substituted with —CH₃. In some embodiments, X8 is Tyr wherein the hydroxyl group of Tyr is substituted with —CH₃, that is Y(Me).

Preferably, X8 is selected from Trp, Y(2-aminoethoxy), and 4-Me-Phe. More preferably, X8 is Trp or Y(2-aminoethoxy).

### X9

X9 is selected from the group consisting of 2-Nal, Trp, 1-Me-Trp, 6-Cl-Trp, 3-(3-Quinolinyl)-Ala, Phe, 4-F-Phe, Glu, Cys, Ala, 6-F-Trp, His, 3-F-Phe, 3,4-Me-Phe, Bip, and {d}6-F-Trp.

In some embodiments, X9 is selected from the group consisting of 2-Nal, Trp, 1-Me-Trp, 6-Cl-Trp, 3-(3-Quinolinyl)-Ala, 4-F-Phe, Ala, 6-F-Trp, His, 3-F-Phe, 3,4-Me-Phe, Bip, and {d}6-F-Trp.

In some embodiments, X9 is selected from the group consisting of 2-Nal, Trp, 1-Me-Trp, 6-Cl-Trp, 3-(3-Quinolinyl)-Ala, Phe, 4-F-Phe, Glu, Cys, and Ala.

In some embodiments, X9 is selected from the group consisting of 2-Nal, Trp, and 3,4-Me-Phe.

In some embodiments, X9 is selected from the group consisting of 2-Nal and Trp.

In some embodiments, X9 is Trp. In some embodiments, X9 is 2-Nal.

### X10

X10 is selected from the group consisting of Leu, D-Leu, 2-Me-Leu, 2-Me-Lys, Trp, Asn, Cys, 4-aminotetrahydro-2H-pyran-4-acetyl, and 2-Me-Val.

In some embodiments, X10 is selected from the group consisting of Leu, D-Leu, 2-Me-Leu, 2-Me-Lys, 4-aminotetrahydro-2H-pyran-4-acetyl, and 2-Me-Val.

In some embodiments, X10 is selected from the group consisting of Leu, D-Leu, 2-Me-Leu, 2-Me-Lys, Trp, Asn, Cys, and 4-aminotetrahydro-2H-pyran-4-acetyl.

In some embodiments, X10 is selected from the group consisting of Leu, 2-Me-Leu, and 2-Me-Val. In some embodiments, X10 is Leu or 2-Me-Leu. In some embodiments, X10 is 2-Me-Leu or 2-Me-Val.

In some embodiments, X10 is Leu. In some embodiments, X10 is 2-Me-Leu. In some embodiments, X10 is 2-Me-Val.

### X12

X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, 3-(4-Pyridyl)-Ala, {d}2,4-diaminobutanoyl([2-(trimethyl-2-aminoethoxy)ethoxy]propyl), D-Gln, D-Glu, D-His, 3-aminopropanoyl, and GABA, or is absent.

In some embodiments, X12 is selected from the group consisting of Arg, D-Arg, 2-Me-Arg, N-Me-Arg, Ser, 4-NH₂-Phe, Tyr, Gly, Glu, Asn, Dab, 3-(3-Pyridyl)-Ala, 3-(4-Pyridyl)-Ala, {d}2,4-diaminobutanoyl([2-(trimethyl-2-aminoethoxy)ethoxy]propyl), D-Gln, D-Glu, D-His, 3-aminopropanoyl, and GABA, or is absent.

In some embodiments, X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.

In some embodiments, X12 is selected from the group consisting of Arg, D-Arg, Dab, and Gly.

In some embodiments, X12 is Arg or Ser. In some embodiments, X12 is Arg or D-Arg. In some embodiments, X12 is Arg or Dab. In some embodiments, X12 is D-Arg or Dab.

In some embodiments, X12 is Arg. In some embodiments, X12 is D-Arg. In some embodiments, X12 is Dab. In some embodiments, X12 is Ser. In some embodiments, X12 is absent.

Preferably, X12 is selected from the group consisting of Arg, D-Arg, and Dab.

In some embodiments, X1 and X12 to X14 are absent, R¹ and R² are absent, and X2 and X11 are amino acid residues that together form a head-to-tail cyclised lactam bridge via the N-terminus of X2 and the C-terminus of X11.

### X13

X13 is absent or is selected from the group consisting of Asn, Gly, 3-(3-Pyridyl)-Ala, 3-(4-Pyridyl)-Ala, 3-(3-Quinolinyl)-Ala, {d}[3-(3-Pyridyl)-Ala], 3-amino-3-(3′-pyridyl)propionyl, 3-F-Phe, 3,5-F-Phe, 4-aminomethyl-2-pyridineacetyl, 2,3-diaminopropanoyl(3-pyridylacetyl), 2,3-diaminopropanoyl(3-pyridylpropionyl), 2,3-diaminopropanoyl(3-fluorobenzoyl), 2,3-diaminopropanoyl(3-fluorophenylacetyl), and 2-Me-3-(3-Pyridyl)-Ala.

In some embodiments, X13 is absent or is selected from the group consisting of Asn, Gly, 3-(3-Pyridyl)-Ala, and 3-(4-Pyridyl)-Ala.

In some embodiments, X13 is absent, 3-(3-Pyridyl)-Ala, or 2-Me-3-(3-Pyridyl)-Ala.

In some embodiments, X13 is absent. In some embodiments, X13 is Asn. In some embodiments, X13 is Gly. In some embodiments, X13 is 3-(3-Pyridyl)-Ala. In some embodiments, X13 is 2-Me-3-(3-Pyridyl)-Ala.

In some embodiments, X1 and X12 to X14 are absent, R¹ and R² are absent, and X2 and X11 are amino acid residues that together form a head-to-tail cyclised lactam bridge via the N-terminus of X2 and the C-terminus of X11.

Preferably, X13 is 3-(3-Pyridyl)-Ala when X14 is absent, such that X13 is the C-terminal amino acid residue. In such embodiments, the potency for hIL23 pSTAT3 is particularly strong, as evidenced by a low IC₅₀ value. See, for example, Compounds 78, 120-124, 147, 148, and 155-158 in Example 3, Table 3-1.

### X14

X14 is absent or is Gly.

In some embodiments, X14 is absent. In some embodiments, X14 is Gly.

Preferably, X14 is absent. In such embodiments, X13 may be 3-(3-Pyridyl)-Ala.

In some embodiments, X1 and X12 to X14 are absent, R¹ and R² are absent, and X2 and X11 are amino acid residues that together form a head-to-tail cyclised lactam bridge via the N-terminus of X2 and the C-terminus of X11.

### X2 and X11

X2 and X11 are amino acid residues who together form a lactam bridge, a dithioether bridge, or a bridge containing a triazole ring.

Preferably, X2 and X11 are amino acid residues who together form a lactam bridge. As shown in Example 3, a lactam bridge between amino acid residues X2 and X11 is more stable than the corresponding dithioether bridge, 1,3-dithio-propan-2-one.

In some embodiments, X1 is absent, R¹ is absent, and X2 and X11 are amino acid residues that together form a lactam bridge or a bridge containing a triazole ring via the N-terminus of X2.

In some embodiments, X1 and X12 to X14 are absent, R¹ and R² are absent, and X2 and X11 are amino acid residues that together form a head-to-tail cyclised lactam bridge via the N-terminus of X2 and the C-terminus of X11.

In some embodiments, X2 is Cys and X11 is Cys who together form a dithioether bridge;
X2 is Glu and X11 is Lys who together form a lactam bridge; or X2 is Lys and X11 is Glu who together form a lactam bridge.

### Amino Acid Residues

A lactam bridge is formed of one amino acid residue comprising an amine group and another amino acid residue comprising a carboxylic acid group. Preferably, the amine and/or carboxylic acid group of the amino acid residue is on the side chain of the amino acid residue, such as Dpr, hLys, Lys, Arg, Orn, Dab, Glu and Asp. Alternatively, the amine and/or carboxylic acid group of the amino acid residue may be the N- or C-terminus of the peptide chain, such as the amine or carboxylic acid of the peptide backbone of any amino acid, or such as bAla, 3-(4-aminophenyl)propanoyl, (3-aminomethyl)benzoyl, (4-aminomethyl)benzoyl, 4-(2-aminoethyl)benzoyl, 2-aminomethyl-phenylacetyl, 3-aminomethyl-phenylacetyl, 4-aminomethyl-phenylacetyl, 6-aminohexanoyl, 6-amino-4-oxahexanoyl, trans-4-aminomethyl-cyclohexyl-1-carbonyl, and (4-(2-aminoethyl)-piperazine-1-yl)-acetyl.

Suitable amino acid residues for X2 and X11 who together form a lactam bridge may be selected from:
Amino acid residues comprising an amine group: Dpr, hLys, Lys, Arg, Orn, bAla, 3-(4-aminophenyl)propanoyl, (3-aminomethyl)benzoyl, (4-aminomethyl)benzoyl, 4-(2-aminoethyl)benzoyl, 2-aminomethyl-phenylacetyl, 3-aminomethyl-phenylacetyl, 4-aminomethyl-phenylacetyl, Dab, 6-aminohexanoyl, 6-amino-4-oxahexanoyl, trans-4-aminomethyl-cyclohexyl-1-carbonyl, (4-(2-aminoethyl)-piperazine-1-yl)-acetyl, 2,4-diaminobutanoyl([2-(trimethyl-2-aminoethoxy)ethoxy]propyl), and 4-methylaminomethyl-phenylacetyl.
Amino acid residues comprising a carboxylic acid group: Glu, Asp, Aad, and Apm.

A dithioether bridge is formed of two amino acid residues comprising sulfur moieties, such as —SH. Preferably, the sulfur moiety of the amino acid residue is on the side chain of the amino acid residue, such as Cys.

Suitable amino acid residues for X2 and X11 who together form a dithioether bridge may be Cys or N-Me-Cys.

A bridge containing a triazole ring is formed of one amino acid residue comprising an azide (—N₃) group and another amino acid residue comprising an alkyne group. In some embodiments, the azide and/or alkyne groups of the amino acid residue is on the side chain of the amino acid residue.

Suitable amino acid residues for X2 and X11 who together form a bridge containing a triazole ring may be selected from:
Amino acid residues comprising an azide group: Ala(N₃), Aha, Orn(Na), Lys(N₃), Lys(N₃), azidoacetic acid, (N₃)-Ala, Dab(azidoacetic acid), and Dab((N₃)-Ala).
Amino acid residues comprising an alkyne group: Pra, Hpg, Bpg, Glu(propargylamine), Dab(3-butynoic acid), and but-3-ynoic acid.

### Bridge Length

The length of the bridge is counted as the number of atoms in a linear chain from the first atom attached to the atom (carbon) adjacent to the carboxylic acid moiety of the amino acid of the first residue (X2 for the bridge between X2 and X11), i.e. attached to the alpha carbon of the relevant residue for most amino acids, up to the first atom attached to the atom (carbon) adjacent to the carboxylic acid moiety of the amino acid of the second residue (X11 for the bridge between X2 and X11).

The contribution to the length of the bridge for amino acid residues and the type of bridge is described below.

In some embodiments, the length of the bridge between X2 and X11 is at least 5 atoms long. In some embodiments, the length of the bridge between X2 and X11 is no longer than 10 atoms long. In some embodiments, the length of the bridge between X2 and X11 is 5 to 10 atoms long, such as 5, 6, 7, 8, 9, or 10 atoms long.

### Lactam Bridge

The inventors have found that replacing a dithioether bridge with a lactam bridge can lead to increased potency (see Example 2) of the IL-23R peptide inhibitor.

Thus, the compound of the invention may comprises a lactam bridge formed between the amino acid residues at positions X2 and X11. For simplicity, positions X2 and X11 will be discussed by reference to the residues nominally present before lactam formation.

One of the residues at positions X2 and X11 is an amino acid residue comprising an amine group and the other is an amino acid residue comprising a carboxylic acid group, wherein a lactam (cyclic amide) is formed between the amine and carboxylic acid groups.

Typically, the amine and/or carboxylic acid group is present on the side chain of the amino acid residue. The amine may be a primary or secondary amine, but is typically a primary amine. Suitable amino acid residues whose side chains can participate in a lactam bridge include Dpr, Dab, Orn, hLys, Lys, and Arg (having side chains comprising amine groups) and Aad, Apm, Glu, and Asp, (having side chains comprising carboxylic acid groups). Any of the amino acids selected from Aad, Apm, Glu, and Asp may in principle form a lactam bridge with any of the amino acid residues selected from the group consisting of Dpr, Dab, Orn, hLys, Lys, and Arg.

Alternatively, the amine and/or carboxylic acid group of the amino acid residue may be the N- or C-terminus of the peptide chain, such as the amine or carboxylic acid of the peptide backbone of any amino acid such as Arg or Asp, or such as bAla, 3-(4-aminophenyl)propanoyl, (3-aminomethyl)benzoyl, (4-aminomethyl)benzoyl, 4-(2-aminoethyl)benzoyl, 2-aminomethyl-phenylacetyl, 3-aminomethyl-phenylacetyl, 4-aminomethyl-phenylacetyl, 6-aminohexanoyl, 6-amino-4-oxahexanoyl, trans-4-aminomethyl-cyclohexyl-1-carbonyl, and (4-(2-aminoethyl)-piperazine-1-yl)-acetyl. Suitable amino acid residues that may participate in a lactam bridge via the N- or C-terminus of the peptide chain is Arg, bAla, 3-(4-aminophenyl)propanoyl, (3-aminomethyl)benzoyl, (4-aminomethyl)benzoyl, 4-(2-aminoethyl)benzoyl, 2-aminomethyl-phenylacetyl, 3-aminomethyl-phenylacetyl, 4-aminomethyl-phenylacetyl, 6-aminohexanoyl, 6-amino-4-oxahexanoyl, trans-4-aminomethyl-cyclohexyl-1-carbonyl, (4-(2-aminoethyl)-piperazine-1-yl)-acetyl, 4-aminomethyl-2-pyridineacetyl, 4-aminomethyl-3-pyridineacetyl, 4-aminomethyl-2-fluoro-phenylacetyl, 4-aminomethyl-3-methyl-phenylacetyl, 4-aminomethyl-3-methoxy-phenylacetyl, 2,4-diaminobutanoyl([2-(trimethyl-2-aminoethoxy)ethoxy]propyl), and 4-methylaminomethyl-phenylacetyl (having a terminal amine group), and Asp (having a terminal carboxylic acid group).

Thus, one of the residues at positions X2 and X11 may be selected from Dpr, Dab, Orn, hLys, Lys, Arg, bAla, 3-(4-aminophenyl)propanoyl, (3-aminomethyl)benzoyl, (4-aminomethyl)benzoyl, 4-(2-aminoethyl)benzoyl, 2-aminomethyl-phenylacetyl, 3-aminomethyl-phenylacetyl, 4-aminomethyl-phenylacetyl, 6-aminohexanoyl, 6-amino-4-oxahexanoyl, trans-4-aminomethyl-cyclohexyl-1-carbonyl, (4-(2-aminoethyl)-piperazine-1-yl)-acetyl, 4-aminomethyl-2-pyridineacetyl, 4-aminomethyl-3-pyridineacetyl, 4-aminomethyl-2-fluoro-phenylacetyl, 4-aminomethyl-3-methyl-phenylacetyl, 4-aminomethyl-3-methoxy-phenylacetyl, 2,4-diaminobutanoyl([2-(trimethyl-2-aminoethoxy)ethoxy]propyl), and 4-methylaminomethyl-phenylacetyl, and the other may be selected from Glu, Asp, Aad, and Apm. In some embodiments, one of the residues at position X2 and X11 may be selected from Lys, Arg, Orn, bAla, 3-(4-aminophenyl)propanoyl, (3-aminomethyl)benzoyl, (4-aminomethyl)benzoyl, 4-(2-aminoethyl)benzoyl, 2-aminomethyl-phenylacetyl, 3-aminomethyl-phenylacetyl, 4-aminomethyl-phenylacetyl, Dab, 6-aminohexanoyl, 6-amino-4-oxahexanoyl, trans-4-aminomethyl-cyclohexyl-1-carbonyl, (4-(2-aminoethyl)-piperazine-1-yl)-acetyl, 4-aminomethyl-2-pyridineacetyl, 4-aminomethyl-3-pyridineacetyl, 4-aminomethyl-2-fluoro-phenylacetyl, 4-aminomethyl-3-methyl-phenylacetyl, 4-aminomethyl-3-methoxy-phenylacetyl, 2,4-diaminobutanoyl([2-(trimethyl-2-aminoethoxy)ethoxy]propyl), and 4-methylaminomethyl-phenylacetyl, and the other may be selected from Glu and Asp.

In some embodiments the amine component of the lactam bridge derives from the amino acid at position X2, whereas the carboxylic acid component of the lactam bridge derives from the amino acid at position X11. Thus X2 may be selected from Dpr, Dab, Orn, hLys, Lys, Arg, bAla, 3-(4-aminophenyl)propanoyl, (3-aminomethyl)benzoyl, (4-aminomethyl)benzoyl, 4-(2-aminoethyl)benzoyl, 2-aminomethyl-phenylacetyl, 3-aminomethyl-phenylacetyl, 4-aminomethyl-phenylacetyl, 6-aminohexanoyl, 6-amino-4-oxahexanoyl, trans-4-aminomethyl-cyclohexyl-1-carbonyl, (4-(2-aminoethyl)-piperazine-1-yl)-acetyl, 4-aminomethyl-2-pyridineacetyl, 4-aminomethyl-3-pyridineacetyl, 4-aminomethyl-2-fluoro-phenylacetyl, 4-aminomethyl-3-methyl-phenylacetyl, 4-aminomethyl-3-methoxy-phenylacetyl, 2,4-diaminobutanoyl([2-(trimethyl-2-aminoethoxy)ethoxy]propyl), and 4-methylaminomethyl-phenylacetyl, and X11 may be selected from Aad, Apm, Glu, and Asp. In some embodiments, X2 may be selected from Lys, Orn, bAla, 3-(4-aminophenyl)propanoyl, (3-aminomethyl)benzoyl, (4-aminomethyl)benzoyl, 4-(2-aminoethyl)benzoyl, 2-aminomethyl-phenylacetyl, 3-aminomethyl-phenylacetyl, 4-aminomethyl-phenylacetyl, 6-aminohexanoyl, 6-amino-4-oxahexanoyl, trans-4-aminomethyl-cyclohexyl-1-carbonyl, (4-(2-aminoethyl)-piperazine-1-yl)-acetyl, 4-aminomethyl-2-pyridineacetyl, 4-aminomethyl-3-pyridineacetyl, 4-aminomethyl-2-fluoro-phenylacetyl, 4-aminomethyl-3-methyl-phenylacetyl, 4-aminomethyl-3-methoxy-phenylacetyl, 2,4-diaminobutanoyl([2-(trimethyl-2-aminoethoxy)ethoxy]propyl), and 4-methylaminomethyl-phenylacetyl, and X11 may be Glu and Asp.

Alternatively, in some embodiments the carboxylic acid component of the lactam bridge derives from the amino acid at position X2, whereas the amine component of the lactam bridge derives from the amino acid at position X11. Thus X2 may be selected from Aad, Apm, Glu, and Asp, and X11 may be selected from Dpr, Dab, Orn, hLys, Lys, Arg, bAla, 3-(4-aminophenyl)propanoyl, (3-aminomethyl)benzoyl, (4-aminomethyl)benzoyl, 4-(2-aminoethyl)benzoyl, 2-aminomethyl-phenylacetyl, 3-aminomethyl-phenylacetyl, 4-aminomethyl-phenylacetyl, 6-aminohexanoyl, 6-amino-4-oxahexanoyl, trans-4-aminomethyl-cyclohexyl-1-carbonyl, (4-(2-aminoethyl)-piperazine-1-yl)-acetyl, 4-aminomethyl-2-pyridineacetyl, 4-aminomethyl-3-pyridineacetyl, 4-aminomethyl-2-fluoro-phenylacetyl, 4-aminomethyl-3-methyl-phenylacetyl, 4-aminomethyl-3-methoxy-phenylacetyl, 2,4-diaminobutanoyl([2-(trimethyl-2-aminoethoxy)ethoxy]propyl), and 4-methylaminomethyl-phenylacetyl. In some embodiments, X2 may be selected from Glu and Asp, and X11 may be selected from Lys, Arg, and Dab.

Suitable pairings of residues at positions X2 and X11 to form a lactam bridge include:

When X2 is the amine component and X11 is the carboxylic acid component:
X2 is Lys and X11 is Glu;
X2 is Orn and X11 is Glu;
X2 is bAla and X11 is Glu;
X2 is 3-(4-aminophenyl)propanoyl and X11 is Glu;
X2 is (3-aminomethyl)benzoyl and X11 is Glu;
X2 is (4-aminomethyl)benzoyl and X11 is Glu;
X2 is 4-(2-aminoethyl)benzoyl and X11 is Glu;
X2 is 2-aminomethyl-phenylacetyl and X11 is Glu;
X2 is 3-aminomethyl-phenylacetyl and X11 is Glu;
X2 is 4-aminomethyl-phenylacetyl and X11 is Glu;
X2 is 6-aminohexanoyl and X11 is Glu;
X2 is 6-amino-4-oxahexanoyl and X11 is Glu;
X2 is trans-4-aminomethyl-cyclohexyl-1-carbonyl and X11 is Glu;
X2 is (4-(2-aminoethyl)-piperazine-1-yl)-acetyl and X11 is Glu;
X2 is (4-(2-aminoethyl)-piperazine-1-yl)-acetyl and X11 is Asp;
X2 is 4-aminomethyl-2-pyridineacetyl and X11 is Glu;
X2 is 4-aminomethyl-3-pyridineacetyl and X11 is Glu;
X2 is 4-aminomethyl-2-fluoro-phenylacetyl and X11 is Glu;
X2 is 4-aminomethyl-2-methyl-phenylacetyl and X11 is Glu;
X2 Is 4-aminomethyl-3-methyl-phenylacetyl and X11 is Glu;
X2 is 4-aminomethyl-3-methoxy-phenylacetyl and X11 is Glu;
X2 is Dab and X11 is Glu;
X2 is 2,4-diaminobutanoyl([2-(trimethyl-2-aminoethoxy)ethoxy]propyl) and X11 is Glu; or
X2 is 2,4-diaminobutanoyl([2-(trimethyl-2-aminoethoxy)ethoxy]propyl) and X11 is Glu.

When X2 is the carboxylic acid component and X11 is the amine component:
X2 is Glu and X11 is Lys;
X2 is Glu and X11 is Dab; or
X2 is Asp and X11 is Arg.

In some embodiments, X2 is (3-aminomethyl)benzoyl and X11 is Glu; X2 is 4-aminomethyl-phenylacetyl and X11 is Glu; X2 is Glu and X11 is Lys; or X2 is Lys and X11 is Glu.

In some embodiments, X2 is (3-aminomethyl)benzoyl and X11 is Glu; X2 is 4-aminomethyl-phenylacetyl and X11 is Glu; or X2 is Lys and X11 is Glu.

In some embodiments, X2 is (3-aminomethyl)benzoyl and X11 is Glu; or X2 is 4-aminomethyl-phenylacetyl and X11 is Glu.

In some embodiments, X2 is Glu and X11 is Lys; or X2 is Lys and X11 is Glu.

Preferably, X2 is Lys and X11 is Glu. The inventors found this combination of amino acid residues at position 2 and I1 increased potency (see Example 2).

The contribution of the side chain to the length of the lactam bridge is counted as the number of atoms in a linear chain from the first atom of the side chain (which is bonded to an atom of the peptide backbone, i.e. to the alpha carbon of the relevant residue for most amino acids) up to and including the atom which participates in the amide bond of the lactam bridge (i.e. the carbon atom of the carboxylic acid functional group or the nitrogen atom of the amine group).

Thus common acid- and amine-containing side chains are considered to have the following side chain lengths:

Amine-containing side chains:

| | |
|---|---|
| Dpr | 2 atoms |
| Dab | 3 atoms |
| Orn | 4 atoms |
| Lys | 5 atoms |
| hLys | 6 atoms |
| Arg | 6 atoms |

Carboxylic acid-containing side chains:

| | |
|---|---|
| Asp | 2 atoms |
| Glu | 3 atoms |
| Aad | 4 atoms |
| Apm | 5 atoms |

Similarly, the contribution of the length of the N- or C-terminus amino acid residue to the length of the lactam bridge is counted as the number of atoms in a linear chain from the first atom attached to the atom (carbon) adjacent to the carboxylic acid moiety of the amino acid residue (i.e. the first atom attached to the alpha carbon of the relevant residue for most amino acids), up to and including the atoms which participate in the amide bond of the lactam bridge (i.e. the carbon atom of the carboxylic acid functional group or the nitrogen atom of the amine group).

Thus the following amino acid residues are considered to have the following lengths:

N-terminus:

| | | |
|---|---|---|
| Asp | 1 | atom |
| Dab | 1 | atom |
| 2,4-diaminobutanoyl([2-(trimethyl-2-aminoethoxy)ethoxy]propyl) | 1 | atom |
| bAla | 2 | atoms |
| (3-aminomethyl)benzoyl | 4 | atoms |
| 2-aminomethyl-phenylacetyl | 4 | atoms |
| (4-aminomethyl)benzoyl | 5 | atoms |
| 3-aminomethyl-phenylacetyl | 5 | atoms |
| 6-aminohexanoyl | 5 | atoms |
| 6-amino-4-oxahexanoyl | 5 | atoms |
| trans-4-aminomethyl-cyclohexyl-1-carbonyl | 5 | atoms |
| 3-(4-aminophenyl)propanoyl | 6 | atoms |
| 4-(2-aminoethyl)benzoyl | 6 | atoms |
| 4-aminomethyl-phenylacetyl | 6 | atoms |
| 4-aminomethyl-2-pyridineacetyl | 6 | atoms |
| 4-aminomethyl-3-pyridineacetyl | 6 | atoms |
| 4-aminomethyl-2-fluoro-phenylacetyl | 6 | atoms |
| 4-aminomethyl-3-methyl-phenylacetyl | 6 | atoms |
| 4-aminomethyl-3-methoxy-phenylacetyl | 6 | atoms |
| 4-methylaminomethyl-phenylacetyl | 6 | atoms |
| (4-(2-aminoethyl)-piperazine-1-yl)-acetyl | 7 | atoms |

C-terminus:

| | | |
|---|---|---|
| Arg | 1 | atom |

The location of the amide bond in the lactam bridge may affect the potency of the compound. The inventors observed that the compound was more active when the amide bond was closer to position 11 (X11) and less active when closer to position 2 (X2) (see Example 2 and Table 2-3b). For example, in Compound 7 where X2 is Lys and X11 is Glu, the amide bond is 4 atoms away from the atom of the peptide backbone of X2 (i.e. to the alpha carbon of Lys) and is 2 atoms away from the atom of the peptide backbone of X11 (i.e. to the alpha carbon of Glu), such that the amide bond is closer to position 11 (X11). For comparison, in Compound 4 where X2 is Glu and X11 is Lys, the amide bond is 2 atoms away from the atom of the peptide backbone of X11 (i.e. to the alpha carbon of Glu) and is 4 atoms away from the atom of the peptide backbone of X2 (i.e. to the alpha carbon of Lys), such that the amide bond is closer to position 2 (X2). Compound 7, where the amide bond is closer to position 11 (X11), is shown to be more active than Compound 4, where the amide bond is closer to position 2 (X2) in Table 2-3b.

Suitable pairings of residues at positions X2 and X11 in which the location of the amide bond in the lactam bridge is closer to position X11 than position X2 once formed include:
X2 is Lys and X11 is Glu;
X2 is Orn and X11 is Glu;
X2 is 3-(4-aminophenyl)propanoyl and X11 is Glu;
X2 is (3-aminomethyl)benzoyl and X11 is Glu;
X2 is (4-aminomethyl)benzoyl and X11 is Glu;
X2 is 4-(2-aminoethyl)benzoyl and X11 is Glu;
X2 is 2-aminomethyl-phenylacetyl and X11 is Glu;
X2 is 3-aminomethyl-phenylacetyl and X11 is Glu;
X2 is 4-aminomethyl-phenylacetyl and X11 is Glu;
X2 is 6-aminohexanoyl and X11 is Glu;
X2 is 6-amino-4-oxahexanoyl and X11 is Glu;
X2 is trans-4-aminomethyl-cyclohexyl-1-carbonyl and X11 is Glu;
X2 is (4-(2-aminoethyl)-piperazine-1-yl)-acetyl and X11 is Glu;
X2 is (4-(2-aminoethyl)-piperazine-1-yl)-acetyl and X11 is Asp;
X2 is 4-aminomethyl-2-pyridineacetyl and X11 is Glu;
X2 is 4-aminomethyl-3-pyridineacetyl and X11 is Glu;
X2 is 4-aminomethyl-2-fluoro-phenylacetyl and X11 is Glu;
X2 is 4-aminomethyl-3-fluoro-phenylacetyl and X11 is Glu;
X2 is 4-aminomethyl-2-methyl-phenylacetyl and X11 is Glu;
X2 is 4-aminomethyl-3-methyl-phenylacetyl and X11 is Glu;
X2 is 4-aminomethyl-2-methoxy-phenylacetyl and X11 is Glu; or
X2 is 4-aminomethyl-3-methoxy-phenylacetyl and X11 is Glu.
X2 is 2,4-diaminobutanoyl([2-(trimethyl-2-aminoethoxy)ethoxy]propyl) and X11 is Glu.

Alternatively, suitable pairings of residues at positions X2 and X11 in which location of the amide bond in the lactam bridge is closer to position X2 than position X11 include:
X2 is bAla and X11 is Glu;
X2 is Glu and X11 is Lys;
X2 is Dab (N-terminus) and X11 is Glu; or
X2 is 2,4-diaminobutanoyl([2-(trimethyl-2-aminoethoxy)ethoxy]propyl) and X11 is Glu.

If the lactam bridge is formed from the side chains of the residues at X2 and X11, the side chain of the residue at position X2 is longer than the side chain of the residue at position
X11, such that the location of the amide bond in the lactam bridge is closer to position 11 (X11) than position 2 (X2) once formed. Thus, in some embodiments the side chain of the residue at position X2 is longer than the side chain of the residue at position X11.

Suitable pairings of residues at positions X2 and X11 in which the side chain at position
X2 is longer than the side chain at position X11 include:
X2 is Lys and X11 is Glu; or
X2 is Orn and X11 is Glu.

Alternatively, in some embodiments the side chain of the residue at position X2 is shorter than the side chain of the residue at position X11, such that the location of the amide bond in the lactam bridge is closer to position 2 (X2) than position 11 (X11) once formed.

Suitable pairings of residues at positions X2 and X11 in which the side chain at position
X2 is shorter than the side chain at position X11 include:
X2 is Glu and X11 is Lys.

Desirably, the length of the lactam bridge after formation of the amide bond (not including any atoms in the peptide backbone) is 5, 6, 7, 8, 9, or 10 atoms; such as 6, 7, 8, or 9 atoms; such as 7 or 8 atoms.

In some embodiments, the length of the lactam bridge after formation of the amide bond (not including any atoms in the peptide backbone) is 5 atoms. A suitable pairing of residues at positions X2 and X11 in which the lactam bridge has a length of 5 atoms include bAla and Glu.

In some embodiments, the length of the lactam bridge after formation of the amide bond (not including any atoms in the peptide backbone) is 6 atoms. A suitable pairing of residues at positions X2 and X11 in which the lactam bridge has a length of 6 atoms include Glu and Dab.

In some embodiments, the length of the lactam bridge after formation of the amide bond (not including any atoms in the peptide backbone) is 7 atoms. Suitable pairings of residues at positions X2 and X11 in which the lactam bridge has a length of 7 atoms include: Orn and Glu; (3-aminomethyl)benzoyl and Glu; and 2-aminomethyl-phenylacetyl and Glu.

Preferably, the length of the lactam bridge after formation of the amide bond (not including any atoms in the peptide backbone) is 8 atoms. Suitable pairings of residues at positions X2 and X11 in which the lactam bridge has a length of 8 atoms include: Glu and Lys; Aad and Orn; Asp and hLys; (4-aminomethyl)benzoyl and Glu; 3-aminomethyl-phenylacetyl and Glu; 6-aminohexanoyl and Glu; 6-amino-4-oxahexanoyl and Glu; and trans-4-aminomethyl-cyclohexyl-1-carbonyl and Glu.

In some embodiments, the length of the lactam bridge after formation of the amide bond (not including any atoms in the peptide backbone) is 9 atoms. Suitable pairings of residues at positions X2 and X11 in which the lactam bridge has a length of 9 atoms include: 3-(4-aminophenyl)propanoyl and Glu; 4-aminomethyl-phenylacetyl and Glu; (4-(2-aminoethyl)-piperazine-1-yl)-acetyl and Asp; 4-(2-aminoethyl)benzoyl and Glu; 4-aminomethyl-2-pyridineacetyl and Glu; 4-aminomethyl-3-pyridineacetyl and Glu; 4-aminomethyl-2-fluoro-phenylacetyl and Glu; 4-aminomethyl-3-methyl-phenylacetyl and Glu; 4-aminomethyl-3-methoxy-phenylacetyl and Glu; and 4-methylaminomethyl-phenylacetyl and Glu.

In some embodiments, the length of the lactam bridge after formation of the amide bond (not including any atoms in the peptide backbone) is 10 atoms. Suitable pairings of residues at positions X2 and X11 in which the lactam bridge has a length of 10 atoms include: (4-(2-aminoethyl)-piperazine-1-yl)-acetyl and Glu.

### Dithioether Bridge

The inventors have found that a linker is required between the thiol side chains of linked [2,11] and [4,7] cysteine residues in the compounds to retain IL-23R inhibitory activity. Replacing the dithioether bridges with disulfide bridges resulted in loss of inhibitory activity (see Example 2).

Thus, the compound of the invention may comprise a dithioether bridge formed between the amino acid residues at positions X2 and X11. For simplicity, positions X2 and X11 will be discussed by reference to the residues nominally present before dithioether formation.

In some embodiments, the dithioether bridge between X2 and X11 is of the formula —S—L—Y—L—S—, wherein:
each S is a sulfur atom and is part of the amino acid residue at X2 and X11;
each L is independently C₁₋₄ alkylene; and
Y is either absent, C(═O), or arylene.

In some embodiments, the dithioether bridge of formula —S—L—Y—L—S— is —SCH₂C(═O)CH₂S—. That is, each L is —CH₂— and Y is C(═O).

In some embodiments, the dithioether bridge of formula —S—L—Y—L—S— is —SCH₂(phenylene)CH₂S—, such as —SCH₂(1,2-phenylene)CH₂S—, —SCH₂(1,3-phenylene)CH₂S—, and —SCH₂(1,4-phenylene)CH₂S—. In some embodiments, the dithioether bridge of formula —S—L—Y—L—S is —SCH₂(1,2-phenylene)CH₂S—. That is, each L is —CH₂— and Y is 1,2-phenylene.

The contribution of the side chain to the length of the dithioether bridge is counted as the number of atoms in a linear chain from the first atom of the side chain (which is bonded to an atom of the peptide backbone, i.e. to the alpha carbon of the relevant residue for most amino acids) up to and including the atom which participates in the dithioether bond of the bridge (i.e. the sulfur atom).

Thus common sulfur-containing side chains are considered to have the following side chain lengths:

| | |
|---|---|
| Cys: | 2 atoms |
| N-Me-Cys | 2 atoms |

In some embodiments, X2 and X11 are each independently selected from Cys and N-Me-Cys.

In some embodiments, X2 is Cys and X11 is Cys.

In some embodiments, X2 is Cys and X11 is N-Me-Cys. In some embodiments, X2 is N-Me-Cys and X11 is Cys.

Preferably, X2 is Cys and X11 is Cys.

The length of the side chains of X2 and X11 are then added to the contribution of the linker between the sulfur moieties of the amino acid residues at positions X2 and X11 to determine the length of the bridge. For embodiments where the dithioether bridge is of the formula —S—L—Y—L—S—, the contribution of the linker is counted as the number of atoms in —L—Y—L—, as the sulfur atoms are already counted in the side chain length.

Desirably, the length of the dithioether bridge after the formation of the dithioether bonds (not including any atoms in the peptide backbone) is 5, 6, 7, 8, 9, or 10 atoms, such as 6, 7, 8, or 9 atoms; such as 7, 8, or 9 atoms, such as 7 or 8 atoms.

In some embodiments, the length of the bridge is 7 atoms long, such as for the bridge —SCH₂C(═O)CH₂S—.

In some embodiments, the length of the bridge is 8 atoms long, such as for the bridge —SCH₂(1,2-phenylene)CH₂S—. In some embodiments, the length of the bridge is 9 atoms long, such as for the bridge —SCH₂(1,3-phenylene)CH₂S—. In some embodiments, the length of the bridge is 10 atoms long, such as for the bridge —SCH₂(1,4-phenylene)CH₂S—.

Preferably, the dithioether bridge is 7 or 8 atoms long.

### Bridge Containing a Triazole Ring

The compound of the invention may comprise a bridge containing a triazole ring formed between the amino acid residues at positions X2 and X11. For simplicity, positions X2 and X11 will be discussed by reference to the residues nominally present before triazole formation.

One of the residues at positions X2 and X11 is an amino acid residue comprising an azide (—N₃) group and the other is an amino acid residue comprising an alkyne group, wherein a triazole (such as a 1,2,3-triazole) is formed between the azide and the alkyne groups. The reaction for the formation of the triazole ring is a Huisgen azide-alkyne 1,3-dipolar cycloaddition. Typically, this reaction forms the 1,4-disubstituted 1,2,3-triazole ring (as opposed to the 1,5-disubstituted 1,2,3-triazole ring) as the major isomer. The 1,5-disubstituted 1,2,3-triazole ring may also be isolated, usually as the minor isomer.

The azide and/or alkyne group may be present on the side chain of the amino acid residue. The alkyne is preferably a terminal alkyne (—C≡CH). Suitable amino acid residues whose side chains can participate in the formation of the triazole ring (such as 1,2,3-triazole ring) include Ala(N₃), Aha, Orn(N₃), and Lys(N₃) (having side chains comprising azide groups) and Pra, Hpg, Bpg, Glu(propargylamine), and Dab(3-butynoic acid) (having side chains comprising alkyne groups).

Alternatively, the azide and/or alkyne group of the amino acid residue may be the N- or C-terminus of the peptide chain. For example, the azide group may be derived from the amine group of the peptide backbone of any amino acid, such as azidoacetic acid and (N₃)-Ala, and may be the N-terminus of the peptide chain. In another example, the alkyne group may be derived from the amine group of the peptide backbone of any amino acid, such as but-3-ynoic acid, and may be the N-terminus of the peptide chain.

Thus, one of the residues at position X2 and X11 may be selected from Ala(N₃), Aha, Orn(N₃), Lys(N₃), azidoacetic acid, (Na)-Ala, Dab(azidoacetic acid), and Dab((N₃)-Ala), and the other may be selected from Pra, Hpg, Bpg, Glu(propargylamine), Dab(3-butynoic acid), and but-3-ynoic acid. In some embodiments, one of the residues at position X2 and X11 is selected from Lys(N₃), azidoacetic acid, (N₃)-Ala, Dab(azidoacetic acid), and Dab((N₃)-Ala), and the other is selected from Pra, Glu(propargylamine), Dab(3-butynoic acid), and but-3-ynoic acid. In some embodiments, one of the residues at position X2 and X11 may be Lys(N₃), and the other may be Pra. In some embodiments, one of the residues at position X2 and X11 may be Dab(azidoacetic acid) or Dab((N₃)-Ala), and the other may be but-3-ynoic acid.

In some embodiments the azide component of the bridge containing a triazole ring derives from the amino acid at position X2, whereas the alkyne component of the bridge containing a triazole ring derives from the amino acid at position X11. Thus X2 may be selected from Ala(N₃), Aha, Orn(Na), Lys(N₃), azidoacetic acid, (N₃)-Ala, Dab(azidoacetic acid), and Dab((N₃)-Ala), and X11 may be selected from Pra, Hpg, Bpg, Glu(propargylamine), Dab(3-butynoic acid), and but-3-ynoic acid. In some embodiments, X2 is selected from Lys(N₃), azidoacetic acid, and (N₃)-Ala; and X11 is selected from Pra, Glu(propargylamine), and Dab(3-butynoic acid). In some embodiments, X2 is Lys(N₃) and X11 is Pra.

Alternatively, in some embodiments the alkyne component of the bridge containing a triazole ring derived from the amino acid at position X2, whereas the azide component of the bridge containing a triazole ring derives from the amino acid at position X11. Thus X2 may be selected from Pra, Hpg, Bpg, Glu(propargylamine), Dab(3-butynoic acid), and but-3-ynoic acid, and X11 may be selected from Ala(N₃), Aha, Orn(Na), Lys(N₃), azidoacetic acid, (N₃)-Ala, Dab(azidoacetic acid), and Dab((N₃)-Ala). In some embodiments, X2 is selected from Pra, and but-3-ynoic acid; and X11 is selected from Dab(azidoacetic acid), and Dab((N₃)-Ala). In some embodiments, X2 is but-3-ynoic acid; and X11 is selected from Dab(azidoacetic acid), and Dab((N₃)-Ala).

Suitable pairings of residues at positions X2 and X11 to form a bridge containing a triazole ring include:

When X2 is the azide component and X11 is the alkyne component:
X2 is Lys(N₃) and X11 is Pra;
X2 is azidoacetic acid and X11 is Glu(propargylamine);
X2 is azidoacetic acid and X11 is Dab(3-butynoic acid);
X2 is (N₃)-Ala and X11 is Glu(propargylamine); or
X2 is (N₃)-Ala and X11 is Dab(3-butynoic acid).

When X2 is the alkyne component and X11 is the azide component:
X2 is Pra and X11 is Dab(azidoacetic acid);
X2 is Pra and X11 is Dab((N₃)-Ala);
X2 is but-3-ynoic acid and X11 is Dab(azidoacetic acid); or
X2 is but-3-ynoic acid and X11 is Dab((N₃)-Ala).

The contribution of the side chain to the length of the bridge containing a triazole ring is counted as the number of atoms in a linear chain from the first atom of the side chain (which is bonded to an atom of the peptide backbone, i.e. to the alpha carbon of the relevant residue for most amino acids) up to and including the atoms which participate in the formation of the triazole ring (i.e. the first nitrogen atom of the azide group attached to the side chain (i.e. —N═N⁺═N⁻) for both 1,4-disubstituted 1,2,3-triazoles and 1,5-disubstituted 1,2,3-triazoles; or the two carbon atoms of the alkyne group for 1,4-disubstituted 1,2,3-triazoles or one carbon atom (i.e. —C≡CH) of the alkyne group for 1,5-disubstituted triazoles).

Thus common azide- and alkyne-containing side chains are considered to have the following side chain lengths:

Azide-containing side chains:

| | |
|---|---|
| Ala(N₃) | 2 atoms |
| Aha | 3 atoms |
| Orn(N₃) | 4 atoms |
| Lys(N₃) | 5 atoms |
| Dab(azidoacetic acid) | 6 atoms |
| Dab((N₃)-Ala) | 6 atoms |

Alkyne-containing side chains (for 1,4-disubstituted 1,2,3-triazoles):

| | |
|---|---|
| Pra | 3 atoms |
| Hpg | 4 atoms |
| Bpg | 5 atoms |
| Glu(propargylamine) | 7 atoms |
| Dab(3-butynoic acid) | 7 atoms |

Alkyne-containing side chains (for 1,5-disubstituted 1,2,3-triazoles):

| | |
|---|---|
| Pra | 2 atoms |
| Hpg | 3 atoms |
| Bpg | 4 atoms |
| Glu(propargylamine) | 6 atoms |
| Dab(3-butynoic acid) | 6 atoms |

Similarly, the contribution of the length of the N- or C-terminus amino acid residue to the length of the bridge containing a triazole ring is counted as the number of atoms in a linear chain from the first atom attached to the atom (carbon) adjacent to the carboxylic acid moiety of the amino acid residue (i.e. the first atom attached to the alpha carbon of the relevant residue for most amino acids), up to and including the atoms which participate in the formation of the triazole ring (i.e. the first nitrogen atom of the azide group attached to the side chain (i.e. —N═N⁺═N⁻) for both 1,4-disubstituted 1,2,3-triazoles and 1,5-disubstituted 1,2,3-triazoles; or the two carbon atoms of the alkyne group for 1,4-disubstituted 1,2,3-triazoles or one carbon atom (i.e. —C≡CH) of the alkyne group for 1,5-disubstituted triazoles).

Thus the following amino acid residues are considered to have the following lengths:

| | |
|---|---|
| N-terminus: | 1 atom |
| azidoacetic acid | 1 atom |
| (N₃)-Ala | |
| but-3-ynoic acid | 2 atoms for 1,4-disubstituted 1,2,3-triazoles |
| | 1 atom for 1,5-disubstituted 1,2,3-triazoles |

The location of the triazole in the bridge may affect the potency of the compound.

Suitable pairings of residues at positions X2 and X11 in which the location of the triazole in the bridge containing a triazole ring is closer to position X11 than position X2 once formed (i.e. the number of atoms in a linear chain from the first atom attached to the atom (carbon) adjacent to the carboxylic acid moiety of the amino acid residue (i.e. the first atom attached to the alpha carbon of the relevant residue for most amino acids) up to the triazole ring), include X2 is Lys(N₃) and X11 is Pra.

Alternatively, suitable pairings of residues at positions X2 and X11 in which location of the triazole in the bridge containing a triazole ring is closer to position X2 than position X11 once formed (i.e. the number of atoms in a linear chain from the first atom attached to the atom (carbon) adjacent to the carboxylic acid moiety of the amino acid residue (i.e. the first atom attached to the alpha carbon of the relevant residue for most amino acids) up to the triazole ring), include:
X2 is Pra and X11 is Dab(azidoacetic acid);
X2 is Pra and X11 is Dab((N₃)-Ala);
X2 is azidoacetic acid and X11 is Glu(propargylamine);
X2 is azidoacetic acid and X11 is Dab(3-butynoic acid);
X2 is (N₃)-Ala and X11 is Glu(propargylamine);
X2 is (N₃)-Ala and X11 is Dab(3-butynoic acid);
X2 is but-3-ynoic acid and X11 is Dab(azidoacetic acid); or
X2 is but-3-ynoic acid and X11 is Dab((N₃)-Ala).

If the bridge containing the triazole ring is formed from the side chains of the residues at X2 and X11, the side chain of the residue at position X2 is longer than the side chain of the residue at position X11, such that the location of the triazole in the bridge containing a triazole ring is closer to position 11 (X11) than position 2 (X2) once formed. Thus, in some embodiments the side chain of the residue at position X2 is longer than the side chain of the residue at position X11.

Suitable pairings of residues at positions X2 and X11 in which the side chain at position
X2 is longer than the side chain at position X11 include X2 is Lys(N₃) and X11 is Pra.

Alternatively, in some embodiments the side chain of the residue at position X2 is shorter than the side chain of the residue at position X11, such that the location of the triazole in the bridge containing a triazole ring is closer to position 2 (X2) than position (X11) once formed.

Suitable pairings of residues at positions X2 and X11 in which the side chain at position
X2 is shorter than the side chain at position X11 include:
X2 is Pra and X11 is Dab(azidoacetic acid); or
X2 is Pra and X11 is Dab((N₃)-Ala).

Desirably, the length of the bridge containing a triazole ring after the formation of the triazole (not including any atoms in the peptide backbone) is 5, 6, 7, 8, 9, or 10 atoms, such as 6, 7, 8, or 9 atoms; such as 7, 8, or 9 atoms, such as 8 or 9 atoms.

In some embodiments, the length of the bridge containing a triazole ring provided by the two side chains after formation of the triazole (not including any atoms in the peptide backbone) is 8 atoms. Suitable pairings of residues at positions X2 and X11 in which the bridge containing a triazole ring has a length of 8 atoms include: Lys(N₃) and Pra; azidoacetic acid and Glu(propargylamine); azidoacetic acid and Dab(3-butynoic acid); (N₃)-Ala and Glu(propargylamine); (N₃)-Ala and Dab(3-butynoic acid); but-3-ynoic acid and Dab(azidoacetic acid); and but-3-ynoic acid and Dab((N₃)-Ala).

In some embodiments, the length of the bridge containing a triazole ring provided by the two side chains after formation of the triazole (not including any atoms in the peptide backbone) is 9 atoms. Suitable pairings of residues at positions X2 and X11 in which the bridge containing a triazole ring has a length of 9 atoms include: Pra and Dab(azidoacetic acid); and Pra and Dab((N₃)-Ala).

### X4 and X7

X4 and X7 are amino acid residues who together form a lactam bridge, a dithioether bridge, or a bridge containing a triazole ring.

Preferably, X4 and X7 are amino acid residues who together form a lactam bridge. A lactam bridge is more stable than the corresponding dithioether bridge, 1,3-dithio-propan-2-one.

### Amino Acid Residues

The definitions for the dithioether bridge, the lactam bridge and the bridge containing a triazole ring is the same as defined for X2 and X11 above.

Suitable amino acid residues for X4 and X7 who together form a dithioether bridge may be Cys or N-Me-Cys.

Suitable amino acid residues for X4 and X7 who together form a lactam bridge may be selected from:
Amino acid residues comprising an amine group: Dpr, hLys, Lys, Arg, Orn, bAla, 3-(4-aminophenyl)propanoyl, (3-aminomethyl)benzoyl, (4-aminomethyl)benzoyl, 4-(2-aminoethyl)benzoyl, 2-aminomethyl-phenylacetyl, 3-aminomethyl-phenylacetyl, 4-aminomethyl-phenylacetyl, Dab, 6-aminohexanoyl, 6-amino-4-oxahexanoyl, trans-4-aminomethyl-cyclohexyl-1-carbonyl, (4-(2-aminoethyl)-piperazine-1-yl)-acetyl, 2,4-diaminobutanoyl([2-(trimethyl-2-aminoethoxy)ethoxy]propyl), and 4-methylaminomethyl-phenylacetyl.
Amino acid residues comprising a carboxylic acid group: Glu, Asp, Aad, and Apm.

Suitable amino acid residues for X4 and X7 who together form a bridge containing a triazole ring may be selected from:
Amino acid residues comprising an azide group: Ala(N₃), Aha, Orn(Na), Lys(N₃), azidoacetic acid, (N₃)-Ala, Dab(azidoacetic acid), and Dab((N₃)-Ala).
Amino acid residues comprising an alkyne group: Pra, Hpg, Bpg, Glu(propargylamine), Dab(3-butynoic acid), and but-3-ynoic acid.

### Bridge Length

The length of the bridge is counted as the number of atoms in a linear chain from the first atom attached to the atom (carbon) adjacent to the carboxylic acid moiety of the amino acid of the first residue (X4 for the bridge between X4 and X7), i.e. attached to the alpha carbon of the relevant residue for most amino acids, up to the first atom attached to the atom (carbon) adjacent to the carboxylic acid moiety of the amino acid of the second residue (X7 for the bridge between X4 and X7).

The contribution to the length of the bridge for amino acid residues and the type of bridge is described below.

In some embodiments, the length of the bridge between X4 and X7 is at least 5 atoms long. In some embodiments, the length of the bridge between X4 and X7 is no longer than 10 atoms long. In some embodiments, the length of the bridge between X4 and X7 is 5 to 10 atoms long, such as 5, 6, 7, 8, 9, or 10 atoms long.

### Dithioether Bridge

The inventors have found that a linker is required between the thiol side chains of linked [2,11] and [4,7] cysteine residues in the compounds to retain IL-23R inhibitory activity. Replacing the dithioether bridges with disulfide bridges resulted in loss of inhibitory activity (see Example 2).

Thus, the compound of the invention may comprise a dithioether bridge formed between the amino acid residues at positions X4 and X7. The dithioether bridge definitions, such as suitable amino acid residues, as described above for X2 and X11 are applicable here, replacing X2 and X11 for X4 and X7. For simplicity, positions X4 and X7 will be discussed by reference to the residues nominally present before dithioether formation.

In some embodiments, the dithioether bridge between X4 and X7 is of the formula —S—L—Y—L—S—, wherein:
each S is a sulfur atom and is part of the amino acid residue at X4 and X7;
each L is independently C₁₋₄ alkylene; and
Y is either absent, C(═O), or arylene.

In some embodiments, the dithioether bridge of formula —S—L—Y—L—S— is —SCH₂C(═O)CH₂S—. That is, each L is —CH₂— and Y is C(═O).

In some embodiments, the dithioether bridge of formula —S—L—Y—L—S— is —SCH₂(phenylene)CH₂S—, such as —SCH₂(1,2-phenylene)CH₂S—, —SCH₂(1,3-phenylene)CH₂S—, and —SCH₂(1,4-phenylene)CH₂S—. In some embodiments, the dithioether bridge of formula —S—L—Y—L—S— is —SCH₂(1,2-phenylene)CH₂S—. That is, each L is —CH₂— and Y is 1,2-phenylene.

In some embodiments, X4 and X7 are each independently selected from Cys and N-Me-Cys.

In some embodiments, X4 is Cys and X7 is Cys.

In some embodiments, X4 is Cys and X7 is N-Me-Cys. In some embodiments, X4 is N-Me-Cys and X7 is Cys.

Preferably, X4 is Cys and X7 is Cys.

In some embodiments, X4 is Cys and X7 is Cys who together form a dithioether bridge.

In some embodiments, X4 is Cys and X7 is Cys who together form a dithioether bridge, wherein the dithioether bridge is of the formula —S—L—Y—L—S—, wherein: each S is a sulfur atom and is part of the amino acid residue at X4 and X7; each L is independently C₁₋₄ alkylene; and Y is either absent, C(═O), or arylene.

In some embodiments, X4 is Cys and X7 is Cys who together form a dithioether bridge, wherein the dithioether bridge is of the formula —SCH₂C(═O)CH₂S—or —SCH₂(phenylene)CH₂S—, wherein each S is a sulfur atom and is part of the amino acid residue at X4 and X7.

In some embodiments, X4 is Cys and X7 is Cys who together form a dithioether bridge, wherein the dithioether bridge is of the formula —SCH₂C(═O)CH₂S—or —SCH₂(1,2-phenylene)CH₂S—, wherein each S is a sulfur atom and is part of the amino acid residue at X4 and X7.

The definitions for the length of the dithioether bridge as described above for X2 and X11 are applicable here, replacing X2 and X11 for X4 and X7. Preferably, the dithioether bridge is 7 or 8 atoms long.

### Lactam Bridge

The compound of the invention may comprise a lactam bridge formed between the amino acid residues at positions X4 and X7. The lactam bridge definitions, such as suitable amino acid residues, as described above for X2 and X11 are applicable here, replacing X2 and X11 for X4 and X7. For simplicity, positions X4 and X7 will be discussed by reference to the residues nominally present before lactam formation.

In some embodiments, one of the residues at position X4 and X7 is Lys, Dpr, Dab, or Orn, and the other is Glu.

In some embodiments the amine component of the lactam bridge derives from the amino acid at position X4, whereas the carboxylic acid component of the lactam bridge derives from the amino acid at position X7. In some embodiments, X4 is selected from Lys, Dpr, Dab, and Orn, and X7 is Glu.

Alternatively, in some embodiments the carboxylic acid component of the lactam bridge derives from the amino acid at position X4, whereas the amine component of the lactam bridge derives from the amino acid at position X7. In some embodiments, X4 may be Glu, and X7 is selected from Lys, Dpr, Dab, and Orn.

Suitable pairings of residues at positions X4 and X7 to form a lactam bridge include:

When X4 is the amine component and X7 is the carboxylic acid component:
X4 is Dpr and X7 is Glu;
X4 is Dab and X7 is Glu; or
X4 is Orn and X7 is Glu.

When X4 is the carboxylic acid component and X7 is the amine component:
X4 is Glu and X7 is Lys;
X4 is Glu and X7 is Dpr;
X4 is Glu and X7 is Orn; or
X4 is Glu and X7 is Dab.

### Preferably, X4 is Glu and X7 is Dab.

The definitions for the length of the lactam bridge as described above for X2 and X11 are applicable here, replacing X2 and X11 for X4 and X7.

The location of the amide bond in the lactam bridge may affect the potency of the compound, as described above for X2 and X11.

Suitable pairings of residues at positions X4 and X7 in which the location of the amide bond in the lactam bridge is closer to position X7 than position X4 once formed include:
X4 is Orn and X7 is Glu; or
X4 is Glu and X7 is Dpr.

Alternatively, suitable pairings of residues at positions X4 and X7 in which location of the amide bond in the lactam bridge is closer to position X4 than position X7 include:
X4 is Dpr and X7 is Glu;
X4 is Glu and X7 is Lys; or
X4 is Glu and X7 is Orn.

Suitable pairings of residues at positions X4 and X7 in which the side chain at position X4 is longer than the side chain at position X7, such that the location of the amide bond in the lactam bridge is closer to position 7 (X7) than position 4 (X4) once formed, include:
X4 is Orn and X7 is Glu; or
X4 is Glu and X7 is Dpr.

Alternatively, suitable pairings of residues at positions X4 and X7 in which the side chain at position X4 is shorter than the side chain at position X7, such that the location of the amide bond in the lactam bridge is closer to position 4 (X4) than position 7 (X7) once formed, include:
X4 is Dpr and X7 is Glu;
X4 is Glu and X7 is Lys; or
X4 is Glu and X7 is Orn.

Desirably, the length of the lactam bridge after formation of the amide bond (not including any atoms in the peptide backbone) is 5, 6, 7, 8, 9, or 10 atoms; such as 6, 7, 8, or 9 atoms; such as 7 or 8 atoms. In some embodiments, the length of the lactam bridge after formation of the amide bond (not including any atoms in the peptide backbone) is 5, 6, or 7 atoms.

In some embodiments, the length of the lactam bridge after formation of the amide bond (not including any atoms in the peptide backbone) is 5 atoms. A suitable pairing of residues at positions X4 and X7 in which the lactam bridge has a length of 5 atoms include Dpr and Glu.

In some embodiments, the length of the lactam bridge after formation of the amide bond (not including any atoms in the peptide backbone) is 6 atoms. A suitable pairing of residues at positions X4 and X7 in which the lactam bridge has a length of 6 atoms include Dab and Glu.

In some embodiments, the length of the lactam bridge after formation of the amide bond (not including any atoms in the peptide backbone) is 7 atoms. A suitable pairing of residues at positions X4 and X7 in which the lactam bridge has a length of 7 atoms include Orn and Glu.

In some embodiments, the length of the lactam bridge after formation of the amide bond (not including any atoms in the peptide backbone) is 8 atoms. Suitable pairings of residues at positions X4 and X7 in which the lactam bridge has a length of 8 atoms include: Glu and Lys; Aad and Om; and Asp and hLys.

### Bridge Containing a Triazole Ring

The compound of the invention may comprise a bridge containing a triazole ring formed between the amino acid residues at positions X4 and X7. The bridge containing a triazole ring definitions, such as suitable amino acid residues, as described above for X2 and X11 are applicable here, replacing X2 and X11 for X4 and X7. For simplicity, positions X4 and X7 will be discussed by reference to the residues nominally present before triazole formation.

In some embodiments, one of the residues at position X4 and X7 is selected from Lys(N₃) and Aha, and the other is Pra.

In some embodiments, X4 is Lys(N₃) and X7 is Pra. In some embodiments, X4 is Aha and X7 is Pra.

Suitable pairings of residues at positions X4 and X7 to form a bridge containing a triazole ring include:

When X4 is the azide component and X7 is the alkyne component:
X4 is Lys(N₃) and X7 is Pra; or
X4 is Aha and X7 is Pra.

Alternatively in some embodiments, X4 is the alkyne component and X7 is the azide component.

The definitions for the length of the bridge containing a triazole ring as described above for X2 and X11 are applicable here, replacing X2 and X11 for X4 and X7.

The location of the triazole in the bridge containing a triazole ring may affect the potency of the compound, as described above for X2 and X11.

Suitable pairings of residues at positions X4 and X7 in which the location of the triazole in the bridge containing a triazole ring is closer to position X7 than position X4 once formed, include X4 is Lys(N₃) and X7 is Pra.

Alternatively in some embodiments, the location of the triazole in the bridge containing a triazole ring is closer to position X4 than position X7 once formed.

Suitable pairings of residues at positions X4 and X7 in which the side chain at position X4 is longer than the side chain at position X7, such that the location of the triazole in the bridge containing a triazole ring is closer to position 7 (X7) than position 4 (X4), include X4 is Lys(N₃) and X7 is Pra.

Alternatively in some embodiments, the side chain at position X4 is shorter than the side chain at position X7.

### L

Each L is independently C₁₋₄ alkylene.

In some embodiments, L is C₁₋₂ alkylene. In some embodiments, L is C₁₋₃ alkylene(methylene or —CH₂—). In some embodiments, L is C₂ alkylene (ethylene or —CH₂CH₂—).

### Y

Y is either absent, C(═O), or arylene.

In some embodiments, Y is C(═O).

In some embodiments, Y is arylene, such as phenylene.

In some embodiments, Y is phenylene selected from 1,2-phenylene, 1,3-phenylene, and 1,4-phenylene. In some embodiments, Y is 1,2-phenylene.

### Synthesis of the Compounds

The invention further provides a method of synthesis of a compound of the invention. The compounds (which may also be referred to as peptides) may suitably be manufactured by standard synthetic methods. Thus, the peptides may be synthesized by, e.g., methods comprising synthesizing the peptide by standard solid-phase or liquid-phase methodology, either stepwise or by fragment assembly, and optionally isolating and purifying the final peptide product. In this context, reference may be made to WO 98/11125 or, inter alia, Fields, G. B. et al., “Principles and Practice of Solid-Phase Peptide Synthesis”; in: Synthetic Peptides, Gregory A. Grant (ed.), Oxford University Press (2^{nd} edition, 2002) and the synthesis examples herein. The method typically further comprises the step of forming an amide bond, forming two thioether bonds with a linker, or forming a triazole between the amino acid residues at positions 2 (X2) and I1 (X11), and optionally further comprising the step of forming an amide bond, forming two thioether bonds with a linker, or forming a triazole between the amino acid residues at positions 4 (X4) and 7 (X7), e.g. as described below. In the case of solid phase synthesis, cyclisation may be performed in situ on the solid phase (e.g. resin), i.e. before removal of the peptide from the solid phase.

The synthesis of some example compounds of the invention are provided in Example 1. Generally, the method for the synthesis of said compound comprises synthesising the compound by solid-phase or liquid-phase peptide synthesis methodology, optionally isolating and/or purifying the final product, and optionally further comprising the step of forming an amide bond, forming two thioether bonds with a linker, or forming a triazole between the amino acid residues at positions 2 and I1, and optionally further comprising the step of forming an amide bond, forming two thioether bonds with a linker, or forming a triazole between the amino acid residues at positions 4 and 7.

### Efficacy of the Compounds

The compounds of the invention are interleukin-23 receptor (IL-23R) inhibitors, i.e. they are capable of binding to, and blocking signalling by, one or more receptors or receptor complexes regarded as physiological receptors for interleukin-23 (IL-23).

Comparative activity may be measured by any suitable means, such as via determination of IC₅₀ values as described below.

Compounds of the present invention may exhibit a number of advantageous properties in relation to other peptide IL-23R inhibitors thereof, such as analogues described in WO 2016/011208, WO 2018/022937, WO 2018/136646, WO 2020/014646, WO 2021/146441, WO 2021/146458, and Heinis et al., 2020. As compared to any of these analogues, compounds of the invention may, for example, exhibit improved effects, e.g., in the form of improved in vitro potency at IL-23R.

Additionally or alternatively, compounds of the invention may exhibit improved gastrointestinal (GI) stability as compared to any of the peptide inhibitors of IL-23R described in the art.

The skilled person will be aware of suitable assay formats, and examples are provided below. For example, the assays may make use of employing measurements on the human IL-23R (see the examples below). Where sequences of precursor proteins are referred to, it should be understood that assays may make use of the mature protein, lacking the signal sequence.

K_{d} values may be used as a numerical measure of the binding affinity at a given receptor. A K_{d} value, also termed the equilibrium dissociation constant, is a measure of how tightly a compound binds to a receptor in a particular assay. A small K_{d} indicates the compound binds tighter with higher affinity to the receptor as compared to a compound with a higher K_{d} value. Thus, for example, a compound having a K_{d}[IL-23R] value lower than the K_{d}[IL-23R] value of another compound inhibitor of IL-23R in a particular assay may be considered to have a stronger binding affinity (or binds more tightly) to IL-23R than that of the other compound inhibitor of IL-23R.

In absence of an experimental method to directly determine the K_{d} of a compound for a receptor, the K_{d} may be estimated by means of calculating the Kᵢ value (inhibition constant). The Kᵢ is determined by the compound's ability to compete with a labelled compound for the receptor. In such a competition assay, the concentration for which half of the labelled compound is displaced from the receptor by the unlabelled compound is termed the IC₅₀ value. The IC₅₀ value is proportional to the affinity of the compound for the receptor (that is, its K_{d} value), but depends on the concentration of the labelled compound used as well as the affinity of the labelled compound for the receptor. Assuming that the binding is reversible, at equilibrium, the Kᵢ value is determined using the Cheng-Prussov equation, which describes the relationship between the Kᵢ and the IC₅₀ as: Kᵢ=IC₅₀/(1+[L_{L}]/K_{dL}), where [L_{L}] is the concentration of labelled compound used and K_{dL} is the equilibrium dissociation constant for the labelled compound (Cheng and Prusoff, 1973).

In some embodiments of compounds of the present invention, the Kᵢ towards IL-23R is below 1000 nM (e.g. 0.001 to 1000 nM).

In some embodiments of compounds of the present invention, the Kᵢ towards IL-23R is below 500 nM (e.g. 0.001 to 500 nM).

In some embodiments of compounds of the present invention, the Kᵢ towards IL-23R is below 100 nM (e.g. 0.001 to 100 nM).

In some embodiments of compounds of the present invention, the Kᵢ towards IL-23R is below 50 nM (e.g. 0.001 to 50 nM).

In some embodiments of compounds of the present invention, the Kᵢ towards IL-23R is below 30 nM (e.g. 0.001 to 30 nM).

In some embodiments of compounds of the present invention, the Kᵢ towards IL-23R is below 20 nM (e.g. 0.001 to 20 nM).

In some embodiments of compounds of the present invention, the K towards IL-23R is below 10 nM (e.g. 0.001 to 10 nM).

In some embodiments of compounds of the present invention, the Kᵢ towards IL-23R is below 5 nM (e.g. 0.001 to 5 nM).

In some embodiments of compounds of the present invention, the Kᵢ towards IL-23R is below 1 nM (e.g. 0.001 to 1 nM).

In some embodiments of compounds of the present invention, the Kᵢ towards IL-23R is below 0.5 nM (e.g. 0.001 to 0.5 nM).

In a functional assay format, measuring the ability of compounds to inhibit IL-23 mediated signalling in a cell-based assay, IC₅₀ values may be used as a numerical measure of inhibitor potency. An IC₅₀ value is a measure of the concentration of a compound required to achieve half of that compound's maximal activity in a particular assay. Thus, for example, a compound having an IC₅₀ [IL-23R] value lower than that of another compound inhibitor of IL-23R in a particular assay may be considered to have a stronger inhibitory potency, presumably by better blocking of the IL-23 mediated signalling, than that of the other peptide inhibitor of IL-23R.

In some embodiments of compounds of the present invention, the IC₅₀ towards IL-23 mediated signalling is below 1000 nM (e.g. 0.001 to 1000 nM).

In some embodiments of compounds of the present invention, the IC₅₀ towards IL-23 mediated signalling is below 500 nM (e.g. 0.001 to 500 nM).

In some embodiments of compounds of the present invention, the IC₅₀ towards IL-23 mediated signalling is below 100 nM (e.g. 0.001 to 100 nM).

In some embodiments of compounds of the present invention, the IC₅₀ towards IL-23 mediated signalling is below 50 nM (e.g. 0.001 to 50 nM).

In some embodiments of compounds of the present invention, the IC₅₀ towards IL-23 mediated signalling is below 30 nM (e.g. 0.001 to 30 nM).

In some embodiments of compounds of the present invention, the IC₅₀ towards IL-23 mediated signalling is below 20 nM (e.g. 0.001 to 20 nM).

In some embodiments of compounds of the present invention, the IC₅₀ towards IL-23 mediated signalling is below 10 nM (e.g. 0.001 to 10 nM).

Such assays may be performed under the conditions described in Example 3 below.

Additionally or alternatively, compounds of the invention may show gastrointestinal (GI) stability, i.e. resistance to degradation in the gastrointestinal tract. This can be measured using a simulated intestinal fluid (SIF) assay. For example, the compounds of the invention may retain at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, or at least 85% of the remaining compound or peptide after incubation for 1 hour, or for 4 hours, e.g. under the conditions described in Example 3.

### Pharmaceutical Compositions

The invention also extends to compositions, such as pharmaceutical compositions, comprising the compounds of the invention. As with all aspects of the invention, it is to be understood that reference to a compound of the invention encompasses reference to pharmaceutically acceptable salts and solvates.

The compounds of the present invention may be formulated as pharmaceutical compositions which are suited for administration with or without storage, and which typically comprise a therapeutically effective amount of at least one peptide of the invention, together with a pharmaceutically acceptable carrier, excipient or vehicle.

The term “pharmaceutically acceptable carrier” includes any of the standard pharmaceutical carriers. Pharmaceutically acceptable carriers for therapeutic use are well known in the pharmaceutical art and are described, for example, in “Remington's Pharmaceutical Sciences”, 17^{th} edition, Alfonso R. Gennaro (Ed.), Mark Publishing Company, Easton, PA, USA, 1985. For example, sterile saline and phosphate-buffered saline at slightly acidic or physiological pH may be used. Suitable pH-buffering agents may, e.g., be phosphate, citrate, acetate, tris(hydroxymethyl)aminomethane (TRIS), N-tris(hydroxymethyl)methyl-3-aminopropanesulfonic acid (TAPS), ammonium bicarbonate, diethanolamine, histidine, arginine, lysine or acetate (e.g. as sodium acetate), or mixtures thereof. The term further encompasses any carrier agents listed in the US Pharmacopeia for use in animals, including humans.

A pharmaceutical composition of the invention may be in unit dosage form. In such form, the composition is divided into unit doses containing appropriate quantities of the active component or components. The unit dosage form may be presented as a packaged preparation, the package containing discrete quantities of the preparation, for example, packaged tablets, capsules or powders in vials or ampoules. The unit dosage form may also be, e.g., a capsule, cachet or tablet in itself, or it may be an appropriate number of any of these packaged forms. A unit dosage form may also be provided in single-dose injectable form, for example in the form of a pen device containing a liquid-phase (typically aqueous) composition. Compositions may be formulated for any suitable route and means of administration. Pharmaceutically acceptable carriers or diluents include those used in formulations suitable for e.g. oral, intravitreal, rectal, vaginal, nasal, topical, enteral or parenteral (including subcutaneous (sc), intramuscular (im), intravenous (iv), intradermal and transdermal) administration or administration by inhalation. The formulations may conveniently be presented in unit dosage form and may be prepared by any of the methods well known in the art of pharmaceutical formulation.

Subcutaneous or transdermal modes of administration may in some cases be suitable for peptides of the invention.

Further embodiments relate to devices, dosage forms and packages used to deliver the pharmaceutical formulations of the present invention. Thus, at least one peptide in a stable or preserved formulation or solution described herein can be administered to a patient in accordance with the present invention via a variety of delivery methods, including by sc or im injection, or by transdermal, pulmonary or transmucosal administration, or by implant, or by use of an osmotic pump, cartridge, micro-pump or other means recognized by a person of skill in the art.

Still further embodiments relate to oral formulations and oral administration. Formulations for oral administration may rely on the co-administration of adjuvants (e.g. resorcinols and/or nonionic surfactants such as polyoxyethylene oleyl ether and n-hexadecylpolyethylene ether) to artificially increase the permeability of the intestinal walls, and/or the co-administration of enzymatic inhibitors (e.g. pancreatic trypsin inhibitors, diisopropylfluorophosphate (DFF) or trasylol) to inhibit enzymatic degradation. The active constituent compound of a solid-type dosage form for oral administration can be mixed with at least one additive, such as sucrose, lactose, cellulose, mannitol, trehalose, raffinose, maltitol, dextran, starches, agar, alginates, chitins, chitosans, pectins, gum tragacanth, gum arabic, gelatin, collagen, casein, albumin, synthetic or semisynthetic polymer, or glyceride. These dosage forms can also contain other type(s) of additives, e.g. an inactive diluting agent, a lubricant (such as magnesium stearate), a paraben, a preserving agent (such as sorbic acid, ascorbic acid or alpha-tocopherol), an antioxidant (such as cysteine), a disintegrant, binder, thickener, buffering agent, pH-adjusting agent, sweetening agent, flavoring agent or perfuming agent.

### Therapeutic Uses

The compounds of the invention, and pharmaceutical compositions comprising said compounds, are useful in a method of prevention or treatment of Inflammatory Bowel Disease (IBD), ulcerative colitis, Crohn's disease, Celiac disease (nontropical Sprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-I, chronic granulomatous disease, glycogen storage disease type 1b, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, ankylosing spondylitis, or graft versus host disease in a subject, and combinations thereof.

The method of prevention or treatment comprises administering to the subject an effective amount of the compound of the invention, or the pharmaceutical composition comprising said compound.

In some embodiments, the conditions may be selected from inflammatory bowel (IBD), Crohn's Disease, ulcerative colitis, and psoriasis.

### Dosages

A typical dosage of a compound as employed in the context of the present invention may be in the range from about 0.0001 to about 100 mg/kg body weight per day, such as from about 0.0005 to about 50 mg/kg body weight per day, such as from about 0.001 to about 10 mg/kg body weight per day, e.g. from about 0.01 to about 1 mg/kg body weight per day, administered in one or more doses, such as from one to three doses. The exact dosage employed will depend, inter alia, on: the nature and severity of the disease or disorder to be treated, on the sex, age, body weight and general condition of the subject to be treated, on possible other, concomitant, disease or disorder that is undergoing or is to undergo treatment, as well as on other factors that will be known to a medical practitioner of skill in the art.

A compound of the invention may be administered continuously (e.g. by intravenous administration or another continuous drug administration method), or may be administered to a subject at intervals, typically at regular time intervals, depending on the desired dosage and the pharmaceutical composition selected by the skilled practitioner for the particular subject. Regular administration dosing intervals include, e.g., once daily, twice daily, once every two, three, four, five or six days, once or twice weekly, once or twice monthly, and the like. Such regular peptide administration regimens may, in certain circumstances such as, e.g., during chronic long-term administration, be advantageously interrupted for a period of time so that the medicated subject reduces the level of, or stops taking, the medication, often referred to as taking a “drug holiday.” Drug holidays are useful for, e.g., maintaining or regaining sensitivity to a drug especially during long-term chronic treatment, or to reduce unwanted side-effects of long-term chronic treatment of the subject with the drug. The timing of a drug holiday depends on the timing of the regular dosing regimen and the purpose for taking the drug holiday (e.g., to regain drug sensitivity and/or to reduce unwanted side effects of continuous, long-term administration). In some embodiments, the drug holiday may be a reduction in the dosage of the drug (e.g. to below the therapeutically effective amount for a certain interval of time). In other embodiments, administration of the drug is stopped for a certain interval of time before administration is started again using the same or a different dosing regimen (e.g. at a lower or higher dose and/or frequency of administration). OA drug holiday of the invention may thus be selected from a wide range of time-periods and dosage regimens. An exemplary drug holiday is two or more days, one or more weeks, or one or more months, up to about 24 months of drug holiday. So, for example, a regular daily dosing regimen with a peptide of the invention may, for example, be interrupted by a drug holiday of a week, or two weeks, or four weeks, after which time the preceding, regular dosage regimen (e.g. a daily or a weekly dosing regimen) is resumed. A variety of other drug holiday regimens are envisioned to be useful for administering peptides of the invention.

Thus, the peptide may be delivered via an administration regime which comprises two or more administration phases separated by respective drug holiday phases.

During each administration phase, the peptide is administered to the recipient subject in a therapeutically effective amount according to a pre-determined administration pattern.

The administration pattern may comprise continuous administration of the drug to the recipient subject over the duration of the administration phase. Alternatively, the administration pattern may comprise administration of a plurality of doses of the peptide to the recipient subject, wherein said doses are spaced by dosing intervals.

A dosing pattern may comprise at least two doses per administration phase, at least five doses per administration phase, at least 10 doses per administration phase, at least 20 doses per administration phase, at least 30 doses per administration phase, or more.

Said dosing intervals may be regular dosing intervals, which may be as set out above, including once daily, twice daily, once every two, three, four, five or six days, once or twice weekly, once or twice monthly, or a regular and even less frequent dosing interval, depending on the particular dosage formulation, bioavailability, and pharmacokinetic profile of the peptide.

An administration phase may have a duration of at least two days, at least a week, at least 2 weeks, at least 4 weeks, at least a month, at least 2 months, at least 3 months, at least 6 months, or more.

Where an administration pattern comprises a plurality of doses, the duration of a possible following drug holiday phase is longer than the dosing interval used in that administration pattern. Where the dosing interval is irregular, the duration of a drug holiday phase may be greater than the mean interval between doses over the course of the administration phase. Alternatively the duration of the drug holiday may be longer than the longest interval between consecutive doses during the administration phase.

The duration of a possible drug holiday phase may be at least twice that of the relevant dosing interval (or mean thereof), at least 3 times, at least 4 times, at least 5 times, at least 10 times, or at least 20 times that of the relevant dosing interval or mean thereof.

Within these constraints, a drug holiday phase may have a duration of at least two days, at least a week, at least 2 weeks, at least 4 weeks, at least a month, at least 2 months, at least 3 months, at least 6 months, or more, depending on the administration pattern during the previous administration phase.

An administration regime entailing the use of drug holiday comprises at least 2 administration phases. Consecutive administration phases are separated by respective drug holiday phases. Thus the administration regime may comprise at least 3, at least 4, at least 5, at least 10, at least 15, at least 20, at least 25, or at least 30 administration phases, or more, each separated by respective drug holiday phases.

Consecutive administration phases may utilise the same administration pattern, although this may not always be desirable or necessary. However, if other drugs or active agents are administered in combination with a peptide of the invention, then typically the same combination of drugs or active agents is given in consecutive administration phases. In certain embodiments, the recipient subject is a human.

### Devices and Kits

In some embodiments, the invention relates to a device comprising a compound or pharmaceutical composition of the invention, for delivery of the compound to a subject.

Via such devices, the compounds described herein can be administered to a patient via a variety of delivery methods, including: intravenous, subcutaneous, intramuscular or intraperitoneal injection; oral administration; transdermal administration; pulmonary or transmucosal administration; administration by implant, osmotic pump, cartridge or micro pump; or by other means recognized by a person of skill in the art.

In some embodiments, the invention relates to a kit comprising a compound of the invention or a pharmaceutical composition of the invention. In certain embodiments, the kit further comprises packaging and/or instructions for use.

The device or kit may be useful for combination therapy as described above. Thus the device or kit may further comprise a further active agent, e.g. in combination with drugs belonging to the drug classes disease modifying drugs (DMARDs), non-steroidal anti-inflammatory drugs (NSAIDs), immunosuppressants, and glucocorticoids; or in combination with other biologics, such as anti-TNFs; anti-integrins such as Vedolizumab; or IL-23 antibodies such as Ustekinumab, Guselkumab, Tildrakuzumab and Risankizumab.

### EXAMPLES

The following examples demonstrate certain specific embodiments of the present invention. The following examples were carried out using standard techniques that are well known and routine to those of skill in the art, except where otherwise described in detail. It is to be understood that these examples are for illustrative purposes only and do not purport to be wholly definitive as to conditions or scope of the invention. As such, they should not be construed as limiting the scope of the present invention in any way.

Abbreviations employed in the examples include:

| | |
|---|---|
| 2-Nal | 3-(2-naphthyl)-L-alanine |
| {d}R or D-Arg | D-arginine |
| {d}L or D-Leu | D-leucine |
| {d}C or D-Cys | D-cysteine |
| {d}Q or D-Gln | D-glutamine |
| {d}E or D-Glu | D-glutamic acid |
| {d}H or D-His | D-histidine |
| alpha-Me-Trp | α-methyl-L-tryptophan |
| 1-Me-Trp | 1-methyl-L-tryptophan |
| 4-F-Trp | 4-fluoro-L-tryptophan |
| 6-F-Trp | 6-fluoro-L-tryptophan |
| {d}6-F-Trp | 6-fluoro-D-tryptophan |
| 6-Cl-Trp | 6-chloro-L-tryptophan |
| 7-Me-Trp | 7-methyl-L-tryptophan |
| 7-F-Trp | 7-fluoro-L-tryptophan |
| 7-Ph-Trp | 7-phenyl-L-tryptophan |
| 7-(Naphth-2-yl)-Trp | 7-(naphth-2-yl)-L-tryptophan |
| 3-(3-Pyridyl)-Ala | 3-(3-pyridyl)-L-alanine |
| 3-(4-Pyridyl)-Ala | 3-(4-pyridyl)-L-alanine |
| {d}[3-(3-Pyridyl)-Ala] | 3-(3-pyridyl)-D-alanine |
| 2-Me-3-(3-Pyridyl)-Ala | (2S)-2-amino-2-methyl-3-(3-pyridyl)propanoic acid |
| 3-(3-Quinolinyl)-Ala | 3-(3-quinolinyl)-L-alanine; also known as |
| | (2S)-2-amino-3-(3-quinolyl)propanoic acid |
| F(3-F) or 3-F-Phe | 3-fluoro-L-phenylalanine |
| F(4-F) or 4-F-Phe | 4-fluoro-L-phenylalanine |
| {d}F(4-F) | 4-fluoro-D-phenylalanine |
| F(4-Cl) or 4-Cl-Phe | 4-chloro-L-phenylalanine |
| F(4-NH₂) or 4-NH₂-Phe | 4-amino-L-phenylalanine |
| F(4-Me) or 4-Me-Phe | 4-methyl-L-phenylalanine |
| F(3,4-Me) or 3,4-Me-Phe | 3,4-dimethyl-L-phenylalanine |
| F(3,5-F) or 3,5-F-Phe | 3,5-difluoro-L-phenylalanine |
| 2-Me-F(4-F) | (2S)-2-amino-3-(4-fluorophenyl)-2-methyl-propanoic |
| 2-Me-Leu | 2-methyl-L-leucine |
| 2-Me-Lys | 2-methyl-L-lysine |
| 2-Me-Arg | 2-methyl-L-arginine |
| 2-Me-Val | 2-methyl-L-valine |
| 2-Me-Phe | alpha-methyl-L-phenylalanine; also known as |
| | (2S)-2-amino-2-methyl-3-phenyl-propanoic acid |
| N-Me-Arg | N2-methyl-L-arginine |
| N-Me-Ser | N-methyl-L-serine |
| N-Me-Cys | N-methyl-L-cysteine |
| N-Me-Trp | Nα-methyl-L-tryptophan; also known as L-abrine |
| N-Me-Gln | N2-methyl-L-glutamine |
| Bip | biphenyl-L-alanine |
| 3,3-Diphenyl-Ala | 3,3-diphenyl-L-alanine; also known as |
| | β-phenyl-L-phenylalanine, or |
| | (S)-2-amino-3,3-diphenylpropionic acid |
| Aad | (2S)-2-aminoadipic acid, also known as |
| | (2S)-2-aminohexanedioic acid, or L-homoglutamic acid |
| Apm | (2S)-2-aminopimelic acid, also known as |
| | (2S)-2-aminoheptanedioic acid, or L-bishomoglutamic acid |
| Dab | (2S)-2,4-diaminobutanoic acid |
| Orn | L-ornithine, also known as 2,5-diaminopentanoic acid |
| hLys | (2S)-2-amino-7-amino-heptanoic acid, also known as |
| | L-homolysine |
| bAla | 3-aminopropionic acid, also known as beta-alanine or |
| | β-alanine |
| beta-homo-Ser | L-β-homoserine |
| Ala(N₃) | 3-azido-L-alanine |
| Aha | azidohomo-L-alanine or 4-azido-L-homoalanine |
| Orn(N₃) | azido-L-ornithine |
| K(N₃) or Lys(N₃) | azido-L-lysine |
| Pra | L-propargylglycine |
| Hpg | L-homopropargylglycine |
| Bpg | L-bishomopropargylglycine |
| Dpr | (2S)-2,3-diaminopropanoic acid, also known as |
| | 3-amino-L-alanine |
| Glu(propargylamine) | (2S)-2-amino-5-oxo-5-(prop-2-ynylamino)pentanoic acid |
| Dab(3-butynoic acid) | (2S)-2-amino-4-(but-3-ynoylamino)butanoic acid |
| Dab(azidoacetic acid) | (2S)-2-amino-4-[(2-azidoacetyl)amino]butanoic acid |
| (N₃)-Ala | (2S)-2-azidopropanoic acid |
| Dab((N₃)-Ala)) | (2S)-2-amino-4-[(2S)-2-azidopropanoyl]amino]butanoic acid |
| Abu | (2S)-2-aminobutyric acid |
| Y(2-aminoethoxy) | (2S)-2-amino-3-[4-(2-aminoethoxy)phenyl]propanoic acid |
| Y(Me) | (2S)-2-amino-3-(4-methoxyphenyl)propanoic acid |
| Q(pyrrolidin) | (2S)-2-amino-5-oxo-5-pyrrolidin-1-yl-pentanoic acid |
| GABA | gamma aminobutyric acid; also known as |
| | 4-aminobutanoic acid |
| Phg | L-2-phenylglycine |
| F(4-NH₂-(2-(trimethyl-2-aminoethoxy)ethoxy)propyl) | |
| 2-[2-[3-[4-[(2S)-2- | |
| amino-2-carboxy-ethyl]anilino]-3-oxo-propoxy]ethoxy]ethyl-trimethyl-ammonium | |
| ^{t}BuOH | tert-Butanol |
| DODT | 2,2′-(Ethylenedioxy)diethanethiol |
| Pd(PPh₃)₄ | tetrakis(triphenylphosphine)palladium(0)) |
| PhSiH₃ | phenylsilane |
| PyBOP | benzotriazol-1-yloxytripyrrolidinophosphonium |
| | hexafluorophosphate) |
| equiv. | equivalents |
| r.t. | room temperature |
| aq. | Aqueous |
| IL-23R | interleukin-23 receptor |
| hIL-23R | human interleukin-23 receptor |
| GI | gastrointestinal |
| SIF | simulated intestinal fluid |
| SGF | simulated gastric fluid |
| Nluc | NanoBRET luciferase assay |
| SD | standard deviation |
| % Eff | percentage efficacy |
| pSTAT3 | phosphorylated signal transducer and activator of |
| | transcription 3 |
| BRET | Bioluminescence Resonance Energy Transfer |
| TAMRA | 5′-tetramethylrhodamine-5-carboxamide |

The following examples are provided to illustrate certain embodiments of the invention and are not intended to limit the scope of the invention.

### Example 1: Synthesis of Compounds

The following compounds were synthesised in Table 1-1 below.

TABLE 1-1
Synthesised compounds

| Compound | Structure |
|---|---|
| 1 | H-DC(1a)SC(2a)WQC(2a)WW[2-Me-Leu]C(1a)R-[NH2] |
| 2 | H-LC(1a)SC(2a)WQC(2a)WWLC(1a)R-[NH2] |
| 3 | H-DC(1a)SC(2a)WEC(2a)WWLC(1a)R-[NH2] |
| 4 | H-DE(1c)SC(2a)WQC(2a)WWLK(1c)R-[NH2] |
| 5 | H-DC(1a)SE(2c)WQK(2c)WWLC(1a)R-[NH2] |
| 6 | D(1c)*SC(2a)WQC(2a)WWLR(1c)* |
| 7 | [Ac]-DK(1c)SC(2a)WQC(2a)WWLE(1c)R-[NH2] |
| 8 | [Ac]-D[Orn](1c)SC(2a)WQC(2a)WWLE(1c)R-[NH2] |
| 9 | H-DE(1c)SC(2a)WQC(2a)WWL[Dab](1c)R-[NH2] |
| 10 | [Ac]-E(1c)SC(2a)WQC(2a)WWLK(1c)R-[NH2] |
| 11 | [Ac]-DE(1c)SC(2a)WQC(2a)WWLK(1c)-[NH2] |
| 12 | [Ac]-E(1c)SC(2a)WQC(2a)WWLK(1c)-[NH2] |
| 13 | [Ac]-DE(1c)SC(2a)AQC(2a)WWLK(1c)R-[NH2] |
| 14 | [Ac]-DE(1c)SC(2a)WQC(2a)AWLK(1c)R-[NH2] |
| 15 | [Ac]-DE(1c)SC(2a)WQC(2a)WALK(1c)R-[NH2] |
| 16 | [Ac]-DE(1c)SC(2a)[7-Me-Trp]QC(2a)WWLK(1c)R-[NH2] |
| 17 | [Ac]-DE(1c)SC(2a)WQC(2a)W[2-Nal]LK(1c)R-[NH2] |
| 18 | [Ac]-K(1c)SC(2a)WQC(2a)W[2-Nal]LE(1c)R-[NH2] |
| 19 | [Ac]-DK(1c)SC(2a)WQC(2a)W[2-Nal]LE(1c)R-[NH2] |
| 20 | [Ac]-DK(1c)SC(2d)WQC(2d)W[2-Nal]LE(1c)R-[NH2] |
| 21 | [Ac]-DK(1c)SC(2e)WQC(2e)W[2-Nal]LE(1c)R-[NH2] |
| 22 | [Ac]-DK(1c)SC(2f)WQC(2f)W[2-Nal]LE(1c)R-[NH2] |
| 23 | [Ac]-DK(1c)S[K(N3)](2g)WQ[Pra](2g)W[2-Nal]LE(1c)R-[NH2] |
| 24 | [Ac]-DK(1c)S[Aha](2g)WQ[Pra](2g)W[2-Nal]LE(1c)R-[NH2] |
| 25 | [Ac]-K(1c)SC(2a)[2-Nal]QC(2a)W[2-Nal]LE(1c)R-[NH2] |
| 26 | [Ac]-K(1c)SC(2a)[Bip]QC(2a)W[2-Nal]LE(1c)R-[NH2] |
| 27 | [Ac]-K(1c)SC(2a)[1-Me-Trp]QC(2a)W[2-Nal]LE(1c)R-[NH2] |
| 28 | [Ac]-DK(1c)SC(2a)WQC(2a)[2-Nal][2-Nal]LE(1c)R-[NH2] |
| 29 | [Ac]-K(1c)SC(2a)WQC(2a)[Y(2-aminoethoxy)][2-Nal]LE(1c)R-[NH2] |
| 30 | [Ac]-K(1c)SC(2a)WQC(2a)H[2-Nal]LE(1c)R-[NH2] |
| 31 | [Ac]-K(1c)SC(2a)WQC(2a)W[F(4-F)]LE(1c)R-[NH2] |
| 32 | [Ac]-K(1c)SC(2a)WQC(2a)W[1-Me-Trp]LE(1c)R-[NH2] |
| 33 | [Ac]-C(1a)TC(2a)WEC(2a)WW[2-Me-Leu]C(1a)S-[NH2] |
| 34 | [Ac]-GK(1c)TC(2a)WEC(2c)WW[2-Me-Leu]E(1c)S-[NH2] |
| 35 | [Ac]-GC(1a)TC(2a)WEC(2a)W[2-Nal][2-Me-Leu]C(1a)R-[NH2] |
| 36 | [bAla](1c)*SC(2a)WQC(2a)W[2-Nal]LE(1c)R-[NH2] |
| 37 | [(3-Aminomethyl)benzoyl](1c)*SC(2a)WQC(2a)W[2-Nal]LE(1c)R-[NH2] |
| 38 | [(3-Aminomethyl)benzoyl](1c)*SC(2a)WQC(2a)[Y(2-aminoethoxy)][2- |
| | Nal][2-Me-Leu]E(1c)R-[NH2] |
| 39 | [(3-Aminomethyl)benzoyl](1c)*SC(2a)WQC(2a)[Y(2-aminoethoxy)][2- |
| | Nal]LE(1c)R-[NH2] |
| 40 | [(3-Aminomethyl)benzoyl](1c)*SC(2a)WQC(2a)W[2-Nal][2-Me- |
| | Leu]E(1c)R-[NH2] |
| 41 | [(3-Aminomethyl)benzoyl](1c)*SC(2a)WQC(2a)[Y(2-aminoethoxy)][2- |
| | Nal][2-Me-Leu]E(1c)[{d}R]-[NH2] |
| 42 | [(3-Aminomethyl)benzoyl](1c)*SC(2a)WQC(2a)[Y(2-aminoethoxy)][2- |
| | Nal][2-Me-Leu]E(1c)[2-Me-Arg]-[NH2] |
| 43 | [(3-Aminomethyl)benzoyl](1c)*SC(2a)WQC(2a)[Y(2-aminoethoxy)][2- |
| | Nal][2-Me-Leu]E(1c)[N-Me-Arg]-[NH2] |
| 44 | [(3-Aminomethyl)benzoyl](1c)*SC(2a)WQC(2a)[Y(2-aminoethoxy)][2- |
| | Nal][2-Me-Leu]E(1c)S-[NH2] |
| 45 | [(3-Aminomethyl)benzoyl](1c)*SC(2a)WQC(2a)[Y(2-aminoethoxy)][2- |
| | Nal][2-Me-Leu]E(1c)[F(4-NH2)]-[NH2] |
| 46 | [(3-Aminomethyl)benzoyl](1c)*SC(2a)WQC(2a)[Y(2-aminoethoxy)][2- |
| | Nal][{d}L]E(1c)Y-[NH2] |
| 47 | [Ac]-K(1c)SC(2a)WQC(2a)[Y(2-aminoethoxy)][2-Nal][2-Me-Leu]E(1c)R- |
| | [NH2] |
| 48 | [Ac]-K(1c)SC(2a)WQC(2a)[Y(2-aminoethoxy)][2-Nal][{d}LJE(1c)R-[NH2] |
| 49 | [(4-Aminomethyl)benzoyl](1c)*SC(2a)WQC(2a)[Y(2-aminoethoxy)][2- |
| | Nal][2-Me-Leu]E(1c)R-[NH2] |
| 50 | [4-(2-Aminoethyl)benzoyl](1c)*SC(2a)WQC(2a)[Y(2-aminoethoxy)][2- |
| | Nal][2-Me-Leu]E(1c)R-[NH2] |
| 51 | [3-(4-Aminophenyl)propanoyl](1c)*SC(2a)WQC(2a)[Y(2-aminoethoxy)][2- |
| | Nal][2-Me-Leu]E(1c)R-[NH2] |
| 52 | [4-Aminomethyl-phenylacetyl](1c)*SC(2a)WQC(2a)[Y(2-aminoethoxy)][2- |
| | Nal][2-Me-Leu]E(1c)R-[NH2] |
| 53 | H-DC(1a)SC(2b)WQC(2b)WWLC(1a)R-[NH2] |
| 54 | [Ac]-K(1c)SC(2b)WQC(2b)[Y(2-aminoethoxy)][2-Nal]LE(1c)R-[NH2] |
| 55 | [Ac]-DE(1c)QC(2a)WQC(2a)YW[2-Me-Leu]K(1c)R-[NH2] |
| 56 | [Ac]-DE(1c)QC(2a)WQC(2a)[2-Nal]W[2-Me-Leu]K(1c)R-[NH2] |
| 57 | [Ac]-DE(1c)TC(2a)WQC(2a)W[2-Nal][2-Me-Leu]K(1c)R-[NH2] |
| 58 | [Ac]-DE(1c)SC(2a)WQC(2a)W[2-Nal][2-Me-Leu]K(1c)ENG-[NH2] |
| 59 | [Ac]-DE(1c)SC(2a)WQC(2a)W[2-Nal][2-Me-Leu]K(1c)NG-[NH2] |
| 60 | [Ac]-DE(1c)SC(2a)WQC(2a)W[2-Nal][2-Me-Leu]K(1c)G-[NH2] |
| 61 | [Ac]-DE(1c)SC(2a)WQC(2a)W[2-Nal][2-Me-Lys]K(1c)ENG-[NH2] |
| 62 | [Ac]-DC(1a)QC(2a)WQC(2a)[2-Nal]W[2-Me-Leu]C(1a)R-[NH2] |
| 63 | [Ac]-DC(1a)QC(2a)WQC(2a)YW[2-Me-Leu]C(1a)R-[NH2] |
| 64 | [Ac]-DC(1a)QC(2a)WQC(2a)WW[2-Me-Leu]C(1a)ENG-[NH2] |
| 65 | [(3-Aminomethyl)benzoyl](1c)*S[Dpr](2c)WQE(2c)[Y(2-aminoethoxy)][2- |
| | Nal][2-Me-Leu]E(1c)R-[NH2] |
| 66 | [(3-Aminomethyl)benzoyl](1c)*SE(2c)WQ[Dpr](2c)[Y(2-aminoethoxy)][2- |
| | Nal][2-Me-Leu]E(1c)R-[NH2] |
| 67 | [(3-Aminomethyl)benzoyl](1c)*SE(2c)WQ[Dab](2c)[Y(2-aminoethoxy)][2- |
| | Nal][2-Me-Leu]E(1c)R-[NH2] |
| 68 | [(3-Aminomethyl)benzoyl](1c)*S[Orn](2c)WQE(2c)[Y(2-aminoethoxy)][2- |
| | Nal][2-Me-Leu]E(1c)R-[NH2] |
| 69 | [(3-Aminomethyl)benzoyl](1c)*SE(2c)WQ[Orn](2c)[Y(2-aminoethoxy)][2- |
| | Nal][2-Me-Leu]E(1c)R-[NH2] |
| 70 | [(3-Aminomethyl)benzoyl](1c)*SC(2a)[7-Me-Trp]QC(2a)[Y(2- |
| | aminoethoxy)][2-Nal][2-Me-Leu]E(1c)R-[NH2] |
| 71 | [(3-Aminomethyl)benzoyl](1c)*SC(2a)[7-Ph-Trp]QC(2a)[Y(2- |
| | aminoethoxy)][2-Nal][2-Me-Leu]E(1c)R-[NH2] |
| 72 | [(3-Aminomethyl)benzoyl](1c)*SC(2a)[7-(Naphth-2-yl)-Trp]QC(2a)[Y(2- |
| | aminoethoxy)][2-Nal][2-Me-Leu]E(1c)R-[NH2] |
| 73 | [(3-Aminomethyl)benzoyl](1c)*SC(2a)WQC(2a)[Y(2-aminoethoxy)][3-(3- |
| | Quinolinyl)-Ala][2-Me-Leu]E(1c)R-[NH2] |
| 74 | [(3-Aminomethyl)benzoyl](1c)*SC(2a)WQC(2a)[Y(2-aminoethoxy)][6-CI- |
| | Trp][2-Me-Leu]E(1c)R-[NH2] |
| 75 | [(3-Aminomethyl)benzoyl](1c)*SC(2a)WQC(2a)[Y(2-aminoethoxy)][2- |
| | Nal][4-aminotetrahydro-2H-pyran-4-acetyl]E(1c)R-[NH2] |
| 76 | [(3-Aminomethyl)benzoyl](1c)*SC(2a)WQC(2a)[Y(2-aminoethoxy)][2- |
| | Nal][2-Me-Leu]E(1c)[3-(3-Pyridyl)-Ala]-[NH2] |
| 77 | [(3-Aminomethyl)benzoyl](1c)*SC(2a)WQC(2a)[Y(2-aminoethoxy)][2- |
| | Nal][2-Me-Leu]E(1c)[3-(4-Pyridyl)-Ala]-[NH2] |
| 78 | [(3-Aminomethyl)benzoyl](1c)*SC(2a)WQC(2a)[Y(2-aminoethoxy)][2- |
| | Nal][2-Me-Leu]E(1c)R[3-(3-Pyridyl)-Ala]-[NH2] |
| 79 | [(3-Aminomethyl)benzoyl](1c)*SC(2a)WQC(2a)[Y(2-aminoethoxy)][2- |
| | Nal][2-Me-Leu]E(1c)R[3-(4-Pyridyl)-Ala]-[NH2] |
| 80 | [4-Aminomethyl-phenylacetyl](1c)*SC(2a)WQC(2a)[Y(2-aminoethoxy)][2- |
| | Nal][2-Me-Leu]E(1c)[{d}R]-[NH2] |
| 81 | [4-Aminomethyl-phenylacetyl](1c)*SC(2a)WQC(2a)[Y(2-aminoethoxy)][2- |
| | Nal][2-Me-Leu]E(1c)S-[NH2] |
| 82 | [4-Aminomethyl-phenylacetyl](1c)*SC(2a)WQC(2a)[Y(2-aminoethoxy)][2- |
| | Nal][2-Me-Leu]E(1c)[Dab]-[NH2] |
| 83 | [4-Aminomethyl-phenylacetyl](1c)*TC(2a)WQC(2a)[Y(2-aminoethoxy)][2- |
| | Nal][2-Me-Leu]E(1c)[{d}R]-[NH2] |
| 84 | [Ac]-K(1c)SC(2a)WQC(2a)[Y(2-aminoethoxy)][2-Nal][2-Me- |
| | Leu]E(1c)[{d}R]-[NH2] |
| 85 | [Ac]-K(1c) TC(2a)WQC(2a)[Y(2-aminoethoxy)][2-Nal][2-Me- |
| | Leu]E(1c)[{d}R]-[NH2] |
| 86 | [Ac]-K(1c)SC(2a)WQC(2a)[Y(2-aminoethoxy)][2-Nal][2-Me-Leu]E(1c)S- |
| | [NH2] |
| 87 | [6-Aminohexanoyl](1c)*SC(2a)WQC(2a)[Y(2-aminoethoxy)][2-Nal][2-Me- |
| | Leu]E(1c)[{d}R]-[NH2] |
| 88 | [6-Amino-4-oxahexanoyl](1c)*SC(2a)WQC(2a)[Y(2-aminoethoxy)][2- |
| | Nal][2-Me-Leu]E(1c) [{d}R]-[NH2] |
| 89 | [trans-4-Aminomethyl-cyclohexyl-1-carbonyl](1c)*[beta-homo- |
| | Ser]C(2a)WQC(2a)[Y(2-aminoethoxy)][2-Nal][2-Me-Leu]E(1c)[{d}R]-[NH2] |
| 90 | [(4-(2-Aminoethyl)-piperazine-1-yl)-acetyl](1c)*SC(2a)WQC(2a)[Y(2- |
| | aminoethoxy)][2-Nal][2-Me-Leu]E(1c)[{d}R]-[NH2] |
| 91 | [(4-(2-Aminoethyl)-piperazine-1-yl)-acetyl](1c)*SC(2a)WQC(2a)[Y(2- |
| | aminoethoxy)][2-Nal][2-Me-Leu]D(1c)[{d}R]-[NH2] |
| 92 | [3-Aminomethyl-phenylacetyl](1c)*SC(2a)WQC(2a)[Y(2-aminoethoxy)][2- |
| | Nal][2-Me-Leu]E(1c)[{d}R]-[NH2] |
| 93 | [2-Aminomethyl-phenylacetyl](1c)*SC(2a)WQC(2a)[Y(2-aminoethoxy)][2- |
| | Nal][2-Me-Leu]E(1c)[{d}R]-[NH2] |
| 94 | [Ac]-[K(N3)](1g)SC(2a)WQC(2a)[Y(2-aminoethoxy)][2-Nal][2-Me- |
| | Leu][Pra](1g)[{d}R]-[NH2] |
| 95 | [Azidoacetic acid](1g)*SC(2a)WQC(2a)[Y(2-aminoethoxy)][2-Nal][2-Me- |
| | Leu][Glu(propargylamine)](1g)[{d}R]-[NH2] |
| 96 | [Azidoacetic acid] (1g)*SC(2a)WQC(2a)[Y(2-aminoethoxy)][2-Nal][2-Me- |
| | Leu][Dab(3-butynoic acid)](1g)[{d}R]-[NH2] |
| 97 | [but-3-ynoic acid] (1g)*SC(2a)WQC(2a)[Y(2-aminoethoxy)][2-Nal][2-Me- |
| | Leu][Dab(azidoacetic acid)](1g)[{d}R]-[NH2] |
| 98 | [(N₃)-Ala](1g)*SC(2a)WQC(2a)[Y(2-aminoethoxy)][2-Nal][2-Me- |
| | Leu][Glu(propargylamine)](1g)[{d}R]-[NH2] |
| 99 | [(N₃)-Ala](1g)*SC(2a)WQC(2a)[Y(2-aminoethoxy)][2-Nal][2-Me- |
| | Leu][Dab(3-butynoic acid)](1g)[{d}R]-[NH2] |
| 100 | [but-3-ynoic acid](1g)*SC(2a)WQC(2a)[Y(2-aminoethoxy)][2-Nal][2-Me- |
| | Leu][Dab((N₃)-Ala))](1g)[{d}R]-[NH2] |
| 101 | [4-Aminomethyl-2-pyridineacetyl](1c)*SC(2a)WQC(2a)[Y(2- |
| | aminoethoxy)][2-Nal][2-Me-Leu]E(1c)[{d}R]-[NH2] |
| 102 | [4-Aminomethyl-3-pyridineacetyl](1c)*SC(2a)WQC(2a)[Y(2- |
| | aminoethoxy)][2-Nal][2-Me-Leu]E(1c)[{d}R]-[NH2] |
| 103 | [4-Aminomethyl-2-fluoro-phenylacetyl](1c)*SC(2a)WQC(2a)[Y(2- |
| | aminoethoxy)][2-Nal][2-Me-Leu]E(1c)[{d}R]-[NH2] |
| 104 | [4-Aminomethyl-3-methyl-phenylacetyl](1c)*SC(2a)WQC(2a)[Y(2- |
| | aminoethoxy)][2-Nal][2-Me-Leu]E(1c)[{d}R]-[NH2] |
| 105 | [4-Aminomethyl-3-methoxy-phenylacetyl](1c)*SC(2a)WQC(2a)[Y(2- |
| | aminoethoxy)][2-Nal][2-Me-Leu]E(1c)[{d}R]-[NH2] |
| 106 | [but-3-ynoic acid](1h)*SC(2a)WQC(2a)[Y(2-aminoethoxy)][2-Nal][2-Me- |
| | Leu][Dab(azidoacetic acid)](1h)[{d}R]-[NH2] |
| 107 | [but-3-ynoic acid](1h)*SC(2a)WQC(2a)[Y(2-aminoethoxy)][2-Nal][2-Me- |
| | Leu][Dab((N₃)-Ala))](1h)[{d}R]-[NH2] |
| 108 | [4-Aminomethyl-phenylacetyl](1c)*SC(2a)WQC(2a)[Y(2-aminoethoxy)][2- |
| | Nal][2-Me-Leu]E(1c)[{d}R]-[OH] |
| 109 | [4-Aminomethyl-phenylacetyl](1c)*SC(2a)WQC(2a)[Y(2-aminoethoxy)][2- |
| | Nal][2-Me-Leu]E(1c)[{d}2,4-Diaminobutanoyl([2-(trimethyl-2- |
| | aminoethoxy)ethoxy]propyl)]-[NH2] |
| 110 | [4-Aminomethyl-phenylacetyl](1c)*SC(2a)WQC(2a)[Dab][2-Nal][2-Me- |
| | Leu]E(1c)[{d}R]-[NH2] |
| 111 | [4-Aminomethyl-phenylacetyl](1c)*SC(2a)WQC(2a)[2,4- |
| | Diaminobutanoyl([2-(trimethyl-2-aminoethoxy)ethoxy]propyl)][2-Nal][2-Me- |
| | Leu]E(1c)[{d}R]-[NH2] |
| 112 | [Dab](1c)*SC(2a)WQC(2a)[Y(2-aminoethoxy)][2-Nal][2-Me- |
| | Leu]E(1c)[{d}R]-[NH2] |
| 113 | [2,4-Diaminobutanoyl([2-(trimethyl-2- |
| | aminoethoxy)ethoxy]propyl)](1c)*SC(2a)WQC(2a)[Y(2-aminoethoxy)][2- |
| | Nal][2-Me-Leu]E(1c)[{d}R]-[NH2] |
| 114 | [4-Aminomethyl-phenylacetyl](1c)*SC(2a)WQC(2a)[F(4-NH2-(2-(trimethyl- |
| | 2-aminoethoxy)ethoxy)propyl)][2-Nal][2-Me-Leu]E(1c)[{d}R]-[NH2] |
| 115 | [4-Aminomethyl-phenylacetyl](1c)*SC(2a)WQC(2a)[Y(2-aminoethoxy)][2- |
| | Nal][2-Me-Leu]E(1c)[{d}Q]-[NH2] |
| 116 | [4-Aminomethyl-phenylacetyl](1c)*SC(2a)WQC(2a)[Y(2-aminoethoxy)][2- |
| | Nal][2-Me-Leu]E(1c)[{d}E]-[NH2] |
| 117 | [4-Aminomethyl-phenylacetyl](1c)*SC(2a)WQC(2a)[Y(2-aminoethoxy)][2- |
| | Nal][2-Me-Leu]E(1c)[{d}H]-[NH2] |
| 118 | [4-Aminomethyl-phenylacetyl](1c)*SC(2a)WQC(2a)[Y(2-aminoethoxy)][2- |
| | Nal][2-Me-Leu]E(1c)[3-Aminopropanoy]]-[OH] |
| 119 | [4-Aminomethyl-phenylacetyl](1c)*SC(2a)WQC(2a)[Y(2-aminoethoxy)][2- |
| | Nal][2-Me-Leu]E(1c)[3-Aminopropanoyl]-[NH2] |
| 120 | [4-Aminomethyl-phenylacetyl](1c)*SC(2a)WQC(2a)[Y(2-aminoethoxy)][2- |
| | Nal][2-Me-Leu]E(1c)[Dab][3-(3-Pyridyl)-Ala]-[NH2] |
| 121 | [4-Aminomethyl-phenylacetyl](1c)*SC(2a)WQC(2a)[Y(2-aminoethoxy)][6- |
| | F-Trp][2-Me-Leu]E(1c)[Dab][3-(3-Pyridyl)-Ala]-[NH2] |
| 122 | [4-Aminomethyl-phenylacetyl](1c)*SE(2c)WQ[Dab](2c)[Y (2- |
| | aminoethoxy)][2-Nal][2-Me-Leu]E(1c)[Dab][3-(3-Pyridyl)-Ala]-[NH2] |
| 123 | [4-Aminomethyl-phenylacetyl](1c)*S[Dab](2c)WQE(2c)[Y(2- |
| | aminoethoxy)][2-Nal][2-Me-Leu]E(1c)[Dab][3-(3-Pyridyl)-Ala]-[NH2] |
| 124 | [4-Aminomethyl-phenylacetyl](1c)*SE(2c)WQ[Orn](2c)[Y (2- |
| | aminoethoxy)][2-Nal][2-Me-Leu]E(1c)[Dab][3-(3-Pyridyl)-Ala]-[NH2] |
| 125 | [4-Aminomethyl-phenylacetyl](1c)*SE(2c)WQ[Dab](2c)[Y(2- |
| | aminoethoxy)][2-Nal][2-Me-Leu]E(1c)[Dab]-[NH2] |
| 126 | [4-Aminomethyl-phenylacetyl](1c)*VE(2c)WQ[Dab](2c)[Y(2- |
| | aminoethoxy][2-Nal][2-Me-Leu]E(1c)[Dab]-[NH2] |
| 127 | [4-Aminomethyl-phenylacetyl](1c)*IE(2c)WQ[Dab](2c)[Y(2- |
| | aminoethoxy)][2-Nal][2-Me-Leu]E(1c)[Dab]-[NH2] |
| 128 | [4-Aminomethyl-phenylacetyl](1c)*SE(2c)WV[Dab](2c)[Y(2- |
| | aminoethoxy)][2-Nal][2-Me-Leu]E(1c)[Dab]-[NH2] |
| 129 | [4-Aminomethyl-phenylacetyl](1c)*SE(2c)WH[Dab](2c)[Y(2- |
| | aminoethoxy)][2-Nal][2-Me-Leu]E(1c)[Dab]-[NH2] |
| 130 | [4-Aminomethyl-phenylacetyl](1c)*SE(2c)WQ[Dab](2c)F[2-Nal][2-Me- |
| | Leu]E(1c)[Dab]-[NH2] |
| 131 | [4-Aminomethyl-phenylacetyl](1c)*SE(2c)WQ[Dab](2c)V[2-Nal][2-Me- |
| | Leu]E(1c)[Dab]-[NH2] |
| 132 | [4-Aminomethyl-phenylacetyl](1c)*SE(2c)WQ[Dab](2c)[F(4-Me)][2-Nal][2- |
| | Me-Leu]E(1c)[Dab]-[NH2] |
| 133 | [4-Aminomethyl-phenylacetyl](1c)*SE(2c)WQ[Dab](2c)[Y(2- |
| | aminoethoxy)]H[2-Me-Leu]E(1c)[Dab]-[NH2] |
| 134 | [4-Aminomethyl-phenylacetyl](1c)*SE(2c)WQ[Dab](2c)[Y(2- |
| | aminoethoxy)][F(3-F)][2-Me-Leu]E(1c)[Dab]-[NH2] |
| 135 | [4-Aminomethyl-phenylacetyl](1c)*SE(2c)WQ[Dab](2c)[Y(2- |
| | aminoethoxy)][F(3,4-Me)][2-Me-Leu]E(1c)[Dab]-[NH2] |
| 136 | [4-Aminomethyl-phenylacetyl](1c)*SE(2c)WQ[Dab](2c)[Y(2- |
| | aminoethoxy)][2-Nal][2-Me-Val]E(1c)[Dab]-[NH2] |
| 137 | [4-Aminomethyl-phenylacetyl](1c)*SE(2c)WQ[Dab](2c)[Y(2- |
| | aminoethoxy)][2-Nal][2-Me-Leu]E(1c)-[NH-3-aminopropanoyl] |
| 138 | [4-Aminomethyl-phenylacetyl](1c)*SE(2c)[4-F-Trp]Q[Dab](2c)[Y(2- |
| | aminoethoxy)][2-Nal][2-Me-Leu]E(1c)[Dab]-[NH2] |
| 139 | [4-Aminomethyl-phenylacetyl](1c)*SE(2c)[7-F-Trp]Q[Dab](2c)[Y(2- |
| | aminoethoxy)][2-Nal][2-Me-Leu]E(1c)[Dab]-[NH2] |
| 140 | [4-Methylaminomethyl-phenylacetyl](1c)*SC(2a)WQC(2a)[Y(2- |
| | aminoethoxy)][2-Nal][2-Me-Leu]E(1c)[Dab]-[NH2] |
| 141 | [4-Aminomethyl-phenylacetyl](1c)*[N-Me-Ser]C(2a)WQC(2a)[Y(2- |
| | aminoethoxy)][2-Nal][2-Me-Leu]E(1c)[Dab]-[NH2] |
| 142 | [4-Aminomethyl-phenylacetyl](1c)*S[N-Me-Cys](2a)WQC(2a)[Y (2- |
| | aminoethoxy)][2-Nal][2-Me-Leu]E(1c)[Dab]-[NH2] |
| 143 | [4-Aminomethyl-phenylacetyl](1c)*SC(2a)[N-Me-Trp]QC(2a)[Y(2- |
| | aminoethoxy)][2-Nal][2-Me-Leu]E(1c)[Dab]-[NH2] |
| 144 | [4-Aminomethyl-phenylacetyl](1c)*SC(2a)W[N-Me-Gln]C(2a)[Y(2- |
| | aminoethoxy)][2-Nal][2-Me-Leu]E(1c)[Dab]-[NH2] |
| 145 | [4-Aminomethyl-phenylacetyl](1c)*[Q(pyrrolidin)]E(2c)WQ[Dab](2c)[Y(2- |
| | aminoethoxy)][2-Nal][2-Me-Leu]E(1c)[Dab]-[NH2] |
| 146 | [4-Aminomethyl-phenylacetyl](1c)*SE(2c)W[Q(pyrrolidin)][Dab](2c)[Y(2- |
| | aminoethoxy)][2-Nal][2-Me-Leu]E(1c)[Dab]-[NH2] |
| 147 | [4-Aminomethyl-phenylacetyl](1c)*SC(2a)WQC(2a)[Y(2-aminoethoxy)][2- |
| | Nal][2-Me-Leu]E(1c)R[3-(3-Pyridyl)-Ala]-[NH2] |
| 148 | [(3-Aminomethyl)benzoyl](1c)*SC(2a)WQC(2a)[Y(2-aminoethoxy)][2- |
| | Nal][2-Me-Leu]E(1c)[Dab][3-(3-Pyridyl)-Ala]-[NH2] |
| 149 | [(3-Aminomethyl)benzoyl](1c)*SC(2a)WQC(2a)][Y(2-aminoethoxy)][2- |
| | Nal][2-Me-Leu]E(1c)R[3-(3-Quinolinyl)-Ala]-[NH2] |
| 150 | [(3-Aminomethyl)benzoyl](1c)*SC(2a)WQC(2a)][Y(2-aminoethoxy)][2- |
| | Nal][2-Me-Leu]E(1c)R[{d}[3-(3-Pyridyl)-Ala]]-[NH2] |
| 151 | [(3-Aminomethyl)benzoyl](1c)*SC(2a)WQC(2a)][Y(2-aminoethoxy)][2- |
| | Nal][2-Me-Leu]E(1c)R[3-Amino-3-(3′-pyridyl)propionyl]-[NH2] |
| 152 | [(3-Aminomethyl)benzoyl](1c)*SC(2a)WQC(2a)][Y(2-aminoethoxy)][2- |
| | Nal][2-Me-Leu]E(1c)R[F(3-F)]-[NH2] |
| 153 | [(3-Aminomethyl)benzoyl](1c)*SC(2a)WQC(2a)][Y(2-aminoethoxy)][2- |
| | Nal][2-Me-Leu]E(1c)R[F(3,5-F)]-[NH2] |
| 154 | [(3-Aminomethyl)benzoyl](1c)*SC(2a)WQC(2a)][Y(2-aminoethoxy)][2- |
| | Nal][2-Me-Leu]E(1c)R[4-Aminomethyl-2-pyridineacetyl]-[NH2] |
| 155 | [Ac]-K(1c)TC(2a)WQC(2a)[Y(2-aminoethoxy)][2-Nal][2-Me- |
| | Leu]E(1c)[Dab][3-(3-Pyridyl)-Ala]-[NH2] |
| 156 | [(3-Aminomethyl)benzoyl](1c)*IE(2c)WQ[Dab](2c)[Y(2-aminoethoxy)][2- |
| | Nal][2-Me-Val]E(1c)[Dab][3-(3-Pyridyl)-Ala]-[NH2] |
| 157 | [4-Aminomethyl-phenylacetyl](1c)*IE(2c)WQ[Dab](2c)[Y(2- |
| | aminoethoxy)][2-Nal][2-Me-Val]E(1c)[Dab][3-(3-Pyridyl)-Ala]-[NH2] |
| 158 | [4-Aminomethyl-phenylacetyl](1c)*IE(2c)WQ[Dab](2c)[Y(2- |
| | aminoethoxy)][F(3,4-Me)][2-Me-Val]E(1c)G[3-(3-Pyridyl)-Ala]-[NH2] |
| 159 | [4-Aminomethyl-phenylacetyl](1c)*IE(2c)WQ[Dab](2c)[F(4-Me)][F(3,4- |
| | Me)][2-Me-Val]E(1c)G[3-(3-Pyridyl)-Ala]-[NH2] |
| 160 | [4-Aminomethyl-phenylacetyl](1c)*IE(2c)WQ[Dab](2c)[F(4-Me)][F(3,4- |
| | Me)][2-Me-Val]E(1c)[GABA][3-(3-Pyridyl)-Ala]-[NH2] |
| 161 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-pyridylacetyl)]-[NH2] |
| 162 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-pyridylpropionyl)]-[NH2] |
| 163 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-fluorobenzoyl)]-[NH2] |
| 164 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-fluorophenylacetyl)]-[NH2] |
| 165 | [4-Aminomethyl-phenylacetyl](1c)*IE(2c)WQ[Dab](2c)[Y(2- |
| | aminoethoxy)][F(3,4-Me)][2-Me-Val]E(1c)[Dab][2-Me-3-(3-Pyridyl)-Ala]- |
| | [NH2] |
| 166 | [4-Aminomethyl-phenylacetyl](1c)*IE(2c)WQ[Dab](2c)[Y (Me)][F(3,4- |
| | Me)][2-Me-Val]E(1c)[Dab][2-Me-3-(3-Pyridyl)-Ala]-[NH2] |
| 167 | [4-Aminomethyl-phenylacetyl](1c)*IE(2c)WQ[Dab](2c)[2-Me-Phe][F(3,4- |
| | Me)][2-Me-Val]E(1c)[Dab][2-Me-3-(3-Pyridyl)-Ala]-[NH2] |
| 168 | [4-Aminomethyl-phenylacetyl](1c)*IE(2c)WQ[Dab](2c)[Bip][F(3,4-Me)][2- |
| | Me-Val]E(1c)[Dab][2-Me-3-(3-Pyridyl)-Ala]-[NH2] |
| 169 | [4-Aminomethyl-phenylacetyl](1c)*IE(2c)WQ[Dab](2c)[2-Me-F(4-F)][F(3,4- |
| | Me)][2-Me-Val]E(1c)[Dab][2-Me-3-(3-Pyridyl)-Ala]-[NH2] |
| 170 | [4-Aminomethyl-phenylacetyl](1c)*IE(2c)WQ[Dab](2c)[{d}F(4-F)][F(3,4- |
| | Me)][2-Me-Val]E(1c)[Dab][2-Me-3-(3-Pyridyl)-Ala]-[NH2] |
| 171 | [4-Aminomethyl-phenylacetyl](1c)*IE(2c)WQ[Dab](2c)[F(4-CI)][F(3,4- |
| | Me)][2-Me-Val]E(1c)[Dab][2-Me-3-(3-Pyridyl)-Ala]-[NH2] |
| 172 | [4-Aminomethyl-phenylacetyl](1c)*IE(2c)WQ[Dab](2c)[alpha-Me- |
| | Trp][F(3,4-Me)][2-Me-Val]E(1c)[Dab][2-Me-3-(3-Pyridyl)-Ala]-[NH2] |
| 173 | [4-Aminomethyl-phenylacetyl](1c)*IE(2c)WQ[Dab](2c)[3,3-Diphenyl- |
| | Ala][F(3,4-Me)][2-Me-Val]E(1c)[Dab][2-Me-3-(3-Pyridyl)-Ala]-[NH2] |
| 174 | [4-Aminomethyl-phenylacetyl](1c)*IE(2c)WQ[Dab](2c)[Phg][F(3,4-Me)][2- |
| | Me-Val]E(1c)[Dab][2-Me-3-(3-Pyridyl)-Ala]-[NH2] |
| 175 | [4-Aminomethyl-phenylacetyl](1c)*IE(2c)WQ[Dab](2c)[F(4-Me)][1-Me- |
| | Trp][2-Me-Val]E(1c)[Dab][2-Me-3-(3-Pyridyl)-Ala]-[NH2] |
| 176 | [4-Aminomethyl-phenylacetyl](1c)*IE(2c)WQ[Dab](2c)[F(4-Me)][Bip][2-Me- |
| | Val]E(1c)[Dab][2-Me-3-(3-Pyridyl)-Ala]-[NH2] |
| 177 | [4-Aminomethyl-phenylacetyl](1c)*IE(2c)WQ[Dab](2c)[F(4-Me)][{d}6-F- |
| | Trp][2-Me-Val]E(1c)[Dab][2-Me-3-(3-Pyridyl)-Ala]-[NH2] |

The bridging amino acid residue is the amino acid residue directly preceding the parentheses below. The square brackets indicate the bridging amino acid residues. For example, [2, 11] is a bridge between amino acid residues 2 and 11. Similarly, [4, 7] is a bridge between amino acid residues 4 and 7.
*= bridge uses the peptide backbone amine or carboxylic acid at the N- or C-terminus, not the side chain amine or carboxylic acid
(1a) = [2, 11] 1,3-dithio-propan-2-one bridge; (1c) = [2, 11] lactam bridge; (1g) = [2, 11] 1,4-disubstituted 1,2,3-triazole bridge; (1h) = [2, 11] 1,5-disubstituted 1,2,3-triazole bridge; (2a) = [4, 7] 1,3-dithio-propan-2-one bridge; (2c) = [4, 7] lactam bridge; (2d) = [4, 7] 1,2-phenylenedimethanethiol bridge; (2e) = [4,7] 1,3-phenylenedimethanethiol bridge; (2f) = [4, 7] 1,4-phenylenedimethanethiol bridge; (2g) = [4, 7] 1,4-disubstituted 1,2,3-triazole bridge.

The bridging amino acid residue is the amino acid residue directly preceding the parentheses below. The square brackets indicate the bridging amino acid residues. For example, [2,11] is a bridge between amino acid residues 2 and I1. Similarly, [4,7] is a bridge between amino acid residues 4 and 7.

*=bridge uses the peptide backbone amine or carboxylic acid at the N- or C-terminus, not the side chain amine or carboxylic acid

(1a)=[2,11]1,3-dithio-propan-2-one bridge; (1c)=[2,11] lactam bridge; (1g)=[2,11]1,4-disubstituted 1,2,3-triazole bridge; (1h)=[2,11]1,5-disubstituted 1,2,3-triazole bridge; (2a)=[4,7]1,3-dithio-propan-2-one bridge; (2c)=[4,7] lactam bridge; (2d)=[4,7]1,2-phenylenedimethanethiol bridge; (2e)=[4,7]1,3-phenylenedimethanethiol bridge; (2f)=[4,7]1,4-phenylenedimethanethiol bridge; (2g)=[4,7]1,4-disubstituted 1,2,3-triazole bridge.

For comparison purposes, three compounds from Heinis et al., 2020 having two 1,3-dithio-propan-2-one bridges were synthesised (Table 1-2).

TABLE 1-2
Synthesised compounds from Heinis et al., 2020

| Compound | Structure |
|---|---|
| I1 (isomer 3) | H-GC(1a)TC(2a)WEC(2a)WWLC(1a)S-[NH2] |
| I3 (isomer 3) | H-DC(1a)SC(2a)WQC(2a)WWLC(1a)R-[NH2] |
| I4 (isomer 3) | H-EC(4a)WLC(5a)YYRNC(5a)NC(4a)M-[NH2] |
| I5 (isomer 3) | H-GC(1a)TC(2a)WEC(2a)WW[2-Me-Leu]C(1a)S-[NH2] |

(1a) = [2,11] 1,3-dithio-propan-2-one bridge; (2a) = [4,7] 1,3-dithio-propan-2-one bridge;
(4a) = [1,11] 1,3-dithio-propan-2-one bridge; (5a) = [4,9] 1,3-dithio-propan-2-one bridge.

Additionally, a compound from the Protagonist patent applications (Compound C in WOK 20116/011208, WOK 2017/011820, and Sayago et al., 2018) having a bridging cystathionine amino acid residue at [2,7] was synthesised (Table 1-3).

TABLE 1-3
Synthesised compound from the Protagonist patent applications

| Compound | Structure |
|---|---|
| Compound C | [Ac]-[Abu](16i)QTWQC(16i)[Y(2-aminoethoxy)]W[2-Me-Lys]ENG-[NH2] |

(16i) = [2,7] thioether bridge. The combination of Abu at position X2, Cys at position X7, and the [2,7] thioether bridge forms a cystathionine as the bridging amino acid residue.

Other reference compounds synthesised are described in Table 1-4.

TABLE 1-4
Synthesised reference compounds

| Compound | Structure |
|---|---|
| Ref 1 | H-DC(1b)SC(2b)WQC(2b)WWLC(1b)R-[NH2] |
| Ref 2 | H-DE(1c)SSWQSWWLK(1c)R-[NH2] |
| Ref 3 | H-DE(1c)STWQYWWLK(1c)R-[NH2] |
| Ref 4 | [Ac]-DE(1c)SMWQMWWLK(1c)R-[NH2] |
| Ref 5 | [Ac]-DE(1c)C(2a)WQC(2a)WWLK(1c)R-[NH2] |
| Ref 6 | [Ac]-DE(1c)SC(2a)WC(2a)WWLK(1c)R-[NH2] |
| Ref 7 | [Ac]-DE(1c)SC(2a)WQC(2a)WWK(1c)R-[NH2] |

(1b) = [2,11] disulfide bridge; (1c) = [2,11] lactam bridge; (2a) = [4,7] 1,3-dithio-propan-2-one bridge; (2b) = [2,11] disulfide bridge.

Unless otherwise specified, reagents and solvents employed in the following were available commercially in standard laboratory reagent or analytical grade, and were used without further purification.

### Apparatus and Synthetic Strategy

Peptides were synthesized batchwise on a peptide synthesiser, such as a CEM Liberty Blue Peptide Synthesizer, according to solid phase peptide synthetic procedures using 9-fluorenylmethyloxycarbonyl (Fmoc) as N-α-amino protecting group and suitable common protection groups for side-chain functionalities.

As polymeric support based resins, such as e.g. TentaGel™, was used. The synthesizer was loaded with resin that prior to usage was swelled in DMF.

Non-naturally occurring amino acids and other suitable building blocks were employed without any changes to the general procedure.

The following optical isomers of particular amino acids (including non-naturally occurring amino acids) were employed in the synthesis of the compounds:

| | |
|---|---|
| 2-Nal: | 3-(2-naphthyl)-L-alanine |
| {d}R or D-Arg | D-arginine |
| {d}L or D-Leu | D-leucine |
| {d}C or D-Cys | D-cysteine |
| {d}Q or D-Gln | D-glutamine |
| {d}E or D-Glu | D-glutamic acid |
| {d}H or D-His | D-histidine |
| alpha-Me-Trp | α-methyl-L-tryptophan |
| 1-Me-Trp | 1-methyl-L-tryptophan |
| 4-F-Trp | 4-fluoro-L-tryptophan |
| 6-F-Trp | 6-fluoro-L-tryptophan |
| {d}6-F-Trp | 6-fluoro-D-tryptophan |
| 6-Cl-Trp | 6-chloro-L-tryptophan |
| 7-Me-Trp | 7-methyl-L-tryptophan |
| 7-F-Trp | 7-fluoro-L-tryptophan |
| 7-Ph-Trp | 7-phenyl-L-tryptophan |
| 7-(Naphth-2-yl)-Trp | 7-(naphth-2-yl)-L-tryptophan |
| 3-(3-Pyridyl)-Ala | 3-(3-pyridyl)-L-alanine |
| 3-(4-Pyridyl)-Ala | 3-(4-pyridyl)-L-alanine |
| {d}[3-(3-Pyridyl)-Ala] | 3-(3-pyridyl)-D-alanine |
| 2-Me-3-(3-Pyridyl)-Ala | (2S)-2-amino-2-methyl-3-(3-pyridyl)propanoic |
| 3-(3-Quinolinyl)-Ala | 3-(3-quinolinyl)-L-alanine; also |
| | (2S)-2-amino-3-(3-quinolyl)propanoic |
| F(3-F) or 3-F-Phe | 3-fluoro-L-phenylalanine |
| F(4-F) or 4-F-Phe | 4-fluoro-L-phenylalanine |
| {d}F(4-F) | 4-fluoro-D-phenylalanine |
| F(4-Cl) or 4-Cl-Phe | 4-chloro-L-phenylalanine |
| F(4-NH2) or 4-NH₂-Phe | 4-amino-L-phenylalanine |
| F(4-Me) or 4-Me-Phe | 4-methyl-L-phenylalanine |
| F(3,4-Me) or 3,4-Me-Phe | 3,4-dimethyl-L-phenylalanine |
| F(3,5-F) or 3,5-F-Phe | 3,5-difluoro-L-phenylalanine |
| 2-Me-F(4-F) | (2S)-2-amino-3-(4-fluorophenyl)-2-methyl-propanoic acid |
| 2-Me-Leu | 2-methyl-L-leucine |
| 2-Me-Lys | 2-methyl-L-lysine |
| 2-Me-Arg | 2-methyl-L-arginine |
| 2-Me-Val | 2-methyl-L-valine |
| 2-Me-Phe | alpha-methyl-L-phenylalanine; also known as |
| | (2S)-2-amino-2-methyl-3-phenyl-propanoic acid |
| N-Me-Arg | N2-methyl-L-arginine |
| N-Me-Ser | N-methyl-L-serine |
| N-Me-Cys | N-methyl-L-cysteine |
| N-Me-Trp | Na-methyl-L-tryptophan; also known as L-abrine |
| N-Me-Gln | N2-methyl-L-glutamine |
| Bip | biphenyl-L-alanine |
| 3,3-Diphenyl-Ala | 3,3-diphenyl-L-alanine; also known as |
| | β-phenyl-L-phenylalanine, or |
| | (S)-2-amino-3,3-diphenylpropionic acid |
| Aad | 2-aminoadipic acid, also known as (2S)-2-aminoadipic acid, |
| | (2S)-2-aminohexanedioic acid, or L-homoglutamic acid |
| Dab | (2S)-2,4-diaminobutanoic acid |
| Orn | L-ornithine, also known as 2,5-diaminopentanoic acid |
| hLys | (2S)-2-amino-7-amino-heptanoic acid, also known as |
| | L-homolysine |
| bAla | 3-aminopropionic acid, also known as beta-alanine or |
| | β-alanine |
| beta-homo-Ser | L-β-homoserine |
| Ala(N₃) | 3-azido-L-alanine |
| Aha | azidohomo-L-alanine or 4-azido-L-homoalanine |
| Orn(N₃) | azido-L-ornithine |
| K(N₃) or Lys(N₃) | azido-L-lysine |
| Pra | L-propargylglycine |
| Hpg | L-homopropargylglycine |
| Bpg | L-bishomopropargylglycine |
| Dpr | (2S)-2,3-diaminopropanoic acid, also known as |
| | 3-amino-L-alanine |
| Glu(propargylamine) | (2S)-2-amino-5-oxo-5-(prop-2-ynylamino)pentanoic acid |
| Dab(3-butynoic acid) | (2S)-2-amino-4-(but-3-ynoylamino)butanoic acid |
| Dab(azidoacetic acid) | (2S)-2-amino-4-[(2-azidoacetyl)amino]butanoic acid |
| (N₃)-Ala | (2S)-2-azidopropanoic acid |
| Dab((N₃)-Ala)) | (2S)-2-amino-4-[(2S)-2-azidopropanoyl]amino]butanoic acid |
| Y(2-aminoethoxy) | (2S)-2-amino-3-[4-(2-aminoethoxy)phenyl]propanoic acid |
| Y(Me) | (2S)-2-amino-3-(4-methoxyphenyl)propanoic acid |
| Q(pyrrolidin) | (2S)-2-amino-5-oxo-5-pyrrolidin-1-yl-pentanoic acid |
| GABA | gamma aminobutyric acid; also known as |
| | 4-aminobutanoic acid |
| Phg | L-2-phenylglycine |

F(4-NH₂-(2-(trimethyl-2-aminoethoxy)ethoxy)propyl) 2-[2-[3-[4-[(2S)-2-amino-2-carboxy-ethyl]anilino]-3-oxo-propoxy]ethoxy]ethyl-trimethyl-ammonium

### Coupling on a CEM Liberty Blue Peptide Synthesizer

A solution of Fmoc-protected amino acid (4 equiv.) was added to the resin together with a coupling reagent solution (4 equiv.) and a solution of base (8 equiv.). The mixture was either heated by the microwave unit to 50° C. and coupled for 10 minutes or coupled with no heat for 60 minutes. During the coupling nitrogen was bubbled through the mixture.

In the case of difficult couplings (e.g. coupling of a residue immediately after an N-methylated amino acid residue or other sterically hindered amino acid residue as recognized by a person of skill in the art) the coupling was repeated one or more times.

### Deprotection:

The Fmoc group was deprotected using piperidine in DMF or other suitable solvents. The deprotection solution was added to the reaction vessel and the mixture was heated for 5 minutes reaching approx. 50° C. After draining the reaction vessel the resin was washed with DMF or other suitable solvents.

### Lactam Formation:

The following procedure for the coupling of Glu and Lys is representative for all lactam formations where the amino acid side chain containing the carboxyl-function is protected with Oall and the amino acid side chain containing the amino group is Alloc-protected. After assembly of the full peptide sequence, deprotection of Glu(Oall) and Lys(Alloc) was performed using Pd(PPh₃)₄(0.05 equiv.) and PhSiH₃ (10 equiv.) in DCM. Subsequently, the lactam bridge was formed between the side-chain carboxylic acid of Glu and side-chain amine of Lys using PyBOP (2 equiv.) and DIPEA (3.0 equiv.) in DMF. Both steps were performed with the peptide still attached to the resin.

Peptides with a lactam from a side-chain to the N-terminal amine were prepared similarly. After assembly of the full peptide sequence, the Fmoc protection group of the N-terminal amine is left intact. Glu(Oall) is deprotected as described with Pd(PPh₃)₄ followed by a Fmoc deprotection (see section “cleavage”). The lactam bridge was formed similarly with PyBOP.

### Cleavage:

The dried peptide resin was treated with TFA and suitable scavengers for approximately 2 hours. The volume of the filtrate was reduced and the crude peptide was precipitated after addition of diethylether. The crude peptide precipitate was washed several times with diethylether and finally dried.

### HPLC Purification of the Crude Peptide:

The crude peptide was purified by preparative reverse phase HPLC using a conventional HPLC apparatus, such as a Gilson GX-281 with 331/332 pump combination, for binary gradient application equipped with a column, such as 5×25 cm Gemini NX 5u C18 110A column, and a fraction collector using a flow 20-40 ml/min with a suitable gradient of buffer A (0.1% Fomic acid, aq.) or A (0.1% TFA, aq.) and buffer B (0.1% Formic acid, 90% MeCN, aq.) or B (0.1% TFA, 90% MeCN, aq.). Fractions were analyzed by analytical HPLC and MS and selected fractions were pooled and lyophilized. The final product was characterized by HPLC and MS.

### Dithioether Formation:

The methodology for dithioether formation from the reaction between two unprotected cysteines and a linker can be performed as previously described in Heinis et al., 2020, or through the modified method below.

The crude intermediate peptide was dissolved in 3:7 mixture of water/acetonitrile (1 mg/mL). DODT (2 equiv.) was added and the mixture was stirred at room temperature for 10 min. The pH was adjusted to pH 8 by addition of 0.2 M ammonium carbonate. The linker (2 equiv.) was added directly to the solution and the mixture was left shaken at room temperature over night. The solution was filtered with 0.45 μm filter and loaded directly on a prep-HPLC column for a final purification.

Linkers used in the synthesized compounds include:
1,3-dibromopropan-2-one, CAS #816-39-7;
1,2-Bis(bromomethyl)benzene, CAS #91-13-4;
1,3-Bis(bromomethyl)benzene, CAS #626-15-3; and
1,4-Bis(bromomethyl)benzene, CAS #623-24-5.

### Triazole Formation:

The crude intermediate peptide was dissolved in H₂O/^{t}BuOH 2:1 (1 mg/mL). 4.4 equivalents of CuSO₄ 5H₂O and 4.4 equivalents of L-Ascorbic acid (CAS 50-81-7) were added into the mixture. The mixture was protected from light and left stirring for 24 hours.

The solution was loaded directly on a prep-HPLC column for a final purification.

### Analytical HPLC:

Final purities were determined by analytic HPLC (Agilent 1100/1200 series) equipped with auto sampler, degasser, 20 μl flow cell and Chromeleon software. The HPLC was operated with a flow of 1.2 ml/min at 40° C. using an analytical column, such as Kinetex 2.6 μm XB-C18 100A 100×4.6 mm column. The compound was detected and quantified at 215 nm. Buffers A (0.1% TFA, aq.) and buffer B (0.1% TFA, 90% MeCN, aq.).

### Mass Spectroscopy:

Final MS analysis was performed on a conventional mass spectrometer, e.g. Waters Xevo G2 Tof, equipped with electrospray detector with lock-mass calibration and MassLynx software. It was operated in positive mode using direct injection and a cone voltage of 15V (1 TOF), 30 V (2 TOF) or 45 V (3 TOF) as specified on the chromatogram. Precision was ppm with a typical resolution of 15,000-20,000.

One of skill in the art will appreciate that standard methods of peptide synthesis may be used to generate the compounds of the invention.

### Example 2: Structure-Activity-Relationship (SAR) of the Compounds

The inventors synthesised peptides I1 (isomer 3), I3 (isomer 3), I4 (isomer 3), and I5 (isomer 3) from Heinis et al., 2020, which were identified through phage display.

Highlighted in the article as hits were peptides I3 and I4 (not protease pressured i.e. low GI stability) and I1 (protease pressured i.e. better GI stability). I1 was further modified with one amino acid, Leu at position 10 to 2-methyl-Leu, to give I5 with more GI stability.

TABLE 2-1
Kᵢ, IC₅₀ and SIF data for I1, I3, I4, and I5 peptides from Heinis et al., 2020

| | Ki IL-23R (nM) | IC₅₀ (pSTAT3) (nM) | SIF (% remaining 4 h) |
|---|---|---|---|
| I1 (isomer 3) | 104 | Tested | 0 |
| I3 (isomer 3) | 38.0 | 407 | 0 |
| I4 (isomer 3) | Tested | Tested | 0 |
| I5 (isomer 3) | 73.5 | 934 | 22 |

Tested = Kᵢ or IC₅₀ estimated higher than 1 μM when tested in assays

In the following, the inventors define [2,11] as a bridge between amino acid residues 2 and 11. Similarly, for [4,7] a bridge between amino acid residues 4 and 7.

The inventors noticed that it was indeed essential for the peptide to have a linker in between the cysteine pairs as the I3 peptide (isomer 3) without the linker and forming two pairs of disulfide bridges (Ref 1) was inactive (Table 2-2b).

TABLE 2-2a
Amino acid sequence of I3 (isomer 3) and Ref 1

| | Amino Acid | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| I3 | D | C | S | C | W | Q | C | W | W | L | C | R |
| (isomer 3) | | (1a) | | (2a) | | | (2a) | | | | (1a) | |
| Ref 1 | D | C | S | C | W | Q | C | W | W | L | C | R |
| | | (1b) | | (2b) | | | (2b) | | | | (1b) | |

(1a) = [2,11] 1,3-dithio-propan-2-one bridge; (2a) = [4,7] 1,3-dithio-propan-2-one bridge; (1b) = [2,11] disulfide bridge; (2b) = [4,7] disulfide bridge.

TABLE 2-2b
Kᵢ and IC₅₀ data for I3 (1,3-dithio-propan-2-one
bridges) and Ref 1 (disulfide bridges)

| | Kᵢ IL-23R (nM) | IC₅₀ (pSTAT3) (nM) |
|---|---|---|
| I3 (isomer 3) | 38.0 | 407 |
| Ref 1 | — | Tested |

Tested = IC₅₀ estimated higher than 1 μM when tested in assays

The inventors found that the [2,11]1,3-dithio-propan-2-one bridge could be replaced with a lactam bridge, but that the potency was dependent on the location and bridge size e.g. Compound 4 and Compound 7 (Table 2-3a).

The location of the amide bond in the lactam bridge affected the potency of the peptide as it was more active when the amide bond was closer to position 11 (Compound 7, Table 2-3b) and less active when closer to position 2 (Compound 4, Table 2-3b).

TABLE 2-3a
Amino acid sequence of I3 (isomer 3), Compound 4, and Compound 7

| | Amino Acid | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| I3 | D | C | S | C | W | Q | C | W | W | L | C | R |
| (isomer 3) | | (1a) | | (2a) | | | (2a) | | | | (1a) | |
| Compound 4 | D | E | S | C | W | Q | C | W | W | L | K | R |
| | | (1c) | | (2a) | | | (2a) | | | | (1c) | |
| Compound 7 | D | K | S | C | W | Q | C | W | W | L | E | R |
| | | (1c) | | (2a) | | | (2a) | | | | (1c) | |

(1a) = [2,11] 1,3-dithio-propan-2-one bridge; (2a) = [4,7] 1,3-dithio-propan-2-one bridge; (1c) = [2,11] lactam bridge.

TABLE 2-3b
Kᵢ and IC₅₀ data for I3 ([2, 11] 1,3-dithio-propan-2-one bridge),
Compound 4, and Compound 7 ([2, 11] lactam bridge)

| | Kᵢ IL-23R (nM) | IC₅₀ (pSTAT3) (nM) |
|---|---|---|
| I3 (isomer 3) | 38.0 | 407 |
| Compound 4 | 143 | Tested |
| Compound 7 | 26.8 | 159 |

Tested = IC₅₀ estimated higher than 1 μM when tested in assays

As shown in Table 2-3b, Compound 7 is thus superior to I3 (isomer 3) as one of the 1,3-dithio-propan-2-one bridges is replaced with a more stable lactam bridge.

The inventors further observed that they could truncate the N-terminal Asp and retain and/or increase potency (Compound 10, Tables 2-4a and 2-4b).

TABLE 2-4a
Amino acid sequence of Compound 4 and Compound 10

| | Amino Acid | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| Compound 4 | D | E | S | C | W | Q | C | W | W | L | K | R |
| | | (1c) | | (2a) | | | (2a) | | | | (1c) | |
| Compound 10 | — | E | S | C | W | Q | C | W | W | L | K | R |
| | | (1c) | | (2a) | | | (2a) | | | | (1c) | |

(1a) = [2,11] 1,3-dithio-propan-2-one bridge; (2a) = [4,7] 1,3-dithio-propan-2-one bridge; (1c) = [2,11] lactam bridge.

TABLE 2-4b
Kᵢ and IC₅₀ data for Compound 4 (D present at position
1) and Compound 10 (D absent at position 1)

| | Kᵢ IL-23R (nM) | IC₅₀ (pSTAT3) (nM) |
|---|---|---|
| Compound 4 | 143 | Tested |
| Compound 10 | 101 | 617 |

Tested = IC₅₀ estimated higher than 1 μM when tested in assays

The inventors evaluated some Trp analogs and found they could optimize the potency by introducing 7-Me-Trp in position 5 (Compound 16) or 2-Nal in position 9 (Compound 17, Tables 2-5a and 2-5b).

TABLE 2-5a
Amino acid sequence of Compound 4, Compound 16, and Compound 17

| | Amino Acid | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| Compound 4 | D | E | S | C | W | Q | C | W | W | L | K | R |
| | | (1c) | | (2a) | | | (2a) | | | | (1c) | |
| Compound 16 | D | E | S | C | 7-Me-Trp | Q | C | W | W | L | K | R |
| | | (1c) | | (2a) | | | (2a) | | | | (1c) | |
| Compound 17 | D | E | S | C | W | Q | C | W | 2-Nal | L | K | R |
| | | (1c) | | (2a) | | | (2a) | | | | (1c) | |

(1a) = [2,11] 1,3-dithio-propan-2-one bridge; (2a) = [4,7] 1,3-dithio-propan-2-one bridge; (1c) = [2,11] lactam bridge.

TABLE 2-5b
Kᵢ and IC₅₀ data for Compound 4 (W), Compound
16 (7-Me-Trp) and Compound 17 (2-Nal)

| | Kᵢ IL-23R (nM) | IC₅₀ (pSTAT3) (nM) |
|---|---|---|
| Compound 4 | 143 | Tested |
| Compound 16 | 95.4 | 461 |
| Compound 17 | 70.7 | 366 |

Tested = IC₅₀ estimated higher than 1 μM when tested in assays

When combining the different optimization parameters, the inventors observe a synergistic optimization of the potency from I3 (isomer 3) to Compound 18 (Tables 2-6a and 2-6b).

TABLE 2-6a
Amino acid sequence of I3 (isomer 3), Compound 19, and Compound 18

| | Amino Acid | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| I3 | D | C | S | C | W | Q | C | W | W | L | C | R |
| (isomer 3) | | (1a) | | (2a) | | | (2a) | | | | (1a) | |
| Compound 19 | D | K | S | C | W | Q | C | W | 2-Nal | L | E | R |
| | | (1c) | | (2a) | | | (2a) | | | | (1c) | |
| Compound 18 | — | K | S | C | W | Q | C | W | 2-Nal | L | E | R |
| | | (1c) | | (2a) | | | (2a) | | | | (1c) | |

(1a) = [2,11] 1,3-dithio-propan-2-one bridge; (2a) = [4,7] 1,3-dithio-propan-2-one bridge; (1c) = [2,11] lactam bridge.

TABLE 2-6b
Kᵢ and IC₅₀ data for I3 ([2, 11] 1,3-dithio-propan-2-one bridge,
D present at position 1), Compound 19 ([2, 11] lactam bridge),
and Compound 18 ([2, 11] lactam bridge and D absent at position 1)

| | Kᵢ IL-23R (nM) | IC₅₀ (pSTAT3) (nM) |
|---|---|---|
| I3 (isomer 3) | 38.0 | 407 |
| Compound 19 | 6.32 | 32.3 |
| Compound 18 | 2.85 | 17.6 |

The inventors demonstrated that the [4,7] bridge also can be replaced with an alternative bridging moiety, for example 1,2-phenylenedimethanethiol, and almost retained potency (Compound 20, Tables 2-7a and 2-7b).

TABLE 2-7a
Amino acid sequence of Compound 19, Compound
20, Compound 21, and Compound 22

| | Amino Acid | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| Compound 19 | D | K | S | C | W | Q | C | W | 2-Nal | L | E | R |
| | | (1c) | | (2a) | | | (2a) | | | | (1c) | |
| Compound 20 | D | K | S | C | W | Q | C | W | 2-Nal | L | E | R |
| | | (1c) | | (2d) | | | (2d) | | | | (1c) | |
| Compound 21 | D | K | S | C | W | Q | C | W | 2-Nal | L | E | R |
| | | (1c) | | (2e) | | | (2e) | | | | (1c) | |
| Compound 22 | D | K | S | C | W | Q | C | W | 2-Nal | L | E | R |
| | | (1c) | | (2f) | | | (2f) | | | | (1c) | |

(1c) = [2,11] lactam bridge; (2a) = [4,7] 1,3-dithio-propan-2-one bridge; (2d) = [4,7] 1,2-phenylenedimethanethiol bridge; (2e) = [4,7] 1,3-phenylenedimethanethiol bridge; (2f) = [4,7] 1,4-phenylenedimethanethiol bridge.

TABLE 2-7b
Kᵢ and IC₅₀ data for Compound 19 ([4,7] 1,3-dithio-propan-2-
one bridge), Compound 20 ([4,7] 1,2-phenylenedimethanethiol
bridge), Compound 21 ([4,7] 1,3-phenylenedimethanethiol
bridge), and Compound 22 ([4,7] 1,4-phenylenedimethanethiol bridge)

| | Kᵢ IL-23R (nM) | IC₅₀ (pSTAT3) (nM) |
|---|---|---|
| Compound 19 | 6.32 | 32.3 |
| Compound 20 | 24.6 | 79.2 |
| Compound 21 | 634 | Tested |
| Compound 22 | 563 | Tested |

The inventors further demonstrated that they could cyclize the peptide directly to the N-terminal via an unnatural amino acid residue at the N-terminus (Compound 37, Tables 8a and 8b).

TABLE 2-8a
Amino acid sequence of Compound 18 and Compound 37

| | Amino Acid | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| Compound 18 | — | K | S | C | W | Q | C | W | 2-Nal | L | E | R |
| | | (1c) | | (2a) | | | (2a) | | | | (1c) | |
| Compound 37 | — | α | S | C | W | Q | C | W | 2-Nal | L | E | R |
| | | (1c) | | (2a) | | | (2a) | | | | (1c) | |

α = (3-Aminomethyl)benzoyl; (1c) = [2,11] lactam bridge; (2a) = [4,7] 1,3-dithio-propan-2-one bridge.

TABLE 2-8b
Kᵢ and IC₅₀ data for Compound 18 (side chain
linkage) and Compound 37 (N-terminal linkage)

| | Kᵢ IL-23R (nM) | IC₅₀ (pSTAT3) (nM) |
|---|---|---|
| Compound 18 | 2.85 | 17.6 |
| Compound 37 | 9.86 | 44.6 |

The inventors further found that modification of position 8, for example with Y(2-aminoethoxy), could improve the potency (Compound 18 vs. Compound 29, Tables 2-9a and 2-9b).

TABLE 2-9a
Amino acid sequence of Compound 18 and Compound 29

| | Amino Acid | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| Compound 18 | — | K | S | C | W | Q | C | W | 2-Nal | L | E | R |
| | | (1c) | | (2a) | | | (2a) | | | | (1c) | |
| Compound 29 | — | K | S | C | W | Q | C | β | 2-Nal | L | E | R |
| | | (1c) | | (2a) | | | (2a) | | | | (1c) | |

β = Y(2-aminoethoxy); (1c) = [2,11] lactam bridge; (2a) = [4,7] 1,3-dithio-propan-2-one bridge.

TABLE 2-9b
Kᵢ and IC₅₀ data for Compound 18 (W)
and Compound 29 (Y(2-aminoethoxy))

| | Kᵢ IL-23R (nM) | IC₅₀ (pSTAT3) (nM) |
|---|---|---|
| Compound 18 | 2.85 | 17.6 |
| Compound 29 | 0.64 | 19.7 |

The inventors further evaluated the direct cyclization to the N-terminal. They demonstrated that the location of the aromatic moiety impacted potency (Compound 37, Tables 2-10a and 2-10b).

TABLE 2-10a
Amino acid sequence of Compound 37, Compound
49, Compound 50, Compound 51, and Compound 52

| | Amino Acid | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| Compound 37 | — | α | S | C | W | Q | C | W | 2-Nal | L | E | R |
| | | (1c) | | (2a) | | | (2a) | | | | (1c) | |
| Compound 49 | — | β | S | C | W | Q | C | W | 2-Nal | L | E | R |
| | | (1c) | | (2a) | | | (2a) | | | | (1c) | |
| Compound 50 | — | γ | S | C | W | Q | C | W | 2-Nal | L | E | R |
| | | (1c) | | (2a) | | | (2a) | | | | (1c) | |
| Compound 51 | — | δ | S | C | W | Q | C | W | 2-Nal | L | E | R |
| | | (1c) | | (2a) | | | (2a) | | | | (1c) | |
| Compound 52 | — | ϕ | S | C | W | Q | C | W | 2-Nal | L | E | R |
| | | (1c) | | (2a) | | | (2a) | | | | (1c) | |

α = (3-Aminomethyl)benzoyl; β = (4-Aminomethyl)benzoyl; γ = 4-(2-Aminoethyl)benzoyl; δ = 3-(4-Aminophenyl)propanoyl; ϕ = 4-Aminomethyl-phenylacetyl; (1c) = [2,11] lactam bridge; (2a) = [4,7] 1,3-dithio-propan-2-one bridge.

TABLE 2-10b
Kᵢ and IC₅₀ data for Compound 37, Compound 49,
Compound 50, Compound 51, and Compound 52

| | Kᵢ IL-23R (nM) | IC₅₀ (pSTAT3) (nM) |
|---|---|---|
| Compound 37 | 9.86 | 44.6 |
| Compound 49 | 1.16 | — |
| Compound 50 | 5.09 | — |
| Compound 51 | 2.00 | — |
| Compound 52 | 0.057 | — |

Overall, the inventors have improved the Kᵢ determined affinity from 38.0 nM to 57 pM from I3 (isomer 3) to Compound 52.

### Example 3: Biological Assays

### Binding Assay for Estimating Binding Affinity of Compounds to Human IL-23R

The compound binding affinity for IL-23R was estimated by the ability of compounds to displace a fluorophore-labelled reference compound from the human IL-23R. The assay principle relies on Bioluminescence Resonance Energy Transfer (BRET) between a fluorophore-labelled reference compound binding to the IL-23R part of a fusion protein consisting of the IL-23R fused to a Nanoluc luciferase enzyme (Nanoluc). The Nanoluc is placed in the N-terminus in close proximity to the binding domain of the ligand. Upon close proximity between the fluorophore of the fluorophore-labelled compound and the Nanoluc of the fusion protein, bioluminescence energy generated from conversion of a Nanoluc substrate, is transferred to the fluorophore resulting in an increase in BRET. In presence of an unlabelled compound, the unlabelled compound displaces the fluorophore-labelled peptide from its binding site resulting in a decrease in BRET. The concentration for which half of the fluorophore-labelled peptide is displaced by the unlabelled compound depends on the affinity of the compound for the IL-23R, and is termed the IC₅₀ concentration. An estimated affinity (K) of the unlabelled compound for the IL-23R may be calculated using the Cheng-Prussov equation, which takes into account the concentration of the fluorophore-labelled compound used, and the affinity of the fluorophore-labelled compound for IL-23R. Assuming that the binding is reversible, at equilibrium, the K value is determined using the Cheng-Prussov equation, which describes the relationship between the the Kᵢ and the IC₅₀ as: Kᵢ=IC₅₀/(1+[L_{L}]/K_{dL}), where [L_{L}] is the concentration of labelled compound used and K_{dL} is the equilibrium dissociation constant for the labelled compound (Cheng and Prusoff, 1973).

The fusion protein was generated by subcloning of the cDNA encoding the mature human IL-23R (primary accession number UniProtKB—Q5VWK5, amino acids 22-629) and a small linker sequence in frame with a mammalian expression plasmid encoding a secretion signal and the Nanoluc protein (N1371, Promega). The plasmid also contained a gene to confer resistance for the antibiotic hygromycin. A cell line stably expressing the Nluc-IL23R fusion protein was generated by transfection of HEK293 cells with the expression plasmid and selected with hygromycin for 3 weeks in Growth medium consisting of DMEM w/Glutamax-I containing, 10% V/V FBS, 1% V/V P/S, 1 mM Sodium Pyruvate, and 1×NEAA, and 0.3 mg/mL hygromycin. The remaining cells were propagated and considered a stable expressing Nluc-IL23R fusion protein pool clone.

Cells expressing the Nluc-IL23R fusion protein were expanded in Growth medium and membranes prepared by homogenization of a cell pellet from 18 T175 flasks (4° C. in subsequent steps). The cell pellet was lysed Tris 10 mM, 7.5, 1 mM EDTA and protease inhibitors (Complete, Roche) and homogenized using a 15 mL glass-dounce by 50 strokes. The homogenate was spun at spin @1500 rpm for 10 min, the supernatant transferred to SV-34 tubes and spun at 40000 g for 20 min at 4° C. to pellet the crude membranes. The supernatant was then removed, the pellet resuspended in 5 mL buffer containing 50 mM HEPES pH 7.4, 5 mM EGTA, and 5 mM MgCl2, and homogenized. Aliquots of the resuspended and homogenized membranes containing the Nluc-IL23R fusion protein were stored at −80° C. until use.

Compounds to be tested for binding to IL-23R were serially diluted in Assay buffer (50 mM HEPES pH 7.4, 5 mM EGTA, 5 mM MgCl2, 0.005% Tween-20, and 0.05% casein) and added to the wells of a white 384-well plate (Corning 3572) in a volume of 6.25 μL, together with 12.5 μL diluted membranes containing the Nluc-IL23R fusion protein (0.42 μg/well) and 6.25 μL fluorescently labelled peptide to a final at a concentration of 3.1 nM, also prepared in Assay buffer. The plate was sealed with light impermeable plate seals, and incubated on a orbital shaker 400 rpm for 2 hours at room temperature. To determine the BRET ratio, the plate seal was removed and 25 μL of the Nanoluc substrate (Promega N1572) diluted 1:500 was added to each well and incubated for 1-2 minutes on an orbital shaker at 400 rpm. Then plate was read on an Envision plate reader equipped with a luminescence mirror module (barcode 404) using filters corresponding to Nanoluc substrate luminescence (M470 filter; 470 nm, bandwidth 24 nm) and TAMRA fluorescence (M595p filter 595 nm, bandwidth 60 nm)). The BRET ratio was calculated as the fluorescence from the TAMRA/nanoluc bioluminescence. The IC₅₀ for each compound was determined by computer-aided curve fitting using a 4-parameter logistic (4PL) non-linear model. The Kᵢ for tested compounds was calculated using the Cheng Prussov equation as described above and are shown in Table 3-1.

The K_{d} was determined by performing a saturation binding experiment, using increasing concentrations of the TAMRA-labelled peptide in absence and presence of a high concentration of unlabelled compound (Stoddart et al., 2015). The K_{d} of the TAMRA-labelled peptide for the IL-23R receptor was determined to be 13.7 nM and the concentration of the TAMRA-labelled peptide in the assay was 3.1 nM.

### Functional Inhibition of IL-23 Mediated STAT3 Signalling by Compounds

The ability of compounds to inhibit IL-23 mediated signalling was determined in the human-derived DB cell line (CRL-2289) (hereafter referred to as DB cells), which endogenously expresses the human IL-23R and human IL-12R β1 subunits. Upon binding of IL-23, the IL-23R forms a heterodimer signalling complex together with IL-12R β1, which through the JAK2/STAT3 pathway promotes phosphorylation of STAT3 to form phospho-STAT3. In the assay, the functional antagonism of IL-23 mediated phospho-STAT3 formation in DB cells by the compound is quantified using reagents capable of measuring the phosphorylation state of Tyr705 of STAT3 in the form of a phospho-STAT3 (Tyr705) MSD (Meso Scale Discovery) kit.

The assay was used to quantify the functional antagonism of a compound and to rank order the inhibitor compounds according to their potency. For compounds tested in this assay, the response was normalized relative to control values to calculate the IC₅₀ and maximal inhibitory response from a concentration response curve of the compound in presence of a fixed concentration of human IL-23.

The assay procedure was as follows. DB cells were maintained in growth medium consisting of RPMI-1640 [Invitrogen 61870-010] supplemented with 10% V/V Foetal Bovine Serum (FBS) [(heat inactivated), Invitrogen 10270-106], and 1% V/V Penicillin-Streptomycin (Pen-Strep) Solution [Invitrogen 15140]. On the day of assay, cells were resuspended in Assay buffer consisting of RPMI-1640 [Invitrogen 61870-010] supplemented with 0.1% w/V BSA [Sigma-Aldrich A9430] to a density of 7.5×10⁶ cells/mL. Compounds to be tested for inhibition of hIL-23 mediated signalling were serially diluted in Assay buffer to 3× the final concentration. A solution of hIL-23 corresponding to 3× the EC₈₀ of hIL-23 (1.7 nM) was also prepared in Assay buffer. To initiate the assay, 20 μL of DB cell suspension (corresponding to 150.000 cells/well) was added to the wells of a 96-well V-bottom Polypropylene plate [Corning 3363] followed by addition of 3×20 μL of the diluted test compounds to separate wells. Following preincubation of the DB cells with inhibitor for 15 min in a cell incubator (37° C., 5% CO₂), 20 μL of the prepared 3× the EC₈₀ of hIL-23 solution was added to each well and incubated for 90 min in a cell incubator (37° C., 5% CO₂). For some wells buffer only or IL-23 only corresponding to the EC₈₀ was added to obtain readouts needed for normalization. To terminate the assay, the plate was spun at 1000 G for 5 min to pellet cells, the supernatant removed using an 8-channel manual pipette, and then added 50 μL/well Complete Lysis Buffer from the MSD STAT3 kit (Cat #K150SVD, Mesoscale) to the cell pellet. To completely lyse cells to release phospho-STAT3 for detection, the plate was shaken for 10 min at room temperature (500 rpm) sealed with aluminum foil and placed at −80° C. for a minimum of 15 min. Detection of the phospho-STAT3 level in the cell lysate from the individual wells was determined using the MSD STAT3 kit (Cat #K150SVD, Mesoscale) and read on a Meso QuickPlex SQ 120 μlate reader (Mesoscale).

For data analysis raw data counts from the Meso QuickPlex SQ 120 μlate reader were normalized relative to the response by the EC₈₀ of hIL-23 alone (no compound added) and the buffer level. Compound potency (IC₅₀) and maximal inhibitory response (% inhibition) were estimated by computer-aided curve fitting using a 4-parameter logistic (4PL) non-linear model. Data for the compound potency (IC₅₀) are shown in Table 3-1.

The in vitro activity results (expressed as Kᵢ hIL-23R and IC₅₀ (pSTAT3) (nM) values) are summarized in Table 3-1 below.

TABLE 3-1
Kᵢ hIL-23R (nM), and IC₅₀ (pSTAT3) (nM) data

| Compound | Kᵢ hIL-23R (nM) | IC₅₀ (pSTAT3) (nM) |
|---|---|---|
| 1 | 79.8 | 783 |
| 2 | 32.3 | 755 |
| 3 | 205 | — |
| 4 | 143 | 847 |
| 5 | Tested | Tested |
| 6 | Tested | Tested |
| 7 | 26.8 | 159 |
| 9 | Tested | Tested |
| 10 | 101 | 617 |
| 11 | 706 | Tested |
| 12 | 373 | Tested |
| 13 | Tested | Tested |
| 14 | Tested | Tested |
| 15 | Tested | Tested |
| 16 | 95.4 | 461 |
| 17 | 70.7 | 366 |
| 18 | 2.85 | 17.6 |
| 19 | 6.33 | 33.2 |
| 20 | 24.6 | 79.2 |
| 21 | 634 | Tested |
| 22 | 563 | Tested |
| 23 | 117 | — |
| 25 | 185 | Tested |
| 26 | Tested | Tested |
| 27 | 1.77 | 15.5 |
| 28 | 4.52 | 27.4 |
| 29 | 0.680 | 19.7 |
| 30 | 5.53 | 68.6 |
| 31 | 76.3 | 968 |
| 32 | 14.2 | 118 |
| 33 | 189 | — |
| 34 | 65.0 | — |
| 35 | 41.2 | — |
| 36 | Tested | Tested |
| 37 | 9.87 | 44.6 |
| 38 | 2.56 | 19.3 |
| 39 | 1.40 | 19.6 |
| 40 | 18.1 | — |
| 41 | 2.31 | 29.4 |
| 42 | 2.76 | 24.7 |
| 44 | 4.87 | 82.3 |
| 45 | 12.0 | — |
| 46 | 48.2 | — |
| 47 | 0.624 | 17.2 |
| 48 | 17.0 | — |
| 49 | 1.16 | 20.3 |
| 50 | 5.10 | 51.1 |
| 51 | 2.00 | 68.1 |
| 52 | 0.0475 | 7.85 |
| 53 | Tested | — |
| 54 | 178 | — |
| 55 | 272 | Tested |
| 56 | — | Tested |
| 57 | 68.0 | 167 |
| 58 | 201 | 597 |
| 59 | 139 | 297 |
| 60 | 212 | 666 |
| 61 | 436 | Tested |
| 62 | Tested | Tested |
| 63 | Tested | Tested |
| 64 | Tested | — |
| 67 | 0.760 | 35.3 |
| 68 | 3.32 | 63.9 |
| 69 | 2.76 | 41.1 |
| 70 | 0.485 | 8.43 |
| 78 | 0.0205 | 0.132 |
| 79 | 2.09 | 21.3 |
| 80 | 0.150 | 10.5 |
| 81 | 0.318 | 20.7 |
| 82 | 0.0665 | 11.1 |
| 84 | 0.922 | 12.5 |
| 85 | 0.321 | 8.54 |
| 87 | 0.439 | 8.89 |
| 90 | 0.706 | 15.8 |
| 108 | 0.0995 | 10.6 |
| 109 | 0.460 | 21.5 |
| 115 | 0.182 | 18.9 |
| 117 | 0.188 | 11.3 |
| 119 | 0.303 | 16.7 |
| 120 | 0.00938 | 0.0542 |
| 121 | 0.00954 | 0.367 |
| 122 | 0.00541 | 0.0277 |
| 123 | 0.00783 | 0.0253 |
| 124 | 0.0107 | 0.117 |
| 125 | 0.599 | 21.2 |
| 126 | 0.718 | 28.0 |
| 136 | 0.170 | 8.8 |
| 137 | 0.518 | 24.1 |
| 138 | 0.723 | 39.4 |
| 139 | 0.612 | 30.2 |
| 145 | 0.723 | 22.7 |
| 146 | 1.01 | 41.5 |
| 147 | 0.0119 | 0.0858 |
| 148 | 0.00897 | 0.0868 |
| 149 | 0.163 | 4.82 |
| 150 | 0.597 | 14.3 |
| 151 | 0.672 | 10.7 |
| 155 | 0.00805 | 0.0305 |
| 156 | 0.0157 | 0.0857 |
| 157 | 0.0117 | 0.0318 |
| 158 | 0.0272 | 0.635 |
| Ref 1 | Tested | Tested |
| Ref 2 | Tested | Tested |
| Ref 3 | Tested | Tested |
| Ref 4 | Tested | Tested |
| Ref 5 | Tested | Tested |
| Ref 6 | Tested | Tested |
| Ref 7 | Tested | Tested |
| I1 (isomer 3) | 104 | Tested |
| I3 (isomer 3) | 38.0 | 407 |
| I4 (isomer 3) | Tested | Tested |
| I5 (isomer 3) | 73.6 | 934 |
| Compound C | 2.11 | 13.8 |

Tested = Kᵢ or IC₅₀ estimated higher than 1 μM when tested in assays

### Determination of Peptide Stability in Simulated Gastric Fluid (SGF) and Simulated Intestinal Fluid (SIF)

SGF and SIF were prepared based on the United States Pharmacopeia specifications (Test Solutions, United States Pharmacopeia 35, NF 30, 2012). SGF was prepared by dissolving sodium chloride (0.2 g) in 50 mL of water. A volume of 0.7 mL of 10 M HCl was added to adjust the pH of the solution to 1.2 and the volume made up to 100 mL with water. 64 mg porcine pepsin (P7125, Sigma Aldrich) was gently dissolved into 20 mL preheated (37° C.) solution (3.2 g/L) immediately prior to the incubation. SIF was prepared by dissolving monobasic potassium phosphate (0.68 g) in 50 mL of water and adjusting the pH to 6.8 with 1 M NaOH. The volume was subsequently made up to 100 mL with water. 200 mg porcine pancreatin (P1625, Sigma Aldrich) was gently dissolved into 20 mL preheated (37° C.) solution (10 g/L) immediately prior to the incubation.

To initiate the incubations, 20 μL of peptide stock solution in 50% v/v isopropanol was deposited at the bottom of a well plate and 580 μL matrix solution was added to give a final substrate concentration of 10 μM. The incubation was performed at 37° C. with gentle shaking. 70 μL aliquots were removed at 0, 1 and 4 hours and quenched into 210 μL ice-cold precipitant solution (95% v/v acetonitrile with 0.1% v/v formic acid). After the last timepoint, the sampling plate was mixed for 10 min on a shaking table and centrifuged 10 min at 2200 g. 70 μL resulting supernatant was diluted with 150 μL water, mixed and centrifugated before analysis by liquid chromatography high resolution mass spectrometry. The zero sample was reinjected after the 4-hour sample to confirm that no drift in instrument sensitivity had occurred during the run. Percent remaining at each timepoint was calculated relative to timepoint zero based on absolute peak areas.

The in vitro SIF results (expressed as % peptide remaining after the specified time period) are summarized in Table 3-2 below.

TABLE 3-2
SIF data

| Compound | SIF (% remaining 1 h) | SIF (% remaining 4 h) |
|---|---|---|
| 1 | 0 | 0 |
| 2 | 0 | 0 |
| 4 | 0 | 0 |
| 7 | 0 | 0 |
| 17 | 0 | 0 |
| 27 | 0 | 0 |
| 37 | 0 | 0 |
| 38 | 29 | 5 |
| 39 | 0 | 0 |
| 40 | 70 | 36 |
| 41 | 83 | 79 |
| 42 | 88 | 85 |
| 43 | 55 | 33 |
| 44 | 79 | 80 |
| 45 | 76 | 34 |
| 46 | 9 | 0 |
| 47 | 2 | 0 |
| 48 | 0 | 0 |
| 57 | 7 | 0 |
| 58 | 80 | 45 |
| 60 | 69 | 12 |
| 78 | 5 | 0 |
| 80 | 92 | 86 |
| 81 | 99 | 77 |
| 82 | 95 | 77 |
| 85 | 93 | 74 |
| 87 | 95 | 81 |
| 90 | 84 | 67 |
| 92 | 106 | 102 |
| 120 | 82 | 39 |
| 121 | 57 | 6 |
| 122 | 73 | 25 |
| 123 | 46 | 4 |
| 124 | 35 | 0 |
| 130 | 1 | 0 |
| 165 | — | 109 |
| 166 | — | 86 |
| 168 | — | 104 |
| 169 | — | 72 |
| 170 | — | 62 |
| 171 | — | 68 |
| 172 | — | 70 |
| 173 | — | 96 |
| 174 | — | 86 |
| 175 | — | 93 |
| 176 | — | 95 |
| 177 | — | 63 |
| Ref 5 | 0 | 0 |
| Ref 6 | 0 | 0 |
| I1 (isomer 3) | 0 | 0 |
| I3 (isomer 3) | 0 | 0 |
| I4 (isomer 3) | 0 | 0 |
| I5 (isomer 3) | 78 | 22 |
| Compound C | 92 | 76 |

The in vitro SGF results (expressed as % peptide remaining after the specified time period) are summarized in Table 3-3 below.

TABLE 3-3
SGF data

| Compound | SGF (% remaining 1 h) | SGF (% remaining 4 h) |
|---|---|---|
| 1 | 86 | 68 |
| 2 | 6 | 1 |
| 4 | 1 | 0 |
| 7 | 0 | 0 |
| 17 | 0 | 0 |
| 27 | 0 | 0 |
| 37 | 2 | 1 |
| 38 | 84 | 84 |
| 39 | 90 | 91 |
| 41 | 93 | 87 |
| 42 | 90 | 89 |
| 44 | 77 | 73 |
| 45 | 76 | 84 |
| 46 | 83 | 66 |
| 48 | 106 | 110 |
| 57 | 21 | 1 |
| 58 | 61 | 8 |
| 60 | 7 | 0 |
| 120 | 84 | 81 |
| 121 | 82 | 80 |
| 122 | 92 | 90 |
| 123 | 100 | 86 |
| 124 | 91 | 89 |
| 130 | 59 | 14 |
| 165 | — | 109 |
| 166 | — | 99 |
| 168 | — | 104 |
| 169 | — | 121 |
| 170 | — | 101 |
| 171 | — | 75 |
| 172 | — | 99 |
| 173 | — | 103 |
| 174 | — | 97 |
| 175 | — | 108 |
| 176 | — | 92 |
| 177 | — | 106 |
| Ref 5 | 1 | 0 |
| Ref 6 | 0 | 0 |
| I1 (isomer 3) | 92 | 94 |
| I3 (isomer 3) | 5 | 0 |
| I4 (isomer 3) | 99 | 99 |
| I5 (isomer 3) | 81 | 54 |
| Compound C | 100 | 100 |

### REFERENCES

Cheng and Prusoff, Biochem. Pharmacol., 1973, 22(23), 3099-3108.
Heinis et al., Nature Biomedical Engineering, 2020, 4, 560-571.
Sayago et al., ACS Med. Chem. Lett., 2018, 9, 912-916.
Stoddart et al., Nat. Methods, 2015, 12, 661-663.
WO 2016/011208
WO 2017/011820
WO 2018/022937
WO 2018/089693
WO 2018/136646
WO 2020/014646
WO 2021/007433
WO 2021/146441
WO 2021/146458
US 2013/0029907

### CLAUSES

1. A compound of the formula:

R¹—Z—R²
wherein
R¹ is H, C₁₋₄ acyl, benzoyl, C₁₋₄ alkyl, or is absent;
R² is NHR³, OH, or is absent, wherein R³ is hydrogen or C₁₋₃ alkyl; and
Z is an amino acid sequence of formula I:

X1-X2-X3-X4-X5-X6-X7-X8-X9-X10-X11-X12-X13-X14 (1)
wherein
X1 is absent or is selected from the group consisting of Asp, Glu, Ser, Cys, and Lys;
X3 is selected from the group consisting of Ser, beta-homo-Ser, Thr, Leu, Cys, and Gln;
X5 is selected from the group consisting of Trp, Tyr, Ala, 1-Me-Trp, 7-Me-Trp, 7-Ph-Trp, 7-(Naphth-2-yl)-Trp, 2-Nal, and Bip;
X6 is selected from the group consisting of Gln, Glu, Tyr and Cys;
X8 is selected from the group consisting of Trp, Tyr, Asn, Ala, His, and 2-Nal, wherein the hydroxyl group of Tyr is optionally substituted with C₁₋₃ alkyl optionally substituted with NH₂;
X9 is selected from the group consisting of 2-Nal, Trp, 1-Me-Trp, 6-Cl-Trp, 3-(3-Quinolinyl)-Ala, Phe, 4-F-Phe, Glu, Cys, and Ala;
X10 is selected from the group consisting of Leu, D-Leu, 2-Me-Leu, 2-Me-Lys, Trp, Asn, Cys, and 4-aminotetrahydro-2H-pyran-4-acetyl;
X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent;
X13 is absent or is selected from the group consisting of Asn, Gly, 3-(3-Pyridyl)-Ala, and 3-(4-Pyridyl)-Ala;
X14 is absent or is Gly;
X2 and X11 are amino acid residues who together form a lactam bridge, a dithioether bridge, or a bridge containing a triazole ring; and
X4 and X7 are amino acid residues who together form a lactam bridge, a dithioether bridge, or a bridge containing a triazole ring;
or a pharmaceutically acceptable salt or solvate thereof;
wherein the compound is not:

I3(isomer 3)H-DC(1a)SC(2a)WQC(2a)WWLC(1a)R-[NH₂];

wherein (1a) is a [2,11]1,3-dithio-propan-2-one bridge and (2a) is a [4,7]1,3-dithio-propan-2-one bridge.
2. The compound according to clause 1, wherein X1 is absent or Asp.
3. The compound according to clause 1 or 2, wherein X1 is absent.
4. The compound according to any one of the preceding clauses, wherein X3 is Ser.
5. The compound according to any one of the preceding clauses, wherein X5 is Trp.
6. The compound according to any one of the preceding clauses, wherein X6 is Gln.
7. The compound according to any one of the preceding clauses, wherein X8 is Tyr wherein the hydroxyl group of Tyr is optionally substituted with —CH₂CH₂NH₂.
8. The compound according to any one of the preceding clauses, wherein X9 is 2-Nal or Trp.
9. The compound according to any one of the preceding clauses, wherein X9 is 2-Nal.
10. The compound according to any one of the preceding clauses, wherein X10 is Leu or 2-Me-Leu.
11. The compound according to any one of the preceding clauses, wherein X10 is Leu.
12. The compound according to any one of the preceding clauses, wherein X12 is Arg.
13. The compound according to any one of the preceding clauses, wherein X13 is absent.
14. The compound according to any one of the preceding clauses, wherein X14 is absent.
15. The compound according to any one of the preceding clauses, wherein R¹ is H, C₁₋₂ acyl, or absent.
16. The compound according to any one of the preceding clauses, wherein R¹ is absent.
17. The compound according to any one of clauses 1 to 15, wherein R¹ is —C(═O)CH₃.
18. The compound according to any one of the preceding clauses, wherein R² is NH₂.
19. The compound according to any one of the preceding clauses, wherein the length of the bridge between X2 and X11 and/or X4 and X7 is 5 to 10 atoms long.
20. The compound according to any one of the preceding clauses, wherein X1 is absent, R¹ is absent, and X2 and X11 are amino acid residues that together form a lactam bridge or a bridge containing a triazole ring via the N-terminus of X2.
21. The compound according to any one of the preceding clauses, wherein X1 and X12 to X14 are absent, R¹ and R² are absent, and X2 and X11 are amino acid residues that together form a head-to-tail cyclised lactam bridge via the N-terminus of X2 and the C-terminus of X11.
22. The compound according to any one of the preceding clauses, wherein the dithioether bridge between X2 and X11 and/or X4 and X7 is of the formula —S—L—Y—L—S—, wherein:
   each S is a sulfur atom and is part of the amino acid residue at X2 and X11 and/or X4 and X7;
   each L is independently C₁₋₄ alkylene; and
   Y is either absent, C(═O), or arylene.
23. The compound according to clause 22, wherein each L is independently C₁₋₂ alkylene.
24. The compound according to clause 22 or 23, wherein each L is methylene.
25. The compound according to any one of clauses 22 to 24, wherein Y is C(═O).
26. The compound according to any one of clauses 22 to 25, wherein Y is arylene selected from phenylene.
27. The compound according to clause 26, wherein Y is phenylene selected from 1,2-phenylene, 1,3-phenylene, and 1,4-phenylene.
28. The compound according to clause 26 or 27, wherein Y is 1,2-phenylene.
29. The compound according to any one of the preceding clauses, wherein the bridge containing a triazole ring between X2 and X11 and/or X4 and X7 contains a 1,2,3-triazole ring.
30. The compound according to any one of the preceding clauses, wherein the bridge containing a triazole ring between X2 and X11 and/or X4 and X7 is attached to positions 1 and 4 of the triazole ring.
31. The compound according to any one of the preceding clauses, wherein X2 and X11 are amino acid residues who together form a lactam bridge.
32. The compound according to clause 31, wherein the location of the amide bond in the lactam bridge is closer to X11 than X2.
33. The compound according to clause 31 or 32, wherein one of the residues at position X2 and X11 is selected from Lys, Arg, Orn, bAla, 3-(4-aminophenyl)propanoyl, (3-aminomethyl)benzoyl, (4-aminomethyl)benzoyl, 4-(2-aminoethyl)benzoyl, 2-aminomethyl-phenylacetyl, 3-aminomethyl-phenylacetyl, 4-aminomethyl-phenylacetyl, 4-aminomethyl-2-pyridineacetyl, 4-aminomethyl-3-pyridineacetyl, 4-aminomethyl-2-fluoro-phenylacetyl, 4-aminomethyl-3-fluoro-phenylacetyl, 4-aminomethyl-2-methyl-phenylacetyl, 4-aminomethyl-3-methyl-phenylacetyl, 4-aminomethyl-2-methoxy-phenylacetyl, 4-aminomethyl-3-methoxy-phenylacetyl, Dab, 6-aminohexanoyl, 6-amino-4-oxahexanoyl, trans-4-aminomethyl-cyclohexyl-1-carbonyl, and (4-(2-aminoethyl)-piperazine-1-yl)-acetyl, and the other is selected from Glu and Asp.
34. The compound according to clause 33, wherein X2 is selected from Lys, Orn, bAla, 3-(4-aminophenyl)propanoyl, (3-aminomethyl)benzoyl, (4-aminomethyl)benzoyl, 4-(2-aminoethyl)benzoyl, 2-aminomethyl-phenylacetyl, 3-aminomethyl-phenylacetyl, 4-aminomethyl-phenylacetyl, 4-aminomethyl-2-pyridineacetyl, 4-aminomethyl-3-pyridineacetyl, 4-aminomethyl-2-fluoro-phenylacetyl, 4-aminomethyl-3-fluoro-phenylacetyl, 4-aminomethyl-2-methyl-phenylacetyl, 4-aminomethyl-3-methyl-phenylacetyl, 4-aminomethyl-2-methoxy-phenylacetyl, 4-aminomethyl-3-methoxy-phenylacetyl, 6-aminohexanoyl, 6-amino-4-oxahexanoyl, trans-4-aminomethyl-cyclohexyl-1-carbonyl, and (4-(2-aminoethyl)-piperazine-1-yl)-acetyl, and X11 is selected from Glu and Asp.
35. The compound according to clause 34, wherein:
   X2 is Lys and X11 is Glu;
   X2 is Orn and X11 is Glu;
   X2 is bAla and X11 is Glu;
   X2 is 3-(4-aminophenyl)propanoyl and X11 is Glu;
   X2 is (3-aminomethyl)benzoyl and X11 is Glu;
   X2 is (4-aminomethyl)benzoyl and X11 is Glu;
   X2 is 4-(2-aminoethyl)benzoyl and X11 is Glu;
   X2 is 2-aminomethyl-phenylacetyl and X11 is Glu;
   X2 is 3-aminomethyl-phenylacetyl and X11 is Glu;
   X2 is 4-aminomethyl-phenylacetyl and X11 is Glu;
   X2 is 6-aminohexanoyl and X11 is Glu;
   X2 is 6-amino-4-oxahexanoyl and X11 is Glu;
   X2 is trans-4-aminomethyl-cyclohexyl-1-carbonyl and X11 is Glu;
   X2 is (4-(2-aminoethyl)-piperazine-1-yl)-acetyl and X11 is Glu;
   X2 is (4-(2-aminoethyl)-piperazine-1-yl)-acetyl and X11 is Asp;
   X2 is 4-aminomethyl-2-pyridineacetyl and X11 is Glu;
   X2 is 4-aminomethyl-3-pyridineacetyl and X11 is Glu;
   X2 is 4-aminomethyl-2-fluoro-phenylacetyl and X11 is Glu;
   X2 is 4-aminomethyl-3-fluoro-phenylacetyl and X11 is Glu;
   X2 is 4-aminomethyl-2-methyl-phenylacetyl and X11 is Glu;
   X2 is 4-aminomethyl-3-methyl-phenylacetyl and X11 is Glu;
   X2 is 4-aminomethyl-2-methoxy-phenylacetyl and X11 is Glu; or
   X2 is 4-aminomethyl-3-methoxy-phenylacetyl and X11 is Glu.
36. The compound according to clause 34 or 35, wherein:
   X2 is Lys and X11 is Glu.
37. The compound according to clause 33, wherein X2 is selected from Glu and Asp, and X11 is selected from Lys, Arg, and Dab.
38. The compound according to clause 37, wherein:
   X2 is Glu and X11 is Lys;
   X2 is Glu and X11 is Dab; or
   X2 is Asp and X11 is Arg.
39. The compound according to any one clauses 1 to 30, wherein X2 and X11 are amino acid residues who together form a dithioether bridge.
40. The compound according to clause 39, wherein X2 is Cys and X11 is Cys.
41. The compound according to any one clauses 1 to 30, wherein X2 and X11 are amino acid residues who together form a bridge containing a triazole ring.
42. The compound according to clause 41, wherein one of the residues at position X2 and X11 is selected from Lys(N₃), azidoacetic acid, (N₃)-Ala, Dab(azidoacetic acid), and Dab((N₃)-Ala), and the other is selected from Pra, Glu(propargylamine), and Dab(3-butynoic acid).
43. The compound according to clause 42, wherein X2 is selected from Lys(N₃), azidoacetic acid, and (N₃)-Ala; and X11 is selected from Pra, Glu(propargylamine), and Dab(3-butynoic acid).
44. The compound according to clause 43, wherein:
   X2 is Lys(N₃) and X11 is Pra;
   X2 is azidoacetic acid and X11 is Glu(propargylamine);
   X2 is azidoacetic acid and X11 is Dab(3-butynoic acid);
   X2 is (N₃)-Ala and X11 is Glu(propargylamine); or
   X2 is (N₃)-Ala and X11 is Dab(3-butynoic acid).
45. The compound according to clause 43 or 44, wherein:
   X2 is Lys(N₃) and X11 is Pra.
46. The compound according to clause 42, wherein X2 is Pra; and X11 is selected from Dab(azidoacetic acid), and Dab((N₃)-Ala).
47. The compound according to clause 46, wherein:
   X2 is Pra and X11 is Dab(azidoacetic acid); or
   X2 is Pra and X11 is Dab((N₃)-Ala).
48. The compound according to any one of the preceding clauses, wherein X4 and X7 are amino acid residues who together form a dithioether bridge.
49. The compound according to clause 48, wherein X4 is Cys and X7 is Cys.
50. The compound according to any one of clauses 1 to 47, wherein X4 and X7 are amino acid residues who together form a lactam bridge.
51. The compound according to clause 50, wherein one of the residues at position X4 and X7 is Lys, Dpr, Dab, or Orn, and the other is Glu.
52. The compound according to clause 51, wherein X4 is selected from Lys, Dpr, Dab, and Orn, and X7 is Glu.
53. The compound according to clause 52, wherein:
   X4 is Dpr and X7 is Glu;
   X4 is Dab and X7 is Glu; or
   X4 is Orn and X7 is Glu.
54. The compound according to clause 51, wherein X4 is Glu, and X7 is selected from Lys, Dpr, Dab, and Orn.
55. The compound according to clause 54, wherein:
   X4 is Glu and X7 is Lys;
   X4 is Glu and X7 is Dpr; or
   X4 is Glu and X7 is Orn.
56. The compound according to any one clauses 1 to 47, wherein X4 and X7 are amino acid residues who together form a bridge containing a triazole ring.
57. The compound according to clause 56, wherein one of the residues at position X4 and X7 is selected from Lys(N₃) and Aha, and the other is Pra.
58. The compound according to clause 57, wherein:
   X4 is Lys(N₃) and X7 is Pra; or
   X4 is Aha and X7 is Pra.
59. The compound according to any one of clauses 1 to 6, 8, 9, and 12 to 58, wherein X8 is Trp; X10 is D-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.
60. The compound according to any one of clauses 1 to 9, and 12 to 58, wherein X8 is Tyr wherein the hydroxyl group of Tyr is optionally substituted with C₁₋₃ alkyl optionally substituted with NH₂; X10 is D-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.
61. The compound according to any one of clauses 1 to 6, 8, 9, and 12 to 58, wherein X8 is Asn; X10 is D-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.
62. The compound according to any one of clauses 1 to 6, 8, 9, and 12 to 58, wherein X8 is Ala; X10 is D-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.
63. The compound according to any one of clauses 1 to 6, 8, 9, and 12 to 58, wherein X8 is His; X10 is D-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.
64. The compound according to any one of clauses 1 to 6, 8, 9, and 12 to 58, wherein X8 is 2-Nal; X10 is D-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.
65. The compound according to any one of clauses 1 to 10, and 12 to 58, wherein X8 is Tyr wherein the hydroxyl group of Tyr is optionally substituted with —CH₂CH₂NH₂; X10 is 2-Me-Leu; and X12 is Arg or is absent.
66. The compound according to any one of clauses 1 to 6, 8 to 10, and 12 to 58, wherein X8 is Trp; X10 is 2-Me-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.
67. The compound according to any one of clauses 1 to 10, and 12 to 58, wherein X8 is Tyr wherein the hydroxyl group of Tyr is optionally substituted with C₁₋₃ alkyl optionally substituted with NH₂; X10 is 2-Me-Leu; and X12 is selected from the group consisting of Arg, D-Arg, 2-Me-Arg, N-Me-Arg, Ser, Phe, 4-NH₂Phe, Tyr, Thr, Met, Gly, Glu, Asn, Dab, 3-(3-Pyridyl)-Ala, and 3-(4-Pyridyl)-Ala, or is absent.
68. The compound according to any one of clauses 1 to 6, 8 to 10, and 12 to 58, wherein X8 is Asn; X10 is 2-Me-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.
69. The compound according to any one of clauses 1 to 6, 8 to 10, and 12 to 58, wherein X8 is Ala; X10 is 2-Me-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.
70. The compound according to any one of clauses 1 to 6, 8 to 10, and 12 to 58, wherein X8 is His; X10 is 2-Me-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.
71. The compound according to any one of clauses 1 to 6, 8 to 10, and 12 to 58, wherein X8 is 2-Nal; X10 is 2-Me-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.
72. The compound according to any one of clauses 1 to 9, and 12 to 58, wherein X8 is Tyr wherein the hydroxyl group of Tyr is optionally substituted with —CH₂CH₂NH₂; X10 is D-Leu; and X12 is Arg or is absent.
73. The compound according to any one of clauses 1 to 10, and 12 to 58, wherein X8 is Tyr wherein the hydroxyl group of Tyr is optionally substituted with —CH₂CH₂NH₂; X10 is 2-Me-Leu; and X12 is Arg or is absent.
74. The compound according to clause 1 wherein Z is an amino acid sequence selected from the group consisting of:

| | |
|---|---|
| SEQ ID NO: 1 | DC(1a)SC(2a)WQC(2a)WW[2-Me-Leu]C(1a)R |
| SEQ ID NO: 2 | LC(1a)SC(2a)WQC(2a)WWLC(1a)R |
| SEQ ID NO: 3 | DC(1a)SC(2a)WEC(2a)WWLC(1a)R |
| SEQ ID NO: 4 | DE(1c)SC(2a)WQC(2a)WWLK(1c)R |
| SEQ ID NO: 5 | DC(1a)SE(2c)WQK(2c)WWLC(1a)R |
| SEQ ID NO: 6 | D(1c)*SC(2a)WQC(2a)WWLR(1c)* |
| SEQ ID NO: 7 | DK(1c)SC(2a)WQC(2a)WWLE(1c)R |
| SEQ ID NO: 8 | D[Orn](1c)SC(2a)WQC(2a)WWLE(1c)R |
| SEQ ID NO: 9 | DE(1c)SC(2a)WQC(2a)WWL[Dab](1c)R |
| SEQ ID NO: 10 | E(1c)SC(2a)WQC(2a)WWLK(1c)R |
| SEQ ID NO: 11 | DE(1c)SC(2a)WQC(2a)WWLK(1c) |
| SEQ ID NO: 12 | E(1c)SC(2a)WQC(2a)WWLK(1c) |
| SEQ ID NO: 13 | DE(1c)SC(2a)AQC(2a)WWLK(1c)R |
| SEQ ID NO: 14 | DE(1c)SC(2a)WQC(2a)AWLK(1c)R |
| SEQ ID NO: 15 | DE(1c)SC(2a)WQC(2a)WALK(1c)R |
| SEQ ID NO: 16 | DE(1c)SC(2a)[7-Me-Trp]QC(2a)WWLK(1c)R |
| SEQ ID NO: 17 | DE(1c)SC(2a)WQC(2a)W[2-Nal]LK(1c)R |
| SEQ ID NO: 18 | K(1c)SC(2a)WQC(2a)W[2-Nal]LE(1c)R |
| SEQ ID NO: 19 | DK(1c)SC(2a)WQC(2a)W[2-Nal]LE(1c)R |
| SEQ ID NO: 20 | DK(1c)SC(2d)WQC(2d)W[2-Nal]LE(1c)R |
| SEQ ID NO: 21 | DK(1c)SC(2e)WQC(2e)W[2-Nal]LE(1c)R |
| SEQ ID NO: 22 | DK(1c)SC(2f)WQC(2f)W[2-Nal]LE(1c)R |
| SEQ ID NO: 23 | DK(1c)S[K(N3)](2g)WQ[Pra](2g)W[2-Nal]LE(1c)R |
| SEQ ID NO: 24 | DK(1c)S[Aha](2g)WQ[Pra](2g)W[2-Nal]LE(1c)R |
| SEQ ID NO: 25 | K(1c)SC(2a)[2-Nal]QC(2a)W[2-Nal]LE(1c)R |
| SEQ ID NO: 26 | K(1c)SC(2a)[Bip]QC(2a)W[2-Nal]LE(1c)R |
| SEQ ID NO: 27 | K(1c)SC(2a)[1-Me-Trp]QC(2a)W[2-Nal]LE(1c)R |
| SEQ ID NO: 28 | DK(1c)SC(2a)WQC(2a)[2-Nal][2-Nal]LE(1c)R |
| SEQ ID NO: 29 | K(1c)SC(2a)WQC(2a)[Y(2-aminoethoxy)][2-Nal]LE(1c)R |
| SEQ ID NO: 30 | K(1c)SC(2a)WQC(2a)H[2-Nal]LE(1c)R |
| SEQ ID NO: 31 | K(1c)SC(2a)WQC(2a)W[F(4-F)]LE(1c)R |
| SEQ ID NO: 32 | K(1c)SC(2a)WQC(2a)W[1-Me-Trp]LE(1c)R |
| SEQ ID NO: 33 | C(1a)TC(2a)WEC(2a)WW[2-Me-Leu]C(1a)S |
| SEQ ID NO: 34 | GK(1c)TC(2a)WEC(2c)WW[2-Me-Leu]E(1c)S |
| SEQ ID NO: 35 | GC(1a)TC(2a)WEC(2a)W[2-Nal][2-Me-Leu]C(1a)R |
| SEQ ID NO: 36 | [bAla](1c)*SC(2a)WQC(2a)W[2-Nal]LE(1c)R |
| SEQ ID NO: 37 | [(3-Aminomethyl)benzoyl](1c)*SC(2a)WQC(2a)W[2-Nal]LE(1c)R |
| SEQ ID NO: 38 | [(3-Aminomethyl)benzoyl](1c)*SC(2a)WQC(2a)[Y(2-aminoethoxy)][2- |
| | Nal][2-Me-Leu]E(1c)R-[NH2] |
| SEQ ID NO: 39 | [(3-Aminomethyl)benzoyl](1c)*SC(2a)WQC(2a)[Y(2-aminoethoxy)][2- |
| | Nal]LE(1c)R |
| SEQ ID NO: 40 | [(3-Aminomethyl)benzoyl](1c)*SC(2a)WQC(2a)W[2-Nal][2-Me-Leu]E(1c)R |
| SEQ ID NO: 41 | [(3-Aminomethyl)benzoyl](1c)*SC(2a)WQC(2a)[Y(2-aminoethoxy)][2- |
| | Nal][2-Me-Leu]E(1c)[{d}R] |
| SEQ ID NO: 42 | [(3-Aminomethyl)benzoyl](1c)*SC(2a)WQC(2a)[Y(2-aminoethoxy)][2- |
| | Nal][2-Me-Leu]E(1c)[2-Me-Arg] |
| SEQ ID NO: 43 | [(3-Aminomethyl)benzoyl](1c)*SC(2a)WQC(2a)[Y(2-aminoethoxy)][2- |
| | Nal][2-Me-Leu]E(1c)[N-Me-Arg] |
| SEQ ID NO: 44 | [(3-Aminomethyl)benzoyl](1c)*SC(2a)WQC(2a)[Y(2-aminoethoxy)][2- |
| | Nal][2-Me-Leu]E(1c)S |
| SEQ ID NO: 45 | [(3-Aminomethyl)benzoyl](1c)*SC(2a)WQC(2a)[Y(2-aminoethoxy)][2- |
| | Nal][2-Me-Leu]E(1c)[F(4-NH2)] |
| SEQ ID NO: 46 | [(3-Aminomethyl)benzoyl](1c)*SC(2a)WQC(2a)[Y(2-aminoethoxy)][2- |
| | Nal][{d}L]E(1c)Y |
| SEQ ID NO: 47 | [Ac]-K(1c)SC(2a)WQC(2a)[Y(2-aminoethoxy)][2-Nal][2-Me-Leu]E(1c)R |
| SEQ ID NO: 48 | [Ac]-K(1c)SC(2a)WQC(2a)[Y(2-aminoethoxy)][2-Nal][{d}L]E(1c)R |
| SEQ ID NO: 49 | [(4-Aminomethyl)benzoyl](1c)*SC(2a)WQC(2a)[Y(2-aminoethoxy)][2- |
| | Nal][2-Me-Leu]E(1c)R |
| SEQ ID NO: 50 | [4-(2-Aminoethyl)benzoyl](1c)*SC(2a)WQC(2a)[Y(2-aminoethoxy)][2- |
| | Nal][2-Me-Leu]E(1c)R |
| SEQ ID NO: 51 | [3-(4-Aminophenyl)propanoyl](1c)*SC(2a)WQC(2a)[Y(2-aminoethoxy)][2- |
| | Nal][2-Me-Leu]E(1c)R |
| SEQ ID NO: 52 | [4-Aminomethyl-phenylacetyl](1c)*SC(2a)WQC(2a)[Y(2-aminoethoxy)][2- |
| | Nal][2-Me-Leu]E(1c)R |
| SEQ ID NO: 53 | DC(1a)SC(2b)WQC(2b)WWLC(1a)R |
| SEQ ID NO: 54 | K(1c)SC(2b)WQC(2b)[Y(2-aminoethoxy)][2-Nal]LE(1c)R |
| SEQ ID NO: 55 | DE(1c)QC(2a)WQC(2a)YW[2-Me-Leu]K(1c)R |
| SEQ ID NO: 56 | DE(1c)QC(2a)WQC(2a)[2-Nal]W[2-Me-Leu]K(1c)R |
| SEQ ID NO: 57 | DE(1c)TC(2a)WQC(2a)W[2-Nal][2-Me-Leu]K(1c) |
| SEQ ID NO: 58 | DE(1c)SC(2a)WQC(2a)W[2-Nal][2-Me-Leu]K(1c)ENG |
| SEQ ID NO: 59 | DE(1c)SC(2a)WQC(2a)W[2-Nal][2-Me-Leu]K(1c)NG |
| SEQ ID NO: 60 | DE(1c)SC(2a)WQC(2a)W[2-Nal][2-Me-Leu]K(1c)G |
| SEQ ID NO: 61 | DE(1c)SC(2a)WQC(2a)W[2-Nal][2-Me-Lys]K(1c)ENG |
| SEQ ID NO: 62 | DC(1a)QC(2a)WQC(2a)[2-Nal]W[2-Me-Leu]C(1a)R |
| SEQ ID NO: 63 | DC(1a)QC(2a)WQC(2a)YW[2-Me-Leu]C(1a)R |
| SEQ ID NO: 64 | DC(1a)QC(2a)WQC(2a)WW[2-Me-Leu]C(1a)ENG |
| SEQ ID NO: 65 | [(3-Aminomethyl)benzoyl](1c)*S[Dpr](2c)WQE(2c)[Y(2-aminoethoxy)][2- |
| | Nal][2-Me-Leu]E(1c)R |
| SEQ ID NO: 66 | [(3-Aminomethyl)benzoyl](1c)*SE(2c)WQ[Dpr](2c)[Y(2-aminoethoxy)][2- |
| | Nal][2-Me-Leu]E(1c)R |
| SEQ ID NO: 67 | [(3-Aminomethyl)benzoyl](1c)*SE(2c)WQ[Dab](2c)[Y (2-aminoethoxy)][2- |
| | Nal][2-Me-Leu]E(1c)R |
| SEQ ID NO: 68 | [(3-Aminomethyl)benzoyl](1c)*S[Orn](2c)WQE(2c)[Y(2-aminoethoxy)][2- |
| | Nal][2-Me-Leu]E(1c)R |
| SEQ ID NO: 69 | [(3-Aminomethyl)benzoyl](1c)*SE(2c)WQ[Orn](2c)[Y(2-aminoethoxy)][2- |
| | Nal][2-Me-Leu]E(1c)R |
| SEQ ID NO: 70 | [(3-Aminomethyl)benzoyl](1c)*SC(2a)[7-Me-Trp]QC(2a)[Y (2- |
| | aminoethoxy)][2-Nal][2-Me-Leu]E(1c)R |
| SEQ ID NO: 71 | [(3-Aminomethyl)benzoyl](1c)*SC(2a)[7-Ph-Trp]QC(2a)[Y(2- |
| | aminoethoxy)][2-Nal][2-Me-Leu]E(1c)R |
| SEQ ID NO: 72 | [(3-Aminomethyl)benzoyl](1c)*SC(2a)[7-(Naphth-2-yl)-Trp]QC(2a)[Y(2- |
| | aminoethoxy)][2-Nal][2-Me-Leu]E(1c)R |
| SEQ ID NO: 73 | [(3-Aminomethyl)benzoyl](1c)*SC(2a)WQC(2a)[Y(2-aminoethoxy)][3-(3- |
| | Quinolinyl)-Ala][2-Me-Leu]E(1c)R |
| SEQ ID NO: 74 | [(3-Aminomethyl)benzoyl](1c)*SC(2a)WQC(2a)[Y(2-aminoethoxy)][6-CI- |
| | Trp][2-Me-Leu]E(1c)R |
| SEQ ID NO: 75 | [(3-Aminomethyl)benzoyl](1c)*SC(2a)WQC(2a)[Y(2-aminoethoxy)][2- |
| | Nal][4-aminotetrahydro-2H-pyran-4-acetyl]E(1c)R |
| SEQ ID NO: 76 | [(3-Aminomethyl)benzoyl](1c)*SC(2a)WQC(2a)[Y(2-aminoethoxy)][2- |
| | Nal][2-Me-Leu]E(1c)[3-(3-Pyridyl)-Ala] |
| SEQ ID NO: 77 | [(3-Aminomethyl)benzoyl](1c)*SC(2a)WQC(2a)[Y(2-aminoethoxy)][2- |
| | Nal][2-Me-Leu]E(1c)[3-(4-Pyridyl)-Ala] |
| SEQ ID NO: 78 | [(3-Aminomethyl)benzoyl](1c)*SC(2a)WQC(2a)[Y(2-aminoethoxy)][2- |
| | Nal][2-Me-Leu]E(1c)R[3-(3-Pyridyl)-Ala] |
| SEQ ID NO: 79 | [(3-Aminomethyl)benzoyl](1c)*SC(2a)WQC(2a)[Y(2-aminoethoxy)][2- |
| | Nal][2-Me-Leu]E(1c)R[3-(4-Pyridyl)-Ala] |
| SEQ ID NO: 80 | [4-Aminomethyl-phenylacetyl](1c)*SC(2a)WQC(2a)[Y(2-aminoethoxy)][2- |
| | Nal][2-Me-Leu]E(1c)[{d}R] |
| SEQ ID NO: 81 | [4-Aminomethyl-phenylacetyl](1c)*SC(2a)WQC(2a)[Y(2-aminoethoxy)][2- |
| | Nal][2-Me-Leu]E(1c)S |
| SEQ ID NO: 82 | [4-Aminomethyl-phenylacetyl](1c)*SC(2a)WQC(2a)[Y(2-aminoethoxy)][2- |
| | Nal][2-Me-Leu]E(1c)[Dab] |
| SEQ ID NO: 83 | [4-Aminomethyl-phenylacetyl](1c)*TC(2a)WQC(2a)[Y(2-aminoethoxy)][2- |
| | Nal][2-Me-Leu]E(1c)[{d}R] |
| SEQ ID NO: 84 | K(1c)SC(2a)WQC(2a)[Y(2-aminoethoxy)][2-Nal][2-Me-Leu]E(1c)[{d}R] |
| SEQ ID NO: 85 | K(1c)TC(2a)WQC(2a)[Y(2-aminoethoxy)][2-Nal][2-Me-Leu]E(1c)[{d}R] |
| SEQ ID NO: 86 | K(1c)SC(2a)WQC(2a)[Y(2-aminoethoxy)][2-Nal][2-Me-Leu]E(1c)S |
| SEQ ID NO: 87 | [6-Aminohexanoyl](1c)*SC(2a)WQC(2a)[Y(2-aminoethoxy)][2-Nal][2-Me- |
| | Leu]E(1c)[{d}R] |
| SEQ ID NO: 88 | [6-Amino-4-oxahexanoyl](1c)*SC(2a)WQC(2a)[Y(2-aminoethoxy)][2- |
| | Nal][2-Me-Leu]E(1c)[{d}R] |
| SEQ ID NO: 89 | [trans-4-Aminomethyl-cyclohexyl-1-carbonyl](1c)*[beta-homo- |
| | Ser]C(2a)WQC(2a)[Y(2-aminoethoxy)][2-Nal][2-Me-Leu]E(1c)[{d}R] |
| SEQ ID NO: 90 | [(4-(2-Aminoethyl)-piperazine-1-yl)-acetyl](1c)*SC(2a)WQC(2a)[Y(2- |
| | aminoethoxy)][2-Nal][2-Me-Leu]E(1c)[{d}R] |
| SEQ ID NO: 91 | [(4-(2-Aminoethyl)-piperazine-1-yl)-acetyl](1c)*SC(2a)WQC(2a)[Y(2- |
| | aminoethoxy)][2-Nal][2-Me-Leu]D(1c)[{d}R] |
| SEQ ID NO: 92 | [3-Aminomethyl-phenylacetyl](1c)*SC(2a)WQC(2a)[Y(2-aminoethoxy)][2- |
| | Nal][2-Me-Leu]E(1c)[{d}R] |
| SEQ ID NO: 93 | [2-Aminomethyl-phenylacetyl](1c)*SC(2a)WQC(2a)[Y(2-aminoethoxy)][2- |
| | Nal][2-Me-Leu]E(1c)[{d}R] |
| SEQ ID NO: 94 | [K(N3)](1g)SC(2a)WQC(2a)[Y(2-aminoethoxy)][2-Nal][2-Me- |
| | Leu][Pra](1g)[{d}R] |
| SEQ ID NO: 95 | [Azidoacetic acid](1g)*SC(2a)WQC(2a)[Y(2-aminoethoxy)][2-Nal][2-Me- |
| | Leu][Glu(propargylamine)](1g)[{d}R] |
| SEQ ID NO: 96 | [Azidoacetic acid](1g)*SC(2a)WQC(2a)[Y(2-aminoethoxy)][2-Nal][2-Me- |
| | Leu][Dab(3-butynoic acid)] (1g)[{d}R] |
| SEQ ID NO: 97 | [but-3-ynoic acid](1g)*SC(2a)WQC(2a)[Y(2-aminoethoxy)][2-Nal][2-Me- |
| | Leu][Dab(azidoacetic acid)](1g)[{d}R] |
| SEQ ID NO: 98 | [(N3)-Ala](1g)*SC(2a)WQC(2a)[Y(2-aminoethoxy)][2-Nal][2-Me- |
| | Leu][Glu(propargylamine)](1g)[{d}R] |
| SEQ ID NO: 99 | [(N3)-Ala](1g)*SC(2a)WQC(2a)[Y(2-aminoethoxy)][2-Nal][2-Me- |
| | Leu][Dab(3-butynoic acid)](1g)[{d}R] |
| SEQ ID NO: 100 | [but-3-ynoic acid](1g)*SC(2a)WQC(2a)[Y(2-aminoethoxy)][2-Nal][2-Me- |
| | Leu][Dab((N3)-Ala))](1g)[{d}R] |
| SEQ ID NO: 101 | [4-Aminomethyl-2-pyridineacetyl](1c)*SC(2a)WQC(2a)[Y(2- |
| | aminoethoxy)][2-Nal][2-Me-Leu]E(1c)[{d}R] |
| SEQ ID NO: 102 | [4-Aminomethyl-3-pyridineacetyl](1c)*SC(2a)WQC(2a)[Y(2- |
| | aminoethoxy)][2-Nal][2-Me-Leu]E(1c)[{d}R] |
| SEQ ID NO: 103 | [4-Aminomethyl-2-fluoro-phenylacetyl](1c)*SC(2a)WQC(2a)[Y (2- |
| | aminoethoxy)][2-Nal][2-Me-Leu]E(1c)[{d}R] |
| SEQ ID NO: 104 | [4-Aminomethyl-3-methyl-phenylacetyl](1c)*SC(2a)WQC(2a)[Y(2- |
| | aminoethoxy)][2-Nal][2-Me-Leu]E(1c)[{d}R] |
| SEQ ID NO: 105 | [4-Aminomethyl-3-methoxy-phenylacetyl](1c)*SC(2a)WQC(2a)[Y(2- |
| | aminoethoxy)][2-Nal][2-Me-Leu]E(1c)[{d}R] |
| | or a pharmaceutically acceptable salt or solvate thereof; |
| | wherein: |

*= bridge uses the peptide backbone amine or carboxylic acid at the N- or C-terminus, not the side chain amine or carboxylic acid
(1a) = [2,11] 1,3-dithio-propan-2-one bridge; (1c) = [2,11] lactam bridge; (1g) = [2,11] 1,4-disubstituted 1,2,3-triazole bridge; (2a) = [4,7] 1,3-dithio-propan-2-one bridge; (2c) = [4,7] lactam bridge; (2d) = [4,7] 1,2-phenylenedimethanethiol bridge; (2e) = [4,7] 1,3-phenylenedimethanethiol bridge; (2f) = [4,7] 1,4-phenylenedimethanethiol bridge; (2g) = [4,7] 1,4-disubstituted 1,2,3-triazole bridge.

75. A compound according to clause 1 which is selected from:

| | |
|---|---|
| 1 | H-DC(1a)SC(2a)WQC(2a)WW[2-Me-Leu]C(1a)R-[NH2] |
| 2 | H-LC(1a)SC(2a)WQC(2a)WWLC(1a)R-[NH2] |
| 3 | H-DC(1a)SC(2a)WEC(2a)WWLC(1a)R-[NH2] |
| 4 | H-DE(1c)SC(2a)WQC(2a)WWLK(1c)R-[NH2] |
| 5 | H-DC(1a)SE(2c)WQK(2c)WWLC(1a)R-[NH2] |
| 6 | D(1c)*SC(2a)WQC(2a)WWLR(1c)* |
| 7 | [Ac]-DK(1c)SC(2a)WQC(2a)WWLE(1c)R-[NH2] |
| 8 | [Ac]-D[Orn](1c)SC(2a)WQC(2a)WWLE(1c)R-[NH2] |
| 9 | H-DE(1c)SC(2a)WQC(2a)WWL[Dab](1c)R-[NH2] |
| 10 | [Ac]-E(1c)SC(2a)WQC(2a)WWLK(1c)R-[NH2] |
| 11 | [Ac]-DE(1c)SC(2a)WQC(2a)WWLK(1c)-[NH2] |
| 12 | [Ac]-E(1c)SC(2a)WQC(2a)WWLK(1c)-[NH2] |
| 13 | [Ac]-DE(1c)SC(2a)AQC(2a)WWLK(1c)R-[NH2] |
| 14 | [Ac]-DE(1c)SC(2a)WQC(2a)AWLK(1c)R-[NH2] |
| 15 | [Ac]-DE(1c)SC(2a)WQC(2a)WALK(1c)R-[NH2] |
| 16 | [Ac]-DE(1c)SC(2a)[7-Me-Trp]QC(2a)WWLK(1c)R-[NH2] |
| 17 | [Ac]-DE(1c)SC(2a)WQC(2a)W[2-Nal]LK(1c)R-[NH2] |
| 18 | [Ac]-K(1c)SC(2a)WQC(2a)W[2-Nal]LE(1c)R-[NH2] |
| 19 | [Ac]-DK(1c)SC(2a)WQC(2a)W[2-Nal]LE(1c)R-[NH2] |
| 20 | [Ac]-DK(1c)SC(2d)WQC(2d)W[2-Nal]LE(1c)R-[NH2] |
| 21 | [Ac]-DK(1c)SC(2e)WQC(2e)W[2-Nal]LE(1c)R-[NH2] |
| 22 | [Ac]-DK(1c)SC(2f)WQC(2f)W[2-Nal]LE(1c)R-[NH2] |
| 23 | [Ac]-DK(1c)S[K(N₃)](2g)WQ[Pra](2g)W[2-Nal]LE(1c)R-[NH2] |
| 24 | [Ac]-DK(1c)S[Aha](2g)WQ[Pra](2g)W[2-Nal]LE(1c)R-[NH2] |
| 25 | [Ac]-K(1c)SC(2a)[2-Nal]QC(2a)W[2-Nal]LE(1c)R-[NH2] |
| 26 | [Ac]-K(1c)SC(2a)[Bip]QC(2a)W[2-Nal]LE(1c)R-[NH2] |
| 27 | [Ac]-K(1c)SC(2a)[1-Me-Trp]QC(2a)W[2-Nal]LE(1c)R-[NH2] |
| 28 | [Ac]-DK(1c)SC(2a)WQC(2a)[2-Nal][2-Nal]LE(1c)R-[NH2] |
| 29 | [Ac]-K(1c)SC(2a)WQC(2a)[Y(2-aminoethoxy)][2-Nal]LE(1c)R-[NH2] |
| 30 | [Ac]-K(1c)SC(2a)WQC(2a)H[2-Nal]LE(1c)R-[NH2] |
| 31 | [Ac]-K(1c)SC(2a)WQC(2a)W[F(4-F)]LE(1c)R-[NH2] |
| 32 | [Ac]-K(1c)SC(2a)WQC(2a)W[1-Me-Trp]LE(1c)R-[NH2] |
| 33 | [Ac]-C(1a)TC(2a)WEC(2a)WW[2-Me-Leu]C(1a)S-[NH2] |
| 34 | [Ac]-GK(1c)TC(2a)WEC(2c)WW[2-Me-Leu]E(1c)S-[NH2] |
| 35 | [Ac]-GC(1a)TC(2a)WEC(2a)W[2-Nal][2-Me-Leu]C(1a)R-[NH2] |
| 36 | [bAla](1c)*SC(2a)WQC(2a)W[2-Nal]LE(1c)R-[NH2] |
| 37 | [(3-Aminomethyl)benzoyl](1c)*SC(2a)WQC(2a)W[2-Nal]LE(1c)R-[NH2] |
| 38 | [(3-Aminomethyl)benzoyl](1c)*SC(2a)WQC(2a)[Y(2-aminoethoxy)][2- |
| | Nal][2-Me-Leu]E(1c)R-[NH2] |
| 39 | [(3-Aminomethyl)benzoyl](1c)*SC(2a)WQC(2a)[Y(2-aminoethoxy)][2- |
| | Nal]LE(1c)R-[NH2] |
| 40 | [(3-Aminomethyl)benzoyl](1c)*SC(2a)WQC(2a)W[2-Nal][2-Me- |
| | Leu]E(1c)R-[NH2] |
| 41 | [(3-Aminomethyl)benzoyl](1c)*SC(2a)WQC(2a)[Y(2-aminoethoxy)][2- |
| | Nal][2-Me-Leu]E(1c)[{d}R]-[NH2] |
| 42 | [(3-Aminomethyl)benzoyl](1c)*SC(2a)WQC(2a)[Y(2-aminoethoxy)][2- |
| | Nal][2-Me-Leu]E(1c)[2-Me-Arg]-[NH2] |
| 43 | [(3-Aminomethyl)benzoyl](1c)*SC(2a)WQC(2a)[Y(2-aminoethoxy)][2- |
| | Nal][2-Me-Leu]E(1c)[N-Me-Arg]-[NH2] |
| 44 | [(3-Aminomethyl)benzoyl](1c)*SC(2a)WQC(2a)[Y(2-aminoethoxy)][2- |
| | Nal][2-Me-Leu]E(1c)S-[NH2] |
| 45 | [(3-Aminomethyl)benzoyl](1c)*SC(2a)WQC(2a)[Y(2-aminoethoxy)][2- |
| | Nal][2-Me-Leu]E(1c)[F(4-NH₂)]-[NH2] |
| 46 | [(3-Aminomethyl)benzoyl](1c)*SC(2a)WQC(2a)[Y(2-aminoethoxy)][2- |
| | Nal][{d}L]E(1c)Y-[NH2] |
| 47 | [Ac]-K(1c)SC(2a)WQC(2a)[Y(2-aminoethoxy)][2-Nal][2-Me-Leu]E(1c)R- |
| | [NH2] |
| 48 | [Ac]-K(1c)SC(2a)WQC(2a)[Y(2-aminoethoxy)][2-Nal][{d}L]E(1c)R-[NH2] |
| 49 | [(4-Aminomethyl)benzoyl](1c)*SC(2a)WQC(2a)[Y(2-aminoethoxy)][2- |
| | Nal][2-Me-Leu]E(1c)R-[NH2] |
| 50 | [4-(2-Aminoethyl)benzoyl](1c)*SC(2a)WQC(2a)[Y(2-aminoethoxy)][2- |
| | Nal][2-Me-Leu]E(1c)R-[NH2] |
| 51 | [3-(4-Aminophenyl)propanoyl](1c)*SC(2a)WQC(2a)[Y(2-aminoethoxy)][2- |
| | Nal][2-Me-Leu]E(1c)R-[NH2] |
| 52 | [4-Aminomethyl-phenylacetyl](1c)*SC(2a)WQC(2a)[Y(2-aminoethoxy)][2- |
| | Nal][2-Me-Leu]E(1c)R-[NH2] |
| 53 | H-DC(1a)SC(2b)WQC(2b)WWLC(1a)R-[NH2] |
| 54 | [Ac]-K(1c)SC(2b)WQC(2b)[Y(2-aminoethoxy)][2-Nal]LE(1c)R-[NH2] |
| 55 | [Ac]-DE(1c)QC(2a)WQC(2a)YW[2-Me-Leu]K(1c)R-[NH2] |
| 56 | [Ac]-DE(1c)QC(2a)WQC(2a)[2-Nal]W[2-Me-Leu]K(1c)R-[NH2] |
| 57 | [Ac]-DE(1c)TC(2a)WQC(2a)W[2-Nal][2-Me-Leu]K(1c)R-[NH2] |
| 58 | [Ac]-DE(1c)SC(2a)WQC(2a)W[2-Nal][2-Me-Leu]K(1c)ENG-[NH2] |
| 59 | [Ac]-DE(1c)SC(2a)WQC(2a)W[2-Nal][2-Me-Leu]K(1c)NG-[NH2] |
| 60 | [Ac]-DE(1c)SC(2a)WQC(2a)W[2-Nal][2-Me-Leu]K(1c)G-[NH2] |
| 61 | [Ac]-DE(1c)SC(2a)WQC(2a)W[2-Nal][2-Me-Lys]K(1c)ENG-[NH2] |
| 62 | [Ac]-DC(1a)QC(2a)WQC(2a)[2-Nal]W[2-Me-Leu]C(1a)R-[NH2] |
| 63 | [Ac]-DC(1a)QC(2a)WQC(2a)YW[2-Me-Leu]C(1a)R-[NH2] |
| 64 | [Ac]-DC(1a)QC(2a)WQC(2a)WW[2-Me-Leu]C(1a)ENG-[NH2] |
| 65 | [(3-Aminomethyl)benzoyl](1c)*S[Dpr](2c)WQE(2c)[Y(2-aminoethoxy)][2- |
| | Nal][2-Me-Leu]E(1c)R-[NH2] |
| 66 | [(3-Aminomethyl)benzoyl](1c)*SE(2c)WQ[Dpr](2c)[Y(2-aminoethoxy)][2- |
| | Nal][2-Me-Leu]E(1c)R-[NH2] |
| 67 | [(3-Aminomethyl)benzoyl](1c)*SE(2c)WQ[Dab](2c)[Y(2-aminoethoxy)][2- |
| | Nal][2-Me-Leu]E(1c)R-[NH2] |
| 68 | [(3-Aminomethyl)benzoyl](1c)*S[Orn](2c)WQE(2c)[Y(2-aminoethoxy)][2- |
| | Nal][2-Me-Leu]E(1c)R-[NH2] |
| 69 | [(3-Aminomethyl)benzoyl](1c)*SE(2c)WQ[Orn](2c)[Y(2-aminoethoxy)][2- |
| | Nal][2-Me-Leu]E(1c)R-[NH2] |
| 70 | [(3-Aminomethyl)benzoyl](1c)*SC(2a)[7-Me-Trp]QC(2a)[Y(2- |
| | aminoethoxy)][2-Nal][2-Me-Leu]E(1c)R-[NH2] |
| 71 | [(3-Aminomethyl)benzoyl](1c)*SC(2a)[7-Ph-Trp]QC(2a)[Y(2- |
| | aminoethoxy)][2-Nal][2-Me-Leu]E(1c)R-[NH2] |
| 72 | [(3-Aminomethyl)benzoyl](1c)*SC(2a)[7-(Naphth-2-yl)-Trp]QC(2a)[Y(2- |
| | aminoethoxy)][2-Nal][2-Me-Leu]E(1c)R-[NH2] |
| 73 | [(3-Aminomethyl)benzoyl](1c)*SC(2a)WQC(2a)[Y(2-aminoethoxy)][3-(3- |
| | Quinolinyl)-Ala][2-Me-Leu]E(1c)R-[NH2] |
| 74 | [(3-Aminomethyl)benzoyl](1c)*SC(2a)WQC(2a)[Y(2-aminoethoxy)][6-Cl- |
| | Trp][2-Me-Leu]E(1c)R-[NH2] |
| 75 | [(3-Aminomethyl)benzoyl](1c)*SC(2a)WQC(2a)[Y(2-aminoethoxy)][2- |
| | Nal][4-aminotetrahydro-2H-pyran-4-acetyl]E(1c)R-[NH2] |
| 76 | [(3-Aminomethyl)benzoyl](1c)*SC(2a)WQC(2a)[Y(2-aminoethoxy)][2- |
| | Nal][2-Me-Leu]E(1c)[3-(3-Pyridyl)-Ala]-[NH2] |
| 77 | [(3-Aminomethyl)benzoyl](1c)*SC(2a)WQC(2a)[Y(2-aminoethoxy)][2- |
| | Nal][2-Me-Leu]E(1c)[3-(4-Pyridyl)-Ala]-[NH2] |
| 78 | [(3-Aminomethyl)benzoyl](1c)*SC(2a)WQC(2a)[Y(2-aminoethoxy)][2- |
| | Nal][2-Me-Leu]E(1c)R[3-(3-Pyridyl)-Ala]-[NH2] |
| 79 | [(3-Aminomethyl)benzoyl](1c)*SC(2a)WQC(2a)[Y(2-aminoethoxy)][2- |
| | Nal][2-Me-Leu]E(1c)R[3-(4-Pyridyl)-Ala]-[NH2] |
| 80 | [4-Aminomethyl-phenylacetyl](1c)*SC(2a)WQC(2a)[Y(2-aminoethoxy)][2- |
| | Nal][2-Me-Leu]E(1c)[{d}R]-[NH2] |
| 81 | [4-Aminomethyl-phenylacetyl](1c)*SC(2a)WQC(2a)[Y(2-aminoethoxy)][2- |
| | Nal][2-Me-Leu]E(1c)S-[NH2] |
| 82 | [4-Aminomethyl-phenylacetyl](1c)*SC(2a)WQC(2a)[Y(2-aminoethoxy)][2- |
| | Nal][2-Me-Leu]E(1c)[Dab]-[NH2] |
| 83 | [4-Aminomethyl-phenylacetyl](1c)*TC(2a)WQC(2a)[Y(2-aminoethoxy)][2- |
| | Nal][2-Me-Leu]E(1c)[{d}R]-[NH2] |
| 84 | [Ac]-K(1c)SC(2a)WQC(2a)[Y(2-aminoethoxy)][2-Nal][2-Me- |
| | Leu]E(1c)[{d}R]-[NH2] |
| 85 | [Ac]-K(1c)TC(2a)WQC(2a)[Y(2-aminoethoxy)][2-Nal][2-Me- |
| | Leu]E(1c)[{d}R]-[NH2] |
| 86 | [Ac]-K(1c)SC(2a)WQC(2a)[Y(2-aminoethoxy)][2-Nal][2-Me-Leu]E(1c)S- |
| | [NH2] |
| 87 | [6-Aminohexanoyl](1c)*SC(2a)WQC(2a)[Y(2-aminoethoxy)][2-Nal][2-Me- |
| | Leu]E(1c)[{d}R]-[NH2] |
| 88 | [6-Amino-4-oxahexanoy]](1c)*SC(2a)WQC(2a)[Y(2-aminoethoxy)][2- |
| | Nal][2-Me-Leu]E(1c)[{d}R]-[NH2] |
| 89 | [trans-4-Aminomethyl-cyclohexyl-1-carbonyl](1c)*[beta-homo- |
| | Ser]C(2a)WQC(2a)[Y(2-aminoethoxy)][2-Nal][2-Me-Leu]E(1c)[{d}R]-[NH2] |
| 90 | [(4-(2-Aminoethyl)-piperazine-1-yl)-acetyl](1c)*SC(2a)WQC(2a)[Y(2- |
| | aminoethoxy)][2-Nal][2-Me-Leu]E(1c)[{d}R]-[NH2] |
| 91 | [(4-(2-Aminoethyl)-piperazine-1-yl)-acetyl](1c)*SC(2a)WQC(2a)[Y(2- |
| | aminoethoxy)][2-Nal][2-Me-Leu]D(1c)[{d}R]-[NH2] |
| 92 | [3-Aminomethyl-phenylacetyl](1c)*SC(2a)WQC(2a)[Y(2-aminoethoxy)][2- |
| | Nal][2-Me-Leu]E(1c)[{d}R]-[NH2] |
| 93 | [2-Aminomethyl-phenylacetyl](1c)*SC(2a)WQC(2a)[Y(2-aminoethoxy)][2- |
| | Nal][2-Me-Leu]E(1c)[{d}R]-[NH2] |
| 94 | [Ac]-[K(N₃)](1g)SC(2a)WQC(2a)[Y(2-aminoethoxy)][2-Nal][2-Me- |
| | Leu][Pra](1g)[{d}R]-[NH2] |
| 95 | [Azidoacetic acid](1g)*SC(2a)WQC(2a)[Y(2-aminoethoxy)][2-Nal][2-Me- |
| | Leu][Glu(propargylamine)](1g)[{d}R]-[NH2] |
| 96 | [Azidoacetic acid](1g)*SC(2a)WQC(2a)[Y(2-aminoethoxy)][2-Nal][2-Me- |
| | Leu][Dab(3-butynoic acid)](1g)[{d}R]-[NH2] |
| 97 | [but-3-ynoic acid](1g)*SC(2a)WQC(2a)[Y(2-aminoethoxy)][2-Nal][2-Me- |
| | Leu][Dab(azidoacetic acid)](1g)[{d}R]-[NH2] |
| 98 | [(N₃)-Ala](1g)*SC(2a)WQC(2a)[Y(2-aminoethoxy)][2-Nal][2-Me- |
| | Leu][Glu(propargylamine)](1g)[{d}R]-[NH2] |
| 99 | [(N₃)-Ala](1g)*SC(2a)WQC(2a)[Y(2-aminoethoxy)][2-Nal][2-Me- |
| | Leu][Dab(3-butynoic acid)](1g)[{d}R]-[NH2] |
| 100 | [but-3-ynoic acid](1g)*SC(2a)WQC(2a)[Y(2-aminoethoxy)][2-Nal][2-Me- |
| | Leu][Dab((N₃)-Ala))](1g)[{d}R]-[NH2] |
| 101 | [4-Aminomethyl-2-pyridineacetyl](1c)*SC(2a)WQC(2a)[Y(2- |
| | aminoethoxy)][2-Nal][2-Me-Leu]E(1c)[{d}R]-[NH2] |
| 102 | [4-Aminomethyl-3-pyridineacetyl](1c)*SC(2a)WQC(2a)[Y(2- |
| | aminoethoxy)][2-Nal][2-Me-Leu]E(1c)[{d}R]-[NH2] |
| 103 | [4-Aminomethyl-2-fluoro-phenylacetyl](1c)*SC(2a)WQC(2a)[Y(2- |
| | aminoethoxy)][2-Nal][2-Me-Leu]E(1c)[{d}R]-[NH2] |
| 104 | [4-Aminomethyl-3-methyl-phenylacetyl](1c)*SC(2a)WQC(2a)[Y(2- |
| | aminoethoxy)][2-Nal][2-Me-Leu]E(1c)[{d}R]-[NH2] |
| 105 | [4-Aminomethyl-3-methoxy-phenylacetyl](1c)*SC(2a)WQC(2a)[Y(2- |
| | aminoethoxy)][2-Nal][2-Me-Leu]E(1c)[{d}R]-[NH2] |

and pharmaceutically acceptable salts and solvates thereof;
wherein:
*= bridge uses the peptide backbone amine or carboxylic acid at the N- or C-terminus, not the side chain amine or carboxylic acid
(1a) = [2,11] 1,3-dithio-propan-2-one bridge; (1c) = [2,11] lactam bridge; (1g) = [2,11] 1,4-disubstituted 1,2,3-triazole bridge; (2a) = [4,7] 1,3-dithio-propan-2-one bridge; (2c) = [4,7] lactam bridge; (2d) = [4,7] 1,2-phenylenedimethanethiol bridge; (2e) = [4,7] 1,3-phenylenedimethanethiol bridge; (2f) = [4,7] 1,4-phenylenedimethanethiol bridge; (2g) = [4,7] 1,4-disubstituted 1,2,3-triazole bridge.

76. A pharmaceutical composition comprising a compound according to any one of the preceding clauses in combination with a pharmaceutically acceptable carrier, excipient or vehicle.
77. A method for the synthesis of a compound according to any one of clauses 1 to 75, comprising synthesising the analogue by solid-phase or liquid-phase peptide synthesis methodology, optionally isolating and/or purifying the final product, and optionally further comprising the step of forming an amide bond, forming two thioether bonds with a linker, or forming a triazole between the amino acid residues at positions X2 and X11, and optionally further comprising the step of forming an amide bond, forming two thioether bonds with a linker, or forming a triazole between the amino acid residues at positions X4 and X7.
78. A compound according to any one of clauses 1 to 75, or a pharmaceutical composition according to clause 76, for use in a method of medical treatment.
79. A compound according to any one of clauses 1 to 75, or a pharmaceutical composition according to clause 76, for use in a method of prevention or treatment of Inflammatory Bowel Disease (IBD), ulcerative colitis, Crohn's disease, Celiac disease (nontropical Sprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-I, chronic granulomatous disease, glycogen storage disease type 1b, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, ankylosing spondylitis, or graft versus host disease in a subject, and combinations thereof.

### Priority Clauses

1. A compound of the formula:

R¹—Z—R²
wherein
R¹ is H, C₁₋₄ acyl, benzoyl, C₁₋₄ alkyl, or is absent;
R² is NHR³, OH, or is absent, wherein R³ is hydrogen or C₁₋₃ alkyl; and
Z is an amino acid sequence of formula I:

X1-X2-X3-X4-X5-X6-X7-X8-X9-X10-X11-X12-X13-X14 (1)
wherein
X1 is absent or is selected from the group consisting of Asp, Glu, Ser, Cys, and Lys;
X3 is selected from the group consisting of Ser, beta-homo-Ser, Thr, Leu, Cys, and Gln;
X5 is selected from the group consisting of Trp, Tyr, Ala, 1-Me-Trp, 7-Me-Trp, 7-Ph-Trp, 7-(Naphth-2-yl)-Trp, 2-Nal, and Bip;
X6 is selected from the group consisting of Gln, Glu, Tyr and Cys;
X8 is selected from the group consisting of Trp, Tyr, Asn, Ala, His, and 2-Nal, wherein the hydroxyl group of Tyr is optionally substituted with C₁₋₃ alkyl optionally substituted with NH₂;
X9 is selected from the group consisting of 2-Nal, Trp, 1-Me-Trp, 6-Cl-Trp, 3-(3-Quinolinyl)-Ala, Phe, 4-F-Phe, Glu, Cys, and Ala;
X10 is selected from the group consisting of Leu, D-Leu, 2-Me-Leu, 2-Me-Lys, Trp, Asn, Cys, and 4-aminotetrahydro-2H-pyran-4-acetyl;
X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent;
X13 is absent or is selected from the group consisting of Asn, Gly, 3-(3-Pyridyl)-Ala, and 3-(4-Pyridyl)-Ala;
X14 is absent or is Gly;
X2 and X11 are amino acid residues who together form a lactam bridge, a dithioether bridge, or a bridge containing a triazole ring; and
X4 and X7 are amino acid residues who together form a lactam bridge, a dithioether bridge, or a bridge containing a triazole ring;
or a pharmaceutically acceptable salt or solvate thereof;
wherein the compound is not:

I3(isomer 3)H-DC(1a)SC(2a)WQC(2a)WWLC(1a)R-[NH₂];

wherein (1a) is a [2,11]1,3-dithio-propan-2-one bridge and (2a) is a [4,7]1,3-dithio-propan-2-one bridge.
2. The compound according to clause 1, wherein X1 is absent or Asp;
   optionally wherein X3 is Ser;
   optionally wherein X5 is Trp;
   optionally wherein X6 is Gln;
   optionally wherein X8 is Tyr wherein the hydroxyl group of Tyr is optionally substituted with —CH₂CH₂NH₂;
   optionally wherein X9 is 2-Nal or Trp;
   optionally wherein X10 is Leu or 2-Me-Leu;
   optionally wherein X12 is Arg;
   optionally wherein X13 is absent;
   optionally wherein X14 is absent;
   optionally wherein R¹ is H, C₁₋₂ acyl, or absent;
   optionally wherein R² is NH₂;
   optionally wherein the length of the bridge between X2 and X11 and/or X4 and X7 is 5 to atoms long.
3. The compound according to any one of the preceding clauses, wherein the dithioether bridge between X2 and X11 and/or X4 and X7 is of the formula —S—L—Y—L—S—, wherein:
   each S is a sulfur atom and is part of the amino acid residue at X2 and X11 and/or X4 and X7;
   each L is independently C₁₋₄ alkylene; and
   Y is either absent, C(═O), or arylene.
4. The compound according to any one of the preceding clauses, wherein the bridge containing a triazole ring between X2 and X11 and/or X4 and X7 contains a 1,2,3-triazole ring;
   optionally wherein the bridge containing a triazole ring between X2 and X11 and/or X4 and X7 is attached to positions 1 and 4 of the triazole ring.
5. The compound according to any one of the preceding clauses, wherein X2 and X11 are amino acid residues who together form a lactam bridge;
   optionally wherein the location of the amide bond in the lactam bridge is closer to X11 than X2;
   optionally wherein one of the residues at position X2 and X11 is selected from Lys, Arg, Orn, bAla, 3-(4-aminophenyl)propanoyl, (3-aminomethyl)benzoyl, (4-aminomethyl)benzoyl, 4-(2-aminoethyl)benzoyl, 2-aminomethyl-phenylacetyl, 3-aminomethyl-phenylacetyl, 4-aminomethyl-phenylacetyl, 4-aminomethyl-2-pyridineacetyl, 4-aminomethyl-3-pyridineacetyl, 4-aminomethyl-2-fluoro-phenylacetyl, 4-aminomethyl-3-fluoro-phenylacetyl, 4-aminomethyl-2-methyl-phenylacetyl, 4-aminomethyl-3-methyl-phenylacetyl, 4-aminomethyl-2-methoxy-phenylacetyl, 4-aminomethyl-3-methoxy-phenylacetyl, Dab, 6-aminohexanoyl, 6-amino-4-oxahexanoyl, trans-4-aminomethyl-cyclohexyl-1-carbonyl, and (4-(2-aminoethyl)-piperazine-1-yl)-acetyl, and the other is selected from Glu and Asp.
6. The compound according to any one clauses 1 to 4, wherein X2 and X11 are amino acid residues who together form a dithioether bridge;
   optionally wherein X2 is Cys and X11 is Cys.
7. The compound according to any one clauses 1 to 4, wherein X2 and X11 are amino acid residues who together form a bridge containing a triazole ring;
   optionally wherein one of the residues at position X2 and X11 is selected from Lys(N₃), azidoacetic acid, (N₃)-Ala, Dab(azidoacetic acid), and Dab((N₃)-Ala), and the other is selected from Pra, Glu(propargylamine), and Dab(3-butynoic acid).
8. The compound according to any one of the preceding clauses, wherein X4 and X7 are amino acid residues who together form a dithioether bridge;
   optionally wherein X4 is Cys and X7 is Cys;
   or
   wherein X4 and X7 are amino acid residues who together form a lactam bridge;
   optionally wherein one of the residues at position X4 and X7 is Lys, Dpr, Dab, or Orn, and the other is Glu.
9. The compound according to any one clauses 1 to 7, wherein X4 and X7 are amino acid residues who together form a bridge containing a triazole ring;
   optionally wherein one of the residues at position X4 and X7 is selected from Lys(N₃) and Aha, and the other is Pra.
10. The compound according to clause 1 wherein Z is an amino acid sequence selected from the group consisting of:

| | |
|---|---|
| SEQ ID NO: 1 | DC(1a)SC(2a)WQC(2a)WW[2-Me-Leu]C(1a)R |
| SEQ ID NO: 2 | LC(1a)SC(2a)WQC(2a)WWLC(1a)R |
| SEQ ID NO: 3 | DC(1a)SC(2a)WEC(2a)WWLC(1a)R |
| SEQ ID NO: 4 | DE(1c)SC(2a)WQC(2a)WWLK(1c)R |
| SEQ ID NO: 5 | DC(1a)SE(2c)WQK(2c)WWLC(1a)R |
| SEQ ID NO: 6 | D(1c)*SC(2a)WQC(2a)WWLR(1c)* |
| SEQ ID NO: 7 | DK(1c)SC(2a)WQC(2a)WWLE(1c)R |
| SEQ ID NO: 8 | D[Orn](1c)SC(2a)WQC(2a)WWLE(1c)R |
| SEQ ID NO: 9 | DE(1c)SC(2a)WQC(2a)WWL[Dab](1c)R |
| SEQ ID NO: 10 | E(1c)SC(2a)WQC(2a)WWLK(1c)R |
| SEQ ID NO: 11 | DE(1c)SC(2a)WQC(2a)WWLK(1c) |
| SEQ ID NO: 12 | E(1c)SC(2a)WQC(2a)WWLK(1c) |
| SEQ ID NO: 13 | DE(1c)SC(2a)AQC(2a)WWLK(1c)R |
| SEQ ID NO: 14 | DE(1c)SC(2a)WQC(2a)AWLK(1c)R |
| SEQ ID NO: 15 | DE(1c)SC(2a)WQC(2a)WALK(1c)R |
| SEQ ID NO: 16 | DE(1c)SC(2a)[7-Me-Trp]QC(2a)WWLK(1c)R |
| SEQ ID NO: 17 | DE(1c)SC(2a)WQC(2a)W[2-Nal]LK(1c)R |
| SEQ ID NO: 18 | K(1c)SC(2a)WQC(2a)W[2-Nal]LE(1c)R |
| SEQ ID NO: 19 | DK(1c)SC(2a)WQC(2a)W[2-Nal]LE(1c)R |
| SEQ ID NO: 20 | DK(1c)SC(2d)WQC(2d)W[2-Nal]LE(1c)R |
| SEQ ID NO: 21 | DK(1c)SC(2e)WQC(2e)W[2-Nal]LE(1c)R |
| SEQ ID NO: 22 | DK(1c)SC(2f)WQC(2f)W[2-Nal]LE(1c)R |
| SEQ ID NO: 23 | DK(1c)S[K(N₃)](2g)WQ[Pra](2g)W[2-Nal]LE(1c)R |
| SEQ ID NO: 24 | DK(1c)S[Aha](2g)WQ[Pra](2g)W[2-Nal]LE(1c)R |
| SEQ ID NO: 25 | K(1c)SC(2a)[2-Nal]QC(2a)W[2-Nal]LE(1c)R |
| SEQ ID NO: 26 | K(1c)SC(2a)[Bip]QC(2a)W[2-Nal]LE(1c)R |
| SEQ ID NO: 27 | K(1c)SC(2a)[1-Me-Trp]QC(2a)W[2-Nal]LE(1c)R |
| SEQ ID NO: 28 | DK(1c)SC(2a)WQC(2a)[2-Nal][2-Nal]LE(1c)R |
| SEQ ID NO: 29 | K(1c)SC(2a)WQC(2a)[Y(2-aminoethoxy)][2-Nal]LE(1c)R |
| SEQ ID NO: 30 | K(1c)SC(2a)WQC(2a)H[2-Nal]LE(1c)R |
| SEQ ID NO: 31 | K(1c)SC(2a)WQC(2a)W[F(4-F)]LE(1c)R |
| SEQ ID NO: 32 | K(1c)SC(2a)WQC(2a)W[1-Me-Trp]LE(1c)R |
| SEQ ID NO: 33 | C(1a)TC(2a)WEC(2a)WW[2-Me-Leu]C(1a)S |
| SEQ ID NO: 34 | GK(1c)TC(2a)WEC(2c)WW[2-Me-Leu]E(1c)S |
| SEQ ID NO: 35 | GC(1a)TC(2a)WEC(2a)W[2-Nal][2-Me-Leu]C(1a)R |
| SEQ ID NO: 36 | [bAla](1c)*SC(2a)WQC(2a)W[2-Nal]LE(1c)R |
| SEQ ID NO: 37 | [(3-Aminomethyl)benzoyl](1c)*SC(2a)WQC(2a)W[2-Nal]LE(1c)R |
| SEQ ID NO: 38 | [(3-Aminomethyl)benzoyl](1c)*SC(2a)WQC(2a)[Y(2-aminoethoxy)][2- |
| | Nal][2-Me-Leu]E(1c)R-[NH2] |
| SEQ ID NO: 39 | [(3-Aminomethyl)benzoyl](1c)*SC(2a)WQC(2a)[Y(2-aminoethoxy)][2- |
| | Nal]LE(1c)R |
| SEQ ID NO: 40 | [(3-Aminomethyl)benzoyl](1c)*SC(2a)WQC(2a)W[2-Nal][2-Me-Leu]E(1c)R |
| SEQ ID NO: 41 | [(3-Aminomethyl)benzoyl](1c)*SC(2a)WQC(2a)[Y(2-aminoethoxy)][2- |
| | Nal][2-Me-Leu]E(1c)[{d}R] |
| SEQ ID NO: 42 | [(3-Aminomethyl)benzoyl](1c)*SC(2a)WQC(2a)[Y(2-aminoethoxy)][2- |
| | Nal][2-Me-Leu]E(1c)[2-Me-Arg] |
| SEQ ID NO: 43 | [(3-Aminomethyl)benzoyl](1c)*SC(2a)WQC(2a)[Y(2-aminoethoxy)][2- |
| | Nal][2-Me-Leu]E(1c)[N-Me-Arg] |
| SEQ ID NO: 44 | [(3-Aminomethyl)benzoyl](1c)*SC(2a)WQC(2a)[Y(2-aminoethoxy)][2- |
| | Nal][2-Me-Leu]E(1c)S |
| SEQ ID NO: 45 | [(3-Aminomethyl)benzoyl](1c)*SC(2a)WQC(2a)[Y(2-aminoethoxy)][2- |
| | Nal][2-Me-Leu]E(1c)[F(4-NH₂)] |
| SEQ ID NO: 46 | [(3-Aminomethyl)benzoyl](1c)*SC(2a)WQC(2a)[Y(2-aminoethoxy)][2- |
| | Nal][{d}L]E(1c)Y |
| SEQ ID NO: 47 | [Ac]-K(1c)SC(2a)WQC(2a)[Y(2-aminoethoxy)][2-Nal][2-Me-Leu]E(1c)R |
| SEQ ID NO: 48 | [Ac]-K(1c)SC(2a)WQC(2a)[Y(2-aminoethoxy)][2-Nal][{d}L]E(1c)R |
| SEQ ID NO: 49 | [(4-Aminomethyl)benzoyl](1c)*SC(2a)WQC(2a)[Y(2-aminoethoxy)][2- |
| | Nal][2-Me-Leu]E(1c)R |
| SEQ ID NO: 50 | [4-(2-Aminoethyl)benzoyl](1c)*SC(2a)WQC(2a)[Y(2-aminoethoxy)][2- |
| | Nal][2-Me-Leu]E(1c)R |
| SEQ ID NO: 51 | [3-(4-Aminophenyl)propanoyl](1c)*SC(2a)WQC(2a)[Y(2-aminoethoxy)][2- |
| | Nal][2-Me-Leu]E(1c)R |
| SEQ ID NO: 52 | [4-Aminomethyl-phenylacetyl](1c)*SC(2a)WQC(2a)[Y(2-aminoethoxy)][2- |
| | Nal][2-Me-Leu]E(1c)R |
| SEQ ID NO: 53 | DC(1a)SC(2b)WQC(2b)WWLC(1a)R |
| SEQ ID NO: 54 | K(1c)SC(2b)WQC(2b)[Y(2-aminoethoxy)][2-Nal]LE(1c)R |
| SEQ ID NO: 55 | DE(1c)QC(2a)WQC(2a)YW[2-Me-Leu]K(1c)R |
| SEQ ID NO: 56 | DE(1c)QC(2a)WQC(2a)[2-Nal]W[2-Me-Leu]K(1c)R |
| SEQ ID NO: 57 | DE(1c)TC(2a)WQC(2a)W[2-Nal][2-Me-Leu]K(1c) |
| SEQ ID NO: 58 | DE(1c)SC(2a)WQC(2a)W[2-Nal][2-Me-Leu]K(1c)ENG |
| SEQ ID NO: 59 | DE(1c)SC(2a)WQC(2a)W[2-Nal][2-Me-Leu]K(1c)NG |
| SEQ ID NO: 60 | DE(1c)SC(2a)WQC(2a)W[2-Nal][2-Me-Leu]K(1c)G |
| SEQ ID NO: 61 | DE(1c)SC(2a)WQC(2a)W[2-Nal][2-Me-Lys]K(1c)ENG |
| SEQ ID NO: 62 | DC(1a)QC(2a)WQC(2a)[2-Nal]W[2-Me-Leu]C(1a)R |
| SEQ ID NO: 63 | DC(1a)QC(2a)WQC(2a)YW[2-Me-Leu]C(1a)R |
| SEQ ID NO: 64 | DC(1a)QC(2a)WQC(2a)WW[2-Me-Leu]C(1a)ENG |
| SEQ ID NO: 65 | [(3-Aminomethyl)benzoyl](1c)*S[Dpr](2c)WQE(2c)[Y(2-aminoethoxy)][2- |
| | Nal][2-Me-Leu]E(1c)R |
| SEQ ID NO: 66 | [(3-Aminomethyl)benzoyl](1c)*SE(2c)WQ[Dpr](2c)[Y(2-aminoethoxy)][2- |
| | Nal][2-Me-Leu]E(1c)R |
| SEQ ID NO: 67 | [(3-Aminomethyl)benzoyl](1c)*SE(2c)WQ[Dab](2c)[Y(2-aminoethoxy)][2- |
| | Nal][2-Me-Leu]E(1c)R |
| SEQ ID NO: 68 | [(3-Aminomethyl)benzoyl](1c)*S[Orn](2c)WQE(2c)[Y(2-aminoethoxy)][2- |
| | Nal][2-Me-Leu]E(1c)R |
| SEQ ID NO: 69 | [(3-Aminomethyl)benzoyl](1c)*SE(2c)WQ[Orn](2c)[Y(2-aminoethoxy)][2- |
| | Nal][2-Me-Leu]E(1c)R |
| SEQ ID NO: 70 | [(3-Aminomethyl)benzoyl](1c)*SC(2a)[7-Me-Trp]QC(2a)[Y(2- |
| | aminoethoxy)][2-Nal][2-Me-Leu]E(1c)R |
| SEQ ID NO: 71 | [(3-Aminomethyl)benzoyl](1c)*SC(2a)[7-Ph-Trp]QC(2a)[Y(2- |
| | aminoethoxy)][2-Nal][2-Me-Leu]E(1c)R |
| SEQ ID NO: 72 | [(3-Aminomethyl)benzoyl](1c)*SC(2a)[7-(Naphth-2-yl)-Trp]QC(2a)[Y(2- |
| | aminoethoxy)][2-Nal][2-Me-Leu]E(1c)R |
| SEQ ID NO: 73 | [(3-Aminomethyl)benzoyl](1c)*SC(2a)WQC(2a)[Y(2-aminoethoxy)][3-(3- |
| | Quinolinyl)-Ala][2-Me-Leu]E(1c)R |
| SEQ ID NO: 74 | [(3-Aminomethyl)benzoyl](1c)*SC(2a)WQC(2a)[Y(2-aminoethoxy)][6-Cl- |
| | Trp][2-Me-Leu]E(1c)R |
| SEQ ID NO: 75 | [(3-Aminomethyl)benzoyl](1c)*SC(2a)WQC(2a)[Y(2-aminoethoxy)][2- |
| | Nal][4-aminotetrahydro-2H-pyran-4-acetyl]E(1c)R |
| SEQ ID NO: 76 | [(3-Aminomethyl)benzoyl](1c)*SC(2a)WQC(2a)[Y(2-aminoethoxy)][2- |
| | Nal][2-Me-Leu]E(1c)[3-(3-Pyridyl)-Ala] |
| SEQ ID NO: 77 | [(3-Aminomethyl)benzoyl](1c)*SC(2a)WQC(2a)[Y(2-aminoethoxy)][2- |
| | Nal][2-Me-Leu]E(1c)[3-(4-Pyridyl)-Ala] |
| SEQ ID NO: 78 | [(3-Aminomethyl)benzoyl](1c)*SC(2a)WQC(2a)[Y(2-aminoethoxy)][2- |
| | Nal][2-Me-Leu]E(1c)R[3-(3-Pyridyl)-Ala] |
| SEQ ID NO: 79 | [(3-Aminomethyl)benzoyl](1c)*SC(2a)WQC(2a)[Y(2-aminoethoxy)][2- |
| | Nal][2-Me-Leu]E(1c)R[3-(4-Pyridyl)-Ala] |
| SEQ ID NO: 80 | [4-Aminomethyl-phenylacetyl](1c)*SC(2a)WQC(2a)[Y(2-aminoethoxy)][2- |
| | Nal][2-Me-Leu]E(1c)[{d}R] |
| SEQ ID NO: 81 | [4-Aminomethyl-phenylacetyl](1c)*SC(2a)WQC(2a)[Y(2-aminoethoxy)][2- |
| | Nal][2-Me-Leu]E(1c)S |
| SEQ ID NO: 82 | [4-Aminomethyl-phenylacetyl](1c)*SC(2a)WQC(2a)[Y(2-aminoethoxy)][2- |
| | Nal][2-Me-Leu]E(1c)[Dab] |
| SEQ ID NO: 83 | [4-Aminomethyl-phenylacetyl](1c)*TC(2a)WQC(2a)[Y(2-aminoethoxy)][2- |
| | Nal][2-Me-Leu]E(1c)[{d}R] |
| SEQ ID NO: 84 | K(1c)SC(2a)WQC(2a)[Y(2-aminoethoxy)][2-Nal][2-Me-Leu]E(1c)[{d}R] |
| SEQ ID NO: 85 | K(1c)TC(2a)WQC(2a)[Y(2-aminoethoxy)][2-Nal][2-Me-Leu]E(1c)[{d}R] |
| SEQ ID NO: 86 | K(1c)SC(2a)WQC(2a)[Y(2-aminoethoxy)][2-Nal][2-Me-Leu]E(1c)S |
| SEQ ID NO: 87 | [6-Aminohexanoyl](1c)*SC(2a)WQC(2a)[Y(2-aminoethoxy)][2-Nal][2-Me- |
| | Leu]E(1c)[{d}R] |
| SEQ ID NO: 88 | [6-Amino-4-oxahexanoyl](1c)*SC(2a)WQC(2a)[Y(2-aminoethoxy)][2- |
| | Nal][2-Me-Leu]E(1c)[{d}R] |
| SEQ ID NO: 89 | [trans-4-Aminomethyl-cyclohexyl-1-carbonyl](1c)*[beta-homo- |
| | Ser]C(2a)WQC(2a)[Y(2-aminoethoxy)][2-Nal][2-Me-Leu]E(1c)[{d}R] |
| SEQ ID NO: 90 | [(4-(2-Aminoethyl)-piperazine-1-yl)-acetyl](1c)*SC(2a)WQC(2a)[Y(2- |
| | aminoethoxy)][2-Nal][2-Me-Leu]E(1c)[{d}R] |
| SEQ ID NO: 91 | [(4-(2-Aminoethyl)-piperazine-1-yl)-acetyl](1c)*SC(2a)WQC(2a)[Y(2- |
| | aminoethoxy)][2-Nal][2-Me-Leu]D(1c)[{d}R] |
| SEQ ID NO: 92 | [3-Aminomethyl-phenylacetyl](1c)*SC(2a)WQC(2a)[Y(2-aminoethoxy)][2- |
| | Nal][2-Me-Leu]E(1c)[{d}R] |
| SEQ ID NO: 93 | [2-Aminomethyl-phenylacetyl](1c)*SC(2a)WQC(2a)[Y(2-aminoethoxy)][2- |
| | Nal][2-Me-Leu]E(1c)[{d}R] |
| SEQ ID NO: 94 | [K(N₃)](1g)SC(2a)WQC(2a)[Y(2-aminoethoxy)][2-Nal][2-Me- |
| | Leu][Pra](1g)[{d}R] |
| SEQ ID NO: 95 | [Azidoacetic acid](1g)*SC(2a)WQC(2a)[Y(2-aminoethoxy)][2-Nal][2-Me- |
| | Leu][Glu(propargylamine)](1g)[{d}R] |
| SEQ ID NO: 96 | [Azidoacetic acid](1g)*SC(2a)WQC(2a)[Y(2-aminoethoxy)][2-Nal][2-Me- |
| | Leu][Dab(3-butynoic acid)](1g)[{d}R] |
| SEQ ID NO: 97 | [but-3-ynoic acid](1g)*SC(2a)WQC(2a)[Y(2-aminoethoxy)][2-Nal][2-Me- |
| | Leu][Dab(azidoacetic acid)](1g)[{d}R] |
| SEQ ID NO: 98 | [(N₃)-Ala](1g)*SC(2a)WQC(2a)[Y(2-aminoethoxy)][2-Nal][2-Me- |
| | Leu][Glu(propargylamine)](1g)[{d}R] |
| SEQ ID NO: 99 | [(N₃)-Ala](1g)*SC(2a)WQC(2a)[Y(2-aminoethoxy)][2-Nal][2-Me- |
| | Leu][Dab(3-butynoic acid)](1g)[{d}R] |
| SEQ ID NO: 100 | [but-3-ynoic acid](1g)*SC(2a)WQC(2a)[Y(2-aminoethoxy)][2-Nal][2-Me- |
| | Leu][Dab((N₃)-Ala))](1g)[{d}R] |
| SEQ ID NO: 101 | [4-Aminomethyl-2-pyridineacetyl](1c)*SC(2a)WQC(2a)[Y(2- |
| | aminoethoxy)][2-Nal][2-Me-Leu]E(1c)[{d}R] |
| SEQ ID NO: 102 | [4-Aminomethyl-3-pyridineacetyl](1c)*SC(2a)WQC(2a)[Y(2- |
| | aminoethoxy)][2-Nal][2-Me-Leu]E(1c)[{d}R] |
| SEQ ID NO: 103 | [4-Aminomethyl-2-fluoro-phenylacetyl](1c)*SC(2a)WQC(2a)[Y(2- |
| | aminoethoxy)][2-Nal][2-Me-Leu]E(1c)[{d}R] |
| SEQ ID NO: 104 | [4-Aminomethyl-3-methyl-phenylacetyl](1c)*SC(2a)WQC(2a)[Y(2- |
| | aminoethoxy)][2-Nal][2-Me-Leu]E(1c)[{d}R] |
| SEQ ID NO: 105 | [4-Aminomethyl-3-methoxy-phenylacetyl](1c)*SC(2a)WQC(2a)[Y(2- |
| | aminoethoxy)][2-Nal][2-Me-Leu]E(1c)[{d}R] |

or a pharmaceutically acceptable salt or solvate thereof;
wherein:
*= bridge uses the peptide backbone amine or carboxylic acid at the N- or C-terminus, not the side chain amine or carboxylic acid
(1a) = [2,11] 1,3-dithio-propan-2-one bridge; (1c) = [2,11] lactam bridge; (1g) = [2,11] 1,4-disubstituted 1,2,3-triazole bridge; (2a) = [4,7] 1,3-dithio-propan-2-one bridge; (2c) = [4,7] lactam bridge; (2d) = [4,7] 1,2-phenylenedimethanethiol bridge; (2e) = [4,7] 1,3-phenylenedimethanethiol bridge; (2f) = [4,7] 1,4-phenylenedimethanethiol bridge; (2g) = [4,7] 1,4-disubstituted 1,2,3-triazole bridge.

11. A compound according to clause 1 which is selected from:

| | |
|---|---|
| 1 | H-DC(1a)SC(2a)WQC(2a)WW[2-Me-Leu]C(1a)R-[NH2] |
| 2 | H-LC(1a)SC(2a)WQC(2a)WWLC(1a)R-[NH2] |
| 3 | H-DC(1a)SC(2a)WEC(2a)WWLC(1a)R-[NH2] |
| 4 | H-DE(1c)SC(2a)WQC(2a)WWLK(1c)R-[NH2] |
| 5 | H-DC(1a)SE(2c)WQK(2c)WWLC(1a)R-[NH2] |
| 6 | D(1c)*SC(2a)WQC(2a)WWLR(1c)* |
| 7 | [Ac]-DK(1c)SC(2a)WQC(2a)WWLE(1c)R-[NH2] |
| 8 | [Ac]-D[Orn](1c)SC(2a)WQC(2a)WWLE(1c)R-[NH2] |
| 9 | H-DE(1c)SC(2a)WQC(2a)WWL[Dab](1c)R-[NH2] |
| 10 | [Ac]-E(1c)SC(2a)WQC(2a)WWLK(1c)R-[NH2] |
| 11 | [Ac]-DE(1c)SC(2a)WQC(2a)WWLK(1c)-[NH2] |
| 12 | [Ac]-E(1c)SC(2a)WQC(2a)WWLK(1c)-[NH2] |
| 13 | [Ac]-DE(1c)SC(2a)AQC(2a)WWLK(1c)R-[NH2] |
| 14 | [Ac]-DE(1c)SC(2a)WQC(2a)AWLK(1c)R-[NH2] |
| 15 | [Ac]-DE(1c)SC(2a)WQC(2a)WALK(1c)R-[NH2] |
| 16 | [Ac]-DE(1c)SC(2a)[7-Me-Trp]QC(2a)WWLK(1c)R-[NH2] |
| 17 | [Ac]-DE(1c)SC(2a)WQC(2a)W[2-Nal]LK(1c)R-[NH2] |
| 18 | [Ac]-K(1c)SC(2a)WQC(2a)W[2-Nal]LE(1c)R-[NH2] |
| 19 | [Ac]-DK(1c)SC(2a)WQC(2a)W[2-Nal]LE(1c)R-[NH2] |
| 20 | [Ac]-DK(1c)SC(2d)WQC(2d)W[2-Nal]LE(1c)R-[NH2] |
| 21 | [Ac]-DK(1c)SC(2e)WQC(2e)W[2-Nal]LE(1c)R-[NH2] |
| 22 | [Ac]-DK(1c)SC(2f)WQC(2f)W[2-Nal]LE(1c)R-[NH2] |
| 23 | [Ac]-DK(1c)S[K(N₃)](2g)WQ[Pra](2g)W[2-Nal]LE(1c)R-[NH2] |
| 24 | [Ac]-DK(1c)S[Aha](2g)WQ[Pra](2g)W[2-Nal]LE(1c)R-[NH2] |
| 25 | [Ac]-K(1c)SC(2a)[2-Nal]QC(2a)W[2-Nal]LE(1c)R-[NH2] |
| 26 | [Ac]-K(1c)SC(2a)[Bip]QC(2a)W[2-Nal]LE(1c)R-[NH2] |
| 27 | [Ac]-K(1c)SC(2a)[1-Me-Trp]QC(2a)W[2-Nal]LE(1c)R-[NH2] |
| 28 | [Ac]-DK(1c)SC(2a)WQC(2a)[2-Nal][2-Nal]LE(1c)R-[NH2] |
| 29 | [Ac]-K(1c)SC(2a)WQC(2a)[Y(2-aminoethoxy)][2-Nal]LE(1c)R-[NH2] |
| 30 | [Ac]-K(1c)SC(2a)WQC(2a)H[2-Nal]LE(1c)R-[NH2] |
| 31 | [Ac]-K(1c)SC(2a)WQC(2a)W[F(4-F)]LE(1c)R-[NH2] |
| 32 | [Ac]-K(1c)SC(2a)WQC(2a)W[1-Me-Trp]LE(1c)R-[NH2] |
| 33 | [Ac]-C(1a)TC(2a)WEC(2a)WW[2-Me-Leu]C(1a)S-[NH2] |
| 34 | [Ac]-GK(1c)TC(2a)WEC(2c)WW[2-Me-Leu]E(1c)S-[NH2] |
| 35 | [Ac]-GC(1a)TC(2a)WEC(2a)W[2-Nal][2-Me-Leu]C(1a)R-[NH2] |
| 36 | [bAla](1c)*SC(2a)WQC(2a)W[2-Nal]LE(1c)R-[NH2] |
| 37 | [(3-Aminomethyl)benzoyl](1c)*SC(2a)WQC(2a)W[2-Nal]LE(1c)R-[NH2] |
| 38 | [(3-Aminomethyl)benzoyl](1c)*SC(2a)WQC(2a)[Y(2-aminoethoxy)][2- |
| | Nal][2-Me-Leu]E(1c)R-[NH2] |
| 39 | [(3-Aminomethyl)benzoyl](1c)*SC(2a)WQC(2a)[Y(2-aminoethoxy)][2- |
| | Nal]LE(1c)R-[NH2] |
| 40 | [(3-Aminomethyl)benzoyl](1c)*SC(2a)WQC(2a)W[2-Nal][2-Me- |
| | Leu]E(1c)R-[NH2] |
| 41 | [(3-Aminomethyl)benzoyl](1c)*SC(2a)WQC(2a)[Y(2-aminoethoxy)][2- |
| | Nal][2-Me-Leu]E(1c)[{d}R]-[NH2] |
| 42 | [(3-Aminomethyl)benzoyl](1c)*SC(2a)WQC(2a)[Y(2-aminoethoxy)][2- |
| | Nal][2-Me-Leu]E(1c)[2-Me-Arg]-[NH2] |
| 43 | [(3-Aminomethyl)benzoyl](1c)*SC(2a)WQC(2a)[Y(2-aminoethoxy)][2- |
| | Nal][2-Me-Leu]E(1c)[N-Me-Arg]-[NH2] |
| 44 | [(3-Aminomethyl)benzoyl](1c)*SC(2a)WQC(2a)[Y(2-aminoethoxy)][2- |
| | Nal][2-Me-Leu]E(1c)S-[NH2] |
| 45 | [(3-Aminomethyl)benzoyl](1c)*SC(2a)WQC(2a)[Y(2-aminoethoxy)][2- |
| | Nal][2-Me-Leu]E(1c)[F(4-NH₂)]-[NH2] |
| 46 | [(3-Aminomethyl)benzoyl](1c)*SC(2a)WQC(2a)[Y(2-aminoethoxy)][2- |
| | Nal][{d}L]E(1c)Y-[NH2] |
| 47 | [Ac]-K(1c)SC(2a)WQC(2a)[Y(2-aminoethoxy)][2-Nal][2-Me-Leu]E(1c)R- |
| | [NH2] |
| 48 | [Ac]-K(1c)SC(2a)WQC(2a)[Y(2-aminoethoxy)][2-Nal][{d}L]E(1c)R-[NH2] |
| 49 | [(4-Aminomethyl)benzoyl](1c)*SC(2a)WQC(2a)[Y(2-aminoethoxy)][2- |
| | Nal][2-Me-Leu]E(1c)R-[NH2] |
| 50 | [4-(2-Aminoethyl)benzoyl](1c)*SC(2a)WQC(2a)[Y(2-aminoethoxy)][2- |
| | Nal][2-Me-Leu]E(1c)R-[NH2] |
| 51 | [3-(4-Aminophenyl)propanoyl](1c)*SC(2a)WQC(2a)[Y(2-aminoethoxy)][2- |
| | Nal][2-Me-Leu]E(1c)R-[NH2] |
| 52 | [4-Aminomethyl-phenylacetyl](1c)*SC(2a)WQC(2a)[Y(2-aminoethoxy)][2- |
| | Nal][2-Me-Leu]E(1c)R-[NH2] |
| 53 | H-DC(1a)SC(2b)WQC(2b)WWLC(1a)R-[NH2] |
| 54 | [Ac]-K(1c)SC(2b)WQC(2b)[Y(2-aminoethoxy)][2-Nal]LE(1c)R-[NH2] |
| 55 | [Ac]-DE(1c)QC(2a)WQC(2a)YW[2-Me-Leu]K(1c)R-[NH2] |
| 56 | [Ac]-DE(1c)QC(2a)WQC(2a)[2-Nal]W[2-Me-Leu]K(1c)R-[NH2] |
| 57 | [Ac]-DE(1c)TC(2a)WQC(2a)W[2-Nal][2-Me-Leu]K(1c)R-[NH2] |
| 58 | [Ac]-DE(1c)SC(2a)WQC(2a)W[2-Nal][2-Me-Leu]K(1c)ENG-[NH2] |
| 59 | [Ac]-DE(1c)SC(2a)WQC(2a)W[2-Nal][2-Me-Leu]K(1c)NG-[NH2] |
| 60 | [Ac]-DE(1c)SC(2a)WQC(2a)W[2-Nal][2-Me-Leu]K(1c)G-[NH2] |
| 61 | [Ac]-DE(1c)SC(2a)WQC(2a)W[2-Nal][2-Me-Lys]K(1c)ENG-[NH2] |
| 62 | [Ac]-DC(1a)QC(2a)WQC(2a)[2-Nal]W[2-Me-Leu]C(1a)R-[NH2] |
| 63 | [Ac]-DC(1a)QC(2a)WQC(2a)YW[2-Me-Leu]C(1a)R-[NH2] |
| 64 | [Ac]-DC(1a)QC(2a)WQC(2a)WW[2-Me-Leu]C(1a)ENG-[NH2] |
| 65 | [(3-Aminomethyl)benzoyl](1c)*S[Dpr](2c)WQE(2c)[Y(2-aminoethoxy)][2- |
| | Nal][2-Me-Leu]E(1c)R-[NH2] |
| 66 | [(3-Aminomethyl)benzoyl](1c)*SE(2c)WQ[Dpr](2c)[Y(2-aminoethoxy)][2- |
| | Nal][2-Me-Leu]E(1c)R-[NH2] |
| 67 | [(3-Aminomethyl)benzoyl](1c)*SE(2c)WQ[Dab](2c)[Y(2-aminoethoxy)][2- |
| | Nal][2-Me-Leu]E(1c)R-[NH2] |
| 68 | [(3-Aminomethyl)benzoyl](1c)*S[Orn](2c)WQE(2c)[Y(2-aminoethoxy)][2- |
| | Nal][2-Me-Leu]E(1c)R-[NH2] |
| 69 | [(3-Aminomethyl)benzoyl](1c)*SE(2c)WQ[Orn](2c)[Y(2-aminoethoxy)][2- |
| | Nal][2-Me-Leu]E(1c)R-[NH2] |
| 70 | [(3-Aminomethyl)benzoyl](1c)*SC(2a)[7-Me-Trp]QC(2a)[Y(2- |
| | aminoethoxy)][2-Nal][2-Me-Leu]E(1c)R-[NH2] |
| 71 | [(3-Aminomethyl)benzoyl](1c)*SC(2a)[7-Ph-Trp]QC(2a)[Y(2- |
| | aminoethoxy)][2-Nal][2-Me-Leu]E(1c)R-[NH2] |
| 72 | [(3-Aminomethyl)benzoyl](1c)*SC(2a)[7-(Naphth-2-yl)-Trp]QC(2a)[Y(2- |
| | aminoethoxy)][2-Nal][2-Me-Leu]E(1c)R-[NH2] |
| 73 | [(3-Aminomethyl)benzoyl](1c)*SC(2a)WQC(2a)[Y(2-aminoethoxy)][3-(3- |
| | Quinolinyl)-Ala][2-Me-Leu]E(1c)R-[NH2] |
| 74 | [(3-Aminomethyl)benzoyl](1c)*SC(2a)WQC(2a)[Y(2-aminoethoxy)][6-Cl- |
| | Trp][2-Me-Leu]E(1c)R-[NH2] |
| 75 | [(3-Aminomethyl)benzoyl](1c)*SC(2a)WQC(2a)[Y(2-aminoethoxy)][2- |
| | Nal][4-aminotetrahydro-2H-pyran-4-acetyl]E(1c)R-[NH2] |
| 76 | [(3-Aminomethyl)benzoyl](1c)*SC(2a)WQC(2a)[Y(2-aminoethoxy)][2- |
| | Nal][2-Me-Leu]E(1c)[3-(3-Pyridyl)-Ala]-[NH2] |
| 77 | [(3-Aminomethyl)benzoyl](1c)*SC(2a)WQC(2a)[Y(2-aminoethoxy)][2- |
| | Nal][2-Me-Leu]E(1c)[3-(4-Pyridyl)-Ala]-[NH2] |
| 78 | [(3-Aminomethyl)benzoyl](1c)*SC(2a)WQC(2a)[Y(2-aminoethoxy)][2- |
| | Nal][2-Me-Leu]E(1c)R[3-(3-Pyridyl)-Ala]-[NH2] |
| 79 | [(3-Aminomethyl)benzoyl](1c)*SC(2a)WQC(2a)[Y(2-aminoethoxy)][2- |
| | Nal][2-Me-Leu]E(1c)R[3-(4-Pyridyl)-Ala]-[NH2] |
| 80 | [4-Aminomethyl-phenylacetyl](1c)*SC(2a)WQC(2a)[Y(2-aminoethoxy)][2- |
| | Nal][2-Me-Leu]E(1c)[{d}R]-[NH2] |
| 81 | [4-Aminomethyl-phenylacetyl](1c)*SC(2a)WQC(2a)[Y(2-aminoethoxy)][2- |
| | Nal][2-Me-Leu]E(1c)S-[NH2] |
| 82 | [4-Aminomethyl-phenylacetyl](1c)*SC(2a)WQC(2a)[Y(2-aminoethoxy)][2- |
| | Nal][2-Me-Leu]E(1c)[Dab]-[NH2] |
| 83 | [4-Aminomethyl-phenylacetyl](1c)*TC(2a)WQC(2a)[Y(2-aminoethoxy)][2- |
| | Nal][2-Me-Leu]E(1c)[{d}R]-[NH2] |
| 84 | [Ac]-K(1c)SC(2a)WQC(2a)[Y(2-aminoethoxy)][2-Nal][2-Me- |
| | Leu]E(1c)[{d}R]-[NH2] |
| 85 | [Ac]-K(1c)TC(2a)WQC(2a)[Y(2-aminoethoxy)][2-Nal][2-Me- |
| | Leu]E(1c)[{d}R]-[NH2] |
| 86 | [Ac]-K(1c)SC(2a)WQC(2a)[Y(2-aminoethoxy)][2-Nal][2-Me-Leu]E(1c)S- |
| | [NH2] |
| 87 | [6-Aminohexanoyl](1c)*SC(2a)WQC(2a)[Y(2-aminoethoxy)][2-Nal][2-Me- |
| | Leu]E(1c)[{d}R]-[NH2] |
| 88 | [6-Amino-4-oxahexanoyl](1c)*SC(2a)WQC(2a)[Y(2-aminoethoxy)][2- |
| | Nal][2-Me-Leu]E(1c)[{d}R]-[NH2] |
| 89 | [trans-4-Aminomethyl-cyclohexyl-1-carbonyl](1c)*[beta-homo- |
| | Ser]C(2a)WQC(2a)[Y(2-aminoethoxy)][2-Nal][2-Me-Leu]E(1c)[{d}R]-[NH2] |
| 90 | [(4-(2-Aminoethyl)-piperazine-1-yl)-acetyl](1c)*SC(2a)WQC(2a)[Y(2- |
| | aminoethoxy)][2-Nal][2-Me-Leu]E(1c)[{d}R]-[NH2] |
| 91 | [(4-(2-Aminoethyl)-piperazine-1-yl)-acetyl](1c)*SC(2a)WQC(2a)[Y(2- |
| | aminoethoxy)][2-Nal][2-Me-Leu]D(1c)[{d}R]-[NH2] |
| 92 | [3-Aminomethyl-phenylacetyl](1c)*SC(2a)WQC(2a)[Y(2-aminoethoxy)][2- |
| | Nal][2-Me-Leu]E(1c)[{d}R]-[NH2] |
| 93 | [2-Aminomethyl-phenylacetyl](1c)*SC(2a)WQC(2a)[Y(2-aminoethoxy)][2- |
| | Nal][2-Me-Leu]E(1c)[{d}R]-[NH2] |
| 94 | [Ac]-[K(N₃)](1g)SC(2a)WQC(2a)[Y(2-aminoethoxy)][2-Nal][2-Me- |
| | Leu][Pra](1g)[{d}R]-[NH2] |
| 95 | [Azidoacetic acid](1g)*SC(2a)WQC(2a)[Y(2-aminoethoxy)][2-Nal][2-Me- |
| | Leu][Glu(propargylamine)](1g)[{d}R]-[NH2] |
| 96 | [Azidoacetic acid](1g)*SC(2a)WQC(2a)[Y(2-aminoethoxy)][2-Nal][2-Me- |
| | Leu][Dab(3-butynoic acid)](1g)[{d}R]-[NH2] |
| 97 | [but-3-ynoic acid](1g)*SC(2a)WQC(2a)[Y(2-aminoethoxy)][2-Nal][2-Me- |
| | Leu][Dab(azidoacetic acid)](1g)[{d}R]-[NH2] |
| 98 | [(N₃)-Ala](1g)*SC(2a)WQC(2a)[Y(2-aminoethoxy)][2-Nal][2-Me- |
| | Leu][Glu(propargylamine)](1g)[{d}R]-[NH2] |
| 99 | [(N₃)-Ala](1g)*SC(2a)WQC(2a)[Y(2-aminoethoxy)][2-Nal][2-Me- |
| | Leu][Dab(3-butynoic acid)](1g)[{d}R]-[NH2] |
| 100 | [but-3-ynoic acid](1g)*SC(2a)WQC(2a)[Y(2-aminoethoxy)][2-Nal][2-Me- |
| | Leu][Dab((N₃)-Ala))](1g)[{d}R]-[NH2] |
| 101 | [4-Aminomethyl-2-pyridineacetyl](1c)*SC(2a)WQC(2a)[Y(2- |
| | aminoethoxy)][2-Nal][2-Me-Leu]E(1c)[{d}R]-[NH2] |
| 102 | [4-Aminomethyl-3-pyridineacetyl](1c)*SC(2a)WQC(2a)[Y(2- |
| | aminoethoxy)][2-Nal][2-Me-Leu]E(1c)[{d}R]-[NH2] |
| 103 | [4-Aminomethyl-2-fluoro-phenylacetyl](1c)*SC(2a)WQC(2a)[Y(2- |
| | aminoethoxy)][2-Nal][2-Me-Leu]E(1c)[{d}R]-[NH2] |
| 104 | [4-Aminomethyl-3-methyl-phenylacetyl](1c)*SC(2a)WQC(2a)[Y(2- |
| | aminoethoxy)][2-Nal][2-Me-Leu]E(1c)[{d}R]-[NH2] |
| 105 | [4-Aminomethyl-3-methoxy-phenylacetyl](1c)*SC(2a)WQC(2a)[Y(2- |
| | aminoethoxy)][2-Nal][2-Me-Leu]E(1c)[{d}R]-[NH2] |

and pharmaceutically acceptable salts and solvates thereof;
wherein:
*= bridge uses the peptide backbone amine or carboxylic acid at the N- or C-terminus, not the side chain amine or carboxylic acid
(1a) = [2,11] 1,3-dithio-propan-2-one bridge; (1c) = [2,11] lactam bridge; (1g) = [2,11] 1,4-disubstituted 1,2,3-triazole bridge; (2a) = [4,7] 1,3-dithio-propan-2-one bridge; (2c) = [4,7] lactam bridge; (2d) = [4,7] 1,2-phenylenedimethanethiol bridge; (2e) = [4,7] 1,3-phenylenedimethanethiol bridge; (2f) = [4,7] 1,4-phenylenedimethanethiol bridge; (2g) = [4,7] 1,4-disubstituted 1,2,3-triazole bridge.

12. A pharmaceutical composition comprising a compound according to any one of the preceding clauses in combination with a pharmaceutically acceptable carrier, excipient or vehicle.
13. A method for the synthesis of a compound according to any one of clauses 1 to 11, comprising synthesising the analogue by solid-phase or liquid-phase peptide synthesis methodology, optionally isolating and/or purifying the final product, and optionally further comprising the step of forming an amide bond, forming two thioether bonds with a linker, or forming a triazole between the amino acid residues at positions X2 and X11, and optionally further comprising the step of forming an amide bond, forming two thioether bonds with a linker, or forming a triazole between the amino acid residues at positions X4 and X7.
14. A compound according to any one of clauses 1 to 11, or a pharmaceutical composition according to clause 12, for use in a method of medical treatment.
15. A compound according to any one of clauses 1 to 11, or a pharmaceutical composition according to clause 12, for use in a method of prevention or treatment of Inflammatory Bowel Disease (IBD), ulcerative colitis, Crohn's disease, Celiac disease (nontropical Sprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-I, chronic granulomatous disease, glycogen storage disease type 1b, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, ankylosing spondylitis, or graft versus host disease in a subject, and combinations thereof.

1. A compound of the formula:
R¹—Z—R²
wherein
R¹ is H, C₁₋₄ acyl, benzoyl, C₁₋₄ alkyl, or is absent;
R² is NHR³, OH, or is absent, wherein R³ is hydrogen or C₁₋₃ alkyl optionally substituted with NH₂; and
Z is an amino acid sequence of formula I:
X1-X2-X3-X4-X5-X6-X7-X8-X9-X10-X11-X12-X13-X14 (1)
wherein
X1 is absent or is selected from the group consisting of Asp, Gly, Leu, Glu, Ser, Cys, and Lys;
X3 is selected from the group consisting of Ser, beta-homo-Ser, Thr, Leu, Cys, Gln, Val, Ile, N-Me-Ser, and Q(pyrrolidin);
X5 is selected from the group consisting of Trp, Tyr, Ala, 1-Me-Trp, 7-Me-Trp, 7-Ph-Trp, 7-(Naphth-2-yl)-Trp, 2-Nal, Bip, 4-F-Trp, 7-F-Trp, and N-Me-Trp;
X6 is selected from the group consisting of Gln, Glu, Tyr, Cys, Val, His, N-Me-Gln, and Q(pyrrolidin);
X8 is selected from the group consisting of Trp, Tyr, Asn, Ala, His, 2-Nal, Dab, 2,4-diaminobutanoyl([2-(trimethyl-2-aminoethoxy)ethoxy]propyl), F(4-NH₂-(2-(trimethyl-2-aminoethoxy)ethoxy)propyl), Phe, Val, 4-Me-Phe, 2-Me-Phe, Bip, 2-Me-F(4-F), {d}F(4-F), 4-Cl-Phe, alpha-Me-Trp, 3,3-Diphenyl-Ala, and Phg, wherein the hydroxyl group of Tyr is optionally substituted with C₁₋₃ alkyl optionally substituted with NH₂;
X9 is selected from the group consisting of 2-Nal, Trp, 1-Me-Trp, 6-Cl-Trp, 3-(3-Quinolinyl)-Ala, Phe, 4-F-Phe, Glu, Cys, Ala, 6-F-Trp, His, 3-F-Phe, 3,4-Me-Phe, Bip, and {d}6-F-Trp;
X10 is selected from the group consisting of Leu, D-Leu, 2-Me-Leu, 2-Me-Lys, Trp, Asn, Cys, 4-aminotetrahydro-2H-pyran-4-acetyl, and 2-Me-Val;
X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, 3-(4-Pyridyl)-Ala, {d}2,4-diaminobutanoyl([2-(trimethyl-2-aminoethoxy)ethoxy]propyl), D-Gln, D-Glu, D-His, 3-aminopropanoyl, and GABA, or is absent;
X13 is absent or is selected from the group consisting of Asn, Gly, 3-(3-Pyridyl)-Ala, 3-(4-Pyridyl)-Ala, 3-(3-Quinolinyl)-Ala, {d}[3-(3-Pyridyl)-Ala], 3-amino-3-(3′-pyridyl)propionyl, 3-F-Phe, 3,5-F-Phe, 4-aminomethyl-2-pyridineacetyl, 2,3-diaminopropanoyl(3-pyridylacetyl), 2,3-diaminopropanoyl(3-pyridylpropionyl), 2,3-diaminopropanoyl(3-fluorobenzoyl), 2,3-diaminopropanoyl(3-fluorophenylacetyl), and 2-Me-3-(3-Pyridyl)-Ala;
X14 is absent or is Gly;
X2 and X11 are amino acid residues who together form a lactam bridge, a dithioether bridge, or a bridge containing a triazole ring; and
X4 and X7 are amino acid residues who together form a lactam bridge, a dithioether bridge, or a bridge containing a triazole ring;
or a pharmaceutically acceptable salt or solvate thereof;
wherein the compound is not:
I3(isomer 3)H-DC(1a)SC(2a)WQC(2a)WWLC(1a)R-[NH₂];
wherein (1a) is a [2,11]1,3-dithio-propan-2-one bridge and (2a) is a [4,7]1,3-dithio-propan-2-one bridge.

2. The compound according to claim 1 of the formula:
R¹—Z—R²
wherein
R¹ is H, C₁₋₄ acyl, benzoyl, C₁₋₄ alkyl, or is absent;
R² is NHR³, OH, or is absent, wherein R³ is hydrogen or C₁₋₃ alkyl; and
Z is an amino acid sequence of formula I:
X1-X2-X3-X4-X5-X6-X7-X8-X9-X10-X11-X12-X13-X14 (1)
wherein
X1 is absent or is selected from the group consisting of Asp, Glu, Ser, Cys, and Lys;
X3 is selected from the group consisting of Ser, beta-homo-Ser, Thr, Leu, Cys, and Gln;
X5 is selected from the group consisting of Trp, Tyr, Ala, 1-Me-Trp, 7-Me-Trp, 7-Ph-Trp, 7-(Naphth-2-yl)-Trp, 2-Nal, and Bip;
X6 is selected from the group consisting of Gln, Glu, Tyr and Cys;
X8 is selected from the group consisting of Trp, Tyr, Asn, Ala, His, and 2-Nal, wherein the hydroxyl group of Tyr is optionally substituted with C₁₋₃ alkyl optionally substituted with NH₂;
X9 is selected from the group consisting of 2-Nal, Trp, 1-Me-Trp, 6-Cl-Trp, 3-(3-Quinolinyl)-Ala, Phe, 4-F-Phe, Glu, Cys, and Ala;
X10 is selected from the group consisting of Leu, D-Leu, 2-Me-Leu, 2-Me-Lys, Trp, Asn, Cys, and 4-aminotetrahydro-2H-pyran-4-acetyl;
X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent;
X13 is absent or is selected from the group consisting of Asn, Gly, 3-(3-Pyridyl)-Ala, and 3-(4-Pyridyl)-Ala;
X14 is absent or is Gly;
X2 and X11 are amino acid residues who together form a lactam bridge, a dithioether bridge, or a bridge containing a triazole ring; and
X4 and X7 are amino acid residues who together form a lactam bridge, a dithioether bridge, or a bridge containing a triazole ring;
or a pharmaceutically acceptable salt or solvate thereof;
wherein the compound is not:
I3(isomer 3)H-DC(1a)SC(2a)WQC(2a)WWLC(1a)R-[NH₂];
wherein (1a) is a [2,11]1,3-dithio-propan-2-one bridge and (2a) is a [4,7]1,3-dithio-propan-2-one bridge.

3. The compound according to claim 1 or 2, wherein X1 is absent or Asp.

4. The compound according to any one of the preceding claims, wherein X1 is absent.

5. The compound according to any one of the preceding claims, wherein X3 is Ser or lie.

6. The compound according to any one of the preceding claims, wherein X5 is Trp or 7-Me-Trp.

7. The compound according to any one of the preceding claims, wherein X6 is Gln or Glu.

8. The compound according to any one of the preceding claims, wherein X8 is Trp, Tyr, or 4-Me-Phe; wherein the hydroxyl group of Tyr is optionally substituted with —CH₂CH₂NH₂.

9. The compound according to any one of the preceding claims, wherein X9 is 2-Nal, Trp, or 3,4-Me-Phe.

10. The compound according to any one of the preceding claims, wherein X9 is 2-Nal.

11. The compound according to any one of the preceding claims, wherein X10 is Leu, 2-Me-Leu, or 2-Me-Val.

12. The compound according to any one of the preceding claims, wherein X10 is Leu, or 2-Me Leu.

13. The compound according to any one of the preceding claims, wherein X12 is Arg, D-Arg, and Dab.

14. The compound according to any one of the preceding claims, wherein X13 is absent, 3-(3-Pyridyl)-Ala, or 2-Me-3-(3-Pyridyl)-Ala.

15. The compound according to any one of the preceding claims, wherein X14 is absent.

16. The compound according to any one of the preceding claims, wherein R¹ is H, C₁₋₂ acyl, or absent.

17. The compound according to any one of the preceding claims, wherein R¹ is absent.

18. The compound according to any one of claims 1 to 16, wherein R¹ is —C(═O)CH₃.

19. The compound according to any one of the preceding claims, wherein R² is NH₂.

20. The compound according to any one of the preceding claims, wherein the length of the bridge between X2 and X11 and/or X4 and X7 is 5 to 10 atoms long.

21. The compound according to any one of the preceding claims, wherein X1 is absent, R¹ is absent, and X2 and X11 are amino acid residues that together form a lactam bridge or a bridge containing a triazole ring via the N-terminus of X2.

22. The compound according to any one of the preceding claims, wherein X1 and X12 to X14 are absent, R¹ and R² are absent, and X2 and X11 are amino acid residues that together form a head-to-tail cyclised lactam bridge via the N-terminus of X2 and the C-terminus of X11.

23. The compound according to any one of the preceding claims, wherein the dithioether bridge between X2 and X11 and/or X4 and X7 is of the formula —S—L—Y—L—S—, wherein:
each S is a sulfur atom and is part of the amino acid residue at X2 and X11 and/or X4 and X7;
each L is independently C₁₋₄ alkylene; and
Y is either absent, C(═O), or arylene.

24. The compound according to claim 23, wherein each L is independently C₁₋₂ alkylene.

25. The compound according to claims 23 or 24, wherein each L is methylene.

26. The compound according to any one of claims 23 to 25, wherein Y is C(═O).

27. The compound according to any one of claims 23 to 25, wherein Y is arylene selected from phenylene.

28. The compound according to claim 27, wherein Y is phenylene selected from 1,2-phenylene, 1,3-phenylene, and 1,4-phenylene.

29. The compound according to claim 27 or 28, wherein Y is 1,2-phenylene.

30. The compound according to any one of the preceding claims, wherein the bridge containing a triazole ring between X2 and X11 and/or X4 and X7 contains a 1,2,3-triazole ring.

31. The compound according to any one of the preceding claims, wherein the bridge containing a triazole ring between X2 and X11 and/or X4 and X7 is attached to positions 1 and 4 of the triazole ring.

32. The compound according to any one of the preceding claims, wherein X2 and X11 are amino acid residues who together form a lactam bridge.

33. The compound according to claim 32, wherein the location of the amide bond in the lactam bridge is closer to X11 than X2.

34. The compound according to claim 32 or 33, wherein one of the residues at position X2 and X11 is selected from Lys, Arg, Orn, bAla, 3-(4-aminophenyl)propanoyl, (3-aminomethyl)benzoyl, (4-aminomethyl)benzoyl, 4-(2-aminoethyl)benzoyl, 2-aminomethyl-phenylacetyl, 3-aminomethyl-phenylacetyl, 4-aminomethyl-phenylacetyl, 4-aminomethyl-2-pyridineacetyl, 4-aminomethyl-3-pyridineacetyl, 4-aminomethyl-2-fluoro-phenylacetyl, 4-aminomethyl-3-fluoro-phenylacetyl, 4-aminomethyl-2-methyl-phenylacetyl, 4-aminomethyl-3-methyl-phenylacetyl, 4-aminomethyl-2-methoxy-phenylacetyl, 4-aminomethyl-3-methoxy-phenylacetyl, Dab, 6-aminohexanoyl, 6-amino-4-oxahexanoyl, trans-4-aminomethyl-cyclohexyl-1-carbonyl, (4-(2-aminoethyl)-piperazine-1-yl)-acetyl, 2,4-diaminobutanoyl([2-(trimethyl-2-aminoethoxy)ethoxy]propyl), and 4-methylaminomethyl-phenylacetyl, and the other is selected from Glu and Asp.

35. The compound according to claim 34, wherein X2 is selected from Lys, Orn, bAla, 3-(4-aminophenyl)propanoyl, (3-aminomethyl)benzoyl, (4-aminomethyl)benzoyl, 4-(2-aminoethyl)benzoyl, 2-aminomethyl-phenylacetyl, 3-aminomethyl-phenylacetyl, 4-aminomethyl-phenylacetyl, 4-aminomethyl-2-pyridineacetyl, 4-aminomethyl-3-pyridineacetyl, 4-aminomethyl-2-fluoro-phenylacetyl, 4-aminomethyl-3-fluoro-phenylacetyl, 4-aminomethyl-2-methyl-phenylacetyl, 4-aminomethyl-3-methyl-phenylacetyl, 4-aminomethyl-2-methoxy-phenylacetyl, 4-aminomethyl-3-methoxy-phenylacetyl, 6-aminohexanoyl, 6-amino-4-oxahexanoyl, trans-4-aminomethyl-cyclohexyl-1-carbonyl, (4-(2-aminoethyl)-piperazine-1-yl)-acetyl, 2,4-diaminobutanoyl([2-(trimethyl-2-aminoethoxy)ethoxy]propyl), and 4-methylaminomethyl-phenylacetyl, and X11 is selected from Glu and Asp.

36. The compound according to claim 35, wherein:
X2 is Lys and X11 is Glu;
X2 is Orn and X11 is Glu;
X2 is bAla and X11 is Glu;
X2 is 3-(4-aminophenyl)propanoyl and X11 is Glu;
X2 is (3-aminomethyl)benzoyl and X11 is Glu;
X2 is (4-aminomethyl)benzoyl and X11 is Glu;
X2 is 4-(2-aminoethyl)benzoyl and X11 is Glu;
X2 is 2-aminomethyl-phenylacetyl and X11 is Glu;
X2 is 3-aminomethyl-phenylacetyl and X11 is Glu;
X2 is 4-aminomethyl-phenylacetyl and X11 is Glu;
X2 is 6-aminohexanoyl and X11 is Glu;
X2 is 6-amino-4-oxahexanoyl and X11 is Glu;
X2 is trans-4-aminomethyl-cyclohexyl-1-carbonyl and X11 is Glu;
X2 is (4-(2-aminoethyl)-piperazine-1-yl)-acetyl and X11 is Glu;
X2 is (4-(2-aminoethyl)-piperazine-1-yl)-acetyl and X11 is Asp;
X2 is 4-aminomethyl-2-pyridineacetyl and X11 is Glu;
X2 is 4-aminomethyl-3-pyridineacetyl and X11 is Glu;
X2 is 4-aminomethyl-2-fluoro-phenylacetyl and X11 is Glu;
X2 is 4-aminomethyl-3-fluoro-phenylacetyl and X11 is Glu;
X2 is 4-aminomethyl-2-methyl-phenylacetyl and X11 is Glu;
X2 is 4-aminomethyl-3-methyl-phenylacetyl and X11 is Glu;
X2 is 4-aminomethyl-2-methoxy-phenylacetyl and X11 is Glu;
X2 is 4-aminomethyl-3-methoxy-phenylacetyl and X11 is Glu;
X2 is Dab and X11 is Glu;
X2 is 2,4-diaminobutanoyl([2-(trimethyl-2-aminoethoxy)ethoxy]propyl) and X11 is Glu; or
X2 is 2,4-diaminobutanoyl([2-(trimethyl-2-aminoethoxy)ethoxy]propyl) and X11 is Glu.

37. The compound according to claim 35 or 36, wherein:
X2 is Lys and X11 is Glu;
X2 is (3-aminomethyl)benzoyl and X11 is Glu; or
X2 is 4-aminomethyl-phenylacetyl and X11 is Glu.

38. The compound according to claim 34, wherein X2 is selected from Glu and Asp, and X11 is selected from Lys, Arg, and Dab.

39. The compound according to claim 38, wherein:
X2 is Glu and X11 is Lys;
X2 is Glu and X11 is Dab; or
X2 is Asp and X11 is Arg.

40. The compound according to any one claims 1 to 31, wherein X2 and X11 are amino acid residues who together form a dithioether bridge.

41. The compound according to claim 40, wherein X2 and X11 are each independently selected from Cys and N-Me-Cys.

42. The compound according to claim 40 or 41, wherein X2 is Cys and X11 is Cys.

43. The compound according to any one claims 1 to 31, wherein X2 and X11 are amino acid residues who together form a bridge containing a triazole ring.

44. The compound according to claim 43, wherein one of the residues at position X2 and X11 is selected from Lys(N₃), azidoacetic acid, (N₃)-Ala, Dab(azidoacetic acid), and Dab((N₃)-Ala), and the other is selected from Pra, Glu(propargylamine), Dab(3-butynoic acid), and but-3-ynoic acid.

45. The compound according to claim 44, wherein X2 is selected from Lys(N₃), azidoacetic acid, and (N₃)-Ala; and X11 is selected from Pra, Glu(propargylamine), and Dab(3-butynoic acid).

46. The compound according to claim 45, wherein:
X2 is Lys(N₃) and X11 is Pra;
X2 is azidoacetic acid and X11 is Glu(propargylamine);
X2 is azidoacetic acid and X11 is Dab(3-butynoic acid);
X2 is (N₃)-Ala and X11 is Glu(propargylamine); or
X2 is (N₃)-Ala and X11 is Dab(3-butynoic acid).

47. The compound according to claim 45 or 46, wherein:
X2 is Lys(N₃) and X11 is Pra.

48. The compound according to claim 44, wherein X2 is selected from Pra, and but-3-ynoic acid; and X11 is selected from Dab(azidoacetic acid), and Dab((N₃)-Ala).

49. The compound according to claim 48, wherein:
X2 is Pra and X11 is Dab(azidoacetic acid);
X2 is Pra and X11 is Dab((N₃)-Ala); or
X2 is but-3-ynoic acid and X11 is Dab(azidoacetic acid); or
X2 is but-3-ynoic acid and X11 is Dab((N₃)-Ala).

50. The compound according to any one of the preceding claims, wherein X4 and X7 are amino acid residues who together form a dithioether bridge.

51. The compound according to claim 50, wherein X4 and X7 are each independently selected from Cys and N-Me-Cys.

52. The compound according to claim 50 or 51, wherein X4 is Cys and X7 is Cys; or X4 is N-Me-Cys and X7 is Cys.

53. The compound according to any one of claims 1 to 49, wherein X4 and X7 are amino acid residues who together form a lactam bridge.

54. The compound according to claim 53, wherein one of the residues at position X4 and X7 is Lys, Dpr, Dab, or Orn, and the other is Glu.

55. The compound according to claim 54, wherein X4 is selected from Lys, Dpr, Dab, and Orn, and X7 is Glu.

56. The compound according to claim 55, wherein:
X4 is Dpr and X7 is Glu;
X4 is Dab and X7 is Glu; or
X4 is Orn and X7 is Glu.

57. The compound according to claim 54, wherein X4 is Glu, and X7 is selected from Lys, Dpr, Dab, and Orn.

58. The compound according to claim 57, wherein:
X4 is Glu and X7 is Lys;
X4 is Glu and X7 is Dpr;
X4 is Glu and X7 is Orn; or
X4 is Glu and X7 is Dab.

59. The compound according to any one claims 1 to 49, wherein X4 and X7 are amino acid residues who together form a bridge containing a triazole ring.

60. The compound according to claim 59, wherein one of the residues at position X4 and X7 is selected from Lys(N₃) and Aha, and the other is Pra.

61. The compound according to claim 60, wherein:
X4 is Lys(N₃) and X7 is Pra; or
X4 is Aha and X7 is Pra.

62. The compound according to any one of claims 1 to 7, 9, 10, and 13 to 61, wherein X8 is Trp; X10 is D-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.

63. The compound according to any one of claims 1 to 10, and 13 to 61, wherein X8 is Tyr wherein the hydroxyl group of Tyr is optionally substituted with C₁₋₃ alkyl optionally substituted with NH₂; X10 is D-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.

64. The compound according to any one of claims 1 to 7, 9, 10, and 13 to 61, wherein X8 is Asn; X10 is D-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.

65. The compound according to any one of claims 1 to 7, 9, 10, and 13 to 61, wherein X8 is Ala; X10 is D-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.

66. The compound according to any one of claims 1 to 7, 9, 10, and 13 to 61, wherein X8 is His; X10 is D-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.

67. The compound according to any one of claims 1 to 7, 9, 10, and 13 to 61, wherein X8 is 2-Nal; X10 is D-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.

68. The compound according to any one of claims 1 to 11, and 13 to 61, wherein X8 is Tyr wherein the hydroxyl group of Tyr is optionally substituted with —CH₂CH₂NH₂; X10 is 2-Me-Leu; and X12 is Arg or is absent.

69. The compound according to any one of claims 1 to 7, 9 to 11, and 13 to 61, wherein X8 is Trp; X10 is 2-Me-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.

70. The compound according to any one of claims 1 to 11, and 13 to 61, wherein X8 is Tyr wherein the hydroxyl group of Tyr is optionally substituted with C₁₋₃ alkyl optionally substituted with NH₂; X10 is 2-Me-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.

71. The compound according to any one of claims 1 to 7, 9 to 11, and 13 to 61, wherein X8 is Asn; X10 is 2-Me-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.

72. The compound according to any one of claims 1 to 7, 9 to 11, and 13 to 61, wherein X8 is Ala; X10 is 2-Me-Leu; and X12 is selected from the group consisting of Arg, D-Arg, 2-Me-Arg, N-Me-Arg, Ser, Phe, 4-NH₂Phe, Tyr, Thr, Met, Gly, Glu, Asn, Dab, 3-(3-Pyridyl)-Ala, and 3-(4-Pyridyl)-Ala, or is absent.

73. The compound according to any one of claims 1 to 7, 9 to 11, and 13 to 61, wherein X8 is His; X10 is 2-Me-Leu; and X12 is selected from the group consisting of Arg, D-Arg, 2-Me-Arg, N-Me-Arg, Ser, Phe, 4-NH₂Phe, Tyr, Thr, Met, Gly, Glu, Asn, Dab, 3-(3-Pyridyl)-Ala, and 3-(4-Pyridyl)-Ala, or is absent.

74. The compound according to any one of claims 1 to 7, 9 to 11, and 13 to 61, wherein X8 is 2-Nal; X10 is 2-Me-Leu; and X12 is selected from the group consisting of Arg, D-Arg, 2-Me-Arg, N-Me-Arg, Ser, Phe, 4-NH₂-Phe, Tyr, Thr, Met, Gly, Glu, Asn, Dab, 3-(3-Pyridyl)-Ala, and 3-(4-Pyridyl)-Ala, or is absent.

75. The compound according to any one of claims 1 to 10, and 13 to 61, wherein X8 is Tyr wherein the hydroxyl group of Tyr is optionally substituted with —CH₂CH₂NH₂; X10 is D-Leu; and X12 is Arg or is absent.

76. The compound according to any one of claims 1 to 11, and 13 to 61, wherein X8 is Tyr wherein the hydroxyl group of Tyr is optionally substituted with —CH₂CH₂NH₂; X10 is 2-Me-Leu; and X12 is Arg or is absent.

77. The compound according to claim 1 wherein Z is an amino acid sequence selected from the group consisting of:

| | |
|---|---|
| SEQ ID NO: 1 | DC(1a)SC(2a)WQC(2a)WW[2-Me-Leu]C(1a)R |
| SEQ ID NO: 2 | LC(1a)SC(2a)WQC(2a)WWLC(1a)R |
| SEQ ID NO: 3 | DC(1a)SC(2a)WEC(2a)WWLC(1a)R |
| SEQ ID NO: 4 | DE(1c)SC(2a)WQC(2a)WWLK(1c)R |
| SEQ ID NO: 5 | DC(1a)SE(2c)WQK(2c)WWLC(1a)R |
| SEQ ID NO: 6 | D(1c)*SC(2a)WQC(2a)WWLR(1c)* |
| SEQ ID NO: 7 | DK(1c)SC(2a)WQC(2a)WWLE(1c)R |
| SEQ ID NO: 8 | D[Orn](1c)SC(2a)WQC(2a)WWLE(1c)R |
| SEQ ID NO: 9 | DE(1c)SC(2a)WQC(2a)WWL[Dab](1c)R |
| SEQ ID NO: 10 | E(1c)SC(2a)WQC(2a)WWLK(1c)R |
| SEQ ID NO: 11 | DE(1c)SC(2a)WQC(2a)WWLK(1c) |
| SEQ ID NO: 12 | E(1c)SC(2a)WQC(2a)WWLK(1c) |
| SEQ ID NO: 13 | DE(1c)SC(2a)AQC(2a)WWLK(1c)R |
| SEQ ID NO: 14 | DE(1c)SC(2a)WQC(2a)AWLK(1c)R |
| SEQ ID NO: 15 | DE(1c)SC(2a)WQC(2a)WALK(1c)R |
| SEQ ID NO: 16 | DE(1c)SC(2a)[7-Me-Trp]QC(2a)WWLK(1c)R |
| SEQ ID NO: 17 | DE(1c)SC(2a)WQC(2a)W[2-Nal]LK(1c)R |
| SEQ ID NO: 18 | K(1c)SC(2a)WQC(2a)W[2-Nal]LE(1c)R |
| SEQ ID NO: 19 | DK(1c)SC(2a)WQC(2a)W[2-Nal]LE(1c)R |
| SEQ ID NO: 20 | DK(1c)SC(2d)WQC(2d)W[2-Nal]LE(1c)R |
| SEQ ID NO: 21 | DK(1c)SC(2e)WQC(2e)W[2-Nal]LE(1c)R |
| SEQ ID NO: 22 | DK(1c)SC(2f)WQC(2f)W[2-Nal]LE(1c)R |
| SEQ ID NO: 23 | DK(1c)S[K(N3)](2g)WQ[Pra](2g)W[2-Nal]LE(1c)R |
| SEQ ID NO: 24 | DK(1c)S[Aha](2g)WQ[Pra](2g)W[2-Nal]LE(1c)R |
| SEQ ID NO: 25 | K(1c)SC(2a)[2-Nal]QC(2a)W[2-Nal]LE(1c)R |
| SEQ ID NO: 26 | K(1c)SC(2a)[Bip]QC(2a)W[2-Nal]LE(1c)R |
| SEQ ID NO: 27 | K(1c)SC(2a)[1-Me-Trp]QC(2a)W[2-Nal]LE(1c)R |
| SEQ ID NO: 28 | DK(1c)SC(2a)WQC(2a)[2-Nal][2-Nal]LE(1c)R |
| SEQ ID NO: 29 | K(1c)SC(2a)WQC(2a)[Y(2-aminoethoxy)][2-Nal]LE(1c)R |
| SEQ ID NO: 30 | K(1c)SC(2a)WQC(2a)H[2-Nal]LE(1c)R |
| SEQ ID NO: 31 | K(1c)SC(2a)WQC(2a)W[F(4-F)]LE(1c)R |
| SEQ ID NO: 32 | K(1c)SC(2a)WQC(2a)W[1-Me-Trp]LE(1c)R |
| SEQ ID NO: 33 | C(1a)TC(2a)WEC(2a)WW[2-Me-Leu]C(1a)S |
| SEQ ID NO: 34 | GK(1c)TC(2a)WEC(2c)WW[2-Me-Leu]E(1c)S |
| SEQ ID NO: 35 | GC(1a)TC(2a)WEC(2a)W[2-Nal][2-Me-Leu]C(1a)R |
| SEQ ID NO: 36 | [bAla](1c)*SC(2a)WQC(2a)W[2-Nal]LE(1c)R |
| SEQ ID NO: 37 | [(3-Aminomethyl)benzoyl](1c)*SC(2a)WQC(2a)W[2-Nal]LE(1c)R |
| SEQ ID NO: 38 | [(3-Aminomethyl)benzoyl](1c)*SC(2a)WQC(2a)[Y(2-aminoethoxy)][2- |
| | Nal][2-Me-Leu]E(1c)R-[NH2] |
| SEQ ID NO: 39 | [(3-Aminomethyl)benzoyl](1c)*SC(2a)WQC(2a)[Y(2-aminoethoxy)][2- |
| | Nal]LE(1c)R |
| SEQ ID NO: 40 | [(3-Aminomethyl)benzoyl](1c)*SC(2a)WQC(2a)W[2-Nal][2-Me-Leu]E(1c)R |
| SEQ ID NO: 41 | [(3-Aminomethyl)benzoyl](1c)*SC(2a)WQC(2a)[Y(2-aminoethoxy)][2- |
| | Nal][2-Me-Leu]E(1c)[{d}R] |
| SEQ ID NO: 42 | [(3-Aminomethyl)benzoyl](1c)*SC(2a)WQC(2a)[Y(2-aminoethoxy)][2- |
| | Nal][2-Me-Leu]E(1c)[2-Me-Arg] |
| SEQ ID NO: 43 | [(3-Aminomethyl)benzoyl](1c)*SC(2a)WQC(2a)[Y(2-aminoethoxy)][2- |
| | Nal][2-Me-Leu]E(1c)[N-Me-Arg] |
| SEQ ID NO: 44 | [(3-Aminomethyl)benzoyl](1c)*SC(2a)WQC(2a)[Y(2-aminoethoxy)][2- |
| | Nal][2-Me-Leu]E(1c)S |
| SEQ ID NO: 45 | [(3-Aminomethyl)benzoyl](1c)*SC(2a)WQC(2a)[Y(2-aminoethoxy)][2- |
| | Nal][2-Me-Leu]E(1c)[F(4-NH2)] |
| SEQ ID NO: 46 | [(3-Aminomethyl)benzoyl](1c)*SC(2a)WQC(2a)[Y(2-aminoethoxy)][2- |
| | Nal][{d}L]E(1c)Y |
| SEQ ID NO: 47 | [Ac]-K(1c)SC(2a)WQC(2a)[Y(2-aminoethoxy)][2-Nal][2-Me-Leu]E(1c)R |
| SEQ ID NO: 48 | [Ac]-K(1c)SC(2a)WQC(2a)[Y(2-aminoethoxy)][2-Nal][{d}L]E(1c)R |
| SEQ ID NO: 49 | [(4-Aminomethyl)benzoyl](1c)*SC(2a)WQC(2a)[Y(2-aminoethoxy)][2- |
| | Nal][2-Me-Leu]E(1c)R |
| SEQ ID NO: 50 | [4-(2-Aminoethyl)benzoyl](1c)*SC(2a)WQC(2a)[Y(2-aminoethoxy)][2- |
| | Nal][2-Me-Leu]E(1c)R |
| SEQ ID NO: 51 | [3-(4-Aminophenyl)propanoyl](1c)*SC(2a)WQC(2a)[Y(2-aminoethoxy)][2- |
| | Nal][2-Me-Leu]E(1c)R |
| SEQ ID NO: 52 | [4-Aminomethyl-phenylacetyl](1c)*SC(2a)WQC(2a)[Y(2-aminoethoxy)][2- |
| | Nal][2-Me-Leu]E(1c)R |
| SEQ ID NO: 53 | DC(1a)SC(2b)WQC(2b)WWLC(1a)R |
| SEQ ID NO: 54 | K(1c)SC(2b)WQC(2b)[Y(2-aminoethoxy)][2-Nal]LE(1c)R |
| SEQ ID NO: 55 | DE(1c)QC(2a)WQC(2a)YW[2-Me-Leu]K(1c)R |
| SEQ ID NO: 56 | DE(1c)QC(2a)WQC(2a)[2-Nal]W[2-Me-Leu]K(1c)R |
| SEQ ID NO: 57 | DE(1c)TC(2a)WQC(2a)W[2-Nal][2-Me-Leu]K(1c) |
| SEQ ID NO: 58 | DE(1c)SC(2a)WQC(2a)W[2-Nal][2-Me-Leu]K(1c)ENG |
| SEQ ID NO: 59 | DE(1c)SC(2a)WQC(2a)W[2-Nal][2-Me-Leu]K(1c)NG |
| SEQ ID NO: 60 | DE(1c)SC(2a)WQC(2a)W[2-Nal][2-Me-Leu]K(1c)G |
| SEQ ID NO: 61 | DE(1c)SC(2a)WQC(2a)W[2-Nal][2-Me-Lys]K(1c)ENG |
| SEQ ID NO: 62 | DC(1a)QC(2a)WQC(2a)[2-Nal]W[2-Me-Leu]C(1a)R |
| SEQ ID NO: 63 | DC(1a)QC(2a)WQC(2a)YW[2-Me-Leu]C(1a)R |
| SEQ ID NO: 64 | DC(1a)QC(2a)WQC(2a)WW[2-Me-Leu]C(1a)ENG |
| SEQ ID NO: 65 | [(3-Aminomethyl)benzoyl](1c)*S[Dpr](2c)WQE(2c)[Y(2-aminoethoxy)][2- |
| | Nal][2-Me-Leu]E(1c)R |
| SEQ ID NO: 66 | [(3-Aminomethyl)benzoyl](1c)*SE(2c)WQ[Dpr](2c)[Y(2-aminoethoxy)][2- |
| | Nal][2-Me-Leu]E(1c)R |
| SEQ ID NO: 67 | [(3-Aminomethyl)benzoyl](1c)*SE(2c)WQ[Dab](2c)[Y(2-aminoethoxy)][2- |
| | Nal][2-Me-Leu]E(1c)R |
| SEQ ID NO: 68 | [(3-Aminomethyl)benzoyl](1c)*S[Orn](2c)WQE(2c)[Y(2-aminoethoxy)][2- |
| | Nal][2-Me-Leu]E(1c)R |
| SEQ ID NO: 69 | [(3-Aminomethyl)benzoyl](1c)*SE(2c)WQ[Orn](2c)[Y(2-aminoethoxy)][2- |
| | Nal][2-Me-Leu]E(1c)R |
| SEQ ID NO: 70 | [(3-Aminomethyl)benzoyl](1c)*SC(2a)[7-Me-Trp]QC(2a)[Y(2- |
| | aminoethoxy)][2-Nal][2-Me-Leu]E(1c)R |
| SEQ ID NO: 71 | [(3-Aminomethyl)benzoyl](1c)*SC(2a)[7-Ph-Trp]QC(2a)[Y(2- |
| | aminoethoxy)][2-Nal][2-Me-Leu]E(1c)R |
| SEQ ID NO: 72 | [(3-Aminomethyl)benzoyl](1c)*SC(2a)[7-(Naphth-2-yl)-Trp]QC(2a)[Y(2- |
| | aminoethoxy)][2-Nal][2-Me-Leu]E(1c)R |
| SEQ ID NO: 73 | [(3-Aminomethyl)benzoyl](1c)*SC(2a)WQC(2a)[Y(2-aminoethoxy)][3-(3- |
| | Quinolinyl)-Ala][2-Me-Leu]E(1c)R |
| SEQ ID NO: 74 | [(3-Aminomethyl)benzoyl](1c)*SC(2a)WQC(2a)[Y(2-aminoethoxy)][6-CI- |
| | Trp][2-Me-Leu]E(1c)R |
| SEQ ID NO: 75 | [(3-Aminomethyl)benzoyl](1c)*SC(2a)WQC(2a)[Y(2-aminoethoxy)][2- |
| | Nal][4-aminotetrahydro-2H-pyran-4-acetyl]E(1c)R |
| SEQ ID NO: 76 | [(3-Aminomethyl)benzoyl](1c)*SC(2a)WQC(2a)[Y(2-aminoethoxy)][2- |
| | Nal][2-Me-Leu]E(1c)[3-(3-Pyridyl)-Ala] |
| SEQ ID NO: 77 | [(3-Aminomethyl)benzoyl](1c)*SC(2a)WQC(2a)[Y(2-aminoethoxy)][2- |
| | Nal][2-Me-Leu]E(1c)[3-(4-Pyridyl)-Ala] |
| SEQ ID NO: 78 | [(3-Aminomethyl)benzoyl](1c)*SC(2a)WQC(2a)[Y(2-aminoethoxy)][2- |
| | Nal][2-Me-Leu]E(1c)R[3-(3-Pyridyl)-Ala] |
| SEQ ID NO: 79 | [(3-Aminomethyl)benzoyl](1c)*SC(2a)WQC(2a)[Y(2-aminoethoxy)][2- |
| | Nal][2-Me-Leu]E(1c)R[3-(4-Pyridyl)-Ala] |
| SEQ ID NO: 80 | [4-Aminomethyl-phenylacetyl](1c)*SC(2a)WQC(2a)[Y(2-aminoethoxy)][2- |
| | Nal][2-Me-Leu]E(1c)[{d}R] |
| SEQ ID NO: 81 | [4-Aminomethyl-phenylacetyl](1c)*SC(2a)WQC(2a)[Y(2-aminoethoxy)][2- |
| | Nal][2-Me-Leu]E(1c)S |
| SEQ ID NO: 82 | [4-Aminomethyl-phenylacetyl](1c)*SC(2a)WQC(2a)[Y(2-aminoethoxy)][2- |
| | Nal][2-Me-Leu]E(1c)[Dab] |
| SEQ ID NO: 83 | [4-Aminomethyl-phenylacetyl](1c)*TC(2a)WQC(2a)[Y(2-aminoethoxy)][2- |
| | Nal][2-Me-Leu]E(1c)[{d}R] |
| SEQ ID NO: 84 | K(1c)SC(2a)WQC(2a)[Y(2-aminoethoxy)][2-Nal][2-Me-Leu]E(1c)[{d}R] |
| SEQ ID NO: 85 | K(1c) TC(2a)WQC(2a)[Y(2-aminoethoxy)][2-Nal][2-Me-Leu]E(1c)[{d}R] |
| SEQ ID NO: 86 | K(1c)SC(2a)WQC(2a)[Y(2-aminoethoxy)][2-Nal][2-Me-Leu]E(1c)S |
| SEQ ID NO: 87 | [6-Aminohexanoyl](1c)*SC(2a)WQC(2a)[Y(2-aminoethoxy)][2-Nal][2-Me- |
| | Leu]E(1c)[{d}R] |
| SEQ ID NO: 88 | [6-Amino-4-oxahexanoyl](1c)*SC(2a)WQC(2a)[Y(2-aminoethoxy)][2- |
| | Nal][2-Me-Leu]E(1c)[{d}R] |
| SEQ ID NO: 89 | [trans-4-Aminomethyl-cyclohexyl-1-carbonyl](1c)*[beta-homo- |
| | Ser]C(2a)WQC(2a)[Y(2-aminoethoxy)][2-Nal][2-Me-Leu]E(1c)[{d}R] |
| SEQ ID NO: 90 | [(4-(2-Aminoethyl)-piperazine-1-yl)-acetyl](1c)*SC(2a)WQC(2a)[Y(2- |
| | aminoethoxy)][2-Nal][2-Me-Leu]E(1c)[{d}R] |
| SEQ ID NO: 91 | [(4-(2-Aminoethyl)-piperazine-1-yl)-acetyl](1c)*SC(2a)WQC(2a)[Y(2- |
| | aminoethoxy)][2-Nal][2-Me-Leu]D(1c)[{d}R] |
| SEQ ID NO: 92 | [3-Aminomethyl-phenylacetyl](1c)*SC(2a)WQC(2a)[Y(2-aminoethoxy)][2- |
| | Nal][2-Me-Leu]E(1c)[{d}R] |
| SEQ ID NO: 93 | [2-Aminomethyl-phenylacetyl](1c)*SC(2a)WQC(2a)[Y(2-aminoethoxy)][2- |
| | Nal][2-Me-Leu]E(1c)[{d}R] |
| SEQ ID NO: 94 | [K(N3)](1g)SC(2a)WQC(2a)[Y(2-aminoethoxy)][2-Nal][2-Me- |
| | Leu][Pra](1g)[{d}R] |
| SEQ ID NO: 95 | [Azidoacetic acid](1g)*SC(2a)WQC(2a)[Y(2-aminoethoxy)][2-Nal][2-Me- |
| | Leu][Glu(propargylamine)] (1g)[{d}R] |
| SEQ ID NO: 96 | [Azidoacetic acid](1g)*SC(2a)WQC(2a)[Y(2-aminoethoxy)][2-Nal][2-Me- |
| | Leu][Dab(3-butynoic acid)](1g)[{d}R] |
| SEQ ID NO: 97 | [but-3-ynoic acid](1g)*SC(2a)WQC(2a)[Y(2-aminoethoxy)][2-Nal][2-Me- |
| | Leu][Dab(azidoacetic acid)](1g)[{d}R] |
| SEQ ID NO: 98 | [(N3)-Ala](1g)*SC(2a)WQC(2a)[Y(2-aminoethoxy)][2-Nal][2-Me- |
| | Leu][Glu(propargylamine)](1g)[{d}R] |
| SEQ ID NO: 99 | [(N3)-Ala](1g)*SC(2a)WQC(2a)[Y(2-aminoethoxy)][2-Nal][2-Me- |
| | Leu][Dab(3-butynoic acid)](1g)[{d}R] |
| SEQ ID NO: 100 | [but-3-ynoic acid](1g)*SC(2a)WQC(2a)[Y(2-aminoethoxy)][2-Nal][2-Me- |
| | Leu][Dab((N3)-Ala))](1g)[{d}R] |
| SEQ ID NO: 101 | [4-Aminomethyl-2-pyridineacetyl](1c)*SC(2a)WQC(2a)[Y(2- |
| | aminoethoxy)][2-Nal][2-Me-Leu]E(1c)[{d}R] |
| SEQ ID NO: 102 | [4-Aminomethyl-3-pyridineacetyl](1c)*SC(2a)WQC(2a)[Y(2- |
| | aminoethoxy)][2-Nal][2-Me-Leu]E(1c)[{d}R] |
| SEQ ID NO: 103 | [4-Aminomethyl-2-fluoro-phenylacetyl](1c)*SC(2a)WQC(2a)[Y(2- |
| | aminoethoxy)][2-Nal][2-Me-Leu]E(1c)[{d}R] |
| SEQ ID NO: 104 | [4-Aminomethyl-3-methyl-phenylacetyl](1c)*SC(2a)WQC(2a)[Y(2- |
| | aminoethoxy)][2-Nal][2-Me-Leu]E(1c)[{d}R] |
| SEQ ID NO: 105 | [4-Aminomethyl-3-methoxy-phenylacetyl](1c)*SC(2a)WQC(2a)[Y(2- |
| | aminoethoxy)][2-Nal][2-Me-Leu]E(1c)[{d}R] |
| SEQ ID NO: 106 | [but-3-ynoic acid](1h)*SC(2a)WQC(2a)[Y(2-aminoethoxy)][2-Nal][2-Me- |
| | Leu][Dab(azidoacetic acid)] (1h)[{d}R] |
| SEQ ID NO: 107 | [but-3-ynoic acid](1h)*SC(2a)WQC(2a)[Y(2-aminoethoxy)][2-Nal][2-Me- |
| | Leu][Dab((N3)-Ala)](1h)[{d}R] |
| SEQ ID NO: 108 | [4-Aminomethyl-phenylacetyl](1c)*SC(2a)WQC(2a)[Y(2-aminoethoxy)][2- |
| | Nal][2-Me-Leu]E(1c)[{d}R] |
| SEQ ID NO: 109 | [4-Aminomethyl-phenylacetyl](1c)*SC(2a)WQC(2a)[Y(2-aminoethoxy)][2- |
| | Nal][2-Me-Leu]E(1c)[{d}2,4-Diaminobutanoyl([2-(trimethyl-2- |
| | aminoethoxy)ethoxy]propyl)] |
| SEQ ID NO: 110 | [4-Aminomethyl-phenylacetyl](1c)*SC(2a)WQC(2a)[Dab][2-Nal][2-Me- |
| | Leu]E(1c)[{d}R] |
| SEQ ID NO: 111 | [4-Aminomethyl-phenylacetyl](1c)*SC(2a)WQC(2a)[2,4- |
| | Diaminobutanoyl([2-(trimethyl-2-aminoethoxy)ethoxy]propyl)][2-Nal][2-Me- |
| | Leu]E(1c)[{d}R] |
| SEQ ID NO: 112 | [Dab](1c)*SC(2a)WQC(2a)[Y(2-aminoethoxy)][2-Nal][2-Me- |
| | Leu]E(1c)[{d}R] |
| SEQ ID NO: 113 | [2,4-Diaminobutanoyl([2-(trimethyl-2- |
| | aminoethoxy)ethoxy]propyl)](1c)*SC(2a)WQC(2a)[Y(2-aminoethoxy)][2- |
| | Nal][2-Me-Leu]E(1c)[{d}R] |
| SEQ ID NO: 114 | [4-Aminomethyl-phenylacetyl](1c)*SC(2a)WQC(2a)[F(4-NH2-(2-(trimethyl- |
| | 2-aminoethoxy)ethoxy)propyl)][2-Nal][2-Me-Leu]E(1c)[{d}R] |
| SEQ ID NO: 115 | [4-Aminomethyl-phenylacetyl](1c)*SC(2a)WQC(2a)[Y(2-aminoethoxy)][2- |
| | Nal][2-Me-Leu]E(1c)[{d}Q] |
| SEQ ID NO: 116 | [4-Aminomethyl-phenylacetyl](1c)*SC(2a)WQC(2a)[Y(2-aminoethoxy)][2- |
| | Nal][2-Me-Leu]E(1c)[{d}E] |
| SEQ ID NO: 117 | [4-Aminomethyl-phenylacetyl](1c)*SC(2a)WQC(2a)[Y(2-aminoethoxy)][2- |
| | Nal][2-Me-Leu]E(1c)[{d}H] |
| SEQ ID NO: 118 | [4-Aminomethyl-phenylacetyl](1c)*SC(2a)WQC(2a)[Y(2-aminoethoxy)][2- |
| | Nal][2-Me-Leu]E(1c)[3-Aminopropanoy]] |
| SEQ ID NO: 119 | [4-Aminomethyl-phenylacetyl](1c)*SC(2a)WQC(2a)[Y(2-aminoethoxy)][2- |
| | Nal][2-Me-Leu]E(1c)[3-Aminopropanoy] |
| SEQ ID NO: 120 | [4-Aminomethyl-phenylacetyl](1c)*SC(2a)WQC(2a)[Y(2-aminoethoxy)][2- |
| | Nal][2-Me-Leu]E(1c)[Dab][3-(3-Pyridyl)-Ala] |
| SEQ ID NO: 121 | [4-Aminomethyl-phenylacetyl](1c)*SC(2a)WQC(2a)[Y(2-aminoethoxy)][6- |
| | F-Trp][2-Me-Leu]E(1c)[Dab][3-(3-Pyridyl)-Ala] |
| SEQ ID NO: 122 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala] |
| SEQ ID NO: 123 | [4-Aminomethyl-phenylacetyl](1c)*S[Dab](2c)WQE(2c)[Y(2- |
| | aminoethoxy)][2-Nal][2-Me-Leu]E(1c)[Dab][3-(3-Pyridyl)-Ala] |
| SEQ ID NO: 124 | [4-Aminomethyl-phenylacetyl](1c)*SE(2c)WQ[Orn](2c)[Y(2- |
| | aminoethoxy)][2-Nal][2-Me-Leu]E(1c)[Dab][3-(3-Pyridyl)-Ala] |
| SEQ ID NO: 125 | [4-Aminomethyl-phenylacetyl](1c)*SE(2c)WQ[Dab](2c)[Y(2- |
| | aminoethoxy)][2-Nal][2-Me-Leu]E(1c)[Dab] |
| SEQ ID NO: 126 | [4-Aminomethyl-phenylacetyl](1c)*VE(2c)WQ[Dab](2c)[Y(2- |
| | aminoethoxy][2-Nal][2-Me-Leu]E(1c)[Dab] |
| SEQ ID NO: 127 | [4-Aminomethyl-phenylacetyl](1c)*IE(2c)WQ[Dab](2c)[Y(2- |
| | aminoethoxy)][2-Nal][2-Me-Leu]E(1c)[Dab] |
| SEQ ID NO: 128 | [4-Aminomethyl-phenylacetyl](1c)*SE(2c)WV[Dab](2c)[Y(2- |
| | aminoethoxy)][2-Nal][2-Me-Leu]E(1c)[Dab] |
| SEQ ID NO: 129 | [4-Aminomethyl-phenylacetyl](1c)*SE(2c)WH[Dab](2c)[Y(2- |
| | aminoethoxy)][2-Nal][2-Me-Leu]E(1c)[Dab] |
| SEQ ID NO: 130 | [4-Aminomethyl-phenylacetyl](1c)*SE(2c)WQ[Dab](2c)F[2-Nal][2-Me- |
| | Leu]E(1c)[Dab] |
| SEQ ID NO: 131 | [4-Aminomethyl-phenylacetyl](1c)*SE(2c)WQ[Dab](2c)V[2-Nal][2-Me- |
| | Leu]E(1c)[Dab] |
| SEQ ID NO: 132 | [4-Aminomethyl-phenylacetyl](1c)*SE(2c)WQ[Dab](2c)[F(4-Me)][2-Nal][2- |
| | Me-Leu]E(1c)[Dab] |
| SEQ ID NO: 133 | [4-Aminomethyl-phenylacetyl](1c)*SE(2c)WQ[Dab](2c)[Y(2- |
| | aminoethoxy)]H[2-Me-Leu]E(1c)[Dab] |
| SEQ ID NO: 134 | [4-Aminomethyl-phenylacetyl](1c)*SE(2c)WQ[Dab](2c)[Y(2- |
| | aminoethoxy)][F(3-F)][2-Me-Leu]E(1c)[Dab] |
| SEQ ID NO: 135 | [4-Aminomethyl-phenylacetyl](1c)*SE(2c)WQ[Dab](2c)[Y(2- |
| | aminoethoxy)][F(3,4-Me)][2-Me-Leu]E(1c)[Dab] |
| SEQ ID NO: 136 | [4-Aminomethyl-phenylacetyl](1c)*SE(2c)WQ[Dab](2c)[Y(2- |
| | aminoethoxy)][2-Nal][2-Me-Val]E(1c)[Dab] |
| SEQ ID NO: 137 | [4-Aminomethyl-phenylacetyl](1c)*SE(2c)WQ[Dab](2c)[Y(2- |
| | aminoethoxy)][2-Nal][2-Me-Leu]E(1c) |
| SEQ ID NO: 138 | [4-Aminomethyl-phenylacetyl](1c)*SE(2c)[4-F-Trp]Q[Dab](2c)[Y (2- |
| | aminoethoxy)][2-Nal][2-Me-Leu]E(1c)[Dab] |
| SEQ ID NO: 139 | [4-Aminomethyl-phenylacetyl](1c)*SE(2c)[7-F-Trp]Q[Dab](2c)[Y (2- |
| | aminoethoxy)][2-Nal][2-Me-Leu]E(1c)[Dab] |
| SEQ ID NO: 140 | [4-Methylaminomethyl-phenylacetyl](1c)*SC(2a)WQC(2a)[Y(2- |
| | aminoethoxy)][2-Nal][2-Me-Leu]E(1c)[Dab] |
| SEQ ID NO: 141 | [4-Aminomethyl-phenylacetyl](1c)*[N-Me-Ser]C(2a)WQC(2a)[Y(2- |
| | aminoethoxy)][2-Nal][2-Me-Leu]E(1c)[Dab] |
| SEQ ID NO: 142 | [4-Aminomethyl-phenylacetyl](1c)*S[N-Me-Cys](2a)WQC(2a)[Y(2- |
| | aminoethoxy)][2-Nal][2-Me-Leu]E(1c)[Dab] |
| SEQ ID NO: 143 | [4-Aminomethyl-phenylacetyl](1c)*SC(2a)[N-Me-Trp]QC(2a)[Y(2- |
| | aminoethoxy)][2-Nal][2-Me-Leu]E(1c)[Dab] |
| SEQ ID NO: 144 | [4-Aminomethyl-phenylacetyl](1c)*SC(2a)W[N-Me-Gln]C(2a)[Y(2- |
| | aminoethoxy)][2-Nal][2-Me-Leu]E(1c)[Dab] |
| SEQ ID NO: 145 | [4-Aminomethyl-phenylacetyl](1c)*[Q(pyrrolidin)]E(2c)WQ[Dab](2c)[Y(2- |
| | aminoethoxy)][2-Nal][2-Me-Leu]E(1c)[Dab] |
| SEQ ID NO: 146 | [4-Aminomethyl-phenylacetyl](1c)*SE(2c)W[Q(pyrrolidin)][Dab](2c)[Y(2- |
| | aminoethoxy)][2-Nal][2-Me-Leu]E(1c)[Dab] |
| SEQ ID NO: 147 | [4-Aminomethyl-phenylacetyl](1c)*SC(2a)WQC(2a)[Y(2-aminoethoxy)][2- |
| | Nal][2-Me-Leu]E(1c)R[3-(3-Pyridyl)-Ala] |
| SEQ ID NO: 148 | [(3-Aminomethyl)benzoyl](1c)*SC(2a)WQC(2a)[Y(2-aminoethoxy)][2- |
| | Nal][2-Me-Leu]E(1c)[Dab][3-(3-Pyridyl)-Ala] |
| SEQ ID NO: 149 | [(3-Aminomethyl)benzoyl](1c)*SC(2a)WQC(2a)][Y(2-aminoethoxy)][2- |
| | Nal][2-Me-Leu]E(1c)R[3-(3-Quinolinyl)-Ala] |
| SEQ ID NO: 150 | [(3-Aminomethyl)benzoyl](1c)*SC(2a)WQC(2a)][Y(2-aminoethoxy)][2- |
| | Nal][2-Me-Leu]E(1c)R[{d}[3-(3-Pyridyl)-Ala]] |
| SEQ ID NO: 151 | [(3-Aminomethyl)benzoyl](1c)*SC(2a)WQC(2a)][Y(2-aminoethoxy)][2- |
| | Nal][2-Me-Leu]E(1c)R[3-Amino-3-(3′-pyridyl)propionyl] |
| SEQ ID NO: 152 | [(3-Aminomethyl)benzoyl](1c)*SC(2a)WQC(2a)][Y(2-aminoethoxy)][2- |
| | Nal][2-Me-Leu]E(1c)R[F(3-F)] |
| SEQ ID NO: 153 | [(3-Aminomethyl)benzoyl](1c)*SC(2a)WQC(2a)][Y(2-aminoethoxy)][2- |
| | Nal][2-Me-Leu]E(1c)R[F(3,5-F)] |
| SEQ ID NO: 154 | [(3-Aminomethyl)benzoyl](1c)*SC(2a)WQC(2a)][Y(2-aminoethoxy)][2- |
| | Nal][2-Me-Leu]E(1c)R[4-Aminomethyl-2-pyridineacetyl] |
| SEQ ID NO: 155 | K(1c)TC(2a)WQC(2a)[Y(2-aminoethoxy)][2-Nal][2-Me-Leu]E(1c)[Dab][3- |
| | (3-Pyridyl)-Ala] |
| SEQ ID NO: 156 | [(3-Aminomethyl)benzoyl](1c)*IE(2c)WQ[Dab](2c)[Y (2-aminoethoxy)][2- |
| | Nal][2-Me-Val]E(1c)[Dab][3-(3-Pyridyl)-Ala] |
| SEQ ID NO: 157 | [4-Aminomethyl-phenylacetyl](1c)*IE(2c)WQ[Dab](2c)[Y(2- |
| | aminoethoxy)][2-Nal][2-Me-Val]E(1c)[Dab][3-(3-Pyridyl)-Ala] |
| SEQ ID NO: 158 | [4-Aminomethyl-phenylacetyl](1c)*IE(2c)WQ[Dab](2c)[Y(2- |
| | aminoethoxy)][F(3,4-Me)][2-Me-Val]E(1c)G[3-(3-Pyridyl)-Ala] |
| SEQ ID NO: 159 | [4-Aminomethyl-phenylacetyl](1c)*IE(2c)WQ[Dab](2c)[F(4-Me)][F(3,4- |
| | Me)][2-Me-Val]E(1c)G[3-(3-Pyridyl)-Ala] |
| SEQ ID NO: 160 | [4-Aminomethyl-phenylacetyl](1c)*IE(2c)WQ[Dab](2c)[F(4-Me)][F(3,4- |
| | Me)][2-Me-Val]E(1c)[GABA][3-(3-Pyridyl)-Ala] |
| SEQ ID NO: 161 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-pyridylacetyl)] |
| SEQ ID NO: 162 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-pyridylpropionyl)] |
| SEQ ID NO: 163 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-fluorobenzoyl)] |
| SEQ ID NO: 164 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-fluorophenylacetyl)] |
| SEQ ID NO: 165 | [4-Aminomethyl-phenylacetyl](1c)*IE(2c)WQ[Dab](2c)[Y(2- |
| | aminoethoxy)][F(3,4-Me)][2-Me-Val]E(1c)[Dab][2-Me-3-(3-Pyridyl)-Ala] |
| SEQ ID NO: 166 | [4-Aminomethyl-phenylacetyl](1c)*IE(2c)WQ[Dab](2c)[Y(Me)][F(3,4- |
| | Me)][2-Me-Val]E(1c)[Dab][2-Me-3-(3-Pyridyl)-Ala] |
| SEQ ID NO: 167 | [4-Aminomethyl-phenylacetyl](1c)*IE(2c)WQ[Dab](2c)[2-Me-Phe] [F(3,4- |
| | Me)][2-Me-Val]E(1c)[Dab][2-Me-3-(3-Pyridyl)-Ala] |
| SEQ ID NO: 168 | [4-Aminomethyl-phenylacetyl](1c)*IE(2c)WQ[Dab](2c)[Bip][F(3,4-Me)][2- |
| | Me-Val]E(1c)[Dab][2-Me-3-(3-Pyridyl)-Ala] |
| SEQ ID NO: 169 | [4-Aminomethyl-phenylacetyl](1c)*IE(2c)WQ[Dab](2c)[2-Me-F(4-F)][F(3,4- |
| | Me)][2-Me-Val]E(1c)[Dab][2-Me-3-(3-Pyridyl)-Ala] |
| SEQ ID NO: 170 | [4-Aminomethyl-phenylacetyl](1c)*IE(2c)WQ[Dab](2c)[{d}F(4-F)][F(3,4- |
| | Me)][2-Me-Val]E(1c)[Dab][2-Me-3-(3-Pyridyl)-Ala] |
| SEQ ID NO: 171 | [4-Aminomethyl-phenylacetyl](1c)*IE(2c)WQ[Dab](2c)[F(4-CI)][F(3,4- |
| | Me)][2-Me-Val]E(1c)[Dab][2-Me-3-(3-Pyridyl)-Ala] |
| SEQ ID NO: 172 | [4-Aminomethyl-phenylacetyl](1c)*IE(2c)WQ[Dab](2c)[alpha-Me- |
| | Trp][F(3,4-Me)][2-Me-Val]E(1c)[Dab][2-Me-3-(3-Pyridyl)-Ala] |
| SEQ ID NO: 173 | [4-Aminomethyl-phenylacetyl](1c)*IE(2c)WQ[Dab](2c)[3,3-Diphenyl- |
| | Ala][F(3,4-Me)][2-Me-Val]E(1c)[Dab][2-Me-3-(3-Pyridyl)-Ala] |
| SEQ ID NO: 174 | [4-Aminomethyl-phenylacetyl](1c)*IE(2c)WQ[Dab](2c)[Phg][F(3,4-Me)][2- |
| | Me-Val]E(1c)[Dab][2-Me-3-(3-Pyridyl)-Ala] |
| SEQ ID NO: 175 | [4-Aminomethyl-phenylacetyl](1c)*IE(2c)WQ[Dab](2c)[F(4-Me)][1-Me- |
| | Trp][2-Me-Val]E(1c)[Dab][2-Me-3-(3-Pyridyl)-Ala] |
| SEQ ID NO: 176 | [4-Aminomethyl-phenylacetyl](1c)*IE(2c)WQ[Dab](2c)[F(4-Me)][Bip][2-Me- |
| | Val]E(1c)[Dab][2-Me-3-(3-Pyridyl)-Ala] |
| SEQ ID NO: 177 | [4-Aminomethyl-phenylacetyl](1c)*IE(2c)WQ[Dab](2c)[F(4-Me)][{d}6-F- |
| | Trp][2-Me-Val]E(1c)[Dab][2-Me-3-(3-Pyridyl)-Ala] |

or a pharmaceutically acceptable salt or solvate thereof;
wherein:
*= bridge uses the peptide backbone amine or carboxylic acid at the N- or C-terminus, not the side chain amine or carboxylic acid
(1a) = [2,11] 1,3-dithio-propan-2-one bridge; (1c) = [2,11] lactam bridge; (1g) = [2,11] 1,4-disubstituted 1,2,3-triazole bridge; (1h) = [2,11] 1,5-disubstituted 1,2,3-triazole bridge; (2a) = [4,7] 1,3-dithio-propan-2-one bridge; (2c) = [4,7] lactam bridge; (2d) = [4,7] 1,2-phenylenedimethanethiol bridge; (2e) = [4,7] 1,3-phenylenedimethanethiol bridge; (2f) = [4,7] 1,4-phenylenedimethanethiol bridge; (2g) = [4,7] 1,4-disubstituted 1,2,3-triazole bridge.

78. A compound according to claim 1 which is selected from:

| | |
|---|---|
| 1 | H-DC(1a)SC(2a)WQC(2a)WW[2-Me-Leu]C(1a)R-[NH2] |
| 2 | H-LC(1a)SC(2a)WQC(2a)WWLC(1a)R-[NH2] |
| 3 | H-DC(1a)SC(2a)WEC(2a)WWLC(1a)R-[NH2] |
| 4 | H-DE(1c)SC(2a)WQC(2a)WWLK(1c)R-[NH2] |
| 5 | H-DC(1a)SE(2c)WQK(2c)WWLC(1a)R-[NH2] |
| 6 | D(1c)*SC(2a)WQC(2a)WWLR(1c)* |
| 7 | [Ac]-DK(1c)SC(2a)WQC(2a)WWLE(1c)R-[NH2] |
| 8 | [Ac]-D[Orn](1c)SC(2a)WQC(2a)WWLE(1c)R-[NH2] |
| 9 | H-DE(1c)SC(2a)WQC(2a)WWL[Dab](1c)R-[NH2] |
| 10 | [Ac]-E(1c)SC(2a)WQC(2a)WWLK(1c)R-[NH2] |
| 11 | [Ac]-DE(1c)SC(2a)WQC(2a)WWLK(1c)-[NH2] |
| 12 | [Ac]-E(1c)SC(2a)WQC(2a)WWLK(1c)-[NH2] |
| 13 | [Ac]-DE(1c)SC(2a)AQC(2a)WWLK(1c)R-[NH2] |
| 14 | [Ac]-DE(1c)SC(2a)WQC(2a)AWLK(1c)R-[NH2] |
| 15 | [Ac]-DE(1c)SC(2a)WQC(2a)WALK(1c)R-[NH2] |
| 16 | [Ac]-DE(1c)SC(2a)[7-Me-Trp]QC(2a)WWLK(1c)R-[NH2] |
| 17 | [Ac]-DE(1c)SC(2a)WQC(2a)W[2-Nal]LK(1c)R-[NH2] |
| 18 | [Ac]-K(1c)SC(2a)WQC(2a)W[2-Nal]LE(1c)R-[NH2] |
| 19 | [Ac]-DK(1c)SC(2a)WQC(2a)W[2-Nal]LE(1c)R-[NH2] |
| 20 | [Ac]-DK(1c)SC(2d)WQC(2d)W[2-Nal]LE(1c)R-[NH2] |
| 21 | [Ac]-DK(1c)SC(2e)WQC(2e)W[2-Nal]LE(1c)R-[NH2] |
| 22 | [Ac]-DK(1c)SC(2f)WQC(2f)W[2-Nal]LE(1c)R-[NH2] |
| 23 | [Ac]-DK(1c)S[K(N3)](2g)WQ[Pra](2g)W[2-Nal]LE(1c)R-[NH2] |
| 24 | [Ac]-DK(1c)S[Aha](2g)WQ[Pra](2g)W[2-Nal]LE(1c)R-[NH2] |
| 25 | [Ac]-K(1c)SC(2a)[2-Nal]QC(2a)W[2-Nal]LE(1c)R-[NH2] |
| 26 | [Ac]-K(1c)SC(2a)[Bip]QC(2a)W[2-Nal]LE(1c)R-[NH2] |
| 27 | [Ac]-K(1c)SC(2a)[1-Me-Trp]QC(2a)W[2-Nal]LE(1c)R-[NH2] |
| 28 | [Ac]-DK(1c)SC(2a)WQC(2a)[2-Nal][2-Nal]LE(1c)R-[NH2] |
| 29 | [Ac]-K(1c)SC(2a)WQC(2a)[Y(2-aminoethoxy)][2-Nal]LE(1c)R-[NH2] |
| 30 | [Ac]-K(1c)SC(2a)WQC(2a)H[2-Nal]LE(1c)R-[NH2] |
| 31 | [Ac]-K(1c)SC(2a)WQC(2a)W[F(4-F)]LE(1c)R-[NH2] |
| 32 | [Ac]-K(1c)SC(2a)WQC(2a)W[1-Me-Trp]LE(1c)R-[NH2] |
| 33 | [Ac]-C(1a)TC(2a)WEC(2a)WW[2-Me-Leu]C(1a)S-[NH2] |
| 34 | [Ac]-GK(1c)TC(2a)WEC(2c)WW[2-Me-Leu]E(1c)S-[NH2] |
| 35 | [Ac]-GC(1a)TC(2a)WEC(2a)W[2-Nal][2-Me-Leu]C(1a)R-[NH2] |
| 36 | [bAla](1c)*SC(2a)WQC(2a)W[2-Nal]LE(1c)R-[NH2] |
| 37 | [(3-Aminomethyl)benzoyl](1c)*SC(2a)WQC(2a)W[2-Nal]LE(1c)R-[NH2] |
| 38 | [(3-Aminomethyl)benzoyl](1c)*SC(2a)WQC(2a)[Y(2-aminoethoxy)][2- |
| | Nal][2-Me-Leu]E(1c)R-[NH2] |
| 39 | [(3-Aminomethyl)benzoyl](1c)*SC(2a)WQC(2a)[Y(2-aminoethoxy)][2- |
| | Nal]LE(1c)R-[NH2] |
| 40 | [(3-Aminomethyl)benzoyl](1c)*SC(2a)WQC(2a)W[2-Nal][2-Me- |
| | Leu]E(1c)R-[NH2] |
| 41 | [(3-Aminomethyl)benzoyl](1c)*SC(2a)WQC(2a)[Y(2-aminoethoxy)][2- |
| | Nal][2-Me-Leu]E(1c)[{d}R]-[NH2] |
| 42 | [(3-Aminomethyl)benzoyl](1c)*SC(2a)WQC(2a)[Y(2-aminoethoxy)][2- |
| | Nal][2-Me-Leu]E(1c)[2-Me-Arg]-[NH2] |
| 43 | [(3-Aminomethyl)benzoyl](1c)*SC(2a)WQC(2a)[Y(2-aminoethoxy)][2- |
| | Nal][2-Me-Leu]E(1c)[N-Me-Arg]-[NH2] |
| 44 | [(3-Aminomethyl)benzoyl](1c)*SC(2a)WQC(2a)[Y(2-aminoethoxy)][2- |
| | Nal][2-Me-Leu]E(1c)S-[NH2] |
| 45 | [(3-Aminomethyl)benzoyl](1c)*SC(2a)WQC(2a)[Y(2-aminoethoxy)][2- |
| | Nal][2-Me-Leu]E(1c)[F(4-NH2)]-[NH2] |
| 46 | [(3-Aminomethyl)benzoyl](1c)*SC(2a)WQC(2a)[Y(2-aminoethoxy)][2- |
| | Nal][{d}L]E(1c)Y-[NH2] |
| 47 | [Ac]-K(1c)SC(2a)WQC(2a)[Y(2-aminoethoxy)][2-Nal][2-Me-Leu]E(1c)R- |
| | [NH2] |
| 48 | [Ac]-K(1c)SC(2a)WQC(2a)[Y(2-aminoethoxy)][2-Nal][{d}LJE(1c)R-[NH2] |
| 49 | [(4-Aminomethyl)benzoyl](1c)*SC(2a)WQC(2a)[Y(2-aminoethoxy)][2- |
| | Nal][2-Me-Leu]E(1c)R-[NH2] |
| 50 | [4-(2-Aminoethyl)benzoyl](1c)*SC(2a)WQC(2a)[Y(2-aminoethoxy)][2- |
| | Nal][2-Me-Leu]E(1c)R-[NH2] |
| 51 | [3-(4-Aminophenyl)propanoyl](1c)*SC(2a)WQC(2a)[Y(2-aminoethoxy)][2- |
| | Nal][2-Me-Leu]E(1c)R-[NH2] |
| 52 | [4-Aminomethyl-phenylacetyl](1c)*SC(2a)WQC(2a)[Y(2-aminoethoxy)][2- |
| | Nal][2-Me-Leu]E(1c)R-[NH2] |
| 53 | H-DC(1a)SC(2b)WQC(2b)WWLC(1a)R-[NH2] |
| 54 | [Ac]-K(1c)SC(2b)WQC(2b)[Y(2-aminoethoxy)][2-Nal]LE(1c)R-[NH2] |
| 55 | [Ac]-DE(1c)QC(2a)WQC(2a)YW[2-Me-Leu]K(1c)R-[NH2] |
| 56 | [Ac]-DE(1c)QC(2a)WQC(2a)[2-Nal]W[2-Me-Leu]K(1c)R-[NH2] |
| 57 | [Ac]-DE(1c)TC(2a)WQC(2a)W[2-Nal][2-Me-Leu]K(1c)R-[NH2] |
| 58 | [Ac]-DE(1c)SC(2a)WQC(2a)W[2-Nal][2-Me-Leu]K(1c)ENG-[NH2] |
| 59 | [Ac]-DE(1c)SC(2a)WQC(2a)W[2-Nal][2-Me-Leu]K(1c)NG-[NH2] |
| 60 | [Ac]-DE(1c)SC(2a)WQC(2a)W[2-Nal][2-Me-Leu]K(1c)G-[NH2] |
| 61 | [Ac]-DE(1c)SC(2a)WQC(2a)W[2-Nal][2-Me-Lys]K(1c)ENG-[NH2] |
| 62 | [Ac]-DC(1a)QC(2a)WQC(2a)[2-Nal]W[2-Me-Leu]C(1a)R-[NH2] |
| 63 | [Ac]-DC(1a)QC(2a)WQC(2a)YW[2-Me-Leu]C(1a)R-[NH2] |
| 64 | [Ac]-DC(1a)QC(2a)WQC(2a)WW[2-Me-Leu]C(1a)ENG-[NH2] |
| 65 | [(3-Aminomethyl)benzoyl](1c)*S[Dpr](2c)WQE(2c)[Y(2-aminoethoxy)][2- |
| | Nal][2-Me-Leu]E(1c)R-[NH2] |
| 66 | [(3-Aminomethyl)benzoyl](1c)*SE(2c)WQ[Dpr](2c)[Y(2-aminoethoxy)][2- |
| | Nal][2-Me-Leu]E(1c)R-[NH2] |
| 67 | [(3-Aminomethyl)benzoyl](1c)*SE(2c)WQ[Dab](2c)[Y(2-aminoethoxy)][2- |
| | Nal][2-Me-Leu]E(1c)R-[NH2] |
| 68 | [(3-Aminomethyl)benzoyl](1c)*S[Orn](2c)WQE(2c)[Y(2-aminoethoxy)][2- |
| | Nal][2-Me-Leu]E(1c)R-[NH2] |
| 69 | [(3-Aminomethyl)benzoyl](1c)*SE(2c)WQ[Orn](2c)[Y(2-aminoethoxy)][2- |
| | Nal][2-Me-Leu]E(1c)R-[NH2] |
| 70 | [(3-Aminomethyl)benzoyl](1c)*SC(2a)[7-Me-Trp]QC(2a)[Y(2- |
| | aminoethoxy)][2-Nal][2-Me-Leu]E(1c)R-[NH2] |
| 71 | [(3-Aminomethyl)benzoyl](1c)*SC(2a)[7-Ph-Trp]QC(2a)[Y(2- |
| | aminoethoxy)][2-Nal][2-Me-Leu]E(1c)R-[NH2] |
| 72 | [(3-Aminomethyl)benzoyl](1c)*SC(2a)[7-(Naphth-2-yl)-Trp]QC(2a)[Y(2- |
| | aminoethoxy)][2-Nal][2-Me-Leu]E(1c)R-[NH2] |
| 73 | [(3-Aminomethyl)benzoyl](1c)*SC(2a)WQC(2a)[Y(2-aminoethoxy)][3-(3- |
| | Quinolinyl)-Ala][2-Me-Leu]E(1c)R-[NH2] |
| 74 | [(3-Aminomethyl)benzoyl](1c)*SC(2a)WQC(2a)[Y(2-aminoethoxy)][6-CI- |
| | Trp][2-Me-Leu]E(1c)R-[NH2] |
| 75 | [(3-Aminomethyl)benzoyl](1c)*SC(2a)WQC(2a)[Y(2-aminoethoxy)][2- |
| | Nal][4-aminotetrahydro-2H-pyran-4-acetyl]E(1c)R-[NH2] |
| 76 | [(3-Aminomethyl)benzoyl](1c)*SC(2a)WQC(2a)[Y(2-aminoethoxy)][2- |
| | Nal][2-Me-Leu]E(1c)[3-(3-Pyridyl)-Ala]-[NH2] |
| 77 | [(3-Aminomethyl)benzoyl](1c)*SC(2a)WQC(2a)[Y(2-aminoethoxy)][2- |
| | Nal][2-Me-Leu]E(1c)[3-(4-Pyridyl)-Ala]-[NH2] |
| 78 | [(3-Aminomethyl)benzoyl](1c)*SC(2a)WQC(2a)[Y(2-aminoethoxy)][2- |
| | Nal][2-Me-Leu]E(1c)R[3-(3-Pyridyl)-Ala]-[NH2] |
| 79 | [(3-Aminomethyl)benzoyl](1c)*SC(2a)WQC(2a)[Y(2-aminoethoxy)][2- |
| | Nal][2-Me-Leu]E(1c)R[3-(4-Pyridyl)-Ala]-[NH2] |
| 80 | [4-Aminomethyl-phenylacetyl](1c)*SC(2a)WQC(2a)[Y(2-aminoethoxy)][2- |
| | Nal][2-Me-Leu]E(1c) [{d}R]-[NH2] |
| 81 | [4-Aminomethyl-phenylacetyl](1c)*SC(2a)WQC(2a)[Y(2-aminoethoxy)][2- |
| | Nal][2-Me-Leu]E(1c)S-[NH2] |
| 82 | [4-Aminomethyl-phenylacetyl](1c)*SC(2a)WQC(2a)[Y(2-aminoethoxy)][2- |
| | Nal][2-Me-Leu]E(1c)[Dab]-[NH2] |
| 83 | [4-Aminomethyl-phenylacetyl](1c)*TC(2a)WQC(2a)[Y(2-aminoethoxy)][2- |
| | Nal][2-Me-Leu]E(1c)[{d}R]-[NH2] |
| 84 | [Ac]-K(1c)SC(2a)WQC(2a)[Y(2-aminoethoxy)][2-Nal][2-Me- |
| | Leu]E(1c)[{d}R]-[NH2] |
| 85 | [Ac]-K(1c) TC(2a)WQC(2a)[Y(2-aminoethoxy)][2-Nal][2-Me- |
| | Leu]E(1c)[{d}R]-[NH2] |
| 86 | [Ac]-K(1c)SC(2a)WQC(2a)[Y(2-aminoethoxy)][2-Nal][2-Me-Leu]E(1c)S- |
| | [NH2] |
| 87 | [6-Aminohexanoyl](1c)*SC(2a)WQC(2a)[Y (2-aminoethoxy)][2-Nal][2-Me- |
| | Leu]E(1c)[{d}R]-[NH2] |
| 88 | [6-Amino-4-oxahexanoyl](1c)*SC(2a)WQC(2a)[Y(2-aminoethoxy)][2- |
| | Nal][2-Me-Leu]E(1c) [{d}R]-[NH2] |
| 89 | [trans-4-Aminomethyl-cyclohexyl-1-carbonyl](1c)*[beta-homo- |
| | Ser]C(2a)WQC(2a)[Y(2-aminoethoxy)][2-Nal][2-Me-Leu]E(1c)[{d}R]-[NH2] |
| 90 | [(4-(2-Aminoethyl)-piperazine-1-yl)-acetyl](1c)*SC(2a)WQC(2a)[Y(2- |
| | aminoethoxy)][2-Nal][2-Me-Leu]E(1c)[{d}R]-[NH2] |
| 91 | [(4-(2-Aminoethyl)-piperazine-1-yl)-acetyl](1c)*SC(2a)WQC(2a)[Y(2- |
| | aminoethoxy)][2-Nal][2-Me-Leu]D(1c)[{d}R]-[NH2] |
| 92 | [3-Aminomethyl-phenylacetyl](1c)*SC(2a)WQC(2a)[Y(2-aminoethoxy)][2- |
| | Nal][2-Me-Leu]E(1c)[{d}R]-[NH2] |
| 93 | [2-Aminomethyl-phenylacetyl](1c)*SC(2a)WQC(2a)[Y(2-aminoethoxy)][2- |
| | Nal][2-Me-Leu]E(1c)[{d}R]-[NH2] |
| 94 | [Ac]-[K(N3)](1g)SC(2a)WQC(2a)[Y(2-aminoethoxy)][2-Nal][2-Me- |
| | Leu][Pra](1g)[{d}R]-[NH2] |
| 95 | [Azidoacetic acid](1g)*SC(2a)WQC(2a)[Y(2-aminoethoxy)][2-Nal][2-Me- |
| | Leu][Glu(propargylamine)](1g)[{d}R]-[NH2] |
| 96 | [Azidoacetic acid] (1g)*SC(2a)WQC(2a)[Y(2-aminoethoxy)][2-Nal][2-Me- |
| | Leu][Dab(3-butynoic acid)](1g)[{d}R]-[NH2] |
| 97 | [but-3-ynoic acid](1g)*SC(2a)WQC(2a)[Y(2-aminoethoxy)][2-Nal][2-Me- |
| | Leu][Dab(azidoacetic acid)](1g)[{d}R]-[NH2] |
| 98 | [(N3)-Ala](1g)*SC(2a)WQC(2a)[Y(2-aminoethoxy)][2-Nal][2-Me- |
| | Leu][Glu(propargylamine)](1g)[{d}R]-[NH2] |
| 99 | [(N3)-Ala](1g)*SC(2a)WQC(2a)[Y(2-aminoethoxy)][2-Nal][2-Me- |
| | Leu][Dab(3-butynoic acid)](1g)[{d}R]-[NH2] |
| 100 | [but-3-ynoic acid](1g)*SC(2a)WQC(2a)[Y(2-aminoethoxy)][2-Nal][2-Me- |
| | Leu][Dab((N3)-Ala))](1g)[{d}R]-[NH2] |
| 101 | [4-Aminomethyl-2-pyridineacetyl](1c)*SC(2a)WQC(2a)[Y(2- |
| | aminoethoxy)][2-Nal][2-Me-Leu]E(1c)[{d}R]-[NH2] |
| 102 | [4-Aminomethyl-3-pyridineacetyl](1c)*SC(2a)WQC(2a)[Y(2- |
| | aminoethoxy)][2-Nal][2-Me-Leu]E(1c)[{d}R]-[NH2] |
| 103 | [4-Aminomethyl-2-fluoro-phenylacetyl](1c)*SC(2a)WQC(2a)[Y(2- |
| | aminoethoxy)][2-Nal][2-Me-Leu]E(1c)[{d}R]-[NH2] |
| 104 | [4-Aminomethyl-3-methyl-phenylacetyl](1c)*SC(2a)WQC(2a)[Y (2- |
| | aminoethoxy)][2-Nal][2-Me-Leu]E(1c)[{d}R]-[NH2] |
| 105 | [4-Aminomethyl-3-methoxy-phenylacetyl](1c)*SC(2a)WQC(2a)[Y(2- |
| | aminoethoxy)][2-Nal][2-Me-Leu]E(1c)[{d}R]-[NH2] |
| 106 | [but-3-ynoic acid](1h)*SC(2a)WQC(2a)[Y(2-aminoethoxy)][2-Nal][2-Me- |
| | Leu][Dab(azidoacetic acid)](1h)[{d}R]-[NH2] |
| 107 | [but-3-ynoic acid](1h)*SC(2a)WQC(2a)[Y(2-aminoethoxy)][2-Nal][2-Me- |
| | Leu][Dab((N3)-Ala))] (1h)[{d}R]-[NH2] |
| 108 | [4-Aminomethyl-phenylacetyl](1c)*SC(2a)WQC(2a)[Y(2-aminoethoxy)][2- |
| | Nal][2-Me-Leu]E(1c)[{d}R]-[OH] |
| 109 | [4-Aminomethyl-phenylacetyl](1c)*SC(2a)WQC(2a)[Y(2-aminoethoxy)][2- |
| | Nal][2-Me-Leu]E(1c)[{d}2,4-Diaminobutanoyl([2-(trimethyl-2- |
| | aminoethoxy)ethoxy]propyl)]-[NH2] |
| 110 | [4-Aminomethyl-phenylacetyl](1c)*SC(2a)WQC(2a)[Dab][2-Nal][2-Me- |
| | Leu]E(1c)[{d}R]-[NH2] |
| 111 | [4-Aminomethyl-phenylacetyl](1c)*SC(2a)WQC(2a)[2,4- |
| | Diaminobutanoyl([2-(trimethyl-2-aminoethoxy)ethoxy]propyl)][2-Nal][2-Me- |
| | Leu]E(1c)[{d}R]-[NH2] |
| 112 | [Dab](1c)*SC(2a)WQC(2a)[Y(2-aminoethoxy)][2-Nal][2-Me- |
| | Leu]E(1c)[{d}R]-[NH2] |
| 113 | [2,4-Diaminobutanoyl([2-(trimethyl-2- |
| | aminoethoxy)ethoxy]propyl)](1c)*SC(2a)WQC(2a)[Y(2-aminoethoxy)][2- |
| | Nal][2-Me-Leu]E(1c)[{d}R]-[NH2] |
| 114 | [4-Aminomethyl-phenylacetyl](1c)*SC(2a)WQC(2a)[F(4-NH2-(2-(trimethyl- |
| | 2-aminoethoxy)ethoxy)propyl)][2-Nal][2-Me-Leu]E(1c)[{d}R]-[NH2] |
| 115 | [4-Aminomethyl-phenylacetyl](1c)*SC(2a)WQC(2a)[Y(2-aminoethoxy)][2- |
| | Nal][2-Me-Leu]E(1c)[{d}Q]-[NH2] |
| 116 | [4-Aminomethyl-phenylacetyl](1c)*SC(2a)WQC(2a)[Y(2-aminoethoxy)][2- |
| | Nal][2-Me-Leu]E(1c)[{d}E]-[NH2] |
| 117 | [4-Aminomethyl-phenylacetyl](1c)*SC(2a)WQC(2a)[Y(2-aminoethoxy)][2- |
| | Nal][2-Me-Leu]E(1c)[{d}H]-[NH2] |
| 118 | [4-Aminomethyl-phenylacetyl](1c)*SC(2a)WQC(2a)[Y(2-aminoethoxy)][2- |
| | Nal][2-Me-Leu]E(1c)[3-Aminopropanoy]]-[OH] |
| 119 | [4-Aminomethyl-phenylacetyl](1c)*SC(2a)WQC(2a)[Y(2-aminoethoxy)][2- |
| | Nal][2-Me-Leu]E(1c)[3-Aminopropanoyl]-[NH2] |
| 120 | [4-Aminomethyl-phenylacetyl](1c)*SC(2a)WQC(2a)[Y(2-aminoethoxy)][2- |
| | Nal][2-Me-Leu]E(1c)[Dab][3-(3-Pyridyl)-Ala]-[NH2] |
| 121 | [4-Aminomethyl-phenylacetyl](1c)*SC(2a)WQC(2a)[Y(2-aminoethoxy)][6- |
| | F-Trp][2-Me-Leu]E(1c)[Dab][3-(3-Pyridyl)-Ala]-[NH2] |
| 122 | [4-Aminomethyl-phenylacetyl](1c)*SE(2c)WQ[Dab](2c)[Y(2- |
| | aminoethoxy)][2-Nal][2-Me-Leu]E(1c)[Dab][3-(3-Pyridyl)-Ala]-[NH2] |
| 123 | [4-Aminomethyl-phenylacetyl](1c)*S[Dab](2c)WQE(2c)[Y(2- |
| | aminoethoxy)][2-Nal][2-Me-Leu]E(1c)[Dab][3-(3-Pyridyl)-Ala]-[NH2] |
| 124 | [4-Aminomethyl-phenylacetyl](1c)*SE(2c)WQ[Orn](2c)[Y (2- |
| | aminoethoxy)][2-Nal][2-Me-Leu]E(1c)[Dab][3-(3-Pyridyl)-Ala]-[NH2] |
| 125 | [4-Aminomethyl-phenylacetyl](1c)*SE(2c)WQ[Dab](2c)[Y (2- |
| | aminoethoxy)][2-Nal][2-Me-Leu]E(1c)[Dab]-[NH2] |
| 126 | [4-Aminomethyl-phenylacetyl](1c)*VE(2c)WQ[Dab](2c)[Y(2- |
| | aminoethoxy][2-Nal][2-Me-Leu]E(1c)[Dab]-[NH2] |
| 127 | [4-Aminomethyl-phenylacetyl](1c)*IE(2c)WQ[Dab](2c)[Y(2- |
| | aminoethoxy)][2-Nal][2-Me-Leu]E(1c)[Dab]-[NH2] |
| 128 | [4-Aminomethyl-phenylacetyl](1c)*SE(2c)WV[Dab](2c)[Y(2- |
| | aminoethoxy)][2-Nal][2-Me-Leu]E(1c)[Dab]-[NH2] |
| 129 | [4-Aminomethyl-phenylacetyl](1c)*SE(2c)WH[Dab](2c)[Y(2- |
| | aminoethoxy)][2-Nal][2-Me-Leu]E(1c)[Dab]-[NH2] |
| 130 | [4-Aminomethyl-phenylacetyl](1c)*SE(2c)WQ[Dab](2c)F[2-Nal][2-Me- |
| | Leu]E(1c)[Dab]-[NH2] |
| 131 | [4-Aminomethyl-phenylacetyl](1c)*SE(2c)WQ[Dab](2c)V[2-Nal][2-Me- |
| | Leu]E(1c)[Dab]-[NH2] |
| 132 | [4-Aminomethyl-phenylacetyl](1c)*SE(2c)WQ[Dab](2c)[F(4-Me)][2-Nal][2- |
| | Me-Leu]E(1c)[Dab]-[NH2] |
| 133 | [4-Aminomethyl-phenylacetyl](1c)*SE(2c)WQ[Dab](2c)[Y (2- |
| | aminoethoxy)]H[2-Me-Leu]E(1c)[Dab]-[NH2] |
| 134 | [4-Aminomethyl-phenylacetyl](1c)*SE(2c)WQ[Dab](2c)[Y(2- |
| | aminoethoxy)][F(3-F)][2-Me-Leu]E(1c)[Dab]-[NH2] |
| 135 | [4-Aminomethyl-phenylacetyl](1c)*SE(2c)WQ[Dab](2c)[Y(2- |
| | aminoethoxy)][F(3,4-Me)][2-Me-Leu]E(1c)[Dab]-[NH2] |
| 136 | [4-Aminomethyl-phenylacetyl](1c)*SE(2c)WQ[Dab](2c)[Y(2- |
| | aminoethoxy)][2-Nal][2-Me-Val]E(1c)[Dab]-[NH2] |
| 137 | [4-Aminomethyl-phenylacetyl](1c)*SE(2c)WQ[Dab](2c)[Y(2- |
| | aminoethoxy)][2-Nal][2-Me-Leu]E(1c)-[NH-3-aminopropanoyl] |
| 138 | [4-Aminomethyl-phenylacetyl](1c)*SE(2c)[4-F-Trp]Q[Dab](2c)[Y(2- |
| | aminoethoxy)][2-Nal][2-Me-Leu]E(1c)[Dab]-[NH2] |
| 139 | [4-Aminomethyl-phenylacetyl](1c)*SE(2c)[7-F-Trp]Q[Dab](2c)[Y(2- |
| | aminoethoxy)][2-Nal][2-Me-Leu]E(1c)[Dab]-[NH2] |
| 140 | [4-Methylaminomethyl-phenylacetyl](1c)*SC(2a)WQC(2a)[Y(2- |
| | aminoethoxy)][2-Nal][2-Me-Leu]E(1c)[Dab]-[NH2] |
| 141 | [4-Aminomethyl-phenylacetyl](1c)*[N-Me-Ser]C(2a)WQC(2a)[Y(2- |
| | aminoethoxy)][2-Nal][2-Me-Leu]E(1c)[Dab]-[NH2] |
| 142 | [4-Aminomethyl-phenylacetyl](1c)*S[N-Me-Cys](2a)WQC(2a)[Y(2- |
| | aminoethoxy)][2-Nal][2-Me-Leu]E(1c)[Dab]-[NH2] |
| 143 | [4-Aminomethyl-phenylacetyl](1c)*SC(2a)[N-Me-Trp]QC(2a)[Y(2- |
| | aminoethoxy)][2-Nal][2-Me-Leu]E(1c)[Dab]-[NH2] |
| 144 | [4-Aminomethyl-phenylacetyl](1c)*SC(2a)W[N-Me-Gln]C(2a)[Y(2- |
| | aminoethoxy)][2-Nal][2-Me-Leu]E(1c)[Dab]-[NH2] |
| 145 | [4-Aminomethyl-phenylacetyl](1c)*[Q(pyrrolidin)]E(2c)WQ[Dab](2c)[Y(2- |
| | aminoethoxy)][2-Nal][2-Me-Leu]E(1c)[Dab]-[NH2] |
| 146 | [4-Aminomethyl-phenylacetyl](1c)*SE(2c)W[Q(pyrrolidin)][Dab](2c)[Y (2- |
| | aminoethoxy)][2-Nal][2-Me-Leu]E(1c)[Dab]-[NH2] |
| 147 | [4-Aminomethyl-phenylacetyl](1c)*SC(2a)WQC(2a)[Y(2-aminoethoxy)][2- |
| | Nal][2-Me-Leu]E(1c)R[3-(3-Pyridyl)-Ala]-[NH2] |
| 148 | [(3-Aminomethyl)benzoyl](1c)*SC(2a)WQC(2a)[Y(2-aminoethoxy)][2- |
| | Nal][2-Me-Leu]E(1c)[Dab][3-(3-Pyridyl)-Ala]-[NH2] |
| 149 | [(3-Aminomethyl)benzoyl](1c)*SC(2a)WQC(2a)][Y (2-aminoethoxy)][2- |
| | Nal][2-Me-Leu]E(1c)R[3-(3-Quinolinyl)-Ala]-[NH2] |
| 150 | [(3-Aminomethyl)benzoyl](1c)*SC(2a)WQC(2a)][Y(2-aminoethoxy)][2- |
| | Nal][2-Me-Leu]E(1c)R[{d}[3-(3-Pyridyl)-Ala]]-[NH2] |
| 151 | [(3-Aminomethyl)benzoyl](1c)*SC(2a)WQC(2a)][Y(2-aminoethoxy)][2- |
| | Nal][2-Me-Leu]E(1c)R[3-Amino-3-(3′-pyridyl)propionyl]-[NH2] |
| 152 | [(3-Aminomethyl)benzoyl](1c)*SC(2a)WQC(2a)][Y(2-aminoethoxy)][2- |
| | Nal][2-Me-Leu]E(1c)R[F(3-F)]-[NH2] |
| 153 | [(3-Aminomethyl)benzoyl](1c)*SC(2a)WQC(2a)][Y(2-aminoethoxy)][2- |
| | Nal][2-Me-Leu]E(1c)R[F(3,5-F)]-[NH2] |
| 154 | [(3-Aminomethyl)benzoyl](1c)*SC(2a)WQC(2a)][Y(2-aminoethoxy)][2- |
| | Nal][2-Me-Leu]E(1c)R[4-Aminomethyl-2-pyridineacetyl]-[NH2] |
| 155 | [Ac]-K(1c)TC(2a)WQC(2a)[Y(2-aminoethoxy)][2-Nal][2-Me- |
| | Leu]E(1c)[Dab][3-(3-Pyridyl)-Ala]-[NH2] |
| 156 | [(3-Aminomethyl)benzoyl](1c)*IE(2c)WQ[Dab](2c)[Y(2-aminoethoxy)][2- |
| | Nal][2-Me-Val]E(1c)[Dab][3-(3-Pyridyl)-Ala]-[NH2] |
| 157 | [4-Aminomethyl-phenylacetyl](1c)*IE(2c)WQ[Dab](2c)[Y(2- |
| | aminoethoxy) ][2-Nal][2-Me-Val]E(1c)[Dab][3-(3-Pyridyl)-Ala]-[NH2] |
| 158 | [4-Aminomethyl-phenylacetyl](1c)*IE(2c)WQ[Dab](2c)[Y(2- |
| | aminoethoxy)][F(3,4-Me)][2-Me-Val]E(1c)G[3-(3-Pyridyl)-Ala]-[NH2] |
| 159 | [4-Aminomethyl-phenylacetyl](1c)*IE(2c)WQ[Dab](2c)[F(4-Me)][F(3,4- |
| | Me)][2-Me-Val]E(1c)G[3-(3-Pyridyl)-Ala]-[NH2] |
| 160 | [4-Aminomethyl-phenylacetyl](1c)*IE(2c)WQ[Dab](2c)[F(4-Me)][F(3,4- |
| | Me)][2-Me-Val]E(1c)[GABA][3-(3-Pyridyl)-Ala]-[NH2] |
| 161 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-pyridylacetyl)]-[NH2] |
| 162 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-pyridylpropionyl)]-[NH2] |
| 163 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-fluorobenzoyl)]-[NH2] |
| 164 | [4-Aminomethyl-phenylacetyl](1c)*IE(2c)WQ[Dab](2c)[F(4-Me)][F(3,4- |
| | Me)][2-Me-Val]E(1c)G[2,3-Diaminopropanoyl(3-fluorophenylacetyl)]-[NH2] |
| 165 | [4-Aminomethyl-phenylacetyl](1c)*IE(2c)WQ[Dab](2c)[Y(2- |
| | aminoethoxy)][F(3,4-Me)][2-Me-Val]E(1c)[Dab][2-Me-3-(3-Pyridyl)-Ala]-[NH2] |
| 166 | [4-Aminomethyl-phenylacetyl](1c)*IE(2c)WQ[Dab](2c)[Y (Me)][F(3,4- |
| | Me)][2-Me-Val]E(1c)[Dab][2-Me-3-(3-Pyridyl)-Ala]-[NH2] |
| 167 | [4-Aminomethyl-phenylacetyl](1c)*IE(2c)WQ[Dab](2c)[2-Me-Phe][F(3,4- |
| | Me)][2-Me-Val]E(1c)[Dab][2-Me-3-(3-Pyridyl)-Ala]-[NH2] |
| 168 | [4-Aminomethyl-phenylacetyl](1c)*IE(2c)WQ[Dab](2c)[Bip][F(3,4-Me)][2- |
| | Me-Val]E(1c)[Dab][2-Me-3-(3-Pyridyl)-Ala]-[NH2] |
| 169 | [4-Aminomethyl-phenylacetyl](1c)*IE(2c)WQ[Dab](2c)[2-Me-F(4-F)][F(3,4- |
| | Me)][2-Me-Val]E(1c)[Dab][2-Me-3-(3-Pyridyl)-Ala]-[NH2] |
| 170 | [4-Aminomethyl-phenylacetyl](1c)*IE(2c)WQ[Dab](2c)[{d}F(4-F)][F(3,4- |
| | Me)][2-Me-Val]E(1c)[Dab][2-Me-3-(3-Pyridyl)-Ala]-[NH2] |
| 171 | [4-Aminomethyl-phenylacetyl](1c)*IE(2c)WQ[Dab](2c)[F(4-CI)][F(3,4- |
| | Me)][2-Me-Val]E(1c)[Dab][2-Me-3-(3-Pyridyl)-Ala]-[NH2] |
| 172 | [4-Aminomethyl-phenylacetyl](1c)*IE(2c)WQ[Dab](2c)[alpha-Me- |
| | Trp][F(3,4-Me)][2-Me-Val]E(1c)[Dab][2-Me-3-(3-Pyridyl)-Ala]-[NH2] |
| 173 | [4-Aminomethyl-phenylacetyl](1c)*IE(2c)WQ[Dab](2c)[3,3-Diphenyl- |
| | Ala][F(3,4-Me)][2-Me-Val]E(1c)[Dab][2-Me-3-(3-Pyridyl)-Ala]-[NH2] |
| 174 | [4-Aminomethyl-phenylacetyl](1c)*IE(2c)WQ[Dab](2c)[Phg][F(3,4-Me)][2- |
| | Me-Val]E(1c)[Dab][2-Me-3-(3-Pyridyl)-Ala]-[NH2] |
| 175 | [4-Aminomethyl-phenylacetyl](1c)*IE(2c)WQ[Dab](2c)[F(4-Me)][1-Me- |
| | Trp][2-Me-Val]E(1c)[Dab][2-Me-3-(3-Pyridyl)-Ala]-[NH2] |
| 176 | [4-Aminomethyl-phenylacetyl](1c)*IE(2c)WQ[Dab](2c)[F(4-Me)][Bip][2-Me- |
| | Val]E(1c)[Dab][2-Me-3-(3-Pyridyl)-Ala]-[NH2] |
| 177 | [4-Aminomethyl-phenylacetyl](1c)*IE(2c)WQ[Dab](2c)[F(4-Me)][{d}6-F- |
| | Trp][2-Me-Val]E(1c)[Dab][2-Me-3-(3-Pyridyl)-Ala]-[NH2] |

and pharmaceutically acceptable salts and solvates thereof;
wherein:
*= bridge uses the peptide backbone amine or carboxylic acid at the N- or C-terminus, not the side chain amine or carboxylic acid
(1a) = [2,11] 1,3-dithio-propan-2-one bridge; (1c) = [2,11] lactam bridge; (1g) = [2,11] 1,4-disubstituted 1,2,3-triazole bridge; (1h) = [2,11] 1,5-disubstituted 1,2,3-triazole bridge; (2a) = [4,7] 1,3-dithio-propan-2-one bridge; (2c) = [4,7] lactam bridge; (2d) = [4,7] 1,2-phenylenedimethanethiol bridge; (2e) = [4,7] 1,3-phenylenedimethanethiol bridge; (2f) = [4,7] 1,4-phenylenedimethanethiol bridge; (2g) = [4,7] 1,4-disubstituted 1,2,3-triazole bridge.

79. A pharmaceutical composition comprising a compound according to any one of the preceding claims in combination with a pharmaceutically acceptable carrier, excipient or vehicle.

80. A method for the synthesis of a compound according to any one of claims 1 to 78, comprising synthesising the analogue by solid-phase or liquid-phase peptide synthesis methodology, optionally isolating and/or purifying the final product, and optionally further comprising the step of forming an amide bond, forming two thioether bonds with a linker, or forming a triazole between the amino acid residues at positions X2 and X11, and optionally further comprising the step of forming an amide bond, forming two thioether bonds with a linker, or forming a triazole between the amino acid residues at positions X4 and X7.

81. A compound according to any one of claims 1 to 78, or a pharmaceutical composition according to claim 79, for use in a method of medical treatment.

82. A compound according to any one of claims 1 to 78, or a pharmaceutical composition according to claim 79, for use in a method of prevention or treatment of Inflammatory Bowel Disease (IBD), ulcerative colitis, Crohn's disease, Celiac disease (nontropical Sprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-I, chronic granulomatous disease, glycogen storage disease type 1b, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, ankylosing spondylitis, or graft versus host disease in a subject, and combinations thereof.

83. The compound or pharmaceutical composition for use according to claim 82, wherein the compound or pharmaceutical composition is for use in a method of prevention or treatment of inflammatory bowel (IBD), Crohn's Disease, ulcerative colitis, and psoriasis.

84. Use of a compound according to any one of claims 1 to 78, or a pharmaceutical composition according to claim 79, in the manufacture of a medicament for the prevention or treatment of Inflammatory Bowel Disease (IBD), ulcerative colitis, Crohn's disease, Celiac disease (nontropical Sprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-I, chronic granulomatous disease, glycogen storage disease type 1b, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, ankylosing spondylitis, or graft versus host disease in a subject, and combinations thereof.

85. The use of the compound or pharmaceutical composition according to claim 84, wherein the use of the compound or pharmaceutical compositions is in the manufacture of a medicament for the prevention or treatment of inflammatory bowel (IBD), Crohn's Disease, ulcerative colitis, and psoriasis.

86. A method of prevention or treatment of Inflammatory Bowel Disease (IBD), ulcerative colitis, Crohn's disease, Celiac disease (nontropical Sprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-I, chronic granulomatous disease, glycogen storage disease type 1b, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, ankylosing spondylitis, or graft versus host disease in a subject, and combinations thereof; which comprises administering to a subject an effective amount of a compound according to any one of claims 1 to 78, or a pharmaceutical composition according to claim 79.

87. The method of prevention or treatment according to claim 86, wherein the method of prevention or treatment is for inflammatory bowel (IBD), Crohn's Disease, ulcerative colitis, and psoriasis.
